SEQUENCE LISTING (148 sequences)

SEQ ID NO: 1 - protein, 167 aa, Escherichia coli
MSEVEFSHEYWMRHALTLAKRAWDEREVPVGAVLVHNNRVIGEGWNRPIGRHDPTAHAEIMALRQGGLVMQNYRLIDATLYVTLEPCVMCAGAMIHSRIGRVVFGARDAKTGAAGSLMDVLHHPGMNHRVEITEGILADECAALLSDFFRMRRQEIKAQKKAQSSTD

SEQ ID NO: 2 - protein, 178 aa, Escherichia coli
MRRAFITGVFFLSEVEFSHEYWMRHALTLAKRAWDEREVPVGAVLVHNNRVIGEGWNRPIGRHDPTAHAEIMALRQGGLVMQNYRLIDATLYVTLEPCVMCAGAMIHSRIGRVVFGARDAKTGAAGSLMDVLHHPGMNHRVEITEGILADECAALLSDFFRMRRQEIKAQKKAQSSTD

SEQ ID NO: 3 - protein, 1853 aa, Artificial Sequence
  Synthetic
MKRTADGSEFESPKKKRKVSSETGPVAVDPTLRRRIEPHEFEVFFDPRELRKETCLLYEIKWGTSHKIWRHSSKNTTKHVEVNFIEKFTSERHFCPSTSCSITWFLSWSPCGECSKAITEFLSQHPNVTLVIYVARLYHHMDQQNRQGLRDLVNSGVTIQIMTAPEYDYCWRNFVNYPPGKEAHWPRYPPLWMKLYALELHAGILGLPPCLNILRRKQPQLTFFTIALQSCHYQRLPPHILWATGLKSGGSSGGSSGSETPGTSESATPESSGGSSGGSDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSGGSGGSGGSTNLSDIIEKETGKQLVIQESILMLPEEVEEVIGNKPESDILVHTAYDESTDENVMLLTSDAPEYKPWALVIQDSNGENKIKMLSGGSGGSGGSTNLSDIIEKETGKQLVIQESILMLPEEVEEVIGNKPESDILVHTAYDESTDENVMLLTSDAPEYKPWALVIQDSNGENKIKMLSGGSKRTADGSEFEPKKKRKV

SEQ ID NO: 4 - protein, 1803 aa, Artificial Sequence
  Synthetic
MKRTADGSEFESPKKKRKVSEVEFSHEYWMRHALTLAKRAWDEREVPVGAVLVHNNRVIGEGWNRPIGRHDPTAHAEIMALRQGGLVMQNYRLIDATLYVTLEPCVMCAGAMIHSRIGRVVFGARDAKTGAAGSLMDVLHHPGMNHRVEITEGILADECAALLSDFFRMRRQEIKAQKKAQSSTDSGGSSGGSSGSETPGTSESATPESSGGSSGGSSEVEFSHEYWMRHALTLAKRARDEREVPVGAVLVLNNRVIGEGWNRAIGLHDPTAHAEIMALRQGGLVMQNYRLIDATLYVTFEPCVMCAGAMIHSRIGRVVFGVRNAKTGAAGSLMDVLHYPGMNHRVEITEGILADECAALLCYFFRMPRQVFNAQKKAQSSTDSGGSSGGSSGSETPGTSESATPESSGGSSGGSDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSGGSKRTADGSEFEPKKKRKV

SEQ ID NO: 5 - protein, 84 aa, Artificial Sequence
  Synthetic
MTNLSDIIEKETGKQLVIQESILMLPEEVEEVIGNKPESDILVHTAYDESTDENVMLLTSDAPEYKPWALVIQDSNGENKIKML

SEQ ID NO: 6 - protein, 2136 aa, Artificial Sequence
  Synthetic
MKRTADGSEFESPKKKRKVSSETGPVAVDPTLRRRIEPHEFEVFFDPRELRKETCLLYEIKWGTSHKIWRHSSKNTTKHVEVNFIEKFTSERHFCPSTSCSITWFLSWSPCGECSKAITEFLSQHPNVTLVIYVARLYHHMDQQNRQGLRDLVNSGVTIQIMTAPEYDYCWRNFVNYPPGKEAHWPRYPPLWMKLYALELHAGILGLPPCLNILRRKQPQLTFFTIALQSCHYQRLPPHILWATGLKSGGSSGGSSGSETPGTSESATPESSGGSSGGSDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSGGSGGSGGSTNLSDIIEKETGKQLVIQESILMLPEEVEEVIGNKPESDILVHTAYDESTDENVMLLTSDAPEYKPWALVIQDSNGENKIKMLSGGSGGSGGSTNLSDIIEKETGKQLVIQESILMLPEEVEEVIGNKPESDILVHTAYDESTDENVMLLTSDAPEYKPWALVIQDSNGENKIKMLSGGSKRTADGSEFEPKKKRKVSGGSPKKKRKVGSGATNFSLLKQAGDVEENPGPMVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYKSGGSPKKKRKV

SEQ ID NO: 7 - protein, 2086 aa, Artificial Sequence
  Synthetic
MKRTADGSEFESPKKKRKVSEVEFSHEYWMRHALTLAKRAWDEREVPVGAVLVHNNRVIGEGWNRPIGRHDPTAHAEIMALRQGGLVMQNYRLIDATLYVTLEPCVMCAGAMIHSRIGRVVFGARDAKTGAAGSLMDVLHHPGMNHRVEITEGILADECAALLSDFFRMRRQEIKAQKKAQSSTDSGGSSGGSSGSETPGTSESATPESSGGSSGGSSEVEFSHEYWMRHALTLAKRARDEREVPVGAVLVLNNRVIGEGWNRAIGLHDPTAHAEIMALRQGGLVMQNYRLIDATLYVTFEPCVMCAGAMIHSRIGRVVFGVRNAKTGAAGSLMDVLHYPGMNHRVEITEGILADECAALLCYFFRMPRQVFNAQKKAQSSTDSGGSSGGSSGSETPGTSESATPESSGGSSGGSDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSGGSKRTADGSEFEPKKKRKVSGGSPKKKRKVGSGATNFSLLKQAGDVEENPGPMVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYKSGGSPKKKRKV

SEQ ID NO: 8 - protein, 1627 aa, Artificial Sequence
  Synthetic
MSSETGPVAVDPTLRRRIEPHEFEVFFDPRELRKETCLLYEINWGGRHSIWRHTSQNTNKHVEVNFIEKFTTERYFCPNTRCSITWFLSWSPCGECSRAITEFLSRYPHVTLFIYIARLYHHADPRNRQGLRDLISSGVTIQIMTEQESGYCWRNFVNYSPSNEAHWPRYPHLWVRLYVLELYCIILGLPPCLNILRRKQPQLTFFTIALQSCHYQRLPPHILWATGLKSGSETPGTSESATPESDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSGGSSGGSPKKKRKV

SEQ ID NO: 9 - protein, 1710 aa, Artificial Sequence
  Synthetic
MSSETGPVAVDPTLRRRIEPHEFEVFFDPRELRKETCLLYEINWGGRHSIWRHTSQNTNKHVEVNFIEKFTTERYFCPNTRCSITWFLSWSPCGECSRAITEFLSRYPHVTLFIYIARLYHHADPRNRQGLRDLISSGVTIQIMTEQESGYCWRNFVNYSPSNEAHWPRYPHLWVRLYVLELYCIILGLPPCLNILRRKQPQLTFFTIALQSCHYQRLPPHILWATGLKSGSETPGTSESATPESDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSGGSTNLSDIIEKETGKQLVIQESILMLPEEVEEVIGNKPESDILVHTAYDESTDENVMLLTSDAPEYKPWALVIQDSNGENKIKMLSGGSPKKKRKV

SEQ ID NO: 10 - protein, 1710 aa, Artificial Sequence
  Synthetic
MSSETGPVAVDPTLRRRIEPHEFEVFFDPRELRKETCLLYEINWGGRHSIWRHTSQNTNKHVEVNFIEKFTTERYFCPNTRCSITWFLSWSPCGECSRAITEFLSRYPHVTLFIYIARLYHHADPRNRQGLRDLISSGVTIQIMTEQESGYCWRNFVNYSPSNEAHWPRYPHLWVRLYVLELYCIILGLPPCLNILRRKQPQLTFFTIALQSCHYQRLPPHILWATGLKSGSETPGTSESATPESDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSGGSTNLSDIIEKETGKQLVIQESILMLPEEVEEVIGNKPESDILVHTAYDESTDENVMLLTSDAPEYKPWALVIQDSNGENKIKMLSGGSPKKKRKV

SEQ ID NO: 11 - protein, 1824 aa, Artificial Sequence
  Synthetic
MSSETGPVAVDPTLRRRIEPHEFEVFFDPRELRKETCLLYEINWGGRHSIWRHTSQNTNKHVEVNFIEKFTTERYFCPNTRCSITWFLSWSPCGECSRAITEFLSRYPHVTLFIYIARLYHHADPRNRQGLRDLISSGVTIQIMTEQESGYCWRNFVNYSPSNEAHWPRYPHLWVRLYVLELYCIILGLPPCLNILRRKQPQLTFFTIALQSCHYQRLPPHILWATGLKSGGSSGGSSGSETPGTSESATPESSGGSSGGSDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSGGSGGSGGSTNLSDIIEKETGKQLVIQESILMLPEEVEEVIGNKPESDILVHTAYDESTDENVMLLTSDAPEYKPWALVIQDSNGENKIKMLSGGSGGSGGSTNLSDIIEKETGKQLVIQESILMLPEEVEEVIGNKPESDILVHTAYDESTDENVMLLTSDAPEYKPWALVIQDSNGENKIKMLSGGSPKKKRK

SEQ ID NO: 12 - protein, 2012 aa, Artificial Sequence
  Synthetic
AKPAKRIKSAAAAYVPQNRDAVITDIKRIGDLQREASRLETEMNDAIAEITEKFAARIAPIKTDIETLSKGVQGWCEANRDELTNGGKVKTANLVTGDVSWRVRPPSVSIRGMDAVMETLERLGLQRFIRTKQEINKEAILLEPKAVAGVAGITVKSGIEDFSIIPFEQEAGISGSETPGTSESATPESSSETGPVAVDPTLRRRIEPHEFEVFFDPRELRKETCLLYEINWGGRHSIWRHTSQNTNKHVEVNFIEKFTTERYFCPNTRCSITWFLSWSPCGECSRAITEFLSRYPHVTLFIYIARLYHHADPRNRQGLRDLISSGVTIQIMTEQESGYCWRNFVNYSPSNEAHWPRYPHLWVRLYVLELYCIILGLPPCLNILRRKQPQLTFFTIALQSCHYQRLPPHILWATGLKSGGSSGGSSGSETPGTSESATPESSGGSSGGSDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSGGSGGSGGSTNLSDIIEKETGKQLVIQESILMLPEEVEEVIGNKPESDILVHTAYDESTDENVMLLTSDAPEYKPWALVIQDSNGENKIKMLSGGSGGSGGSTNLSDIIEKETGKQLVIQESILMLPEEVEEVIGNKPESDILVHTAYDESTDENVMLLTSDAPEYKPWALVIQDSNGENKIKMLSGGSPKKKRK

SEQ ID NO: 13 - protein, 1544 aa, Staphylococcus aureus
MSSETGPVAVDPTLRRRIEPHEFEVFFDPRELRKETCLLYEINWGGRHSIWRHTSQNTNKHVEVNFIEKFTTERYFCPNTRCSITWFLSWSPCGECSRAITEFLSRYPHVTLFIYIARLYHHADPRNRQGLRDLISSGVTIQIMTEQESGYCWRNFVNYSPSNEAHWPRYPHLWVRLYVLELYCIILGLPPCLNILRRKQPQLTFFTIALQSCHYQRLPPHILWATGLKSGGSSGGSSGSETPGTSESATPESSGGSSGGSGKRNYILGLAIGITSVGYGIIDYETRDVIDAGVRLFKEANVENNEGRRSKRGARRLKRRRRHRIQRVKKLLFDYNLLTDHSELSGINPYEARVKGLSQKLSEEEFSAALLHLAKRRGVHNVNEVEEDTGNELSTKEQISRNSKALEEKYVAELQLERLKKDGEVRGSINRFKTSDYVKEAKQLLKVQKAYHQLDQSFIDTYIDLLETRRTYYEGPGEGSPFGWKDIKEWYEMLMGHCTYFPEELRSVKYAYNADLYNALNDLNNLVITRDENEKLEYYEKFQIIENVFKQKKKPTLKQIAKEILVNEEDIKGYRVTSTGKPEFTNLKVYHDIKDITARKEIIENAELLDQIAKILTIYQSSEDIQEELTNLNSELTQEEIEQISNLKGYTGTHNLSLKAINLILDELWHTNDNQIAIFNRLKLVPKKVDLSQQKEIPTTLVDDFILSPVVKRSFIQSIKVINAIIKKYGLPNDIIIELAREKNSKDAQKMINEMQKRNRQTNERIEEIIRTTGKENAKYLIEKIKLHDMQEGKCLYSLEAIPLEDLLNNPFNYEVDHIIPRSVSFDNSFNNKVLVKQEENSKKGNRTPFQYLSSSDSKISYETFKKHILNLAKGKGRISKTKKEYLLEERDINRFSVQKDFINRNLVDTRYATRGLMNLLRSYFRVNNLDVKVKSINGGFTSFLRRKWKFKKERNKGYKHHAEDALIIANADFIFKEWKKLDKAKKVMENQMFEEKQAESMPEIETEQEYKEIFITPHQIKHIKDFKDYKYSHRVDKKPNRELINDTLYSTRKDDKGNTLIVNNLNGLYDKDNDKLKKLINKSPEKLLMYHHDPQTYQKLKLIMEQYGDEKNPLYKYYEETGNYLTKYSKKDNGPVIKKIKYYGNKLNAHLDITDDYPNSRNKVVKLSLKPYRFDVYLDNGVYKFVTVKNLDVIKKENYYEVNSKCYEEAKKLKKISNQAEFIASFYNNDLIKINGELYRVIGVNNDLLNRIEVNMIDITYREYLENMNDKRPPRIIKTIASKTQSIKKYSTDILGNLYEVKSKKHPQIIKKGGSPKKKRKVSSDYKDHDGDYKDHDIDYKDDDDKSGGSGGSGGSTNLSDIIEKETGKQLVIQESILMLPEEVEEVIGNKPESDILVHTAYDESTDENVMLLTSDAPEYKPWALVIQDSNGENKIKMLSGGSGGSGGSTNLSDIIEKETGKQLVIQESILMLPEEVEEVIGNKPESDILVHTAYDESTDENVMLLTSDAPEYKPWALVIQDSNGENKIKMLSGGSPKKKRKV

SEQ ID NO: 14 - protein, 1733 aa, Staphylococcus aureus
MAKPAKRIKSAAAAYVPQNRDAVITDIKRIGDLQREASRLETEMNDAIAEITEKFAARIAPIKTDIETLSKGVQGWCEANRDELTNGGKVKTANLVTGDVSWRVRPPSVSIRGMDAVMETLERLGLQRFIRTKQEINKEAILLEPKAVAGVAGITVKSGIEDFSIIPFEQEAGISGSETPGTSESATPESSSETGPVAVDPTLRRRIEPHEFEVFFDPRELRKETCLLYEINWGGRHSIWRHTSQNTNKHVEVNFIEKFTTERYFCPNTRCSITWFLSWSPCGECSRAITEFLSRYPHVTLFIYIARLYHHADPRNRQGLRDLISSGVTIQIMTEQESGYCWRNFVNYSPSNEAHWPRYPHLWVRLYVLELYCIILGLPPCLNILRRKQPQLTFFTIALQSCHYQRLPPHILWATGLKSGGSSGGSSGSETPGTSESATPESSGGSSGGSGKRNYILGLAIGITSVGYGIIDYETRDVIDAGVRLFKEANVENNEGRRSKRGARRLKRRRRHRIQRVKKLLFDYNLLTDHSELSGINPYEARVKGLSQKLSEEEFSAALLHLAKRRGVHNVNEVEEDTGNELSTKEQISRNSKALEEKYVAELQLERLKKDGEVRGSINRFKTSDYVKEAKQLLKVQKAYHQLDQSFIDTYIDLLETRRTYYEGPGEGSPFGWKDIKEWYEMLMGHCTYFPEELRSVKYAYNADLYNALNDLNNLVITRDENEKLEYYEKFQIIENVFKQKKKPTLKQIAKEILVNEEDIKGYRVTSTGKPEFTNLKVYHDIKDITARKEIIENAELLDQIAKILTIYQSSEDIQEELTNLNSELTQEEIEQISNLKGYTGTHNLSLKAINLILDELWHTNDNQIAIFNRLKLVPKKVDLSQQKEIPTTLVDDFILSPVVKRSFIQSIKVINAIIKKYGLPNDIIIELAREKNSKDAQKMINEMQKRNRQTNERIEEIIRTTGKENAKYLIEKIKLHDMQEGKCLYSLEAIPLEDLLNNPFNYEVDHIIPRSVSFDNSFNNKVLVKQEENSKKGNRTPFQYLSSSDSKISYETFKKHILNLAKGKGRISKTKKEYLLEERDINRFSVQKDFINRNLVDTRYATRGLMNLLRSYFRVNNLDVKVKSINGGFTSFLRRKWKFKKERNKGYKHHAEDALIIANADFIFKEWKKLDKAKKVMENQMFEEKQAESMPEIETEQEYKEIFITPHQIKHIKDFKDYKYSHRVDKKPNRELINDTLYSTRKDDKGNTLIVNNLNGLYDKDNDKLKKLINKSPEKLLMYHHDPQTYQKLKLIMEQYGDEKNPLYKYYEETGNYLTKYSKKDNGPVIKKIKYYGNKLNAHLDITDDYPNSRNKVVKLSLKPYRFDVYLDNGVYKFVTVKNLDVIKKENYYEVNSKCYEEAKKLKKISNQAEFIASFYNNDLIKINGELYRVIGVNNDLLNRIEVNMIDITYREYLENMNDKRPPRIIKTIASKTQSIKKYSTDILGNLYEVKSKKHPQIIKKGGSPKKKRKVSSDYKDHDGDYKDHDIDYKDDDDKSGGSGGSGGSTNLSDIIEKETGKQLVIQESILMLPEEVEEVIGNKPESDILVHTAYDESTDENVMLLTSDAPEYKPWALVIQDSNGENKIKMLSGGSGGSGGSTNLSDIIEKETGKQLVIQESILMLPEEVEEVIGNKPESDILVHTAYDESTDENVMLLTSDAPEYKPWALVIQDSNGENKIKMLSGGSPKKKRKV

SEQ ID NO: 15 - protein, 1775 aa, Artificial Sequence
  Synthetic
MSEVEFSHEYWMRHALTLAKRAWDEREVPVGAVLVHNNRVIGEGWNRPIGRHDPTAHAEIMALRQGGLVMQNYRLIDATLYVTLEPCVMCAGAMIHSRIGRVVFGARDAKTGAAGSLMDVLHHPGMNHRVEITEGILADECAALLSDFFRMRRQEIKAQKKAQSSTDSGGSSGGSSGSETPGTSESATPESSGGSSGGSSEVEFSHEYWMRHALTLAKRARDEREVPVGAVLVLNNRVIGEGWNRAIGLHDPTAHAEIMALRQGGLVMQNYRLIDATLYVTFEPCVMCAGAMIHSRIGRVVFGVRNAKTGAAGSLMDVLHYPGMNHRVEITEGILADECAALLCYFFRMPRQVFNAQKKAQSSTDSGGSSGGSSGSETPGTSESATPESSGGSSGGSDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSGGSPKKKRKV

SEQ ID NO: 16 - protein, 5 aa, Artificial Sequence
  Synthetic
NGGNG

SEQ ID NO: 17 - protein, 7 aa, Artificial Sequence
  Synthetic
NNAGAAW

SEQ ID NO: 18 - protein, 1367 aa, Artificial Sequence
  Synthetic
DKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 19 - protein, 1367 aa, Artificial Sequence
  Synthetic
DKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 20 - protein, 1367 aa, Streptococcus pyogenes
DKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 21 - RNA, 81 nt, Artificial Sequence
  Synthetic
guuuuagagcuagaaauagcaaguuaaaauaaggcuaguccguuaucaacuugaaaaaguggcaccgagucggugcuuuuu

SEQ ID NO: 22 - skipped

SEQ ID NO: 23 - protein, 7 aa, Artificial Sequence
  Synthetic
PKKKRKV

SEQ ID NO: 24 - protein, 30 aa, Artificial Sequence
  Synthetic
MDSLLMNRRKFLYQFKNVRWAKGRRETYLC

SEQ ID NO: 25 - protein, 18 aa, Artificial Sequence
  Synthetic
KRTADGSEFESPKKKRKV

SEQ ID NO: 26 - protein, 17 aa, Artificial Sequence
  Synthetic
KRTADGSEFEPKKKRKV

SEQ ID NO: 27 - protein, 16 aa, Artificial Sequence
  Synthetic
SGSETPGTSESATPES

SEQ ID NO: 28 - protein, 32 aa, Artificial Sequence
  Synthetic
SGGSSGGSSGSETPGTSESATPESSGGSSGGS

SEQ ID NO: 29 - protein, 10 aa, Artificial Sequence
  Synthetic
SGGSGGSGGS

SEQ ID NO: 30 - protein, 4 aa, Artificial Sequence
  Synthetic
SGGS

SEQ ID NO: 31 - DNA, 20 bp, Artificial Sequence
  Synthetic
agcaagtatgatgagcaagc

SEQ ID NO: 32 - DNA, 23 bp, Artificial Sequence
  Synthetic
aaaacagatgctctgagaaaggc

SEQ ID NO: 33 - DNA, 21 bp, Artificial Sequence
  Synthetic
taggaacagctccggtctaca

SEQ ID NO: 34 - DNA, 20 bp, Artificial Sequence
  Synthetic
aatgcctcgccctgcttcgg

SEQ ID NO: 35 - DNA, 24 bp, Artificial Sequence
  Synthetic
ccaatacagagaagtgcttaaagg

SEQ ID NO: 36 - DNA, 22 bp, Artificial Sequence
  Synthetic
cttggaggctttgctcatttct

SEQ ID NO: 37 - DNA, 23 bp, Artificial Sequence
  Synthetic
cccatcagtgtgctgtattcagg

SEQ ID NO: 38 - DNA, 20 bp, Artificial Sequence
  Synthetic
ggccttctttgtctcttttg

SEQ ID NO: 39 - DNA, 21 bp, Artificial Sequence
  Synthetic
aacaggctctgaaattgtggc

SEQ ID NO: 40 - DNA, 23 bp, Artificial Sequence
  Synthetic
gctggcctcataaaatgagttag

SEQ ID NO: 41 - DNA, 20 bp, Artificial Sequence
  Synthetic
gttctggccagggcaatcag

SEQ ID NO: 42 - DNA, 22 bp, Artificial Sequence
  Synthetic
cctgagactttgctgaagttgc

SEQ ID NO: 43 - DNA, 20 bp, Artificial Sequence
  Synthetic
aataccaccctcactgggct

SEQ ID NO: 44 - DNA, 21 bp, Artificial Sequence
  Synthetic
cagattggaaacaagaggtcc

SEQ ID NO: 45 - DNA, 46 bp, Artificial Sequence
  Synthetic
tttatctgattacgacgtcgatgccattgtaccccaatcctttttg

SEQ ID NO: 46 - DNA, 46 bp, Artificial Sequence
  Synthetic
caaaaaggattggggtacaatggcatcgacgtcgtaatcagataaa

SEQ ID NO: 47 - DNA, 20 bp, Streptococcus pyogenes
gagacgattaatgcgtctcg

SEQ ID NO: 48 - DNA, 20 bp, Artificial Sequence
  Synthetic
aattatggagtatgtgtctg

SEQ ID NO: 49 - DNA, 20 bp, Artificial Sequence
  Synthetic
gatgacaggcaggggcaccg

SEQ ID NO: 50 - DNA, 20 bp, Artificial Sequence
  Synthetic
gaacacaaagcatagactgc

SEQ ID NO: 51 - DNA, 20 bp, Artificial Sequence
  Synthetic
aacgagacagaaagtcaaca

SEQ ID NO: 52 - DNA, 20 bp, Artificial Sequence
  Synthetic
tcagtttccatgtagttgag

SEQ ID NO: 53 - DNA, 20 bp, Artificial Sequence
  Synthetic
tatgtacccagtagtcattc

SEQ ID NO: 54 - DNA, 20 bp, Artificial Sequence
  Synthetic
attctaccagaggtacaagg

SEQ ID NO: 55 - DNA, 20 bp, Artificial Sequence
  Synthetic
ttgaaccagccttgcatccc

SEQ ID NO: 56 - DNA, 20 bp, Artificial Sequence
  Synthetic
gggtattcaattaggaaaag

SEQ ID NO: 57 - DNA, 20 bp, Artificial Sequence
  Synthetic
gactcccacacattaataat

SEQ ID NO: 58 - DNA, 20 bp, Artificial Sequence
  Synthetic
gcttaggtaaacaaagcagc

SEQ ID NO: 59 - DNA, 20 bp, Artificial Sequence
  Synthetic
attttggaataggtgtggtg

SEQ ID NO: 60 - DNA, 20 bp, Artificial Sequence
  Synthetic
attcagtatgatattggctg

SEQ ID NO: 61 - DNA, 20 bp, Artificial Sequence
  Synthetic
cctaggaatccaacttacaa

SEQ ID NO: 62 - DNA, 20 bp, Artificial Sequence
  Synthetic
aaaaagagtccaggaccaga

SEQ ID NO: 63 - DNA, 20 bp, Artificial Sequence
  Synthetic
caggcgtgagccaccgcgcc

SEQ ID NO: 64 - DNA, 20 bp, Staphylococcus aureus
gagacgattaatgcgtctcg

SEQ ID NO: 65 - DNA, 20 bp, Artificial Sequence
  Synthetic
gaggcacatccaacagttag

SEQ ID NO: 66 - DNA, 28 bp, Artificial Sequence
  Synthetic
ctttccctacacgacgctcttccgatct

SEQ ID NO: 67 - DNA, 28 bp, Artificial Sequence
  Synthetic
ggagttcagacgtgtgctcttccgatct

SEQ ID NO: 68 - DNA, 24 bp, Artificial Sequence
  Synthetic
agactcccacacattaataatggg

SEQ ID NO: 69 - DNA, 22 bp, Artificial Sequence
  Synthetic
tgatttggggtggagagttctg

SEQ ID NO: 70 - DNA, 24 bp, Artificial Sequence
  Synthetic
agtgcaatcaaactagaactcagg

SEQ ID NO: 71 - DNA, 23 bp, Artificial Sequence
  Synthetic
ccctctacacactgctttgaatg

SEQ ID NO: 72 - DNA, 24 bp, Artificial Sequence
  Synthetic
agtgcaatcaaactagaactcagg

SEQ ID NO: 73 - DNA, 23 bp, Artificial Sequence
  Synthetic
ccctctacacactgctttgaatg

SEQ ID NO: 74 - DNA, 22 bp, Artificial Sequence
  Synthetic
aagagtccaggaccagatggat

SEQ ID NO: 75 - DNA, 21 bp, Artificial Sequence
  Synthetic
cccggctttggtatcagaatg

SEQ ID NO: 76 - DNA, 23 bp, Artificial Sequence
  Synthetic
cttatccaccatgatcaagtggg

SEQ ID NO: 77 - DNA, 26 bp, Artificial Sequence
  Synthetic
ctgcatctattgagataatcatgtgg

SEQ ID NO: 78 - DNA, 20 bp, Artificial Sequence
  Synthetic
gttctggccagggcaatcag

SEQ ID NO: 79 - DNA, 22 bp, Artificial Sequence
  Synthetic
cctgagactttgctgaagttgc

SEQ ID NO: 80 - DNA, 20 bp, Artificial Sequence
  Synthetic
aactgcaaggcggcaacgag

SEQ ID NO: 81 - DNA, 20 bp, Artificial Sequence
  Synthetic
agaggtggagcctacagagg

SEQ ID NO: 82 - DNA, 22 bp, Artificial Sequence
  Synthetic
ccaatacaggagcacccagatt

SEQ ID NO: 83 - DNA, 20 bp, Artificial Sequence
  Synthetic
tgatttggggtggagagttc

SEQ ID NO: 84 - DNA, 20 bp, Artificial Sequence
  Synthetic
cagaactctccaccccaaat

SEQ ID NO: 85 - DNA, 20 bp, Artificial Sequence
  Synthetic
cctgagttctagtttgattg

SEQ ID NO: 86 - DNA, 20 bp, Artificial Sequence
  Synthetic
ccacatgattatctcaatag

SEQ ID NO: 87 - DNA, 20 bp, Artificial Sequence
  Synthetic
gagggcatccctgtcttgtg

SEQ ID NO: 88 - DNA, 20 bp, Artificial Sequence
  Synthetic
gcaacttcagcaaagtctca

SEQ ID NO: 89 - DNA, 22 bp, Artificial Sequence
  Synthetic
gtagttctccttgaagaggtcc

SEQ ID NO: 90 - DNA, 22 bp, Artificial Sequence
  Synthetic
ccaatacaggagcacccagatt

SEQ ID NO: 91 - DNA, 23 bp, Artificial Sequence
  Synthetic
ccctctacacactgctttgaatg

SEQ ID NO: 92 - DNA, 20 bp, Artificial Sequence
  Synthetic
ccacatgattatctcaatag

SEQ ID NO: 93 - DNA, 22 bp, Artificial Sequence
  Synthetic
gtagttctccttgaagaggtcc

SEQ ID NO: 94 - DNA, 21 bp, Artificial Sequence
  Synthetic
cagaactctccaccccaaatc

SEQ ID NO: 95 - DNA, 21 bp, Artificial Sequence
  Synthetic
cccggctttggtatcagaatg

SEQ ID NO: 96 - DNA, 21 bp, Artificial Sequence
  Synthetic
cagaactctccaccccaaatc

SEQ ID NO: 97 - DNA, 23 bp, Artificial Sequence
  Synthetic
ccctctacacactgctttgaatg

SEQ ID NO: 98 - DNA, 20 bp, Artificial Sequence
  Synthetic
aataccaccctcactgggct

SEQ ID NO: 99 - DNA, 21 bp, Artificial Sequence
  Synthetic
cagattggaaacaagaggtcc

SEQ ID NO: 100 - protein, 1052 aa, Artificial Sequence
  Synthetic
KRNYILGLAIGITSVGYGIIDYETRDVIDAGVRLFKEANVENNEGRRSKRGARRLKRRRRHRIQRVKKLLFDYNLLTDHSELSGINPYEARVKGLSQKLSEEEFSAALLHLAKRRGVHNVNEVEEDTGNELSTKEQISRNSKALEEKYVAELQLERLKKDGEVRGSINRFKTSDYVKEAKQLLKVQKAYHQLDQSFIDTYIDLLETRRTYYEGPGEGSPFGWKDIKEWYEMLMGHCTYFPEELRSVKYAYNADLYNALNDLNNLVITRDENEKLEYYEKFQIIENVFKQKKKPTLKQIAKEILVNEEDIKGYRVTSTGKPEFTNLKVYHDIKDITARKEIIENAELLDQIAKILTIYQSSEDIQEELTNLNSELTQEEIEQISNLKGYTGTHNLSLKAINLILDELWHTNDNQIAIFNRLKLVPKKVDLSQQKEIPTTLVDDFILSPVVKRSFIQSIKVINAIIKKYGLPNDIIIELAREKNSKDAQKMINEMQKRNRQTNERIEEIIRTTGKENAKYLIEKIKLHDMQEGKCLYSLEAIPLEDLLNNPFNYEVDHIIPRSVSFDNSFNNKVLVKQEEASKKGNRTPFQYLSSSDSKISYETFKKHILNLAKGKGRISKTKKEYLLEERDINRFSVQKDFINRNLVDTRYATRGLMNLLRSYFRVNNLDVKVKSINGGFTSFLRRKWKFKKERNKGYKHHAEDALIIANADFIFKEWKKLDKAKKVMENQMFEEKQAESMPEIETEQEYKEIFITPHQIKHIKDFKDYKYSHRVDKKPNRELINDTLYSTRKDDKGNTLIVNNLNGLYDKDNDKLKKLINKSPEKLLMYHHDPQTYQKLKLIMEQYGDEKNPLYKYYEETGNYLTKYSKKDNGPVIKKIKYYGNKLNAHLDITDDYPNSRNKVVKLSLKPYRFDVYLDNGVYKFVTVKNLDVIKKENYYEVNSKCYEEAKKLKKISNQAEFIASFYNNDLIKINGELYRVIGVNNDLLNRIEVNMIDITYREYLENMNDKRPPRIIKTIASKTQSIKKYSTDILGNLYEVKSKKHPQIIKKG

SEQ ID NO: 101 - protein, 1052 aa, Artificial Sequence
  Synthetic
KRNYILGLAIGITSVGYGIIDYETRDVIDAGVRLFKEANVENNEGRRSKRGARRLKRRRRHRIQRVKKLLFDYNLLTDHSELSGINPYEARVKGLSQKLSEEEFSAALLHLAKRRGVHNVNEVEEDTGNELSTKEQISRNSKALEEKYVAELQLERLKKDGEVRGSINRFKTSDYVKEAKQLLKVQKAYHQLDQSFIDTYIDLLETRRTYYEGPGEGSPFGWKDIKEWYEMLMGHCTYFPEELRSVKYAYNADLYNALNDLNNLVITRDENEKLEYYEKFQIIENVFKQKKKPTLKQIAKEILVNEEDIKGYRVTSTGKPEFTNLKVYHDIKDITARKEIIENAELLDQIAKILTIYQSSEDIQEELTNLNSELTQEEIEQISNLKGYTGTHNLSLKAINLILDELWHTNDNQIAIFNRLKLVPKKVDLSQQKEIPTTLVDDFILSPVVKRSFIQSIKVINAIIKKYGLPNDIIIELAREKNSKDAQKMINEMQKRNRQTNERIEEIIRTTGKENAKYLIEKIKLHDMQEGKCLYSLEAIPLEDLLNNPFNYEVDHIIPRSVSFDNSFNNKVLVKQEENSKKGNRTPFQYLSSSDSKISYETFKKHILNLAKGKGRISKTKKEYLLEERDINRFSVQKDFINRNLVDTRYATRGLMNLLRSYFRVNNLDVKVKSINGGFTSFLRRKWKFKKERNKGYKHHAEDALIIANADFIFKEWKKLDKAKKVMENQMFEEKQAESMPEIETEQEYKEIFITPHQIKHIKDFKDYKYSHRVDKKPNRELINDTLYSTRKDDKGNTLIVNNLNGLYDKDNDKLKKLINKSPEKLLMYHHDPQTYQKLKLIMEQYGDEKNPLYKYYEETGNYLTKYSKKDNGPVIKKIKYYGNKLNAHLDITDDYPNSRNKVVKLSLKPYRFDVYLDNGVYKFVTVKNLDVIKKENYYEVNSKCYEEAKKLKKISNQAEFIASFYNNDLIKINGELYRVIGVNNDLLNRIEVNMIDITYREYLENMNDKRPPRIIKTIASKTQSIKKYSTDILGNLYEVKSKKHPQIIKKG

SEQ ID NO: 102 - protein, 1052 aa, Staphylococcus aureus
KRNYILGLDIGITSVGYGIIDYETRDVIDAGVRLFKEANVENNEGRRSKRGARRLKRRRRHRIQRVKKLLFDYNLLTDHSELSGINPYEARVKGLSQKLSEEEFSAALLHLAKRRGVHNVNEVEEDTGNELSTKEQISRNSKALEEKYVAELQLERLKKDGEVRGSINRFKTSDYVKEAKQLLKVQKAYHQLDQSFIDTYIDLLETRRTYYEGPGEGSPFGWKDIKEWYEMLMGHCTYFPEELRSVKYAYNADLYNALNDLNNLVITRDENEKLEYYEKFQIIENVFKQKKKPTLKQIAKEILVNEEDIKGYRVTSTGKPEFTNLKVYHDIKDITARKEIIENAELLDQIAKILTIYQSSEDIQEELTNLNSELTQEEIEQISNLKGYTGTHNLSLKAINLILDELWHTNDNQIAIFNRLKLVPKKVDLSQQKEIPTTLVDDFILSPVVKRSFIQSIKVINAIIKKYGLPNDIIIELAREKNSKDAQKMINEMQKRNRQTNERIEEIIRTTGKENAKYLIEKIKLHDMQEGKCLYSLEAIPLEDLLNNPFNYEVDHIIPRSVSFDNSFNNKVLVKQEENSKKGNRTPFQYLSSSDSKISYETFKKHILNLAKGKGRISKTKKEYLLEERDINRFSVQKDFINRNLVDTRYATRGLMNLLRSYFRVNNLDVKVKSINGGFTSFLRRKWKFKKERNKGYKHHAEDALIIANADFIFKEWKKLDKAKKVMENQMFEEKQAESMPEIETEQEYKEIFITPHQIKHIKDFKDYKYSHRVDKKPNRELINDTLYSTRKDDKGNTLIVNNLNGLYDKDNDKLKKLINKSPEKLLMYHHDPQTYQKLKLIMEQYGDEKNPLYKYYEETGNYLTKYSKKDNGPVIKKIKYYGNKLNAHLDITDDYPNSRNKVVKLSLKPYRFDVYLDNGVYKFVTVKNLDVIKKENYYEVNSKCYEEAKKLKKISNQAEFIASFYNNDLIKINGELYRVIGVNNDLLNRIEVNMIDITYREYLENMNDKRPPRIIKTIASKTQSIKKYSTDILGNLYEVKSKKHPQIIKKG

SEQ ID NO: 103 - skipped

SEQ ID NO: 104 - protein, 37 aa, Artificial Sequence
  Synthetic
MEYPYDVPDYAAEYPYDVPDYAAEYPYDVPDYAAKLE

SEQ ID NO: 105 - protein, 167 aa, Artificial Sequence
  Synthetic
MSEVEFSHEYWMRHALTLAKRARDEREVPVGAVLVLNNRVIGEGWNRAIGLHDPTAHAEIMALRQGGLVMQNYRLIDATLYVTFEPCVMCAGAMIHSRIGRVVFGVRNAKTGAAGSLMDVLHYPGMNHRVEITEGILADECAALLCYFFRMPRQVFNAQKKAQSSTD

SEQ ID NO: 106 - protein, 1775 aa, Artificial Sequence
  Synthetic
MSEVEFSHEYWMRHALTLAKRAWDEREVPVGAVLVHNNRVIGEGWNRPIGRHDPTAHAEIMALRQGGLVMQNYRLIDATLYVTLEPCVMCAGAMIHSRIGRVVFGARDAKTGAAGSLMDVLHHPGMNHRVEITEGILADECAALLSDFFRMRRQEIKAQKKAQSSTDSGGSSGGSSGSETPGTSESATPESSGGSSGGSSEVEFSHEYWMRHALTLAKRARDEREVPVGAVLVLNNRVIGEGWNRAIGLHDPTAHAEIMALRQGGLVMQNYRLIDATLYVTFEPCVMCAGAMIHSRIGRVVFGVRNAKTGAAGSLMDVLHYPGMNHRVEITEGILADECAALLCYFFRMPRQVFNAQKKAQSSTDSGGSSGGSSGSETPGTSESATPESSGGSSGGSDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSGGSPKKKRKV

SEQ ID NO: 107 - protein, 1824 aa, Artificial Sequence
  Synthetic
MSSETGPVAVDPTLRRRIEPHEFEVFFDPRELRKETCLLYEINWGGRHSIWRHTSQNTNKHVEVNFIEKFTTERYFCPNTRCSITWFLSWSPCGECSRAITEFLSRYPHVTLFIYIARLYHHADPRNRQGLRDLISSGVTIQIMTEQESGYCWRNFVNYSPSNEAHWPRYPHLWVRLYVLELYCIILGLPPCLNILRRKQPQLTFFTIALQSCHYQRLPPHILWATGLKSGGSSGGSSGSETPGTSESATPESSGGSSGGSDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSGGSGGSGGSTNLSDIIEKETGKQLVIQESILMLPEEVEEVIGNKPESDILVHTAYDESTDENVMLLTSDAPEYKPWALVIQDSNGENKIKMLSGGSGGSGGSTNLSDIIEKETGKQLVIQESILMLPEEVEEVIGNKPESDILVHTAYDESTDENVMLLTSDAPEYKPWALVIQDSNGENKIKMLSGGSPKKKRK

SEQ ID NO: 108 - protein, 2012 aa, Artificial Sequence
  Synthetic
AKPAKRIKSAAAAYVPQNRDAVITDIKRIGDLQREASRLETEMNDAIAEITEKFAARIAPIKTDIETLSKGVQGWCEANRDELTNGGKVKTANLVTGDVSWRVRPPSVSIRGMDAVMETLERLGLQRFIRTKQEINKEAILLEPKAVAGVAGITVKSGIEDFSIIPFEQEAGISGSETPGTSESATPESSSETGPVAVDPTLRRRIEPHEFEVFFDPRELRKETCLLYEINWGGRHSIWRHTSQNTNKHVEVNFIEKFTTERYFCPNTRCSITWFLSWSPCGECSRAITEFLSRYPHVTLFIYIARLYHHADPRNRQGLRDLISSGVTIQIMTEQESGYCWRNFVNYSPSNEAHWPRYPHLWVRLYVLELYCIILGLPPCLNILRRKQPQLTFFTIALQSCHYQRLPPHILWATGLKSGGSSGGSSGSETPGTSESATPESSGGSSGGSDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSGGSGGSGGSTNLSDIIEKETGKQLVIQESILMLPEEVEEVIGNKPESDILVHTAYDESTDENVMLLTSDAPEYKPWALVIQDSNGENKIKMLSGGSGGSGGSTNLSDIIEKETGKQLVIQESILMLPEEVEEVIGNKPESDILVHTAYDESTDENVMLLTSDAPEYKPWALVIQDSNGENKIKMLSGGSPKKKRK

SEQ ID NO: 109 - DNA, 46 bp, Artificial Sequence
  Synthetic
gactcccacacattaataatgggcctgaatgactactgggtacata

SEQ ID NO: 110 - DNA, 34 bp, Artificial Sequence
  Synthetic
gactcccacacattaattgactactgggtacata

SEQ ID NO: 111 - DNA, 31 bp, Artificial Sequence
  Synthetic
gactcccacattaatgactactgggtacata

SEQ ID NO: 112 - DNA, 32 bp, Artificial Sequence
  Synthetic
gactcccacaataattgactactgggtacata

SEQ ID NO: 113 - DNA, 31 bp, Artificial Sequence
  Synthetic
gactcccacaataatgactactgggtacata

SEQ ID NO: 114 - DNA, 32 bp, Artificial Sequence
  Synthetic
gactcccacattaatttactactgggtacata

SEQ ID NO: 115 - DNA, 46 bp, Artificial Sequence
  Synthetic
ccacaccacacctattccaaaatattctaccagaggtacaaggagg

SEQ ID NO: 116 - DNA, 19 bp, Artificial Sequence
  Synthetic
ccacaccacaccaaggagg

SEQ ID NO: 117 - DNA, 18 bp, Artificial Sequence
  Synthetic
ccacaccacacaaggagg

SEQ ID NO: 118 - DNA, 20 bp, Artificial Sequence
  Synthetic
ccacaccacaccaaaggagg

SEQ ID NO: 119 - DNA, 17 bp, Artificial Sequence
  Synthetic
ccacaccacacaggagg

SEQ ID NO: 120 - DNA, 18 bp, Artificial Sequence
  Synthetic
ccacaccacaccaggagg

SEQ ID NO: 121 - DNA, 50 bp, Artificial Sequence
  Synthetic
ccacagccaatatcatactgaatcctaggaatccaacttacaagggatgt

SEQ ID NO: 122 - DNA, 20 bp, Artificial Sequence
  Synthetic
ccacccacagcaagggatgt

SEQ ID NO: 123 - DNA, 17 bp, Artificial Sequence
  Synthetic
ccacccacaagggatgt

SEQ ID NO: 124 - DNA, 16 bp, Artificial Sequence
  Synthetic
ccacccacagggatgt

SEQ ID NO: 125 - DNA, 12 bp, Artificial Sequence
  Synthetic
ccacccacaggt

SEQ ID NO: 126 - DNA, 11 bp, Artificial Sequence
  Synthetic
ccacccacagt

SEQ ID NO: 127 - DNA, 46 bp, Artificial Sequence
  Synthetic
ccacaccacacctattccaaaatcctgaatgactactgggtacata

SEQ ID NO: 128 - DNA, 46 bp, Artificial Sequence
  Synthetic
ggtgtggtgtggataaggttttaggacttactgatgacccatgtat

SEQ ID NO: 129 - DNA, 23 bp, Artificial Sequence
  Synthetic
aacgagacagaaagtcaacaagg

SEQ ID NO: 130 - DNA, 23 bp, Artificial Sequence
  Synthetic
attctaccagaggtacaaggagg

SEQ ID NO: 131 - DNA, 23 bp, Artificial Sequence
  Synthetic
aacggggcagaaagtcaacaagg

SEQ ID NO: 132 - DNA, 23 bp, Artificial Sequence
  Synthetic
attctgccggaggtacaaggagg

SEQ ID NO: 133 - DNA, 23 bp, Artificial Sequence
  Synthetic
aacgagatagaaagtcaacaagg

SEQ ID NO: 134 - DNA, 23 bp, Artificial Sequence
  Synthetic
attttattagaggtacaaggagg

SEQ ID NO: 135 - DNA, 23 bp, Artificial Sequence
  Synthetic
ccgctcaactacatggaaactga

SEQ ID NO: 136 - DNA, 23 bp, Artificial Sequence
  Synthetic
cctgggatgcaaggctggttcaa

SEQ ID NO: 137 - DNA, 23 bp, Artificial Sequence
  Synthetic
ccgctcaactacatggaaaccga

SEQ ID NO: 138 - DNA, 23 bp, Artificial Sequence
  Synthetic
cctgggatgcaaggccggcccaa

SEQ ID NO: 139 - DNA, 23 bp, Artificial Sequence
  Synthetic
ccgctcaactacataaaaactga

SEQ ID NO: 140 - DNA, 23 bp, Artificial Sequence
  Synthetic
cctgggatgcaaggctaattcaa

SEQ ID NO: 141 - DNA, 23 bp, Artificial Sequence
  Synthetic
cctgaatgactactgggtacata

SEQ ID NO: 142 - DNA, 23 bp, Artificial Sequence
  Synthetic
gggtattcaattaggaaaagagg

SEQ ID NO: 143 - DNA, 23 bp, Artificial Sequence
  Synthetic
cctgaatgactactgggcacata

SEQ ID NO: 144 - DNA, 23 bp, Artificial Sequence
  Synthetic
gggtgttcaattaggaaaagagg

SEQ ID NO: 145 - DNA, 23 bp, Artificial Sequence
  Synthetic
cctgaatgactactaaatacata

SEQ ID NO: 146 - DNA, 23 bp, Artificial Sequence
  Synthetic
gggtatttaattaggaaaagagg

SEQ ID NO: 147 - DNA, 23 bp, Artificial Sequence
  Synthetic
attctaccagaggtacaaggagg

SEQ ID NO: 148 - DNA, 23 bp, Artificial Sequence
  Synthetic
attttattagaggtacaaggagg

(19) United States
(12) Patent Application Publication
(10) Pub. No.: US 2022/0177877 A1
(43) Pub. Date: Jun. 9, 2022

(54) HIGHLY MULTIPLEXED BASE EDITING

(71) Applicant: President and Fellows of Harvard College, Cambridge, MA (US)

(72) Inventors: George M. Church, Cambridge, MA (US); Oscar Castanon Velasco, Cambridge, MA (US); Cory J. Smith, Brookline, MA (US)

(73) Assignee: President and Fellows of Harvard College, Cambridge, MA (US)

(21) Appl. No.: 17/593,020

(22) PCT Filed: Mar. 4, 2020
(86) PCT No.: PCT/US2020/020965

§ 371 Date: Sep. 3, 2021

Related U.S. Application Data
(60) Provisional application No. 62/813,672, filed on Mar. 4, 2019.

(51) Int. Cl. C12N 15/11; C12N 9/78; C12N 9/22

(52) U.S. Cl. CPC C12N 15/111; C12N 9/78; C12N 2310/20; C12Y 305/04004; C12N 9/22

(57) ABSTRACT

The present disclosure provides highly multiplexed base editing methods and compositions that minimize the induction of DNA damage sensors in eukaryotic cells and maintain cell viability. The disclosed base editing methods improve the survival of eukaryotic cells after large-scale genome editing. These methods are based upon the discovery that use of a dead Cas9 base editor and optimal cell conditions during and after base editing enhances cells' tolerance to and survival following thousands of edits to the genome. Optimal cell conditions after base editing include the use of a combination of small molecule factors and/or inhibitors. These methods are facilitated by the design and use of tens to hundreds to thousands of gRNAs for guiding the base editor to the target sequences. The disclosed methods are capable of inducing between ten and 300,000 edits to the genome of a eukaryotic cell. Further disclosed are pharmaceutical compositions and compositions of eukaryotic cells comprising fusion proteins and a plurality of unique gRNAs, and a combination of small molecule factors and inhibitors. Also disclosed are kits for the generation of the fusion protein-gRNA complexes described herein.

### BACKGROUND OF THE INVENTION

The Human Genome Project completed the first draft of the human genome sequence in 2004. Since the initiation of this effort, the quality and cost of DNA sequencing technologies have improved exponentially. A human genome can now be sequenced in a few hours for a few hundred dollars, while it took more than 20 years and about 3 billion dollars to complete the first human genome sequence. The capacity to “write” or “recode” DNA at the genomic scale—i.e., achieve large-scale DNA editing and synthesis—has greatly improved in recent years. However, it has been outpaced by advances in high-throughput DNA sequencing development. In this context, similar to the Human Genome Project, initiatives such as Genome Project Write (GP-Write), which was launched in 2016, aim to reduce drastically the cost of designing, synthesizing, assembling and testing genomes.¹ Magnifying the ability to write DNA could transform the field of human health by enabling the engineering of virus-, cancer-, or aging-resistant cell lines, development of universal donor cell therapies,² and generation of xeno-compatible or synthetic organs,³ among other countless applications. In addition, DNA writing could help the scientific community probe the physiological and pathological relevance of the “dark matter of the genome”—the non-coding sequences which include Transposable Elements (TEs)—whose functions are still widely unknown but often associated with diseases.^{4,5}

In an early proof of concept study demonstrating genome-wide recoding of an entire living organism by multiplex automated genome engineering (MAGE), all 321 occurrences of the UAG stop codon in Escherichia coli MG1655 were replaced with UAA stop codons.⁶ As current DNA-editing technologies are unable to successfully re-write genomes at the scale of hundreds to thousands of loci, the recoding of mammalian organisms poses a great challenge. Developing DNA editing tools capable of large-scale modifications could reveal a path towards achieving genome-wide recoding.

Developing genome editing tools capable of large-scale modifications may also lead to an improved understanding of the physio-pathology of TEs, such as Alu,¹⁷ Long Interspersed Elements-1 (LINE-1),^{18,20} or Human Endogenous RetroViruses (HERVs),²¹ by enabling causal investigation of their functions. These DNA sequences are highly abundant and have homology to 45% of the native human genome.²² While originally characterized as “junk DNA,” TEs are now recognized as having shaped evolution of the human genome, and their residual transposition activity has been linked to human physiology and disease.

### SUMMARY OF THE INVENTION

Before large-scale genome editors can be used to recode organisms or study high copy number TEs, they will need to overcome two main hurdles: 1) the delivery of multiple modular guide RNAs (gRNAs) in a single large batch (or bolus) or over iterative treatments with subsets of targets; and 2) the cytotoxicity associated with genome-wide DNA modifications by way of making cuts at hundreds to thousands of loci.²⁹ Overall, there exists a need in the art for DNA nucleobase (or “base”) editing methods capable of multiplexed editing of hundreds to thousands of repetitive eukaryotic genomic loci without inducing high toxicity levels. A need similarly exists for highly multiplexed base editing methods that can edit tens, hundreds or greater than hundreds of unique genomic loci without inducing high toxicity levels.

When considering edits across multiple distinct loci, multiplex genome editing using CRISPR-Cas9 requires the simultaneous presence of multiple gRNAs inside the cell to be edited, which presents a major obstacle to successful multiplex editing. No single method currently exists to effectively deliver or express multiple guides with the efficiency and scale needed for massively multiplexed genome editing.

To satisfy these needs in the art, the present disclosure provides highly multiplexed base editing methods and compositions that minimize the induction of DNA damage sensors in cells and thereby maintain cell viability. These methods are capable of i) editing hundreds to tens of thousands of repetitive genomic loci, and ii) editing tens to hundreds of unique genomic loci without inducing high toxicity levels. The present disclosure is aimed to satisfy a need in the art for the reduction of editing-associated cytotoxicity due to double-stranded breaks (DSBs) and single-strand breaks (SSBs) generated by current DNA editors.

The design, synthesis, and testing of an ongoing large-scale E. coli genome recoding project to remove a total of seven codons out of the 64 possible 3-letter codes, involving the alteration of ˜62,214 codons is currently underway⁷ and in theory will provide pan-virus resistance by altering the highly conserved genetic code. Non-standard amino acids could also be introduced along with synthetic derivatives aimed toward new functionality and control over synthetic circuits and biological systems.⁶ To achieve a similar goal in human cells would require an estimated modification of 4438 to 9811 loci to recode all instances of one of the three stop codons.⁸

The discovery and widespread implementation of the CRISPR/Cas system⁹⁻¹¹ has dramatically expanded the toolbox for genome engineering and has revolutionized the future prospects of basic biological research, data storage in living systems,¹² agricultural science,¹³ and medicine.¹⁴ An advantage of CRISPR/Cas-based genome editors over prior approaches is the capacity to multiplex by using several guide RNAs (gRNAs). This not only enables the screening of libraries of guides in a single cell population but also the targeting of up to six unique loci at once;¹⁵ however, the efficiency at each site decreases when compared to that of a single guide transfection.

The recent development of DNA base editors by fusion of a deaminase to Cas9 enables gRNA targeted single nucleotide deamination for C:G base pair conversion to a T:A pair using cytidine base editors³⁰ (CBEs) or A:T base pair conversion to G:C using adenine base editors (ABEs) within a specific target window.³¹ Base editing has been broadly demonstrated with high efficiency in a range of species including human zygotes.³² Using appropriately designed gRNAs, C→T conversions may be used to generate stop codons to knock-out protein coding genes of interest.²⁷ Random genome-wide off-target SNVs have been reported when using CBEs that appear to be independent of gRNA binding sites^{54,55}, additionally gRNA off-targets have been reported when using BEs^{56, 57}. Additional improvements in base editing purity—the frequency of desired base conversion within a target window—have been achieved by fusing bacterial mu-gam protein to the base editor to generate Cas9 nickase-cytidine deaminase-gam, or nCBE4-gam.³³ The first generation of CBEs used dead Cas9 (dCas9) as the targeting system, but low efficiencies caused a shift to the use of Cas9 nickase (nCas9) in all generations beyond dead Cas9—cytidine base editor version 2, or dCBE2 (Table 3).

Current uses of the CRISPR/Cas9 system in multiplexed editing incur toxicity because Cas9 generates double-stranded DNA breaks (DSBs).²⁷ The system triggers the recruitment of endogenous repair processes that can correct DSBs with stochastic or user-specified variations, but high numbers of concurrent DSBs can overwhelm these processes and cause cell death. However, two types of CRISPR/Cas9 “base editors” (BEs) have recently been developed (Table 3) by fusing variants of Cas9 that are either “dead” (dCas9; both nuclease domains inactivated) or “nicking” (nCas9; one nuclease domain inactivated), in which the DSB-generating nuclease domains are disabled and tethered to a nucleotide deaminase: cytidine base editors (CBEs: either dCBEs or nCBEs³⁰) employ cytidine deaminases and convert C:G base pairs to T:A, while adenine base editors (ABEs: either dABEs or nABEs³¹) within a specific target window. As described below, cytidine base editors editor convert a C:G Watson-Crick nucleobase pair to a T:A Watson-Crick nucleobase pair (or a U:A Watson-Crick nucleobase pair); and adenine base editors convert an A:T Watson-Crick nucleobase pair to a G:C Watson-Crick nucleobase pair.

The present disclosure provides novel multiplexed base editing methods based on a CRISPR/Cas9 system that utilize dCas9 to minimize toxicity induced by SSBs and DSBs. Such a strategy improves the survival of highly-edited clones and provides for higher numbers of simultaneously-edited loci within a single eukaryotic cell than described in the prior art. Further, this strategy facilitates the editing of single targets in sensitive cell types, such as human induced Pluripotent Stem Cell (hiPSCs), where even single DSB can lead to apoptosis.⁵¹

The methods of the present disclosure improve the survival of eukaryotic cells following large-scale genome editing. These methods are based upon the discovery that use of a dead Cas9 base editor and optimal cell conditions during and after base editing enhances cells' tolerance to and survival after thousands of edits to the genome. In addition, optimal cell conditions after base editing include the addition of a combination of anti-apoptotic factors, growth factors and inhibitors of base excision repair, mismatch repair and/or non-homologous end joining. The methods of the present disclosure expand multiplexed base editing toward the upper limits of eukaryotic cells' amenability to genome-wide modifications, including the editing of thousands of transpable element (TE) sites concurrently in a single human cells. These methods are facilitated by the design and use of tens to hundreds to thousands of gRNAs for guiding the base editor to the target sequences.

To extend the frontiers of genome editing and enable the radical redesign of mammalian genomes, a set of dead-Cas9 base editor (dBEs) variants were developed that allow editing at tens of thousands of loci per cell by overcoming cell death associated with DNA double-strand breaks (DSBs) and single-strand breaks (SSBs). A set of gRNAs targeting repetitive elements was used, ranging in target copy number from about 31 to 124,000 per cell. dBEs enabled survival after large-scale base editing, allowing targeted deamination at up to ˜13,200 and ˜2610 loci, respectively, in HEK 293T and induced pluripotent stem cells, three orders of magnitude greater than previously reported. These dBEs satisfy needs in the art for overcoming on-target mutation and toxicity barriers that prevent survival after large-scale genome engineering.

The present disclosure provides for methods of base editing comprising: contacting a nucleic acid molecule (e.g. DNA) with a plurality of fusion proteins, wherein each of the fusion proteins of the plurality comprises (i) a nuclease inactive Cas9 (dCas9) domain and (ii) a deaminase domain, and a guide RNA (gRNA) bound to the dCas9 domain, wherein at least five of the fusion proteins of the plurality are each bound to a unique gRNA comprising a different guide sequence of at least 5, 7, or 10 contiguous nucleotides that is complementary to a target sequence in the genomic DNA of a eukaryotic cell. In certain embodiments, at least 10, 15, 20, 50, 100, 500, 1000, 5000, 10 000, 50,000, 100000, 150,000, 200,000, 300,000, 500,000 or more of the fusion proteins of the plurality are each bound to a unique gRNA comprising a different guide sequence of at least 10 contiguous nucleotides that is complementary to a target sequence.

In certain embodiments, the deaminase domain is a cytidine deaminase, e.g. an apolipoprotein B mRNA-editing complex 1 (APOBEC1) deaminase domain. Alternatively, the deaminase domain may be an adenosine deaminase.

The present disclosure also provides embodiments in which the DNA binding domain is a transcription activator-like (TAL) effector domain.

The fusion proteins utilized in the disclosed methods may further comprises an inhibitor of base excision repair (“iBER”) domain. In particular embodiments, fusion proteins containing a cytidine deaminase domain may further contain an iBER domain that comprise a uracil glycosylase inhibitor (UGI) domain.

In various embodiments, at least ten, twenty, thirty, fifty or more of the fusion proteins of the plurality are each bound to a unique gRNA that is complementary to a target sequence in the genomic DNA of a eukaryotic cell. In various embodiments, the step of contacting comprises editing more than 50, more than 100, more than 200, more than 500, more than 1,000, more than 2,000, more than 3,000, more than 5,000, more than 10,000, more than 20,000, more than 30,000, more than 50,000, more than 75,000, more than 100,000, or more than 300,000 target sequences in the genomic DNA of the eukaryotic cell.

In various embodiments, the eukaryotic cell of the disclosed methods is a vertebrate cell, e.g. a mammalian cell. In certain embodiments, the eukaryotic cell is a human cell, e.g. a human iPS or ES cell.

In various embodiments, the step of contacting results in a base editing efficiency of at least 35%, 40%, 50%, 60%, 70%, 80%, 85%, 90%, 95%, 98%, or 99%. In certain embodiments, the step of contacting results in low toxicity when administered to a population of cells. In particular embodiments, less than 30%, less than 20%, less than 15%, less than 10%, less than 5%, or less than 1% cell death in the population of cells is observed. In various embodiments, the step of contacting results in a low level of DNA damage when administered to a population of cells, e.g. at least 60%, at least 65%, at least 70%, at least 75%, at least 80%, at least 85%, at least 90%, at least 95%, or at least 99% of the cells are viable 24 hours after the step of contacting.

In certain embodiments, the base editing methods of the present disclosure further comprise contacting the eukaryotic cell with an anti-apoptotic molecule to promote cell survival. In certain particular embodiments, the anti-apoptotic molecule is pifithrin-α (PFA) or pifithrin-μ (PFμ). In various embodiments, the methods further comprise contacting the eukaryotic cell with a growth factor, e.g. basic fibroblast growth factor (bFGF). In other embodiments, the methods further comprise contacting the eukaryotic cell with an inhibitor of mismatch repair (MMR), e.g. cadmium chloride; or an inhibitor of non-homologous end joining (NHEJ). In certain embodiments, the methods further comprise conditionally knocking out a gene in the cell encoding a protein involved in NHEJ or MMR, e.g. the gene encoding the MutSα complex or the gene encoding the MutLα complex.

In other embodiments, the present disclosure provides base editing methods comprising: contacting a nucleic acid molecule with a fusion protein comprising (i) a nuclease inactive Cas9 (dCas9) domain and (ii) a deaminase domain, and a guide RNA (gRNA) bound to the dCas9 domain, wherein the guide RNA comprises a sequence of at least 10 contiguous nucleotides that is complementary to a target sequence, and wherein at least 25 copies of a target sequence are present in the genomic DNA of a eukaryotic cell. In certain embodiments, the target sequence is a repetitive element. In certain embodiments, the gRNA is a single-guide RNA (sgRNA), e.g. a promiscuous gRNA.

In various other embodiments, the disclosure provides compositions of eukaryotic cells comprising a plurality of the fusion proteins described herein. In particular embodiments, these compositions further comprise an anti-apoptotic molecule, a growth factor, and/or an inhibitor of MMR.

In other embodiments, the disclosure provides pharmaceutical compositions comprising any of the fusion proteins described herein and a gRNA, wherein at least five of the fusion proteins of the plurality are each bound to a unique gRNA, and a pharmaceutically acceptable excipient. In particular embodiments, the pharmaceutical compositions further comprise one or more of an anti-apoptotic molecule, a growth factor, and an inhibitor of mismatch repair. In certain embodiments, administration of the pharmaceutical compositions results in low toxicity when administered to a population of cells.

In other embodiments, the disclosure provides kits comprising a nucleic acid construct that includes (i) a nucleic acid sequence encoding comprising a plurality of fusion proteins described herein, (ii) a heterologous promoter that drives expression of the sequence of (a); and (iii) an expression construct encoding a plurality of unique guide RNA backbones, wherein the construct comprises a cloning site positioned to allow the cloning of a nucleic acid sequence identical or complementary to a target sequence into each of the guide RNA backbones.

The details of one or more embodiments of the invention are set forth herein. Other features, objects, and advantages of the invention will be apparent from the Detailed Description, Examples, Figures, and Claims. References cited in this application are incorporated herein by reference in their entireties.

### Definitions

As used herein and in the claims, the singular forms “a,” “an,” and “the” include the singular and the plural reference unless the context clearly indicates otherwise. Thus, for example, a reference to “an agent” includes a single agent and a plurality of such agents.

“Ancestral sequence reconstruction (ASR)” refers to the process of analyzing modern sequences within an evolutionary/phylogenetic context to infer the ancestral sequences at particular nodes of a tree using an ASR algorithm. ASR algorithms are known in the art. Proteins that have been reconstructed through ancestral sequencing are herein denoted with an “Anc” prefix (e.g. AncBE4max).

“Base editing” refers to genome editing technology that involves the conversion of a specific nucleic acid base into another at a targeted genomic locus. In certain embodiments, this can be achieved without requiring double-stranded DNA breaks (DSB), or single stranded breaks (i.e., nicking). To date, other genome editing techniques, including CRISPR-based systems, begin with the introduction of a DSB at a locus of interest. Subsequently, cellular DNA repair enzymes mend the break, commonly resulting in random insertions or deletions (indels) of bases at the site of the DSB. However, when the introduction or correction of a point mutation at a target locus is desired rather than stochastic disruption of the entire gene, these genome editing techniques are unsuitable, as correction rates are low (e.g. typically 0.1% to 5%), with the major genome editing products being indels. In order to increase the efficiency of gene correction without simultaneously introducing random indels, the present inventors previously modified the CRISPR/Cas9 system to directly convert one DNA base into another without DSB formation. See, Komor, A. C., et al., Programmable editing of a target base in genomic DNA without double-stranded DNA cleavage. Nature 533, 420-424 (2016), the entire contents of which is incorporated by reference herein.

The following base editors, which effect transitions (pyrimidine to pyrimidine, or purine to purine) mutations are relevant to the methods disclosed herein.
Cytidine base editor (or “CBE”). This type of editor converts a C:G Watson-Crick nucleobase pair to a T:A Watson-Crick nucleobase pair (or a U:A Watson-Crick nucleobase pair). Because the corresponding Watson-Crick paired bases are also interchanged as a result of the conversion, this category of base editor may also be referred to as a guanine base editor (or “GBE”).
Adenine base editor (or “ABE”). This type of editor converts an A:T Watson-Crick nucleobase pair to a G:C Watson-Crick nucleobase pair. Because the corresponding Watson-Crick paired bases are also interchanged as a result of the conversion, this category of base editor may also be referred to as a thymine base editor (or “TBE”).

The term “base editor (BE)” as used herein, refers to the CRISPR-mediated fusion proteins that are utilized in the multiplexed base editing methods described herein. In some embodiments, the base editor comprises a nuclease-inactive Cas9 (dCas9) fused to a deaminase which binds a nucleic acid in a guide RNA-programmed manner via the formation of an R-loop, but does not cleave the nucleic acid. For example, the dCas9 domain of the fusion protein may include a D10A and a H840A mutation (which renders Cas9 capable of cleaving only one strand of a nucleic acid duplex), as described in PCT/US2016/058344 (filed on Oct. 22, 2016, and published as WO 2017/070632, on Apr. 27, 2017), which is incorporated herein by reference in its entirety. The DNA cleavage domain of S. pyogenes Cas9 includes two subdomains, the HNH nuclease subdomain and the RuvC1 subdomain. The HNH subdomain cleaves the strand complementary to the gRNA (the “targeted strand”, or the strand in which editing or deamination occurs), whereas the RuvC1 subdomain cleaves the non-complementary strand containing the PAM sequence (the “non-edited strand”). The RuvC1 mutant D10A generates a nick in the targeted strand, while the HNH mutant H840A generates a nick on the non-edited strand (see Jinek et al., Science, 337:816-821(2012); Qi et al., Cell. 28; 152(5):1173-83 (2013)).

The term “base editor” encompasses the CRISPR-mediated fusion proteins utilized in the multiplexed base editing methods described herein as well as any base editor known or described in the art at the time of this filing or developed in the future. Reference is made to Rees & Liu, Base editing: precision chemistry on the genome and transcriptome of living cells, Nat. Rev. Genet. 2018; 19(12):770-788; as well as. U.S. Patent Publication No. 2018/0073012, published Mar. 15, 2018, which issued as U.S. Pat. No. 10,113,163, on Oct. 30, 2018; U.S. Patent Publication No. 2017/0121693, published May 4, 2017, which issued as U.S. Pat. No. 10,167,457 on Jan. 1, 2019; International Publication No. WO 2017/070633, published Apr. 27, 2017; U.S. Patent Publication No. 2015/0166980, published Jun. 18, 2015; U.S. Pat. No. 9,840,699, issued Dec. 12, 2017; and U.S. Pat. No. 10,077,453, issued Sep. 18, 2018, the contents of each of which are incorporated herein by reference in their entireties.

The term “Cas9” or “Cas9 nuclease” or “Cas9 domain” refers to a CRISPR-associated protein 9, or variant thereof, and embraces any naturally occurring Cas9 from any organism, any naturally-occurring Cas9 equivalent or fragment thereof, any Cas9 homolog, ortholog, or paralog from any organism, and any variant of a Cas9, naturally-occurring or engineered. The term Cas9 is not meant to be particularly limiting and may be referred to as a “Cas9 or variant thereof.” Exemplary Cas9 proteins are described herein and also described in the art. The present disclosure is unlimited with regard to the particular Cas9 that is employed in the CRISPR-mediated fusion proteins utilized in the disclosure.

As used herein, the term “dCas9” refers to a nuclease-inactive Cas9 or nuclease-dead Cas9, or a variant thereof, and embraces any naturally occurring dCas9 from any organism, any naturally-occurring dCas9 equivalent or functional fragment thereof, any dCas9 homolog, ortholog, or paralog from any organism, and any variant of a dCas9, naturally-occurring or engineered. The term dCas9 is not meant to be particularly limiting and may be referred to as a “dCas9 or variant thereof.” Exemplary dCas9 proteins and method for making dCas9 proteins are further described herein and/or are described in the art and are incorporated herein by reference. Any suitable mutation which inactivates both Cas9 endonucleases, such as D10A and H840A mutations in the wild-type S. pyogenes Cas9 amino acid sequence, or D10A and N580A mutations in the wild-type S. aureus Cas9 amino acid sequence, may be used to form the dCas9.

As used herein, the term “nCas9” or “Cas9 nickase” refers to a Cas9 or a variant thereof, which cleaves or nicks only one of the strands of a target cut site thereby introducing a nick in a double strand DNA molecule rather than creating a double strand break. This can be achieved by introducing appropriate mutations in a wild-type Cas9 which inactivates one of the two endonuclease activities of the Cas9. Any suitable mutation which inactivates one Cas9 endonuclease activity but leaves the other intact is contemplated, such as one of D10A or H840A mutations in the wild-type S. pyogenes Cas9 amino acid sequence, or a D10A mutation in the wild-type S. aureus Cas9 amino acid sequence, may be used to form the nCas9.

The term “deaminase” or “deaminase domain” refers to a protein or enzyme that catalyzes a deamination reaction. In some embodiments, the deaminase is an adenine deaminase, which catalyzes the hydrolytic deamination of adenine or adenosine. In other embodiments, the deaminase or deaminase domain is a cytidine deaminase, catalyzing the hydrolytic deamination of cytidine to uracil. In some embodiments, the adenosine deaminase catalyzes the hydrolytic deamination of adenine or adenosine in deoxyribonucleic acid (DNA) to inosine. In some embodiments, the cytidine deaminase catalyzes the hydrolytic deamination of cytidine in DNA.

The deaminases provided herein may be from any organism, such as a bacterium. In some embodiments, the deaminase or deaminase domain is a variant of a naturally-occurring deaminase from an organism. In some embodiments, the deaminase or deaminase domain does not occur in nature. For example, in some embodiments, the deaminase or deaminase domain is at least 50%, at least 55%, at least 60%, at least 65%, at least 70%, at least 75% at least 80%, at least 85%, at least 90%, at least 95%, at least 96%, at least 97%, at least 98%, at least 99%, or at least 99.5% identical to a naturally-occurring deaminase.

The cytidine deaminases (e.g. engineered cytidine deaminases, evolved cytidine deaminases) described herein may be enzymes that convert cytidine (C) to uracil (U) in DNA. If DNA replication occurs before uracil repair, the replication machinery may treat the uracil as thymine (T), leading to a C:G to T:A base pair conversion. In some embodiments, the cytidine deaminases utilized in the base editor are apolipoprotein B mRNA-editing complex 1 (APOBEC1) deaminases, e.g. rat APOBEC1 deaminases.

The adenosine deaminases (e.g. engineered adenosine deaminases, evolved adenosine deaminases) provided herein may be may be enzymes that convert adenine (A) to guanine (G) in DNA, leading to an A:T to G:C base pair conversion. In some embodiments, the adenosine deaminase is derived from a bacterium, such as, E. coli, S. aureus, S. typhi, S. putrefaciens, H. influenzae, or C. crescentus. In some embodiments, the adenosine deaminase is a TadA deaminase. In some embodiments, the TadA deaminase is an E. coli TadA deaminase (ecTadA). In some embodiments, the TadA deaminase is a truncated E. coli TadA deaminase. For example, the truncated ecTadA may be missing one or more N-terminal amino acids relative to a full-length ecTadA. In some embodiments, the truncated ecTadA may be missing 1, 2, 3, 4, 5, 6, 7, 8, 9, 10, 11, 12, 13, 14, 15, 6, 17, 18, 19, or 20 N-terminal amino acid residues relative to the full length ecTadA. In some embodiments, the truncated ecTadA may be missing 1, 2, 3, 4, 5, 6, 7, 8, 9, 10, 11, 12, 13, 14, 15, 6, 17, 18, 19, or 20 C-terminal amino acid residues relative to the full length ecTadA. In some embodiments, the ecTadA deaminase does not comprise an N-terminal methionine. Reference is made to U.S. Patent Publication No. 2018/0073012, published Mar. 15, 2018, which is incorporated herein by reference.

As used herein, the term “DNA binding protein” or “DNA binding protein domain” refers to any protein that localizes to and binds a specific target DNA nucleotide sequence (e.g. a gene locus of a genome). This term embraces RNA-programmable proteins, which associate (e.g. form a complex) with one or more nucleic acid molecules (i.e., which includes, for example, guide RNA in the case of Cas systems) that direct or otherwise program the protein to localize to a specific target DNA nucleotide sequence that is complementary to the one or more DNA molecules (or a portion or region thereof) associated with the protein. Exemplary RNA-programmable proteins are CRISPR-Cas9 proteins, as well as Cas9 equivalents, homologs, orthologs, or paralogs, whether naturally occurring or non-naturally occurring (e.g. engineered or modified), and may include a Cas9 equivalent from any type of CRISPR system (e.g. type II, V, VI), including Cpf1 (a type-V CRISPR-Cas systems), C2c1 (a type V CRISPR-Cas system), C2c2 (a type VI CRISPR-Cas system), C2c3 (a type V CRISPR-Cas system), dCas9, GeoCas9, CjCas9, Cas12a, Cas12b, Cas12c, Cas12d, Cas12g, Cas12h, Cas12i, Cas13d, Cas14, Argonaute, and nCas9. Further Cas-equivalents are described in Makarova et al., “C2c2 is a single-component programmable RNA-guided RNA-targeting CRISPR effector,” Science 2016; 353(6299), the contents of which are incorporated herein by reference. The term also embraces transcripation activator-like (TAL) effector (or TALE) proteins, which use one or more cofactor proteins (e.g. FokI cofactor proteins) that may be directly attached by a linker or delivered separately, that direct or otherwise program the fusion protein to localize to a specific target DNA nucleotide sequence. In particular embodiments, the TALE effector is truncated at the N- or C-terminus. Reference is made to Zhang F. et al., Efficient construction of sequence-specific TAL effectors for modulating mammalian transcription, Nat Biotechnol. 2011 February; 29(2):149-53 and Yang L. et al., Engineering and optimising deaminase fusions for genome editing, Nat Commun. 2016 Nov. 2; 7:13330, each of which is incorporated herein by reference in its entirety.

The term “effective amount,” as used herein, refers to an amount of a biologically active agent that is sufficient to elicit a desired biological response. For example, in some embodiments, an effective amount of a composition may refer to the amount of the composition that is sufficient to edit a target site of a nucleotide sequence, e.g. a genome. In some embodiments, an effective amount of a composition provided herein, e.g. of a composition comprising a nuclease-inactive Cas9 domain, a deaminase domain, a gRNA and optionally a growth factor and anti-apoptotic factor, may refer to the amount of the composition that is sufficient to induce editing of a target site specifically bound and edited by the fusion protein. In some embodiments, an effective amount of a composition provided herein may refer to the amount of the composition sufficient to induce editing having the following characteristics: >50% product purity, <5% indels, and an editing window of 2-8 nucleotides. As will be appreciated by the skilled artisan, the effective amount of an agent, e.g. a composition or a fusion protein-gRNA complex, may vary depending on various factors as, for example, on the desired biological response, e.g. on the specific allele, genome, or target site to be edited, on the cell or tissue being targeted, and on the agent being used.

The term “fusion protein,” as used herein, refers to a hybrid polypeptide which comprises protein domains from at least two different proteins. One protein may be located at the amino-terminal (N-terminal) portion of the fusion protein or at the carboxy-terminal (C-terminal) protein thus forming an “amino-terminal fusion protein” or a “carboxy-terminal fusion protein,” respectively. A protein may comprise different domains, for example, a nucleic acid binding domain (e.g. the gRNA binding domain of Cas9 that directs the binding of the protein to a target site) and a nucleic acid cleavage domain or a catalytic domain of a nucleic-acid editing protein. Any of the proteins provided herein may be produced by any method known in the art. For example, the proteins provided herein may be produced via recombinant protein expression and purification, which is especially suited for fusion proteins comprising a peptide linker. Methods for recombinant protein expression and purification are well known, and include those described by Green and Sambrook, Molecular Cloning: A Laboratory Manual (4^{th} ed., Cold Spring Harbor Laboratory Press, Cold Spring Harbor, N.Y. (2012)), the entire contents of which are incorporated herein by reference.

The term “linker,” as used herein, refers to a chemical group or a molecule linking two molecules or domains, e.g. dCas9 and a deaminase. Typically, the linker is positioned between, or flanked by, two groups, molecules, or other domains and connected to each one via a covalent bond, thus connecting the two. In some embodiments, the linker is an amino acid or a plurality of amino acids (e.g. a peptide or protein). In some embodiments, the linker is an organic molecule, group, polymer, or chemical domain. Chemical groups include, but are not limited to, disulfide, hydrazone, and azide domains. In some embodiments, the linker is 5-100 amino acids in length, for example, 5, 6, 7, 8, 9, 10, 11, 12, 13, 14, 15, 16, 17, 18, 19, 20, 21, 22, 23, 24, 25, 26, 27, 28, 29, 30, 30-35, 35-40, 40-45, 45-50, 50-60, 60-70, 70-80, 80-90, 90-100, 100-150, or 150-200 amino acids in length. Longer or shorter linkers are also contemplated. In some embodiments, the linker is an XTEN linker. In some embodiments, the linker is a 32-amino acid linker. In other embodiments, the linker is a 30-, 31-, 33- or 34-amino acid linker.

As used herein, the term “low toxicity” refers to the maintenance of a viability above 60% in a population of cells following application of a multiplexed base editing method or administration of a composition disclosed herein. The term may also refer to prevention of apoptosis (cell death) in a population of cells of more than 40%. For instance, a multiplexed genome editing method that leads to less than 30% (e.g. 25%, 20%, 15%, 10%, or 5%) cell death exhibits low toxicity. Cell toxicity may be assessed by an appropriate staining assay, e.g. Annexin V and propidium iodide staining assays, and subsequent flow cytometry (e.g. FACS).

As used herein, the term “low level of DNA damage” refers to an extent of DNA damage that is tolerable by a population of cells before significant apoptosis is observed. Apoptosis may be significant when it exceeds 40% (e.g. 45%, 50%, 55%, 60%, or 65%) death in the cell population. Degree of apoptosis may be assessed by an appropriate staining assay, e.g. Annexin V and propidium iodide staining assays, and subsequent flow cytometry (e.g. FACS). The effects of DSBs on DNA may be assayed by antibody staining for gamma H2AX histone modification. SSBs may be detected by single cell gel electrophoresis (e.g. a Comet assay).

The term “mutation,” as used herein, refers to a substitution of a residue within a sequence, e.g. a nucleic acid or amino acid sequence, with another residue; a deletion or insertion of one or more residues within a sequence; or a substitution of a residue within a sequence of a genome in a subject to be corrected. Mutations are typically described herein by identifying the original residue followed by the position of the residue within the sequence and by the identity of the newly substituted residue. Various methods for making the amino acid substitutions (mutations) provided herein are well known in the art, and are provided by, for example, Green and Sambrook, Molecular Cloning: A Laboratory Manual (4^{th} ed., Cold Spring Harbor Laboratory Press, Cold Spring Harbor, N.Y. (2012)). Mutations can include a variety of categories, such as single base polymorphisms, microduplication regions, indel, and inversions, and is not meant to be limiting in any way. Mutations can include “loss-of-function” mutations which is a result of a mutation that reduces or abolishes a protein activity. Most loss-of-function mutations are recessive, because in a heterozygote the second chromosome copy carries an unmutated version of the gene coding for a fully functional protein whose presence compensates for the effect of the mutation. There are some exceptions where a loss-of-function mutation is dominant, one example being haploinsufficiency, where the organism is unable to tolerate the approximately 50% reduction in protein activity suffered by the heterozygote. This is the explanation for a few genetic diseases in humans, including Marfan syndrome, which results from a mutation in the gene for the connective tissue protein called fibrillin. Mutations also embrace “gain-of-function” mutations, which is one which confers an abnormal activity on a protein or cell that is otherwise not present in a normal condition. Many gain-of-function mutations are in regulatory sequences rather than in coding regions, and can therefore have a number of consequences. For example, a mutation might lead to one or more genes being expressed in the wrong tissues, these tissues gaining functions that they normally lack. Alternatively the mutation could lead to overexpression of one or more genes involved in control of the cell cycle, thus leading to uncontrolled cell division and hence to cancer. Because of their nature, gain-of-function mutations are usually dominant.

The terms “non-naturally occurring” or “engineered” are used interchangeably and indicate the involvement of the hand of man. These terms, when referring to nucleic acid molecules or polypeptides (e.g. Cas9 or deaminases) mean that the nucleic acid molecule or the polypeptide is at least substantially free from at least one other component with which they are naturally associated in nature and/or as found in nature (e.g. an amino acid sequence not found in nature).

The term “nucleic acid,” as used herein, refers to RNA as well as single and/or double-stranded DNA. Nucleic acids may be naturally occurring, for example, in the context of a genome, a transcript, an mRNA, tRNA, rRNA, siRNA, snRNA, a plasmid, cosmid, chromosome, chromatid, or other naturally occurring nucleic acid molecule. On the other hand, a nucleic acid molecule may be a non-naturally occurring molecule, e.g. a recombinant DNA or RNA, an artificial chromosome, an engineered genome, or fragment thereof, or a synthetic DNA, RNA, DNA/RNA hybrid, or including non-naturally occurring nucleotides or nucleosides. Furthermore, the terms “nucleic acid,” “DNA,” “RNA,” and/or similar terms include nucleic acid analogs, e.g. analogs having other than a phosphodiester backbone. Nucleic acids may be purified from natural sources, produced using recombinant expression systems and optionally purified, chemically synthesized, etc. Where appropriate, e.g. in the case of chemically synthesized molecules, nucleic acids may comprise nucleoside analogs such as analogs having chemically modified bases or sugars, and backbone modifications. A nucleic acid sequence is presented in the 5′ to 3′ direction unless otherwise indicated. In some embodiments, a nucleic acid is or comprises natural nucleosides (e.g. adenosine, thymidine, guanosine, cytidine, uridine, deoxyadenosine, deoxythymidine, deoxyguanosine, and deoxycytidine); nucleoside analogs (e.g. 2-aminoadenosine, 2-thiothymidine, inosine, pyrrolo-pyrimidine, 3-methyl adenosine, 5-methylcytidine, 2-aminoadenosine, C5-bromouridine, C5-fluorouridine, C5-iodouridine, C5-propynyl-uridine, C5-propynyl-cytidine, C5-methylcytidine, 2-aminoadenosine, 7-deazaadenosine, 7-deazaguanosine, 8-oxoadenosine, 8-oxoguanosine, 0(6)-methylguanine, and 2-thiocytidine); chemically modified bases; biologically modified bases (e.g. methylated bases); intercalated bases; modified sugars (e.g. 2′-fluororibose, ribose, 2′-deoxyribose, arabinose, and hexose); and/or modified phosphate groups (e.g. phosphorothioates and 5′-N-phosphoramidite linkages).

A nuclear localization signal or sequence (NLS) is an amino acid sequence that tags, designates, or otherwise marks a protein for import into the cell nucleus by nuclear transport. Typically, this signal consists of one or more short sequences of positively charged lysines or arginines exposed on the protein surface. Different nuclear localized proteins may share the same NLS. An NLS has the opposite function of a nuclear export signal (NES), which targets proteins out of the nucleus. Thus, a single nuclear localization signal can direct the entity with which it is associated to the nucleus of a cell. Such sequences can be of any size and composition, for example more than 25, 25, 15, 12, 10, 8, 7, 6, 5, or 4 amino acids, but will preferably comprise at least a four to eight amino acid sequence known to function as a nuclear localization signal (NLS).

As used herein, the term “promiscuous gRNA” refers to a single guide RNA (sgRNA) that is complementary to multiple locations (e.g. sequences) within a nucleic acid molecule and is thus able to target these multiple locations (e.g. sequences) simultaneously. A promiscuous gRNA may be complementary to 25, 50, 75, 100, 250, 500, 1,000, 3,000 or more than 3,000 locations within a nucleic acid molecule. Reference is made to Ferreira R. et al., Exploiting off-targeting in guide-RNAs for CRISPR systems for simultaneous editing of multiple genes, FEBS Lett. 2017; 591(20):3288-3295, incorporated herein by reference in its entirety.

The term “promoter” is art-recognized and refers to a nucleic acid molecule with a sequence recognized by the cellular transcription machinery and able to initiate transcription of a downstream gene. A promoter can be constitutively active, meaning that the promoter is always active in a given cellular context, or conditionally active, meaning that the promoter is only active in the presence of a specific condition. For example, a conditional promoter may only be active in the presence of a specific protein that connects a protein associated with a regulatory element in the promoter to the basic transcriptional machinery, or only in the absence of an inhibitory molecule. A subclass of conditionally active promoters are inducible promoters that require the presence of a small molecule “inducer” for activity. Examples of inducible promoters include, but are not limited to, arabinose-inducible promoters, Tet-on promoters, and tamoxifen-inducible promoters. A variety of constitutive, conditional, and inducible promoters are well known to the skilled artisan, and the skilled artisan will be able to ascertain a variety of such promoters useful in carrying out the instant invention, which is not limited in this respect. In various embodiments, the disclosure provides vectors with appropriate promoters for driving expression of the nucleic acid sequences encoding the fusion proteins (or one or more individual components thereof).

The term “subject,” as used herein, refers to an individual organism, for example, an individual mammal. In some embodiments, the subject is a human. In some embodiments, the subject is a non-human mammal. In some embodiments, the subject is a non-human primate. In some embodiments, the subject is a rodent. In some embodiments, the subject is a sheep, a goat, a cattle, a cat, or a dog. In some embodiments, the subject is a vertebrate, an amphibian, a reptile, a fish, an insect, a fly, or a nematode. In some embodiments, the subject is a research animal. In some embodiments, the subject is genetically engineered, e.g. a genetically engineered non-human subject. The subject may be of either sex and at any stage of development.

The term “target site” refers to a sequence within a nucleic acid molecule that is edited by a fusion protein (e.g. a dCas9-deaminase fusion protein provided herein). The target site further refers to the sequence within a nucleic acid molecule to which a complex of the fusion protein and gRNA binds.

The terms “treatment,” “treat,” and “treating,” refer to a clinical intervention aimed to reverse, alleviate, delay the onset of, or inhibit the progress of a disease, disorder, or condition, or one or more symptoms thereof, as described herein. As used herein, the terms “treatment,” “treat,” and “treating” refer to a clinical intervention aimed to reverse, alleviate, delay the onset of, or inhibit the progress of a disease, disorder, or condition, or one or more symptoms thereof, as described herein. In some embodiments, treatment may be administered after one or more symptoms have developed and/or after a disease has been diagnosed. In other embodiments, treatment may be administered in the absence of symptoms, e.g. to prevent or delay onset of a symptom or inhibit onset or progression of a disease. For example, treatment may be administered to a susceptible individual prior to the onset of symptoms (e.g. in light of a history of symptoms and/or in light of genetic or other susceptibility factors). Treatment may also be continued after symptoms have resolved, for example, to prevent or delay their prevention or recurrence.

As used herein, e.g. for the purposes of reporting a specific number of loci, the terms “unique loci” and “unique genomic loci” refer to distinct genomic sequences (e.g. distinct coding sequences) wherein all copies of a distinct sequence in the genome are collectively counted (or reported) only once; in contrast, each copy of a “non-unique locus” or “repetitive element” is counted for purposes of reporting a specific number of loci.

As used herein, the term “variant” refers to a protein having characteristics that deviate from what occurs in nature that retains at least one functional i.e. binding, interaction, or enzymatic ability and/or therapeutic property thereof. A “variant” is at least about 70% identical, at least about 80% identical, at least about 90% identical, at least about 95% identical, at least about 96% identical, at least about 97% identical, at least about 98% identical, at least about 99% identical, at least about 99.5% identical, or at least about 99.9% identical to the wild type protein. For instance, a variant of Cas9 may comprise a Cas9 that has one or more changes in amino acid residues as compared to a wild type Cas9 amino acid sequence. As another example, a variant of a deaminase may comprise a deaminase that has one or more changes in amino acid residues as compared to a wild type deaminase amino acid sequence, e.g. following ancestral sequence reconstruction of the deaminase. These changes include chemical modifications, including substitutions of different amino acid residues truncations, covalent additions (e.g. of a tag), and any other mutations. This term also embraces fragments of a wild type protein.

The level or degree of which the property is retained may be reduced relative to the wild type protein but is typically the same or similar in kind. Generally, variants are overall very similar, and, in many regions, identical to the amino acid sequence of the protein described herein. A skilled artisan will appreciate how to make and use variants that maintain all, or at least some, of a functional ability or property.

The variant proteins may comprise, or alternatively consist of, an amino acid sequence which is at least 80%, 85%, 90%, 95%, 96%, 97%, 98%, 99%, or 100%, identical to, for example, the amino acid sequence of a wild-type protein, or any protein provided herein (e.g. Cas9 protein, fusion protein, and fusion protein protein). Further polypeptides encompassed by the invention are polypeptides encoded by polynucleotides which hybridize to the complement of a nucleic acid molecule encoding a protein such as a Cas9 protein under stringent hybridization conditions (e.g. hybridization to filter bound DNA in 6× Sodium chloride/Sodium citrate (SSC) at about 45 degrees Celsius, followed by one or more washes in 0.2.times.SSC, 0.1% SDS at about 50-65 degrees Celsius), under highly stringent conditions (e.g. hybridization to filter bound DNA in 6× sodium chloride/Sodium citrate (SSC) at about 45 degrees Celsius, followed by one or more washes in 0.1×SSC, 0.2% SDS at about 68 degrees Celsius), or under other stringent hybridization conditions which are known to those of skill in the art (see, for example, Ausubel, F. M. et al., eds., 1989 Current Protocol in Molecular Biology, Green publishing associates, Inc., and John Wiley & Sons Inc., New York, at pages 6.3.1-6.3.6 and 2.10.3).

By a polypeptide having an amino acid sequence at least, for example, 95% “identical” to a query amino acid sequence, it is intended that the amino acid sequence of the subject polypeptide is identical to the query sequence except that the subject polypeptide sequence may include up to five amino acid alterations per each 100 amino acids of the query amino acid sequence. In other words, to obtain a polypeptide having an amino acid sequence at least 95% identical to a query amino acid sequence, up to 5% of the amino acid residues in the subject sequence may be inserted, deleted, or substituted with another amino acid. These alterations of the reference sequence may occur at the amino- or carboxy-terminal positions of the reference amino acid sequence or anywhere between those terminal positions, interspersed either individually among residues in the reference sequence or in one or more contiguous groups within the reference sequence.

As a practical matter, whether any particular polypeptide is at least 80%, 85%, 90%, 95%, 96%, 97%, 98%, or 99% identical to, for instance, the amino acid sequence of a protein such as a Cas9 protein, can be determined conventionally using known computer programs. A preferred method for determining the best overall match between a query sequence (a sequence of the present invention) and a subject sequence, also referred to as a global sequence alignment, can be determined using the FASTDB computer program based on the algorithm of Brutlag et al. (Comp. App. Biosci. 6:237-245 (1990)). In a sequence alignment the query and subject sequences are either both nucleotide sequences or both amino acid sequences. The result of said global sequence alignment is expressed as percent identity. Preferred parameters used in a FASTDB amino acid alignment are: Matrix=PAM 0, k-tuple=2, Mismatch Penalty=1, Joining Penalty=20, Randomization Group Length=0, Cutoff Score=1, Window Size=sequence length, Gap Penalty=5, Gap Size Penalty=0.05, Window Size=500 or the length of the subject amino acid sequence, whichever is shorter.

If the subject sequence is shorter than the query sequence due to N- or C-terminal deletions, not because of internal deletions, a manual correction must be made to the results. This is because the FASTDB program does not account for N- and C-terminal truncations of the subject sequence when calculating global percent identity. For subject sequences truncated at the N- and C-termini, relative to the query sequence, the percent identity is corrected by calculating the number of residues of the query sequence that are N- and C-terminal of the subject sequence, which are not matched/aligned with a corresponding subject residue, as a percent of the total bases of the query sequence. Whether a residue is matched/aligned is determined by results of the FASTDB sequence alignment. This percentage is then subtracted from the percent identity, calculated by the above FASTDB program using the specified parameters, to arrive at a final percent identity score. This final percent identity score is what is used for the purposes of the present invention. Only residues to the N- and C-termini of the subject sequence, which are not matched/aligned with the query sequence, are considered for the purposes of manually adjusting the percent identity score. That is, only query residue positions outside the farthest N- and C-terminal residues of the subject sequence.

As used herein, the term “wild type” is a term of the art understood by skilled persons and means the typical form of an organism, strain, gene or characteristic as it occurs in nature as distinguished from mutant or variant forms.

These and other exemplary substituents are described in more detail in the Detailed Description, Examples, and claims. The invention is not intended to be limited in any manner by the above exemplary listing of substituents.

### BRIEF DESCRIPTION OF THE DRAWINGS

FIGS. 1A-1D show utilizing high copy repetitive elements for the development of an extremely safe DNA editor. FIG. 1A shows a summary of HERV, LINE-1, and Alu. Representation of TEs with qPCR primer sites shown in red and gRNAs shown in green. FIG. 1B shows a qPCR estimation of LINE-1 copy number across the element compared to single copy number controls in PGP1 and HEK 293T. Errors bars display standard deviation, n=3. FIG. 1C shows genome wide distribution of HL1gR4. FIG. 1D shows HL1gR4 copy number and PAM distribution.

FIGS. 2A-2C show that CRISPR-Cas9 based genome editing at high copy number repetitive elements is detectable but ultimately lethal. FIG. 2A is a schematic of LINE-1 including the two protein coding genes ORF-1 and ORF-2. Three dual gRNA deletions were designed to disrupt the EN and RT domains of ORF-2. FIG. 2B shows LINE-1 gRNAs transfected with Cas9. Displayed are single transfections with 95% confidence intervals for a proportion as the error bars. FIG. 2C is a gel image visualizing dual gRNA deletion bands compared to wild type control bands.

FIGS. 3A-3D show that nBEs targeting LINE-1 enables survival of stable cell lines with hundreds of edits. FIG. 3A shows base editing in HEK 293T cells two days after transfection comparing nCBE3 vs. nCBE4-gam. FACS single cells are plotted as individual points representing targeted base editing nucleotide deamination. Red line indicates the median, and the blue line the mean. FIG. 3B shows single cell live culture growth and stable cell line generation at day 11 and 30. FIG. 3C shows base editing activity across the CBE target window of ˜3-9. Comparing day two and 30 for analysis of initial editing activity in most highly edited clones. FIG. 3D shows LINE-1 deamination analyzed from either RNA or genomic DNA. SEM's are displayed as error bars, n=2. “% of reads” refer to percentage of oligonucleotide sequences (reads) exhibiting the relevant variable, as measured by Illumina MiSeq sequencing.

FIGS. 4A-4C show that dBEs improve survival of highly edited cells with thousands of edits genome wide. FIG. 4A shows nBE compared to dBE in 293T single cells, each represented as a single data point. Base editing is displayed as either target C->T or A->G conversion for CBE and ABE, respectively. The red line indicates the median, and the blue line the mean. FIG. 4B shows live single cell analysis at day 14 of the same experiment.

FIG. 4C shows deamination frequency over time comparing dBE to nBE from day one to day ten. Error bars represent SEM, n=3. “% of reads” refer to percentage of oligonucleotide sequences (reads) exhibiting the relevant variable.

FIGS. 5A-5C show a “survival cocktail” and conditions for clonal derivation of iPSCs after large-scale genome engineering. FIG. 5A depicts human iPSC transfection timeline and survival cocktail conditions. FIG. 5B shows eighteen-hour single cell direct NGS analysis of dABE targeting LINE-1. The red line indicates the median and the blue line the mean. FIG. 5C shows live cell colony analysis of surviving iPSCs at day 11 post transfection.

FIGS. 6A-6B show dual gRNA LINE-1 deletions. FIG. 6A shows primers used to amplify dual gRNA pairs targeting LINE-1 with full length and expected deletion product sizes shown. gRNAs are represented as green with PAMs in yellow boxes, and primers are shown with thick arrows FIG. 6B shows dual gRNA deletion frequency displaying the expected cut points near each gRNA. Green nucleotides are within the gRNA sequence, red are inserted nucleotides, and “-” are deletions. The sizes of deletions and percentage among sequencing reads are displayed to the right. Top to bottom, left to right, the sequences in this figure correspond to SEQ ID NOs. 109-126.

FIGS. 7A-7C show single cell analysis of dual gRNA deletions targeting LINE-1 FIG. 7A shows gRNA targets used for the shEN dual deletion. Top to bottom, left to right, the sequences in this figure correspond to SEQ ID NOs. 127-128. FIG. 7B shows gel visualization of dual gRNA deletions bands in FACS single cells with a summary table. FIG. 7C shows the percentage of single cells with dual gRNA deletions.

FIGS. 8A-8C show nBE targeting LINE-1 FIG. 8A depicts LINE-1 gRNA targets outlined in dark boxes with PAMs in light-colored boxes. Expected ABE and CBE deamination products are displayed below with altered bases in blue and orange, respectively. Top to bottom, left to right, the sequences in this figure correspond to SEQ ID NOs. 129-146. FIG. 8B shows targeted deamination frequency at C8 using nCBE3. FIG. 8C shows targeted deamination frequency at A6 using nABE (ABE7.10, Addgene #102919).

FIGS. 9A-9B show nCBE3 vs Cas9 targeting Alu in HEK 293T cells. FIG. 9A shows microscope images of rapid cell death in cells that express Cas9 along with a gRNA that targets a high copy number locus. HEK 293T cells were transfected with a gRNA targeting the Alu consensus sequence along with either Cas9 that generates a DSB or nCBE3 which generates a single stranded break. Cells were imaged 72 hours after transfection. As a control, a non-human targeting gRNA was used to determine to background survival after transfection under the same conditions. FIG. 9B shows total cell count comparing the Alu gRNA in blue and the nonhuman gRNA in orange that was transfected with Cas9 GFP or no nuclease.

FIGS. 10A-10B show utilizing high copy repetitive elements for the testing of an extremely safe DNA editor. FIG. 10A shows the experimental design for two rounds of base editing at LINE-1. gRNA target is outlined in a dark box with a PAM outlined in a light-colored box. C->T deamination targets are colored in red. Top to bottom, left to right, the sequences in this figure correspond to SEQ ID NOs. 147-148. FIG. 10B shows targeted deamination frequency at C8 using nCBE4-gam over two rounds of transfection and clonal isolation. The top graph is a direct cell analysis of the same.

FIG. 11 shows base editing activity at HERV. Targeted deamination frequencies at C12 are shown using a set of Sa Cas9-BEs over three time points (day 1, 4 and 7). Error bars represent SEM, n=3.

FIGS. 12A-12D show base editing activities and purities of dBEs vs nBEs at a single locus target. FIG. 12A shows targeted deamination of C6 to T and associated indel frequencies using a set of CBEs and gRNA S1. Error bars represent sem, n=3. FIG. 12B shows targeted deamination of A5 to G and associated indel frequencies using ABEs. FIG. 12C shows base editing purity analysis of C6 using CBEs. FIG. 12D shows base editing purity analysis for A5 using ABEs.

FIG. 13 shows dABE targeting LINE-1 single cell analysis. Base editing in HEK 293T cells after transfection is shown, comparing nABE vs. dABE at days 2 and 14. FACS single cells are plotted as individual points representing targeted base editing nucleotide deamination. Red line indicates the median and the blue line the mean.

FIG. 14 shows base editing window comparing ABE vs. CBE and nCas9-BE vs dCas9-BE in the top edited live single cell isolated stable cell line.

FIG. 15 shows base editing purity of deamination and conversion of target cytosine nucleotides to thymine (T) using dCBE4-gam and nCBE4-gam (left); and purity of conversion of adenine nucleotides to guanine (G) using dABE and nABE (right) in HEK 293T targeting LINE-1.

FIG. 16 shows base editing efficiency across gRNA target sequence at day seven in HEK 293T using dCBE4-gam, nCBE4-gam, dABE, and nABE targeting LINE-1. HL1gR4 gRNA was used as a control.

FIG. 17 shows karyotype analysis after nCBE4-gam editing.

FIG. 18 shows karyotype analysis after nCBE4-gam editing.

FIGS. 19A-19B show that TE gRNAs are highly toxic in human iPSCs. FIG. 19A shows microscope images of PGP1 iPSCs transfected with pCas9_GFP and TE gRNAs_mCherry, or gRNA pairs. FIG. 19B shows the percentage GFP+ cells over time after transfection with TE or control gRNAs.

FIGS. 20A-20C show Annexin V and propidium iodide staining assays for cytotoxicity. FIG. 20A shows apoptosis cell death analysis using Annexin V targeting LINE-1. FIG. 20B shows necrosis cell death analysis using propidium iodide. FIG. 20C shows indel mutagenesis analysis from the previous experiment.

FIGS. 21A-21B show TE gRNA human reference alignment. FIG. 21A shows genome wide distribution of gRNA Alu (left) and Alu copy number and PAM distribution (right). FIG. 21B shows genome wide distribution of gRNA HERV env11 (left) and HERV copy number and PAM distribution (right).

FIGS. 22A-22D show LINE-1 RNA expression in knockout clones. FIG. 22A shows base editing activity detected in RNA transcripts of clone K (cK), clone K-A5 (cKA) and clone K-D4 (cKD5) within the gRNA target sequence. FIG. 22B shows the percentage of LINE-1 reads relative to total number of reads. Error bars represent standard deviation between biological duplicates. FIG. 22C shows a summary of differentially expressed genes as determined by the exact test. FIG. 22D shows a multidimensional scaling plot where distance corresponds to leading log-fold count changes between the RNA samples.

FIGS. 23A-23D show genome wide off-target analysis. FIG. 23A shows a whole genome sequencing analysis of the top edited 293T HL1gR4 edited clones from each of the four BEs tested. This displays the mutation spectrum observed for C*G>T*A mutations for each sample when compared to pre293T. Each represents a single clone. FIG. 23B shows the mutation spectrum observed for T*A>C*G mutations each sample when compared to pre293T. FIG. 23C shows on-target LINE-1 deamination for CBE clones and controls. FIG. 23D shows on-target LINE-1 deamination for ABE clones and controls

FIGS. 24A-24C show a genome wide off-target RNA analysis. FIG. 24A shows an RNA sequencing analysis compared to targeted LINE-1 amplicon sequencing for 293T cell transfected with BE and gRNA after two days. FIG. 24B shows an RNA-seq off target analysis displays the mutation spectrum observed for T*A>C*G mutations each sample.

FIG. 24C shows the C*G>T*A mutation spectrum of CBE edited clones after 30-70 days.

### DETAILED DESCRIPTION OF CERTAIN EMBODIMENTS OF THE INVENTION

The present disclosure provides for the multiplexed editing of nucleobases comprising the step of contacting of one or more complexes of a fusion protein and guide RNA with a nucleic acid molecule, e.g. a genomic DNA, while enhancing for survival of edited clones. Target sequences in the nucleic acid is modified are a manner that induces multiplexed single-base editing. Accordingly, some methods of the present disclosure are directed to editing target sequences using DNA binding proteins and guide RNAs described herein to provide multiplex genetic engineering of cells. Remarkably, the disclosed methods demonstrate low toxicity (e.g. low levels of apoptosis) in eukaryotic cells after concurrent editing of multiple loci (e.g. thousands of loci) per cell at high editing efficiencies.

Multiple nucleic acid sequences can be modified by one or more steps of introducing into a cell, which expresses a base editor fusion protein, and nucleic acids encoding a plurality of RNAs, such as by co-transformation, wherein the RNAs are expressed, and wherein each RNA in the plurality guides the fusion protein to a particular target site of the nucleic acid, and the enzyme modifies the nucleic acid. According to these aspects, many alterations or modification of the nucleic acid in the cell are created concurrently, or in a single iteration (or cycle). In certain aspects, cycling, or repeating of the step of contacting the nucleic acid with a complex of a DNA binding protein and guide RNA, results in multiplexed genetic modification of a cell at multiple loci, i.e., a cell having multiple genetic modifications. In certain embodiments, the nucleic acid is the genomic DNA of a eukaryotic cell. Related multiplexed base editing protocols are disclosed in International Publication Nos. WO 2017/062723, published on Apr. 13, 2017, and WO 2018/156824, published on Aug. 30, 2018; and U.S. Patent Publication No. 2016/0168592, published Jun. 16, 2016, each of which is herein incorporated by reference in its entirety.

LINE-1 sequences—which constitute about 17% of the genome—contain two open reading frames (ORFs): i) ORF-1 which binds the LINE-1 RNA and shuttles it back to the nucleus for retrotransposition, and ii) ORF-2 which functions as an endonuclease and reverse transcriptase. LINE-1 expression is largely suppressed in most somatic cells,²³ but can be highly active in disrupting gene expression in neurons.^{20,24} Researchers have explored the potential roles of LINE-1 sequences in neuronal diversity, brain development,^{18,25} and neurological diseases (e.g. ataxia telangiectasia¹⁹ and Rett syndrome²⁶). Even though the co-expression of LINE-1 elements and neural differentiation factors has been reported, it is still unclear whether such retrotransposons take advantage of a specific cell environment to replicate themselves, or whether LINE-1 is directly involved in these phenotypes. However, use of classical knock-out strategies to study such high copy number targets in mammalian genomes is not feasible due to the high toxicity of double-stranded breaks (DSBs).²⁷ This toxicity introduces additional challenges into the study of LINE-1 sequences.²⁸

Recently, the genome-wide knock-out of Porcine Endogenous RetroViruses (PERVs) in a transformed pig cell line at each of 62 transposable element loci was reported.³ The presence of multiple DSBs induced by this knock-out triggered apoptotic responses and limited the number of surviving, completely modified clones. Cells experiencing the most edits were likely depleted from the population via apoptosis. Thus, while a small number of clones with all PERV knockouts was isolated (8% of cells showed 60-100% PERV knockout rates), the overwhelming majority of surviving cells had less than 10% of PERV sequences edited. This genotoxicity-driven selective process raises concerns over the functionality of edited clones. Given the high expected toxicity of multiple DSBs, surviving clones might be expected to carry mutations that enable evasion of genotoxicity-driven apoptotic death, including through p53. Two years following this publication, a live pig was born with genome-wide knock-out of all 25 PERVs.¹⁶

### Conditions for Induction of Optimal Multiplexed Base Editing

The methods of the present disclosure can be applied to perform safer and more effective editing of higher copy number biological elements. In some embodiments, these methods comprise targeted viral genome multiplexed editing methods. Such methods include knock-outs of endogenous viruses, such as DNA viruses, HIV, HBV, Herpesviruses, and retroviruses, in the genome of a given eukaryotic cell. In particular embodiments, retroviruses (e.g. porcine ERVs and human ERVs) may be inactivated or destroyed via the methods disclosed herein. As shown with Cas9-targeting of HIV and HBV proviral genomes, the ability of viruses to rapidly evolve allows evasion of single-target approaches via mutations conferring resistance to modifications.^{52,53} However, effective multiplexed antiviral targeting can negate evasion at any single target site.

Aspects of the present invention are directed to the use of CRISPR/Cas9 for nucleic acid engineering. Specifically, the clustered regularly interspaced short palindromic repeats (CRISPR) and CRISPR associated genes (Cas genes), referred to herein as the CRISPR/Cas system, has been adapted as an efficient gene targeting technology, e.g. for multiplexed genome editing. Demonstrated herein is that CRISPR/Cas mediated gene editing allows the simultaneous inactivation of hundreds to tens of thousands of copies of an Alu, LINE-1, HERV-W, or HERV-K sequence with high efficiency. For instance, co-injection or transfection of Cas9 and guide RNA (gRNA) targeting HERVs into cells induced editing with an efficiency of up to 99%. Certain embodiments of the base editing methods described herein generate cells with inactivation of 1, 2, 3, 4, 5, or more HERV genes with an efficiency of between 20% and 100%, e.g. at least 20%, 30%, 40%, 50%, 60%, 70%, 80%, 85%, 90%, 95%, or more, e.g. up to 96%, 97%, 98%, 99%, or more.

Foremost, and generally, the methods involve the transfection into the target cell (i.e. the cell containing the genome to be edited) of nucleic acid constructs (e.g. plasmids) that each (or together) encode the components of the plurality of distinct complexes of fusion protein-gRNAs, wherein each gRNA comprises a distinct guide sequence that has complementarity to a distinct target sequence. The constructs are incorporated into the genome of the target cell, and copies of the fusion protein and gRNA are expressed. A nuclear localization sequence domain may be incorporated into the fusion protein to maximize localization of the fusion protein to the nucleus.

To induce base editing in the transfected target cell, the dCas9 domain of the fusion protein stimulates homologous recombination in the target cell. The guide RNA displaces the non-complementary strand and hybridizes with the target sequence. In this manner, a complex is formed between the dCas9 domain, a guide RNA and the target DNA. A double-stranded break is naturally introduced in the target DNA by the dCas9 domain. Once positioned next to a target cytosine (or adenine) of the target sequence, the deaminase domain hydrolytically deaminates the cytosine (or adenine) to uracil (or inosine). The products of this reaction form a mismatch with the base-paired guanine (or thymine) of the displaced, non-edited strand. The concerted action of the target cell's mismatch repair-associated proteins may convert the uracil (or inosine) lesion to a thymine (or guanine). The ultimate result of base editing is a conversion of cytosine to thymine (or of adenine to guanine).

It should be appreciated that any fusion protein, e.g. any of the fusion proteins provided herein, may be introduced into the cell in any suitable way, either stably or transiently. In some embodiments, a fusion protein may be transfected into the cell. In some embodiments, the cell may be transduced or transfected with a nucleic acid construct that encodes the fusion protein. For example, a cell may be transduced (e.g. with a virus encoding a fusion protein) or transfected (e.g. with a plasmid encoding a fusion protein) with a nucleic acid that encodes the fusion protein. Alternatively, a cell may be introduced with the fusion protein itself. Such transduction may be a stable or transient transduction. In some embodiments, cells expressing a base editing fusion protein, or comprising a fusion protein, may be transduced or transfected with one or more gRNA molecules, for example, when the fusion protein comprises a Cas9 (e.g. dCas9) domain. In some embodiments, a plasmid expressing a fusion protein may be introduced into cells through electroporation, transient (e.g. lipofection) or stable genome integration (e.g. piggybac), viral transduction, or other methods known to those of skill in the art.

In the disclosed methods, target cells may be incubated with the fusion protein-gRNA complexes for two days, or 48 hours, after transfection to achieve multiplexed base editing. Target cells may be incubated for 30 hours, 40 hours, 54 hours, 60 hours, or 72 hours after transfection. Target cells may be incubated with the fusion protein-gRNA complexes for four days, five days, seven days, nine days, eleven days, or thirteen days or more after transfection.

In the foregoing Examples, to identify suitable conditions for survival and editing efficiency, a direct single cell sequencing approach was conducted in parallel to live cell formation. Stem cells are particularly sensitive to DNA damage and require small molecule modifications to optimize transfection for genome editing.

In a particular embodiment, experimental conditions for preparing human induced plutipotent stem cells for transfection include: 1) harvesting cells at 80% confluency; 2) minimizing the volume of DNA/RNA/protein reactants delivered to below 10% of total electroporation volume; 3) mimizing time between harvesting cells and performing transfection; and 4) seeding cells into high confluency to promote survival of highly transfected cells. See S. M. Byrne & G. M. Church, Curr Protoc Stem Cell Biol, in press, herein incorporated by reference. In other embodiments, cells are harvested at 70% confluency, 75% confluency, 77% confluency, 82% confluency, or 85% confluency. Further, the volume of DNA/RNA/protein reactants delivered to the cells may comprise below 9%, below 8%, below 7%, below 6%, or below 5% of total electroporation volume.

By inducing base editing with the conditions disclosed, and supplementing post-transfection conditions with a combination of anti-aptoptotic molecules and growth factors as herein disclosed, target cells (e.g. hIPSCs) may exhibit multiplexed editing frequencies of 12%, 13%, 13.75%, 14%, 14.5% or greater. These frequencies correspond to ˜2200 to 3000 sites genome-wide, exceeding by three orders of magnitude the number of simultaneous edits previously recorded in iPSCs.³⁵

Beyond current standard practices in genome editing, implemented semi-automated delivery of genome editing reagents and harvesting with the RAININ E4 XLS set of programmable pipettes was implemented. To facilitate survival of stem cells after electroporation, CloneR media supplement may be combined with a small molecule p53 inhibitor (anti-apoptotic molecule) and growth factor, as described herein. Reference is made to https://www.stemcell.com/products/cloner.html#section-data-and-publications, incorporated herein by reference.

### Fusion Proteins to be Utilized in the Invention

Some aspects of the disclosure provide fusion proteins that include a DNA binding protein that is capable of binding to a specific target sequence of a nucleic acid (e.g. DNA). Such DNA binding proteins may be nucleic acid programmable DNA binding proteins, which bind to a target nucleic acid sequence via an oligonucleotide (e.g. guide RNA) that has complementarity thereto. Alternatively, the DNA binding protein may bind directly to a nucleic acid sequence without requiring an oligonucleotide-targeting molecule. For example, the DNA binding protein may comprise one or more TAL effectors, which recognize certain DNA sequences. In certain aspects, the DNA binding protein of the fusion proteins disclosed herein is a nuclease inactive dCas9 domain. In other aspects, the DNA binding protein is a Cas9 nickase, or nCas9. In particular embodiments, the Cas9 nickase (nCas9) domain is derived from S. pyogenes or S. aureus. In particular embodiments, the nCas9 comprises an amino acid sequence that is at least 80%, 85%, 90%, 95%, 98%, or 99% identical to SEQ ID NO: 19 or 101. In various other aspects, the DNA binding protein comprises, without limitation, CasX, CasY, Cpf1, C2c1, C2c2, C2C3, GeoCas9, CjCas9, Cas12a, Cas12b, Cas12c, Cas12d, Cas12g, Cas12h, Cas12i, Cas13d, Cas14, and Argonaute.

In still other aspects, the DNA binding protein domain of the fusion protein is a transcription activator-like (TAL) effector domain. In some aspects, the TAL effector domain is truncated at the N- and/or C-terminus. In particular embodiments, the disclosed fusion proteins comprising a TAL effector domain use one or more cofactor proteins (e.g. FokI endonucleases) that direct or otherwise program the protein to localize to a specific target DNA nucleotide sequence based on a recognition sequence in the DNA-binding domain of the cofactor protein. In some embodiments, the disclosed fusion proteins comprise a cofactor protein domain (e.g. FokI endonuclease domain)—i.e. a domain that is incorporated into the fusion protein itself. In other embodiments, the cofactor proteins may be added separately during the step of contacting the target sequence in the disclosed methods.

Use of dCas9 and nCas9 Domains

In various embodiments, the nuclease inactive Cas9 (dCas9) domain comprises the amino acid sequence provided in SEQ ID NO: 18. In various embodiments, the dCas9 comprises the amino acid sequence provided in SEQ ID NO: 100. In particular embodiments, the nuclease inactive Cas9 (dCas9) domain comprises an amino acid sequence that is at least 80%, 85%, 90%, 95%, 98%, or 99% identical to SEQ ID NO: 18 or SEQ ID NO: 100. In certain embodiments, the nuclease inactive Cas9 (dCas9) comprises the amino acid sequence of SEQ ID NO: 18 or SEQ ID NO: 100.

The dCas9 domain comprise an D10A and an H840A mutation in the amino acid sequence provided in SEQ ID NO: 20 (S. pyogenes wild-type Cas9), or the corresponding mutations D10A and N580A in the amino acid sequence provided in SEQ ID NO: 102 (S. aureus wild-type Cas9).

In other embodiments, the DNA binding domain comprises a Cas9 nickase domain. The Cas9 nickase (nCas9) domain may comprise the amino acid sequence provided in SEQ ID NOs: 19 or 101. In various embodiments, the nCas9 comprises the amino acid sequence provided in SEQ ID NO: 101. In particular embodiments, the nCas9 domain comprises an amino acid sequence that is at least 80%, 85%, 90%, 95%, 98%, or 99% identical to SEQ ID NO: 101. In certain embodiments, the nCas9 comprises the amino acid sequence of SEQ ID NO: 101.

Cytidine Base Editors

Fusion proteins useful for the methods disclosed herein include cytidine base editors (CBEs), in which the deaminase domain is a cytidine deaminase. In particular embodiments, the deaminase domain is an apolipoprotein B mRNA-editing complex 1 (APOBEC1) deaminase domain. In particular embodiments, a rat APOBEC1 (rAPOBEC1) is used. In other embodiments, a human APOBEC1 is used. Other cytidine deaminases, such as APOBEC2 deaminase, APOBEC3A deaminase, APOBEC3B deaminase, APOBEC3C deaminase, APOBEC3D deaminase, APOBEC3F deaminase, APOBEC3G deaminase, and APOBEC3H deaminase, an activation-induced deaminase (AID), a cytidine deaminase 1 from Petromyzon marinus (pmCDA1), an ACF1/ASE deaminase, or a variant thereof. Reference is made to U.S. Patent Publication No. 2017/0121693, published May 4, 2017, which issued as U.S. Pat. No. 10,167,457 on Jan. 1, 2019, U.S. Patent Publication No. 2015/0166980, published Jun. 18, 2015, and U.S. Pat. No. 9,840,699, issued Dec. 12, 2017, each of which are incorporated herein by reference.

The cytidine base editors utilized in the disclosed methods may further comprise an inhibitor of base excision repair (“iBER”) domain. In particular embodiments, the iBER domain may comprise a uracil glycosylase inhibitor (UGI) domain. In particular, the uracil glycosylase inhibitor domain prevents a U:G mismatch (or G:T mismatch) from being repaired back to the original C:G (or A:T) base pair. In some embodiments, the fusion protein comprises a catalytically inactive inosine-specific nuclease domain, such as a UGI domain. A UGI domain comprises an amino acid sequence that is at least 80%, 85%, 90%, 95%, 98%, 99%, 99.5%, or 99.9% identical to the amino acid sequence:

(SEQ ID NO: 5)
MTNLSDIIEKETGKQLVIQESILMLPEEVEEVIGNKPESDILVHTAYDE
STDENVMLLTSDAPEYKPWALVIQDSNGENKIKML.

Configurations of the cytidine base editors utilized in the methods disclosed herein may comprise dCas9 and/or UGI domains that comprise fusion proteins having the general structure NH₂-[dCas9]-[cytidine deaminase domain]-COOH, NH₂-[cytidine deaminase domain]-[dCas9]-COOH, NH₂-[dCas9]-[cytidine deaminase domain]-[uracil glycosylase inhibitor]-COOH, or NH₂-[cytidine deaminase domain]-[dCas9]-[uracil glycosylase inhibitor]-COOH; wherein each instance of “]-[” comprises an optional linker, e.g. a peptide linker.

Configurations of the cytidine base editors utilized in the methods disclosed herein may comprise nCas9 and/or UGI domains that comprise fusion proteins having the general structure NH₂-[nCas9]-[cytidine deaminase domain]-COOH, NH₂-[cytidine deaminase domain]-[nCas9]-COOH, NH₂-[nCas9]-[cytidine deaminase domain]-[uracil glycosylase inhibitor]-COOH, or NH₂-[cytidine deaminase domain]-[nCas9]-[uracil glycosylase inhibitor]-COOH; wherein each instance of “]-[” comprises an optional linker, e.g. a peptide linker.

The cytidine base editors (CBE) utilized in the disclosed methods may further comprise one, two, or more than two nuclear localization sequences (NLS). Configurations of such base editors (having a dCas9 domain) may comprise fusion proteins having the structure NH₂-[dCas9]-[cytidine deaminase domain]-[NLS]-COOH, NH₂-[dCas9]-[cytidine deaminase domain]-[NLS]-[NLS]-COOH, NH₂-[cytidine deaminase domain]-[dCas9]-[NLS]-COOH, NH₂-[cytidine deaminase domain]-[dCas9]-[NLS]-[NLS]-COOH, NH₂-[dCas9]-[cytidine deaminase domain]-[uracil glycosylase inhibitor]-[NLS]-COOH, NH₂-[dCas9]-[cytidine deaminase domain]-[uracil glycosylase inhibitor]-[NLS]-[NLS]-COOH, NH₂-[cytidine deaminase domain]-[dCas9]-[uracil glycosylase inhibitor]-[NLS]-COOH, NH₂-[cytidine deaminase domain]-[dCas9]-[uracil glycosylase inhibitor]-[NLS]-[NLS]-COOH; NH₂-[NLS]-[dCas9]-[cytidine deaminase domain]-COOH, NH₂-[NLS]-[dCas9]-[NLS]-[cytidine deaminase domain]-COOH, NH₂-[NLS]-[NLS]-[dCas9]-[cytidine deaminase domain]-COOH, NH₂-[NLS]-[cytidine deaminase domain]-[dCas9]-COOH, NH₂-[NLS]-[NLS]-[cytidine deaminase domain]-[dCas9]-COOH, or NH₂-[NLS]-[cytidine deaminase domain]-[NLS]-[dCas9]-COOH, wherein each instance of “]-[” comprises an optional linker, e.g. a peptide linker.

The cytidine base editors may further comprise a human influenza hemagglutinin (HA) tag at the C-terminus. In particular, a triple hemagglutinin (3×HA) tag may be utilized. The 3×HA tag may comprise the amino acid sequence

(SEQ ID NO: 104)
MEYPYDVPDYAAEYPYDVPDYAAEYPYDVPDYAAKLE.

Configurations of such base editors (e.g. those having a dCas9 domain) may comprise fusion proteins having the structure NH₂-[dCas9]-[cytidine deaminase domain]-[NLS]-[3×HA]-COOH, NH₂-[dCas9]-[cytidine deaminase domain]-[NLS]-[NLS]-3×HA]-COOH, NH₂-[cytidine deaminase domain]-[dCas9]-[NLS]-[3×HA]-COOH, NH₂-[cytidine deaminase domain]-[dCas9]-[NLS]-[NLS]-[3×HA]-COOH, NH₂-[dCas9]-[cytidine deaminase domain]-[uracil glycosylase inhibitor]-[NLS]-[3×HA]-COOH, NH₂-[dCas9]-[cytidine deaminase domain]-[uracil glycosylase inhibitor]-[NLS]-[NLS]-[3×HA]-COOH, NH₂-[cytidine deaminase domain]-[dCas9]-[uracil glycosylase inhibitor]-[NLS]-[3×HA]-COOH, NH₂-[cytidine deaminase domain]-[dCas9]-[uracil glycosylase inhibitor]-[NLS]-[NLS]-[3×HA]-COOH; NH₂-[NLS]-[dCas9]-[cytidine deaminase domain]-[3×HA]-COOH, NH₂-[NLS]-[dCas9]-[NLS]-[cytidine deaminase domain]-[NLS]-[NLS]-[3×HA]-COOH, NH₂-[NLS]-[NLS]-[dCas9]-[cytidine deaminase domain]-[3×HA]-COOH, NH₂-[NLS]-[cytidine deaminase domain]-[dCas9]-[3×HA]-COOH, NH₂-[NLS][NLS]-[cytidine deaminase domain]-[dCas9]-[3×HA]-COOH, or NH₂-[NLS]-[cytidine deaminase domain]-[NLS]-[dCas9]-[3×HA]-COOH, wherein each instance of “]-[” comprises an optional linker, e.g. a peptide linker.

Adenine Base Editors

Fusion proteins useful for the methods disclosed herein include adenine base editors (ABEs), in which the deaminase domain is a adenosine deaminase. In various embodiments, the adenosine deaminase domain comprises the amino acid sequence of SEQ ID NO: 1, 2 or 106.

In some embodiments, the adenosine deaminase is derived from a bacterium, such as, E. coli, S. aureus, S. typhi, S. putrefaciens, H. influenzae, or C. crescentus. In some embodiments, the adenosine deaminase is a TadA deaminase. In some embodiments, the TadA deaminase is an E. coli TadA deaminase (ecTadA). In some embodiments, the TadA deaminase is a truncated E. coli TadA deaminase. For example, the truncated ecTadA may be missing one or more N-terminal amino acids relative to a full-length ecTadA. In some embodiments, the truncated ecTadA may be missing 1, 2, 3, 4, 5, 6, 7, 8, 9, 10, 11, 12, 13, 14, 15, 6, 17, 18, 19, or 20 N-terminal amino acid residues relative to the full length ecTadA. In some embodiments, the truncated ecTadA may be missing 1, 2, 3, 4, 5, 6, 7, 8, 9, 10, 11, 12, 13, 14, 15, 6, 17, 18, 19, or 20 C-terminal amino acid residues relative to the full length ecTadA. In some embodiments, the ecTadA deaminase does not comprise an N-terminal methionine. Reference is made to U.S. Patent Publication No. 2018/0073012, published Mar. 15, 2018, which issued as U.S. Pat. No. 10,113,163, on Oct. 30, 2018, and U.S. Patent Publication No. 2015/0166980, published Jun. 18, 2015, each of which is incorporated herein by reference.

In some embodiments, the adenosine deaminase is an N-terminal truncated E. coli TadA (ecTadA). In certain embodiments, the adenosine deaminase comprises a sequence that has at least 85%, at least 90%, at least 95%, or at least 99% sequence identity to the following amino acid sequence:

(SEQ ID NO: 1)
MSEVEFSHEYWMRHALTLAKRAWDEREVPVGAVLVHNNRVIGEGWNRPI
GRHDPTAHAEIMALRQGGLVMQNYRLIDATLYVTLEPCVMCAGAMIHSR
IGRVVFGARDAKTGAAGSLMDVLHHPGMNHRVEITEGILADECAALLSD
FFRMRRQEIKAQKKAQSSTD.

In some embodiments, the adenosine deaminase is a full-length E. coli TadA (“ecTadA(wt)”) deaminase. For example, in certain embodiments, the adenosine deaminase comprises a sequence that has at least 85%, at least 90%, at least 95%, or at least 99% sequence identity to the following amino acid sequence:

(SEQ ID NO: 2)
MRRAFITGVFFLSEVEFSHEYWMRHALTLAKRAWDEREVPVGAVLVHNN
RVIGEGWNRPIGRHDPTAHAEIMALRQGGLVMQNYRLIDATLYVTLEPC
VMCAGAMIHSRIGRVVFGARDAKTGAAGSLMDVLHHPGMNHRVEITEGI
LADECAALLSDFFRMRRQEIKAQKKAQSSTD.

In some embodiments, the adenosine deaminase comprises a D108N mutation in SEQ ID NO: 1, or a corresponding mutation in a homologous or orthologous adenosine deaminase. In other embodiments, the adenosine deaminase further comprises an A106V mutation in SEQ ID NO: 1, or a corresponding mutation in a homologous or orthologous adenosine deaminase. Exemplary adenine base editors disclosed herein, such as ecTadA(D108N)-XTEN-dCas9, catalyze adenine deamination reactions in eukaryotic cells (e.g. HEK 293T mammalian cells). In certain examples, the fusion proteins disclosed herein have the general structure ecTadA*-XTEN-dCas9 (e.g. “ecTadA*(7.10)”), where ecTadA* represents an ecTadA variant comprising A106V and D108N mutations in the amino acid sequence of SEQ ID NO: 1. Thus, in some embodiments, the adenosine deaminase comprises the amino acid sequence (A106V and D108N mutations are underlined):

(SEQ ID NO: 105)
MSEVEFSHEYWMRHALTLAKRARDEREVPVGAVLVLNNRVIGEGWNRAI
GLHDPTAHAEIMALRQGGLVMQNYRLIDATLYVTFEPCVMCAGAMIHSR
IGRVVFGVRNAKTGAAGSLMDVLHYPGMNHRVEITEGILADECAALLCY
FFRMPRQVFNAQKKAQSSTD

Configurations of the adenine base editors utilized in the methods disclosed herein may comprise a dCas9 domain, and may comprise fusion proteins having the structure NH₂-[dCas9]-[adenine deaminase domain]-COOH, NH₂-[adenine deaminase domain]-[dCas9]-COOH, NH₂-[dCas9]-[adenine deaminase domain]-[NLS]-COOH, NH₂-[dCas9]-[adenine deaminase domain]-[NLS]-[NLS]-COOH, NH₂-[adenine deaminase domain]-[dCas9]-[NLS]-COOH, NH₂-[adenine deaminase domain]-[dCas9]-[NLS]-[NLS]-COOH, NH₂-[NLS]-[dCas9]-[adenine deaminase domain]-COOH, NH₂-[NLS]-[dCas9]-[NLS]-[adenine deaminase domain]-COOH, NH₂-[NLS]-[NLS]-[dCas9]-[adenine deaminase domain]-COOH, NH₂-[NLS]-[adenine deaminase domain]-[dCas9]-COOH, NH₂-[NLS]-[NLS]-[adenine deaminase domain]-[dCas9]-COOH, or NH₂-[NLS]-[adenine deaminase domain]-[NLS]-[dCas9]-COOH, wherein each instance of “]-[” comprises an optional linker, e.g. a peptide linker.

Configurations of the adenine base editors utilized in the methods disclosed herein may comprise an nCas9 domain, and may comprise fusion proteins having the structure NH₂-[nCas9]-[adenine deaminase domain]-COOH, NH₂-[adenine deaminase domain]-[nCas9]-COOH, NH₂-[nCas9]-[adenine deaminase domain]-[NLS]-COOH, NH₂-[nCas9]-[adenine deaminase domain]-[NLS]-[NLS]-COOH, NH₂-[adenine deaminase domain]-[nCas9]-[NLS]-COOH, NH₂-[adenine deaminase domain]-[nCas9]-[NLS]-[NLS]-COOH, NH₂-[NLS]-[nCas9]-[adenine deaminase domain]-COOH, NH₂-[NLS]-[nCas9]-[NLS]-[adenine deaminase domain]-COOH, NH₂-[NLS]-[NLS]-[nCas9]-[adenine deaminase domain]-COOH, NH₂-[NLS]-[adenine deaminase domain]-[nCas9]-COOH, NH₂-[NLS]-[NLS]-[adenine deaminase domain]-[nCas9]-COOH, or NH₂-[NLS]-[adenine deaminase domain]-[NLS]-[nCas9]-COOH, wherein each instance of “]-[” comprises an optional linker, e.g. a peptide linker.

The adenine base editors may further comprise a human influenza hemagglutinin (HA) tag at the C-terminus. In particular, a triple hemagglutinin (3×HA) tag may be utilized.

Configurations of such base editors (e.g. those having a dCas9 domain) may comprise fusion proteins having the structure NH₂-[dCas9]-[adenine deaminase domain]-[3×HA]-COOH, NH₂-[adenine deaminase domain]-[dCas9]-[3×HA]-COOH, NH₂-[dCas9]-[adenine deaminase domain]-[NLS]-[3×HA]-COOH, NH₂-[dCas9]-[adenine deaminase domain]-[NLS]-[NLS]-[3×HA]-COOH, NH₂-[adenine deaminase domain]-[dCas9]-[NLS]-[3×HA]-COOH, NH₂-[adenine deaminase domain]-[dCas9]-[NLS]-[NLS]-[3×HA]-COOH, NH₂-[NLS]-[dCas9]-[adenine deaminase domain]-[3×HA]-COOH, NH₂-[NLS]-[dCas9]-[NLS]-[adenine deaminase domain]-[3×HA]-COOH, NH₂-[NLS]-[NLS]-[dCas9]-[adenine deaminase domain]-[3×HA]-COOH, NH₂-[NLS]-[adenine deaminase domain]-[dCas9]-[3×HA]-COOH, NH₂-[NLS]-[NLS]-[adenine deaminase domain]-[dCas9]-[3×HA]-COOH, or NH₂-[NLS]-[adenine deaminase domain]-[NLS]-[dCas9]-[3×HA]-COOH, wherein each instance of “]-[” comprises an optional linker, e.g. a peptide linker.

Some aspects of the disclosure provide base editor fusion proteins comprising a dCas9 domain and a deaminase. Exemplary fusion proteins include, without limitation, the following fusion proteins:

AncBE4max:
(SEQ ID NO: 3)
MKRTADGSEFESPKKKRKVSSETGPVAVDPTLRRRIEPHEFEVFFDPRELR
KETCLLYEIKWGTSHKIWRHSSKNTTKHVEVNFIEKFTSERHFCPSTSCSITWFLSWSP
CGECSKAITEFLSQHPNVTLVIYVARLYHHMDQQNRQGLRDLVNSGVTIQIMTAPEYD
YCWRNFVNYPPGKEAHWPRYPPLWMKLYALELHAGILGLPPCLNILRRKQPQLTFFTI
ALQSCHYQRLPPHILWATGLKSGGSSGGSSGSETPGTSESATPESSGGSSGGSDKKYSI
GLDIGTNSVGWAVITDEYKVPSKKFKVLGNTDRHSIKKNLIGALLFDSGETAEATRLK
RTARRRYTRRKNRICYLQEIFSNEMAKVDDSFFHRLEESFLVEEDKKHERHPIFGNIVD
EVAYHEKYPTIYHLRKKLVDSTDKADLRLIYLALAHMIKFRGHFLIEGDLNPDNSDVD
KLFIQLVQTYNQLFEENPINTASGVDAKAILSARLSKSRRLENLIAQLPGEKKNGLFGN
LIALSLGLTPNFKSNFDLAEDAKLQLSKDTYDDDLDNLLAQIGDQYADLFLAAKNLS
DAILLSDILRVNTEITKAPLSASMIKRYDEHHQDLTLLKALVRQQLPEKYKEIFFDQSK
NGYAGYIDGGASQEEFYKFIKPILEKMDGTEELLVKLNREDLLRKQRTFDNGSIPHQI
HLGELHAILRRQEDFYPFLKDNREKIEKILTFRIPYYVGPLARGNSRFAWMTRKSEETI
TPWNFEEVVDKGASAQSFIERMTNFDKNLPNEKVLPKHSLLYEYFTVYNELTKVKYV
TEGMRKPAFLSGEQKKAIVDLLFKTNRKVTVKQLKEDYFKKIECFDSVETSGVEDRF
NASLGTYHDLLKIIKDKDFLDNEENEDILEDIVLTLTLFEDREMIEERLKTYAHLFDDK
VMKQLKRRRYTGWGRLSRKLINGIRDKQSGKTILDFLKSDGFANRNFMQLIHDDSLT
FKEDIQKAQVSGQGDSLHEHIANLAGSPAIKKGILQTVKVVDELVKVMGRHKPENIVI
EMARENQTTQKGQKNSRERMKRIEEGIKELGSQILKEHPVENTQLQNEKLYLYYLQN
GRDMYVDQELDINRLSDYDVDHIVPQSFLKDDSIDNKVLTRSDKNRGKSDNVPSEEV
VKKMKNYWRQLLNAKLITQRKFDNLTKAERGGLSELDKAGFIKRQLVETRQITKHV
AQILDSRMNTKYDENDKLIREVKVITLKSKLVSDFRKDFQFYKVREINNYHHAHDAY
LNAVVGTALIKKYPKLESEFVYGDYKVYDVRKMIAKSEQEIGKATAKYFFYSNIMNF
FKTEITLANGEIRKRPLIETNGETGEIVWDKGRDFATVRKVLSMPQVNIVKKTEVQTG
GFSKESILPKRNSDKLIARKKDWDPKKYGGFDSPTVAYSVLVVAKVEKGKSKKLKSV
KELLGITIMERSSFEKNPIDFLEAKGYKEVKKDLIIKLPKYSLFELENGRKRMLASAGE
LQKGNELALPSKYVNFLYLASHYEKLKGSPEDNEQKQLFVEQHKHYLDEIIEQISEFS
KRVILADANLDKVLSAYNKHRDKPIREQAENIIHLFTLTNLGAPAAFKYFDTTIDRKR
YTSTKEVLDATLIHQSITGLYETRIDLSQLGGDSGGSGGSGGSTNLSDIIEKETGKQLVI
QESILMLPEEVEEVIGNKPESDILVHTAYDESTDENVMLLTSDAPEYKPWALVIQDSNG
ENKIKMLSGGSGGSGGSTNLSDIIEKETGKQLVIQESILMLPEEVEEVIGNKPESDILVH
TAYDESTDENVMLLTSDAPEYKPWALVIQDSNGENKIKMLSGGSKRTADGSEFEPKK
KRKV
ABEmax:
(SEQ ID NO: 4)
MKRTADGSEFESPKKKRKVSEVEFSHEYWMRHALTLAKRAWDEREVPV
GAVLVHNNRVIGEGWNRPIGRHDPTAHAEIMALRQGGLVMQNYRLIDATLYVTLEPC
VMCAGAMIHSRIGRVVFGARDAKTGAAGSLMDVLHHPGMNHRVEITEGILADECAA
LLSDFFRMRRQEIKAQKKAQSSTDSGGSSGGSSGSETPGTSESATPESSGGSSGGSSEV
EFSHEYWMRHALTLAKRARDEREVPVGAVLVLNNRVIGEGWNRAIGLHDPTAHAEI
MALRQGGLVMQNYRLIDATLYVTFEPCVMCAGAMIHSRIGRVVFGVRNAKTGAAGS
LMDVLHYPGMNHRVEITEGILADECAALLCYFFRMPRQVFNAQKKAQSSTDSGGSS
GGSSGSETPGTSESATPESSGGSSGGSDKKYSIGLAIGTNSVGWAVITDEYKVPSKKFK
VLGNTDRHSIKKNLIGALLFDSGETAEATRLKRTARRRYTRRKNRICYLQEMSNEMA
KVDDSFFHRLEESFLVEEDKKHERHPIFGNIVDEVAYHEKYPTIYHLRKKLVDSTDKA
DLRLIYLALAHMIKFRGHFLIEGDLNPDNSDVDKLFIQLVQTYNQLFEENPINASGVD
AKAILSARLSKSRRLENLIAQLPGEKKNGLFGNLIALSLGLTPNFKSNFDLAEDAKLQ
LSKDTYDDDLDNLLAQIGDQYADLFLAAKNLSDAILLSDILRVNTEITKAPLSASMIK
RYDEHHQDLTLLKALVRQQLPEKYKEIFFDQSKNGYAGYIDGGASQEEFYKFIKPILE
KMDGTEELLVKLNREDLLRKQRTFDNGSIPHQIHLGELHAILRRQEDFYPFLKDNREK
IEKILTFRIPYYVGPLARGNSRFAWMTRKSEETITPWNFEEVVDKGASAQSFIERMTNF
DKNLPNEKVLPKHSLLYEYFTVYNELTKVKYVTEGMRKPAFLSGEQKKAIVDLLFKT
NRKVTVKQLKEDYFKKIECFDSVEISGVEDRFNASLGTYHDLLKIIKDKDFLDNEENE
DILEDIVLTLTLFEDREMIEERLKTYAHLFDDKVMKQLKRRRYTGWGRLSRKLINGIR
DKQSGKTILDFLKSDGFANRNFMQLIHDDSLTFKEDIQKAQVSGQGDSLHEHIANLA
GSPAIKKGILQTVKVVDELVKVMGRHKPENIVIEMARENQTTQKGQKNSRERMKRIE
EGIKELGSQILKEHPVENTQLQNEKLYLYYLQNGRDMYVDQELDINRLSDYDVDHIV
PQSFLKDDSIDNKVLTRSDKNRGKSDNVPSEEVVKKMKNYWRQLLNAKLITQRKFD
NLTKAERGGLSELDKAGFIKRQLVETRQITKHVAQILDSRMNTKYDENDKLIREVKVI
TLKSKLVSDFRKDFQFYKVREINNYHHAHDAYLNAVVGTALIKKYPKLESEFVYGDY
KVYDVRKMIAKSEQEIGKATAKYFFYSNIMNFFKTEITLANGEIRKRPLIETNGETGEI
VWDKGRDFATVRKVLSMPQVNIVKKTEVQTGGFSKESILPKRNSDKLIARKKDWDP
KKYGGFDSPTVAYSVLVVAKVEKGKSKKLKSVKELLGITIMERSSFEKNPIDFLEAKG
YKEVKKDLIIKLPKYSLFELENGRKRMLASAGELQKGNELALPSKYVNFLYLASHYE
KLKGSPEDNEQKQLFVEQHKHYLDEIIEQISEFSKRVILADANLDKVLSAYNKHRDKP
IREQAENIIHLFTLTNLGAPAAFKYFDTTIDRKRYTSTKEVLDATLIHQSITGLYETRIDL
SQLGGDSGGSKRTADGSEFEPKKKRKV
AncBE4max-P2A-GFP:
(SEQ ID NO: 6)
MKRTADGSEFESPKKKRKVSSETGPVAVDPTLRRRIEPHEFEVFFDPRELR
KETCLLYEIKWGTSHKIWRHSSKNTTKHVEVNFIEKFTSERHFCPSTSCSITWFLSWSP
CGECSKAITEFLSQHPNVTLVIYVARLYHHMDQQNRQGLRDLVNSGVTIQIMTAPEYD
YCWRNFVNYPPGKEAHWPRYPPLWMKLYALELHAGILGLPPCLNILRRKQPQLTFFTI
ALQSCHYQRLPPHILWATGLKSGGSSGGSSGSETPGTSESATPESSGGSSGGSDKKYSI
GLDIGTNSVGWAVITDEYKVPSKKFKVLGNTDRHSIKKNLIGALLFDSGETAEATRLK
RTARRRYTRRKNRICYLQEIFSNEMAKVDDSFFHRLEESFLVEEDKKHERHPIFGNIVD
EVAYHEKYPTIYHLRKKLVDSTDKADLRLIYLALAHMIKFRGHFLIEGDLNPDNSDVD
KLFIQLVQTYNQLFEENPINASGVDAKAILSARLSKSRRLENLIAQLPGEKKNGLFGN
LIALSLGLTPNFKSNFDLAEDAKLQLSKDTYDDDLDNLLAQIGDQYADLFLAAKNLS
DAILLSDILRVNTEITKAPLSASMIKRYDEHHQDLTLLKALVRQQLPEKYKEIFFDQSK
NGYAGYIDGGASQEEFYKFIKPILEKMDGTEELLVKLNREDLLRKQRTFDNGSIPHQI
HLGELHAILRRQEDFYPFLKDNREKIEKILTFRIPYYVGPLARGNSRFAWMTRKSEETI
TPWNFEEVVDKGASAQSFIERMTNFDKNLPNEKVLPKHSLLYEYFTVYNELTKVKYV
TEGMRKPAFLSGEQKKAIVDLLFKTNRKVTVKQLKEDYFKKIECFDSVEISGVEDRF
NASLGTYHDLLKIIKDKDFLDNEENEDILEDIVLTLTLFEDREMIEERLKTYAHLFDDK
VMKQLKRRRYTGWGRLSRKLINGIRDKQSGKTILDFLKSDGFANRNFMQLIHDDSLT
FKEDIQKAQVSGQGDSLHEHIANLAGSPAIKKGILQTVKVVDELVKVMGRHKPENIVI
EMARENQTTQKGQKNSRERMKRIEEGIKELGSQILKEHPVENTQLQNEKLYLYYLQN
GRDMYVDQELDINRLSDYDVDHIVPQSFLKDDSIDNKVLTRSDKNRGKSDNVPSEEV
VKKMKNYWRQLLNAKLITQRKFDNLTKAERGGLSELDKAGFIKRQLVETRQITKHV
AQILDSRMNTKYDENDKLIREVKVITLKSKLVSDFRKDFQFYKVREINNYHHAHDAY
LNAVVGTALIKKYPKLESEFVYGDYKVYDVRKMIAKSEQEIGKATAKYFFYSNIMNF
FKTEITLANGEIRKRPLIETNGETGEIVWDKGRDFATVRKVLSMPQVNIVKKTEVQTG
GFSKESILPKRNSDKLIARKKDWDPKKYGGFDSPTVAYSVLVVAKVEKGKSKKLKSV
KELLGITIMERSSFEKNPIDFLEAKGYKEVKKDLIIKLPKYSLFELENGRKRMLASAGE
LQKGNELALPSKYVNFLYLASHYEKLKGSPEDNEQKQLFVEQHKHYLDEIIEQISEFS
KRVILADANLDKVLSAYNKHRDKPIREQAENIIHLFTLTNLGAPAAFKYFDTTIDRKR
YTSTKEVLDATLIHQSITGLYETRIDLSQLGGDSGGSGGSGGSTNLSDIIEKETGKQLVI
QESILMLPEEVEEVIGNKPESDILVHTAYDESTDENVMLLTSDAPEYKPWALVIQDSNG
ENKIKMLSGGSGGSGGSTNLSDIIEKETGKQLVIQESILMLPEEVEEVIGNKPESDILVH
TAYDESTDENVMLLTSDAPEYKPWALVIQDSNGENKIKMLSGGSKRTADGSEFEPKK
KRKVSGGSPKKKRKVGSGATNFSLLKQAGDVEENPGPMVSKGEELFTGVVPILVELD
GDVNGHKFSVSGEGEGDATYGKLTLKFICTTGKLPVPWPTLVTTLTYGVQCFSRYPD
HMKQHDFFKSAMPEGYVQERTIFFKDDGNYKTRAEVKFEGDTLVNRIELKGIDFKED
GNILGHKLEYNYNSHNVYIMADKQKNGIKVNFKIRHNIEDGSVQLADHYQQNTPIGD
GPVLLPDNHYLSTQSALSKDPNEKRDHMVLLEFVTAAGITLGMDELYKSGGSPKKKR
KV
ABEmax-P2A-GFP:
(SEQ ID NO: 7)
MKRTADGSEFESPKKKRKVSEVEFSHEYWMRHALTLAKRAWDEREVPV
GAVLVHNNRVIGEGWNRPIGRHDPTAHAEIMALRQGGLVMQNYRLIDATLYVTLEPC
VMCAGAMIHSRIGRVVFGARDAKTGAAGSLMDVLHHPGMNHRVEITEGILADECAA
LLSDFFRMRRQEIKAQKKAQSSTDSGGSSGGSSGSETPGTSESATPESSGGSSGGSSEV
EFSHEYWMRHALTLAKRARDEREVPVGAVLVLNNRVIGEGWNRAIGLHDPTAHAEI
MALRQGGLVMQNYRLIDATLYVTFEPCVMCAGAMIHSRIGRVVFGVRNAKTGAAGS
LMDVLHYPGMNHRVEITEGILADECAALLCYFFRMPRQVFNAQKKAQSSTDSGGSS
GGSSGSETPGTSESATPESSGGSSGGSDKKYSIGLAIGTNSVGWAVITDEYKVPSKKFK
VLGNTDRHSIKKNLIGALLFDSGETAEATRLKRTARRRYTRRKNRICYLQEMSNEMA
KVDDSFFHRLEESFLVEEDKKHERHPIFGNIVDEVAYHEKYPTIYHLRKKLVDSTDKA
DLRLIYLALAHMIKFRGHFLIEGDLNPDNSDVDKLFIQLVQTYNQLFEENPINASGVD
AKAILSARLSKSRRLENLIAQLPGEKKNGLFGNLIALSLGLTPNFKSNFDLAEDAKLQ
LSKDTYDDDLDNLLAQIGDQYADLFLAAKNLSDAILLSDILRVNTEITKAPLSASMIK
RYDEHHQDLTLLKALVRQQLPEKYKEIFFDQSKNGYAGYIDGGASQEEFYKFIKPILE
KMDGTEELLVKLNREDLLRKQRTFDNGSIPHQIHLGELHAILRRQEDFYPFLKDNREK
IEKILTFRIPYYVGPLARGNSRFAWMTRKSEETITPWNFEEVVDKGASAQSFIERMTNF
DKNLPNEKVLPKHSLLYEYFTVYNELTKVKYVTEGMRKPAFLSGEQKKAIVDLLFKT
NRKVTVKQLKEDYFKKIECFDSVEISGVEDRFNASLGTYHDLLKIIKDKDFLDNEENE
DILEDIVLTLTLFEDREMIEERLKTYAHLFDDKVMKQLKRRRYTGWGRLSRKLINGIR
DKQSGKTILDFLKSDGFANRNFMQLIHDDSLTFKEDIQKAQVSGQGDSLHEHIANLA
GSPAIKKGILQTVKVVDELVKVMGRHKPENIVIEMARENQTTQKGQKNSRERMKRIE
EGIKELGSQILKEHPVENTQLQNEKLYLYYLQNGRDMYVDQELDINRLSDYDVDHIV
PQSFLKDDSIDNKVLTRSDKNRGKSDNVPSEEVVKKMKNYWRQLLNAKLITQRKFD
NLTKAERGGLSELDKAGFIKRQLVETRQITKHVAQILDSRMNTKYDENDKLIREVKVI
TLKSKLVSDFRKDFQFYKVREINNYHHAHDAYLNAVVGTALIKKYPKLESEFVYGDY
KVYDVRKMIAKSEQEIGKATAKYFFYSNIMNFFKTEITLANGEIRKRPLIETNGETGEI
VWDKGRDFATVRKVLSMPQVNIVKKTEVQTGGFSKESILPKRNSDKLIARKKDWDP
KKYGGFDSPTVAYSVLVVAKVEKGKSKKLKSVKELLGITIMERSSFEKNPIDFLEAKG
YKEVKKDLIIKLPKYSLFELENGRKRMLASAGELQKGNELALPSKYVNFLYLASHYE
KLKGSPEDNEQKQLFVEQHKHYLDEIIEQISEFSKRVILADANLDKVLSAYNKHRDKP
IREQAENIIHLFTLTNLGAPAAFKYFDTTIDRKRYTSTKEVLDATLIHQSITGLYETRIDL
SQLGGDSGGSKRTADGSEFEPKKKRKVSGGSPKKKRKVGSGATNFSLLKQAGDVEE
NPGPMVSKGEELFTGVVPILVELDGDVNGHKFSVSGEGEGDATYGKLTLKFICTTGK
LPVPWPTLVTTLTYGVQCFSRYPDHMKQHDFFKSAMPEGYVQERTIFFKDDGNYKTR
AEVKFEGDTLVNRIELKGIDFKEDGNILGHKLEYNYNSHNVYIMADKQKNGIKVNFK
IRHNIEDGSVQLADHYQQNTPIGDGPVLLPDNHYLSTQSALSKDPNEKRDHMVLLEF
VTAAGITLGMDELYKSGGSPKKKRKV
CBE1 (rAPOBEC1-XTEN-dCas9-NLS)
(SEQ ID NO: 8)
MSSETGPVAVDPTLRRRIEPHEFEVFFDPRELRKETCLLYEINWGGRHSIW
RHTSQNTNKHVEVNFIEKFTTERYFCPNTRCSITWFLSWSPCGECSRAITEFLSRYPHV
TLFIYIARLYHHADPRNRQGLRDLISSGVTIQIMTEQESGYCWRNFVNYSPSNEAHWP
RYPHLWVRLYVLELYCIILGLPPCLNILRRKQPQLTFFTIALQSCHYQRLPPHILWATGL
KSGSETPGTSESATPESDKKYSIGLAIGTNSVGWAVITDEYKVPSKKFKVLGNTDRHSI
KKNLIGALLFDSGETAEATRLKRTARRRYTRRKNRICYLQEIFSNEMAKVDDSFFHRL
EESFLVEEDKKHERHPIFGNIVDEVAYHEKYPTIYHLRKKLVDSTDKADLRLIYLALA
HMIKFRGHFLIEGDLNPDNSDVDKLFIQLVQTYNQLFEENPINASGVDAKAILSARLS
KSRRLENLIAQLPGEKKNGLFGNLIALSLGLTPNFKSNFDLAEDAKLQLSKDTYDDDL
DNLLAQIGDQYADLFLAAKNLSDAILLSDILRVNTEITKAPLSASMIKRYDEHHQDLTL
LKALVRQQLPEKYKEIFFDQSKNGYAGYIDGGASQEEFYKFIKPILEKMDGTEELLVK
LNREDLLRKQRTFDNGSIPHQIHLGELHAILRRQEDFYPFLKDNREKIEKILTFRIPYYV
GPLARGNSRFAWMTRKSEETITPWNFEEVVDKGASAQSFIERMTNFDKNLPNEKVLP
KHSLLYEYFTVYNELTKVKYVTEGMRKPAFLSGEQKKAIVDLLFKTNRKVTVKQLK
EDYFKKIECFDSVETSGVEDRFNASLGTYHDLLKIIKDKDFLDNEENEDILEDIVLTLTL
FEDREMIEERLKTYAHLFDDKVMKQLKRRRYTGWGRLSRKLINGIRDKQSGKTILDF
LKSDGFANRNFMQLIHDDSLTFKEDIQKAQVSGQGDSLHEHIANLAGSPAIKKGILQT
VKVVDELVKVMGRHKPENIVIEMARENQTTQKGQKNSRERMKRIEEGIKELGSQILK
EHPVENTQLQNEKLYLYYLQNGRDMYVDQELDINRLSDYDVDAIVPQSFLKDDSIDN
KVLTRSDKNRGKSDNVPSEEVVKKMKNYWRQLLNAKLITQRKFDNLTKAERGGLSE
LDKAGFIKRQLVETRQITKHVAQILDSRMNTKYDENDKLIREVKVITLKSKLVSDFRK
DFQFYKVREINNYHHAHDAYLNAVVGTALIKKYPKLESEFVYGDYKVYDVRKMIAK
SEQEIGKATAKYFFYSNIMNFFKTEITLANGEIRKRPLIETNGETGEIVWDKGRDFATV
RKVLSMPQVNIVKKTEVQTGGFSKESILPKRNSDKLIARKKDWDPKKYGGFDSPTVA
YSVLVVAKVEKGKSKKLKSVKELLGITIMERSSFEKNPIDFLEAKGYKEVKKDLIIKLP
KYSLFELENGRKRMLASAGELQKGNELALPSKYVNFLYLASHYEKLKGSPEDNEQK
QLFVEQHKHYLDEIIEQISEFSKRVILADANLDKVLSAYNKHRDKPIREQAENIIHLFTL
TNLGAPAAFKYFDTTIDRKRYTSTKEVLDATLIHQSITGLYETRIDLSQLGGDSGGSSG
GSPKKKRKV
CBE2 (rAPOBEC1-XTEN-dCas9-UGI-NLS)
(SEQ ID NO: 9)
MSSETGPVAVDPTLRRRIEPHEFEVFFDPRELRKETCLLYEINWGGRHSIW
RHTSQNTNKHVEVNFIEKFTTERYFCPNTRCSITWFLSWSPCGECSRAITEFLSRYPHV
TLFIYIARLYHHADPRNRQGLRDLISSGVTIQIMTEQESGYCWRNFVNYSPSNEAHWP
RYPHLWVRLYVLELYCIILGLPPCLNILRRKQPQLTFFTIALQSCHYQRLPPHILWATGL
KSGSETPGTSESATPESDKKYSIGLAIGTNSVGWAVITDEYKVPSKKFKVLGNTDRHSI
KKNLIGALLFDSGETAEATRLKRTARRRYTRRKNRICYLQEIFSNEMAKVDDSFFHRL
EESFLVEEDKKHERHPIFGNIVDEVAYHEKYPTIYHLRKKLVDSTDKADLRLIYLALA
HMIKFRGHFLIEGDLNPDNSDVDKLFIQLVQTYNQLFEENPINASGVDAKAILSARLS
KSRRLENLIAQLPGEKKNGLFGNLIALSLGLTPNFKSNFDLAEDAKLQLSKDTYDDDL
DNLLAQIGDQYADLFLAAKNLSDAILLSDILRVNTEITKAPLSASMIKRYDEHHQDLTL
LKALVRQQLPEKYKEIFFDQSKNGYAGYIDGGASQEEFYKFIKPILEKMDGTEELLVK
LNREDLLRKQRTFDNGS1PHQIHLGELHAILRRQEDFYPFLKDNREKIEKILTFR1PYYV
GPLARGNSRFAWMTRKSEETITPWNFEEVVDKGASAQSFIERMTNFDKNLPNEKVLP
KHSLLYEYFTVYNELTKVKYVTEGMRKPAFLSGEQKKAIVDLLFKTNRKVTVKQLK
EDYFKKIECFDSVETSGVEDRFNASLGTYHDLLKIIKDKDFLDNEENEDILEDIVLTLTL
FEDREMIEERLKTYAHLFDDKVMKQLKRRRYTGWGRLSRKLINGIRDKQSGKTILDF
LKSDGFANRNFMQLIHDDSLTFKEDIQKAQVSGQGDSLHEHIANLAGSPAIKKGILQT
VKVVDELVKVMGRHKPENIVIEMARENQTTQKGQKNSRERMKRIEEGIKELGSQILK
EHPVENTQLQNEKLYLYYLQNGRDMYVDQELDINRLSDYDVDAIVPQSFLKDDSIDN
KVLTRSDKNRGKSDNVPSEEVVKKMKNYWRQLLNAKLITQRKFDNLTKAERGGLSE
LDKAGFIKRQLVETRQITKHVAQILDSRMNTKYDENDKLIREVKVITLKSKLVSDFRK
DFQFYKVREINNYHHAHDAYLNAVVGTALIKKYPKLESEFVYGDYKVYDVRKMIAK
SEQEIGKATAKYFFYSNIMNFFKTEITLANGEIRKRPLIETNGETGEIVWDKGRDFATV
RKVLSMPQVNIVKKTEVQTGGFSKESILPKRNSDKLIARKKDWDPKKYGGFDSPTVA
YSVLVVAKVEKGKSKKLKSVKELLGITIMERSSFEKNPIDFLEAKGYKEVKKDLIIKLP
KYSLFELENGRKRMLASAGELQKGNELALPSKYVNFLYLASHYEKLKGSPEDNEQK
QLFVEQHKHYLDEIIEQISEFSKRVILADANLDKVLSAYNKHRDKPIREQAENIIHLFTL
TNLGAPAAFKYFDTTIDRKRYTSTKEVLDATLIHQSITGLYETRIDLSQLGGDSGGSTN
LSDIIEKETGKQLVIQESILMLPEEVEEVIGNKPESDILVHTAYDESTDENVMLLTSDAP
EYKPWALVIQDSNGENKIKMLSGGSPKKKRKV
CBE3 (rAPOBEC1-XTEN-nCas9-UGI-NLS)
(SEQ ID NO: 10)
MSSETGPVAVDPTLRRRIEPHEFEVFFDPRELRKETCLLYEINWGGRHSIW
RHTSQNTNKHVEVNFIEKFTTERYFCPNTRCSITWFLSWSPCGECSRAITEFLSRYPHV
TLFIYIARLYHHADPRNRQGLRDLISSGVTIQIMTEQESGYCWRNFVNYSPSNEAHWP
RYPHLWVRLYVLELYCIILGLPPCLNILRRKQPQLTFFTIALQSCHYQRLPPHILWATGL
KSGSETPGTSESATPESDKKYSIGLAIGTNSVGWAVITDEYKVPSKKFKVLGNTDRHSI
KKNLIGALLFDSGETAEATRLKRTARRRYTRRKNRICYLQEIFSNEMAKVDDSFFHRL
EESFLVEEDKKHERHPIFGNIVDEVAYHEKYPTIYHLRKKLVDSTDKADLRLIYLALA
HMIKFRGHFLIEGDLNPDNSDVDKLFIQLVQTYNQLFEENPINASGVDAKAILSARLS
KSRRLENLIAQLPGEKKNGLFGNLIALSLGLTPNFKSNFDLAEDAKLQLSKDTYDDDL
DNLLAQIGDQYADLFLAAKNLSDAILLSDILRVNTEITKAPLSASMIKRYDEHHQDLTL
LKALVRQQLPEKYKEIFFDQSKNGYAGYIDGGASQEEFYKFIKPILEKMDGTEELLVK
LNREDLLRKQRTFDNGS1PHQIHLGELHAILRRQEDFYPFLKDNREKIEKILTFR1PYYV
GPLARGNSRFAWMTRKSEETITPWNFEEVVDKGASAQSFIERMTNFDKNLPNEKVLP
KHSLLYEYFTVYNELTKVKYVTEGMRKPAFLSGEQKKAIVDLLFKTNRKVTVKQLK
EDYFKKIECFDSVETSGVEDRFNASLGTYHDLLKIIKDKDFLDNEENEDILEDIVLTLTL
FEDREMIEERLKTYAHLFDDKVMKQLKRRRYTGWGRLSRKLINGIRDKQSGKTILDF
LKSDGFANRNFMQLIHDDSLTFKEDIQKAQVSGQGDSLHEHIANLAGSPAIKKGILQT
VKVVDELVKVMGRHKPENIVIEMARENQTTQKGQKNSRERMKRIEEGIKELGSQILK
EHPVENTQLQNEKLYLYYLQNGRDMYVDQELDINRLSDYDVDHIVPQSFLKDDSIDN
KVLTRSDKNRGKSDNVPSEEVVKKMKNYWRQLLNAKLITQRKFDNLTKAERGGLSE
LDKAGFIKRQLVETRQITKHVAQILDSRMNTKYDENDKLIREVKVITLKSKLVSDFRK
DFQFYKVREINNYHHAHDAYLNAVVGTALIKKYPKLESEFVYGDYKVYDVRKMIAK
SEQEIGKATAKYFFYSNIMNFFKTEITLANGEIRKRPLIETNGETGEIVWDKGRDFATV
RKVLSMPQVNIVKKTEVQTGGFSKESILPKRNSDKLIARKKDWDPKKYGGFDSPTVA
YSVLVVAKVEKGKSKKLKSVKELLGITIMERSSFEKNPIDFLEAKGYKEVKKDLIIKLP
KYSLFELENGRKRMLASAGELQKGNELALPSKYVNFLYLASHYEKLKGSPEDNEQK
QLFVEQHKHYLDEIIEQISEFSKRVILADANLDKVLSAYNKHRDKPIREQAENIIHLFTL
TNLGAPAAFKYFDTTIDRKRYTSTKEVLDATLIHQSITGLYETRIDLSQLGGDSGGSTN
LSDIIEKETGKQLVIQESILMLPEEVEEVIGNKPESDILVHTAYDESTDENVMLLTSDAP
EYKPWALVIQDSNGENKIKMLSGGSPKKKRKV
CBE4:
(SEQ ID NO: 11)
MSSETGPVAVDPTLRRRIEPHEFEVFFDPRELRKETCLLYEINWGGRHSIW
RHTSQNTNKHVEVNFIEKFTTERYFCPNTRCSITWFLSWSPCGECSRAITEFLSRYPHV
TLFIYIARLYHHADPRNRQGLRDLISSGVTIQIMTEQESGYCWRNFVNYSPSNEAHWP
RYPHLWVRLYVLELYCIILGLPPCLNILRRKQPQLTFFTIALQSCHYQRLPPHILWATGL
KSGGSSGGSSGSETPGTSESATPESSGGSSGGSDKKYSIGLAIGTNSVGWAVITDEYKV
PSKKFKVLGNTDRHSIKKNLIGALLFDSGETAEATRLKRTARRRYTRRKNRICYLQEIF
SNEMAKVDDSFFHRLEESFLVEEDKKHERHPIFGNIVDEVAYHEKYPTIYHLRKKLVD
STDKADLRLIYLALAHMIKFRGHFLIEGDLNPDNSDVDKLFIQLVQTYNQLFEENPIN
ASGVDAKAILSARLSKSRRLENLIAQLPGEKKNGLFGNLIALSLGLTPNFKSNFDLAE
DAKLQLSKDTYDDDLDNLLAQIGDQYADLFLAAKNLSDAILLSDILRVNTEITKAPLS
ASMIKRYDEHHQDLTLLKALVRQQLPEKYKEIFFDQSKNGYAGYIDGGASQEEFYKFI
KPILEKMDGTEELLVKLNREDLLRKQRTFDNGSIPHQIHLGELHAILRRQEDFYPFLK
DNREKIEKILTFRIPYYVGPLARGNSRFAWMTRKSEETITPWNFEEVVDKGASAQSFIE
RMTNFDKNLPNEKVLPKHSLLYEYFTVYNELTKVKYVTEGMRKPAFLSGEQKKAIV
DLLFKTNRKVTVKQLKEDYFKKIECFDSVEISGVEDRFNASLGTYHDLLKIIKDKDFL
DNEENEDILEDIVLTLTLFEDREMIEERLKTYAHLFDDKVMKQLKRRRYTGWGRLSR
KLINGIRDKQSGKTILDFLKSDGFANRNFMQLIHDDSLTFKEDIQKAQVSGQGDSLHE
HIANLAGSPAIKKGILQTVKVVDELVKVMGRHKPENIVIEMARENQTTQKGQKNSRE
RMKRIEEGIKELGSQILKEHPVENTQLQNEKLYLYYLQNGRDMYVDQELDINRLSDY
DVDHIVPQSFLKDDSIDNKVLTRSDKNRGKSDNVPSEEVVKKMKNYWRQLLNAKLI
TQRKFDNLTKAERGGLSELDKAGFIKRQLVETRQIIKHVAQILDSRMNTKYDENDKLI
REVKVITLKSKLVSDFRKDFQFYKVREINNYHHAHDAYLNAVVGTALIKKYPKLESEF
VYGDYKVYDVRKMIAKSEQEIGKATAKYFFYSNIMNFFKTEITLANGEIRKRPLIETN
GETGEIVWDKGRDFATVRKVLSMPQVNIVKKTEVQTGGFSKESILPKRNSDKLIARK
KDWDPKKYGGFDSPTVAYSVLVVAKVEKGKSKKLKSVKELLGITIMERSSFEKNPIDF
LEAKGYKEVKKDLIIKLPKYSLFELENGRKRMLASAGELQKGNELALPSKYVNFLYL
ASHYEKLKGSPEDNEQKQLFVEQHKHYLDEIIEQISEFSKRVILADANLDKVLSAYNK
HRDKPIREQAENIIHLFTLTNLGAPAAFKYFDTTIDRKRYTSTKEVLDATLIHQSITGLY
ETRIDLSQLGGDSGGSGGSGGSTNLSDIIEKETGKQLVIQESILMLPEEVEEVIGNKPES
DILVHTAYDESTDENVMLLTSDAPEYKPWALVIQDSNGENKIKMLSGGSGGSGGSTN
LSDIIEKETGKQLVIQESILMLPEEVEEVIGNKPESDILVHTAYDESTDENVMLLTSDAP
EYKPWALVIQDSNGENKIKMLSGGSPKKKRK
CBE4-Gam:
(SEQ ID NO: 12)
AKPAKRIKSAAAAYVPQNRDAVITDIKRIGDLQREASRLETEMNDAIAEIT
EKFAARIAPIKTDIETLSKGVQGWCEANRDELTNGGKVKTANLVTGDVSWRVRPPSV
SIRGMDAVMETLERLGLQRFIRTKQEINKEAILLEPKAVAGVAGITVKSGIEDFSIIPFEQ
EAGISGSETPGTSESATPESSSETGPVAVDPTLRRRIEPHEFEVFFDPRELRKETCLLYEI
NWGGRHSIWRHTSQNTNKHVEVNFIEKFTTERYFCPNTRCSITWFLSWSPCGECSRAI
TEFLSRYPHVTLFIYIARLYHHADPRNRQGLRDLISSGVTIQIMTEQESGYCWRNFVN
YSPSNEAHWPRYPHLWVRLYVLELYCIILGLPPCLNILRRKQPQLTFFTIALQSCHYQR
LPPHILWATGLKSGGSSGGSSGSETPGTSESATPESSGGSSGGSDKKYSIGLAIGTNSVG
WAVITDEYKVPSKKFKVLGNTDRHSIKKNLIGALLFDSGETAEATRLKRTARRRYTRR
KNRICYLQEIFSNEMAKVDDSFFHRLEESFLVEEDKKHERHPIFGNIVDEVAYHEKYPT
IYHLRKKLVDSTDKADLRLIYLALAHMIKFRGHFLIEGDLNPDNSDVDKLFIQLVQTY
NQLFEENPINASGVDAKAILSARLSKSRRLENLIAQLPGEKKNGLFGNLIALSLGLTPN
FKSNFDLAEDAKLQLSKDTYDDDLDNLLAQIGDQYADLFLAAKNLSDAILLSDILRV
NTEITKAPLSASMIKRYDEHHQDLTLLKALVRQQLPEKYKEIFFDQSKNGYAGYIDGG
ASQEEFYKFIKPILEKMDGTEELLVKLNREDLLRKQRTFDNGSIPHQIHLGELHAILRR
QEDFYPFLKDNREKIEKILTFRIPYYVGPLARGNSRFAWMTRKSEETITPWNFEEVVD
KGASAQSFIERMTNFDKNLPNEKVLPKHSLLYEYFTVYNELTKVKYVTEGMRKPAFL
SGEQKKAIVDLLFKTNRKVTVKQLKEDYFKKIECFDSVEISGVEDRFNASLGTYHDL
LKIIKDKDFLDNEENEDILEDIVLTLTLFEDREMIEERLKTYAHLFDDKVMKQLKRRR
YTGWGRLSRKLINGIRDKQSGKTILDFLKSDGFANRNFMQLIHDDSLTFKEDIQKAQV
SGQGDSLHEHIANLAGSPAIKKGILQTVKVVDELVKVMGRHKPENIVIEMARENQTT
QKGQKNSRERMKRIEEGIKELGSQILKEHPVENTQLQNEKLYLYYLQNGRDMYVDQ
ELDINRLSDYDVDHIVPQSFLKDDSIDNKVLTRSDKNRGKSDNVPSEEVVKKMKNY
WRQLLNAKLITQRKFDNLTKAERGGLSELDKAGFIKRQLVETRQITKHVAQILDSRM
NTKYDENDKLIREVKVITLKSKLVSDFRKDFQFYKVREINNYHHAHDAYLNAVVGTA
LIKKYPKLESEFVYGDYKVYDVRKMIAKSEQEIGKATAKYFFYSNIMNFFKTEITLAN
GEIRKRPLIETNGETGEIVWDKGRDFATVRKVLSMPQVNIVKKTEVQTGGFSKESILP
KRNSDKLIARKKDWDPKKYGGFDSPTVAYSVLVVAKVEKGKSKKLKSVKELLGITIM
ERSSFEKNPIDFLEAKGYKEVKKDLIIKLPKYSLFELENGRKRMLASAGELQKGNELA
LPSKYVNFLYLASHYEKLKGSPEDNEQKQLFVEQHKHYLDEIIEQISEFSKRVILADAN
LDKVLSAYNKHRDKPIREQAENIIHLFTLTNLGAPAAFKYFDTTIDRKRYTSTKEVLD
ATLIHQSITGLYETRIDLSQLGGDSGGSGGSGGSTNLSDIIEKETGKQLVIQESILMLPEE
VEEVIGNKPESDILVHTAYDESTDENVMLLTSDAPEYKPWALVIQDSNGENKIKMLSG
GSGGSGGSTNLSDIIEKETGKQLVIQESILMLPEEVEEVIGNKPESDILVHTAYDESTDE
NVMLLTSDAPEYKPWALVIQDSNGENKIKMLSGGSPKKKRK
S. aureus CBE4:
(SEQ ID NO: 13)
MSSETGPVAVDPTLRRRIEPHEFEVFFDPRELRKETCLLYEINWGGRHSIW
RHTSQNTNKHVEVNFIEKFTTERYFCPNTRCSITWFLSWSPCGECSRAITEFLSRYPHV
TLFIYIARLYHHADPRNRQGLRDLISSGVTIQIMTEQESGYCWRNFVNYSPSNEAHWP
RYPHLWVRLYVLELYCIILGLPPCLNILRRKQPQLTFFTIALQSCHYQRLPPHILWATGL
KSGGSSGGSSGSETPGTSESATPESSGGSSGGSGKRNYILGLAIGITSVGYGIIDYETRD
VIDAGVRLFKEANVENNEGRRSKRGARRLKRRRRHRIQRVKKLLFDYNLLTDHSELS
GINPYEARVKGLSQKLSEEEFSAALLHLAKRRGVHNVNEVEEDTGNELSTKEQISRNS
KALEEKYVAELQLERLKKDGEVRGSINRFKTSDYVKEAKQLLKVQKAYHQLDQSFID
TYIDLLETRRTYYEGPGEGSPFGWKDIKEWYEMLMGHCTYFPEELRSVKYAYNADL
YNALNDLNNLVITRDENEKLEYYEKFQIIENVFKQKKKPTLKQIAKEILVNEEDIKGY
RVTSTGKPEFTNLKVYHDIKDITARKEIIENAELLDQIAKILTIYQSSEDIQEELTNLNSE
LTQEEIEQISNLKGYTGTHNLSLKAINLILDELWHTNDNQIAIFNRLKLVPKKVDLSQQ
KEIPTTLVDDFILSPVVKRSFIQSIKVINAIIKKYGLPNDIIIELAREKNSKDAQKMINEM
QKRNRQTNERIEEIIRTTGKENAKYLIEKIKLHDMQEGKCLYSLEAIPLEDLLNNPFNY
EVDHIIPRSVSFDNSFNNKVLVKQEENSKKGNRTPFQYLSSSDSKISYETFKKHILNLA
KGKGRISKTKKEYLLEERDINRFSVQKDFINRNLVDTRYATRGLMNLLRSYFRVNNLD
VKVKSINGGFTSFLRRKWKFKKERNKGYKHHAEDALIIANADFIFKEWKKLDKAKK
VMENQMFEEKQAESMPEIETEQEYKEIFITPHQIKHIKDFKDYKYSHRVDKKPNRELI
NDTLYSTRKDDKGNTLIVNNLNGLYDKDNDKLKKLINKSPEKLLMYHHDPQTYQKL
KLIMEQYGDEKNPLYKYYEETGNYLTKYSKKDNGPVIKKIKYYGNKLNAHLDITDD
YPNSRNKVVKLSLKPYRFDVYLDNGVYKFVTVKNLDVIKKENYYEVNSKCYEEAK
KLKKISNQAEFIASFYNNDLIKINGELYRVIGVNNDLLNRIEVNMIDITYREYLENMND
KRPPRIIKTIASKTQSIKKYSTDILGNLYEVKSKKHPQIIKKGGSPKKKRKVSSDYKDH
DGDYKDHDIDYKDDDDKSGGSGGSGGSTNLSDIIEKETGKQLVIQESILMLPEEVEEV
IGNKPESDILVHTAYDESTDENVMLLTSDAPEYKPWALVIQDSNGENKIKMLSGGSGG
SGGSTNLSDIIEKETGKQLVIQESILMLPEEVEEVIGNKPESDILVHTAYDESTDENVML
LTSDAPEYKPWALVIQDSNGENKIKMLSGGSPKKKRKV
S. aureus BE4-Gam:
(SEQ ID NO: 14)
MAKPAKRIKSAAAAYVPQNRDAVITDIKRIGDLQREASRLETEMNDAIAEI
TEKFAARIAPIKTDIETLSKGVQGWCEANRDELTNGGKVKTANLVTGDVSWRVRPPS
VSIRGMDAVMETLERLGLQRFIRTKQEINKEAILLEPKAVAGVAGITVKSGIEDFSIIPFE
QEAGISGSETPGTSESATPESSSETGPVAVDPTLRRRIEPHEFEVFFDPRELRKETCLLYE
INWGGRHSIWRHTSQNTNKHVEVNFIEKFTTERYFCPNTRCSITWFLSWSPCGECSRA
ITEFLSRYPHVTLFIYIARLYHHADPRNRQGLRDLISSGVTIQIMTEQESGYCWRNFVN
YSPSNEAHWPRYPHLWVRLYVLELYCIILGLPPCLNILRRKQPQLTFFTIALQSCHYQR
LPPHILWATGLKSGGSSGGSSGSETPGTSESATPESSGGSSGGSGKRNYILGLAIGITSV
GYGIIDYETRDVIDAGVRLFKEANVENNEGRRSKRGARRLKRRRRHRIQRVKKLLFD
YNLLTDHSELSGINPYEARVKGLSQKLSEEEFSAALLHLAKRRGVHNVNEVEEDTGN
ELSTKEQISRNSKALEEKYVAELQLERLKKDGEVRGSINRFKTSDYVKEAKQLLKVQ
KAYHQLDQSFIDTYIDLLETRRTYYEGPGEGSPFGWKDIKEWYEMLMGHCTYFPEEL
RSVKYAYNADLYNALNDLNNLVITRDENEKLEYYEKFQIIENVFKQKKKPTLKQIAKE
ILVNEEDIKGYRVTSTGKPEFTNLKVYHDIKDITARKEIIENAELLDQIAKILTIYQSSED
IQEELTNLNSELTQEEIEQISNLKGYTGTHNLSLKAINLILDELWHTNDNQIAIFNRLKL
VPKKVDLSQQKEIPTTLVDDFILSPVVKRSFIQSIKVINAIIKKYGLPNDIIIELAREKNS
KDAQKMINEMQKRNRQTNERIEEIIRTTGKENAKYLIEKIKLHDMQEGKCLYSLEAIP
LEDLLNNPFNYEVDHIIPRSVSFDNSFNNKVLVKQEENSKKGNRTPFQYLSSSDSKISY
ETFKKHILNLAKGKGRISKTKKEYLLEERDINRFSVQKDFINRNLVDTRYATRGLMNL
LRSYFRVNNLDVKVKSINIGGFTSFLRRKWKFKKERNKGYKHHAEDALIIANADFIFK
EWKKLDKAKKVMENQMFEEKQAESMPEIETEQEYKEIFITPHQIKHIKDFKDYKYSH
RVDKKPNRELINDTLYSTRKDDKGNTLIVNNLNGLYDKDNDKLKKLINKSPEKLLMY
HHDPQTYQKLKLIMEQYGDEKNPLYKYYEETGNYLTKYSKKDNGPVIKKIKYYGNK
LNAHLDITDDYPNSRNKVVKLSLKPYRFDVYLDNGVYKFVTVKNLDVIKKENYYEV
NSKCYEEAKKLKKISNQAEFIASFYNNDLIKINGELYRVIGVNNDLLNRIEVNMIDITY
REYLENMNDKRPPRIIKTIASKTQSIKKYSTDILGNLYEVKSKKHPQIIKKGGSPKKKR
KVSSDYKDHDGDYKDHDIDYKDDDDKSGGSGGSGGSTNLSDIIEKETGKQLVIQESI
LMLPEEVEEVIGNKPESDILVHTAYDESTDENVMLLTSDAPEYKPWALVIQDSNGENK
IKMLSGGSGGSGGSTNLSDIIEKETGKQLVIQESILMLPEEVEEVIGNKPESDILVHTAY
DESTDENVMLLTSDAPEYKPWALVIQDSNGENKIKMLSGGSPKKKRKV
nABE7.10 (ecTadA(wt)-linker(32aa)-ecTadA*(7.10)-linker(32aa)-nCas9-NLS):
(SEQ ID NO: 15)
MSEVEFSHEYWMRHALTLAKRAWDEREVPVGAVLVHNNRVIGEGWNRPI
GRHDPTAHAEIMALRQGGLVMQNYRLIDATLYVTLEPCVMCAGAMIHSRIGRVVFG
ARDAKTGAAGSLMDVLHHPGMNHRVEITEGILADECAALLSDFFRMRRQEIKAQKK
AQSSTDSGGSSGGSSGSETPGTSESATPESSGGSSGGSSEVEFSHEYWMRHALTLAKR
ARDEREVPVGAVLVLNNRVIGEGWNRAIGLHDPTAHAEIMALRQGGLVMQNYRLID
ATLYVTFEPCVMCAGAMIHSRIGRVVFGVRNAKTGAAGSLMDVLHYPGMNHRVEIT
EGILADECAALLCYFFRMPRQVFNAQKKAQSSTDSGGSSGGSSGSETPGTSESATPES
SGGSSGGSDKKYSIGLAIGTNSVGWAVITDEYKVPSKKFKVLGNTDRHSIKKNLIGAL
LFDSGETAEATRLKRTARRRYTRRKNRICYLQEIFSNEMAKVDDSFFHRLEESFLVEED
KKHERHPIFGNIVDEVAYHEKYPTIYHLRKKLVDSTDKADLRLIYLALAHMIKFRGHF
LIEGDLNPDNSDVDKLFIQLVQTYNQLFEENPINTASGVDAKAILSARLSKSRRLENLIA
QLPGEKKNGLFGNLIALSLGLTPNFKSNFDLAEDAKLQLSKDTYDDDLDNLLAQIGD
QYADLFLAAKNLSDAILLSDILRVNTEITKAPLSASMIKRYDEHHQDLTLLKALVRQQ
LPEKYKEIFFDQSKNGYAGYIDGGASQEEFYKFIKPILEKMDGTEELLVKLNREDLLR
KQRTFDNGSIPHQIHLGELHAILRRQEDFYPFLKDNREKIEKILTFRIPYYVGPLARGNS
RFAWMTRKSEETITPWNFEEVVDKGASAQSFIERMTNFDKNLPNEKVLPKHSLLYEY
FTVYNELTKVKYVTEGMRKPAFLSGEQKKAIVDLLFKTNRKVTVKQLKEDYFKKIEC
FDSVETSGVEDRFNASLGTYHDLLKIIKDKDFLDNEENEDILEDIVLTLTLFEDREMIEE
RLKTYAHLFDDKVMKQLKRRRYTGWGRLSRKLINGIRDKQSGKTILDFLKSDGFANR
NFMQLIHDDSLTFKEDIQKAQVSGQGDSLHEHIANLAGSPAIKKGILQTVKVVDELVK
VMGRHKPENIVIEMARENQTTQKGQKNSRERMKRIEEGIKELGSQILKEHPVENTQL
QNEKLYLYYLQNGRDMYVDQELDINRLSDYDVDHIVPQSFLKDDSIDNKVLTRSDKN
RGKSDNVPSEEVVKKMKNYWRQLLNAKLITQRKFDNLTKAERGGLSELDKAGFIKR
QLVETRQITKHVAQILDSRMNTKYDENDKLIREVKVITLKSKLVSDFRKDFQFYKVRE
INNYHHAHDAYLNAVVGTALIKKYPKLESEFVYGDYKVYDVRKMIAKSEQEIGKATA
KYFFYSNIMNFFKTEITLANGEIRKRPLIETNGETGEIVWDKGRDFATVRKVLSMPQV
NIVKKTEVQTGGFSKESILPKRNSDKLIARKKDWDPKKYGGFDSPTVAYSVLVVAKV
EKGKSKKLKSVKELLGITIMERSSFEKNPIDFLEAKGYKEVKKDLIIKLPKYSLFELEN
GRKRMLASAGELQKGNELALPSKYVNFLYLASHYEKLKGSPEDNEQKQLFVEQHKH
YLDEIIEQISEFSKRVILADANLDKVLSAYNKHRDKPIREQAENIIHLFTLTNLGAPAAF
KYFDTTIDRKRYTSTKEVLDATLIHQSITGLYETRIDLSQLGGDSGGSPKKKRKV
dABE7.10 (ecTadA(wt)-linker(32aa)-ecTadA*(7.10)-linker(32aa)-nCas9-NLS):
(SEQ ID NO: 106)
MSEVEFSHEYWMRHALTLAKRAWDEREVPVGAVLVHNNRVIGEGWNRPI
GRHDPTAHAEIMALRQGGLVMQNYRLIDATLYVTLEPCVMCAGAMIHSRIGRVVFG
ARDAKTGAAGSLMDVLHHPGMNHRVEITEGILADECAALLSDFFRMRRQEIKAQKK
AQSSTDSGGSSGGSSGSETPGTSESATPESSGGSSGGSSEVEFSHEYWMRHALTLAKR
ARDEREVPVGAVLVLNNRVIGEGWNRAIGLHDPTAHAEIMALRQGGLVMQNYRLID
ATLYVTFEPCVMCAGAMIHSRIGRVVFGVRNAKTGAAGSLMDVLHYPGMNHRVEIT
EGILADECAALLCYFFRMPRQVFNAQKKAQSSTDSGGSSGGSSGSETPGTSESATPES
SGGSSGGSDKKYSIGLAIGTNSVGWAVITDEYKVPSKKFKVLGNTDRHSIKKNLIGAL
LFDSGETAEATRLKRTARRRYTRRKNRICYLQEIFSNEMAKVDDSFFHRLEESFLVEED
KKHERHPIFGNIVDEVAYHEKYPTIYHLRKKLVDSTDKADLRLIYLALAHMIKFRGHF
LIEGDLNPDNSDVDKLFIQLVQTYNQLFEENPINTASGVDAKAILSARLSKSRRLENLIA
QLPGEKKNGLFGNLIALSLGLTPNFKSNFDLAEDAKLQLSKDTYDDDLDNLLAQIGD
QYADLFLAAKNLSDAILLSDILRVNTEITKAPLSASMIKRYDEHHQDLTLLKALVRQQ
LPEKYKEIFFDQSKNGYAGYIDGGASQEEFYKFIKPILEKMDGTEELLVKLNREDLLR
KQRTFDNGSIPHQIHLGELHAILRRQEDFYPFLKDNREKIEKILTFRIPYYVGPLARGNS
RFAWMTRKSEETITPWNFEEVVDKGASAQSFIERMTNFDKNLPNEKVLPKHSLLYEY
FTVYNELTKVKYVTEGMRKPAFLSGEQKKAIVDLLFKTNRKVTVKQLKEDYFKKIEC
FDSVETSGVEDRFNASLGTYHDLLKIIKDKDFLDNEENEDILEDIVLTLTLFEDREMIEE
RLKTYAHLFDDKVMKQLKRRRYTGWGRLSRKLINGIRDKQSGKTILDFLKSDGFANR
NFMQLIHDDSLTFKEDIQKAQVSGQGDSLHEHIANLAGSPAIKKGILQTVKVVDELVK
VMGRHKPENIVIEMARENQTTQKGQKNSRERMKRIEEGIKELGSQILKEHPVENTQL
QNEKLYLYYLQNGRDMYVDQELDINRLSDYDVDAIVPQSFLKDDSIDNKVLTRSDKN
RGKSDNVPSEEVVKKMKNYWRQLLNAKLITQRKFDNLTKAERGGLSELDKAGFIKR
QLVETRQITKHVAQILDSRMNTKYDENDKLIREVKVITLKSKLVSDFRKDFQFYKVRE
INNYHHAHDAYLNAVVGTALIKKYPKLESEFVYGDYKVYDVRKMIAKSEQEIGKATA
KYFFYSNIMNFFKTEITLANGEIRKRPLIETNGETGEIVWDKGRDFATVRKVLSMPQV
NIVKKTEVQTGGFSKESILPKRNSDKLIARKKDWDPKKYGGFDSPTVAYSVLVVAKV
EKGKSKKLKSVKELLGITIMERSSFEKNPIDFLEAKGYKEVKKDLIIKLPKYSLFELEN
GRKRMLASAGELQKGNELALPSKYVNFLYLASHYEKLKGSPEDNEQKQLFVEQHKH
YLDEIIEQISEFSKRVILADANLDKVLSAYNKHRDKPIREQAENIIHLFTLTNLGAPAAF
KYFDTTIDRKRYTSTKEVLDATLIHQSITGLYETRIDLSQLGGDSGGSPKKKRKV
dCBE4:
(SEQ ID NO: 107)
MSSETGPVAVDPTLRRRIEPHEFEVFFDPRELRKETCLLYEINWGGRHSIW
RHTSQNTNKHVEVNFIEKFTTERYFCPNTRCSITWFLSWSPCGECSRAITEFLSRYPHV
TLFIYIARLYHHADPRNRQGLRDLISSGVTIQIMTEQESGYCWRNFVNYSPSNEAHWP
RYPHLWVRLYVLELYCIILGLPPCLNILRRKQPQLTFFTIALQSCHYQRLPPHILWATGL
KSGGSSGGSSGSETPGTSESATPESSGGSSGGSDKKYSIGLAIGTNSVGWAVITDEYKV
PSKKFKVLGNTDRHSIKKNLIGALLFDSGETAEATRLKRTARRRYTRRKNRICYLQEIF
SNEMAKVDDSFFHRLEESFLVEEDKKHERHPIFGNIVDEVAYHEKYPTIYHLRKKLVD
STDKADLRLIYLALAHMIKFRGHFLIEGDLNPDNSDVDKLFIQLVQTYNQLFEENPIN
ASGVDAKAILSARLSKSRRLENLIAQLPGEKKNGLFGNLIALSLGLTPNFKSNFDLAE
DAKLQLSKDTYDDDLDNLLAQIGDQYADLFLAAKNLSDAILLSDILRVNTEITKAPLS
ASMIKRYDEHHQDLTLLKALVRQQLPEKYKEIFFDQSKNGYAGYIDGGASQEEFYKFI
KPILEKMDGTEELLVKLNREDLLRKQRTFDNGSIPHQIHLGELHAILRRQEDFYPFLK
DNREKIEKILTFRIPYYVGPLARGNSRFAWMTRKSEETITPWNFEEVVDKGASAQSFIE
RMTNFDKNLPNEKVLPKHSLLYEYFTVYNELTKVKYVTEGMRKPAFLSGEQKKAIV
DLLFKTNRKVTVKQLKEDYFKKIECFDSVEISGVEDRFNASLGTYHDLLKIIKDKDFL
DNEENEDILEDIVLTLTLFEDREMIEERLKTYAHLFDDKVMKQLKRRRYTGWGRLSR
KLINGIRDKQSGKTILDFLKSDGFANRNFMQLIHDDSLTFKEDIQKAQVSGQGDSLHE
HIANLAGSPAIKKGILQTVKVVDELVKVMGRHKPENIVIEMARENQTTQKGQKNSRE
RMKRIEEGIKELGSQILKEHPVENTQLQNEKLYLYYLQNGRDMYVDQELDINRLSDY
DVDAIVPQSFLKDDSIDNKVLTRSDKNRGKSDNVPSEEVVKKMKNYWRQLLNAKLI
TQRKFDNLTKAERGGLSELDKAGFIKRQLVETRQIIKHVAQILDSRMNTKYDENDKLI
REVKVITLKSKLVSDFRKDFQFYKVREINNYHHAHDAYLNAVVGTALIKKYPKLESEF
VYGDYKVYDVRKMIAKSEQEIGKATAKYFFYSNIMNFFKTEITLANGEIRKRPLIETN
GETGEIVWDKGRDFATVRKVLSMPQVNIVKKTEVQTGGFSKESILPKRNSDKLIARK
KDWDPKKYGGFDSPTVAYSVLVVAKVEKGKSKKLKSVKELLGITIMERSSFEKNPIDF
LEAKGYKEVKKDLIIKLPKYSLFELENGRKRMLASAGELQKGNELALPSKYVNFLYL
ASHYEKLKGSPEDNEQKQLFVEQHKHYLDEIIEQISEFSKRVILADANLDKVLSAYNK
HRDKPIREQAENIIHLFTLTNLGAPAAFKYFDTTIDRKRYTSTKEVLDATLIHQSITGLY
ETRIDLSQLGGDSGGSGGSGGSTNLSDIIEKETGKQLVIQESILMLPEEVEEVIGNKPES
DILVHTAYDESTDENVMLLTSDAPEYKPWALVIQDSNGENKIKMLSGGSGGSGGSTN
LSDIIEKETGKQLVIQESILMLPEEVEEVIGNKPESDILVHTAYDESTDENVMLLTSDAP
EYKPWALVIQDSNGENKIKMLSGGSPKKKRK
dCBE4-Gam:
(SEQ ID NO: 108)
AKPAKRIKSAAAAYVPQNRDAVITDIKRIGDLQREASRLETEMNDAIAEIT
EKFAARIAPIKTDIETLSKGVQGWCEANRDELTNGGKVKTANLVTGDVSWRVRPPSV
SIRGMDAVMETLERLGLQRFIRTKQEINKEAILLEPKAVAGVAGITVKSGIEDFSIIPFEQ
EAGISGSETPGTSESATPESSSETGPVAVDPTLRRRIEPHEFEVFFDPRELRKETCLLYEI
NWGGRHSIWRHTSQNTNKHVEVNFIEKFTTERYFCPNTRCSITWFLSWSPCGECSRAI
TEFLSRYPHVTLFIYIARLYHHADPRNRQGLRDLISSGVTIQIMTEQESGYCWRNFVN
YSPSNEAHWPRYPHLWVRLYVLELYCIILGLPPCLNILRRKQPQLTFFTIALQSCHYQR
LPPHILWATGLKSGGSSGGSSGSETPGTSESATPESSGGSSGGSDKKYSIGLAIGTNSVG
WAVITDEYKVPSKKFKVLGNTDRHSIKKNLIGALLFDSGETAEATRLKRTARRRYTRR
KNRICYLQEIFSNEMAKVDDSFFHRLEESFLVEEDKKHERHPIFGNIVDEVAYHEKYPT
IYHLRKKLVDSTDKADLRLIYLALAHMIKFRGHFLIEGDLNPDNSDVDKLFIQLVQTY
NQLFEENPINASGVDAKAILSARLSKSRRLENLIAQLPGEKKNGLFGNLIALSLGLTPN
FKSNFDLAEDAKLQLSKDTYDDDLDNLLAQIGDQYADLFLAAKNLSDAILLSDILRV
NTEITKAPLSASMIKRYDEHHQDLTLLKALVRQQLPEKYKEIFFDQSKNGYAGYIDGG
ASQEEFYKFIKPILEKMDGTEELLVKLNREDLLRKQRTFDNGSIPHQIHLGELHAILRR
QEDFYPFLKDNREKIEKILTFRIPYYVGPLARGNSRFAWMTRKSEETITPWNFEEVVD
KGASAQSFIERMTNFDKNLPNEKVLPKHSLLYEYFTVYNELTKVKYVTEGMRKPAFL
SGEQKKAIVDLLFKTNRKVTVKQLKEDYFKKIECFDSVEISGVEDRFNASLGTYHDL
LKIIKDKDFLDNEENEDILEDIVLTLTLFEDREMIEERLKTYAHLFDDKVMKQLKRRR
YTGWGRLSRKLINGIRDKQSGKTILDFLKSDGFANRNFMQLIHDDSLTFKEDIQKAQV
SGQGDSLHEHIANLAGSPAIKKGILQTVKVVDELVKVMGRHKPENIVIEMARENQTT
QKGQKNSRERMKRIEEGIKELGSQILKEHPVENTQLQNEKLYLYYLQNGRDMYVDQ
ELDINRLSDYDVDAIVPQSFLKDDSIDNKVLTRSDKNRGKSDNVPSEEVVKKMKNY
WRQLLNAKLITQRKFDNLTKAERGGLSELDKAGFIKRQLVETRQITKHVAQILDSRM
NTKYDENDKLIREVKVITLKSKLVSDFRKDFQFYKVREINNYHHAHDAYLNAVVGTA
LIKKYPKLESEFVYGDYKVYDVRKMIAKSEQEIGKATAKYFFYSNIMNFFKTEITLAN
GEIRKRPLIETNGETGEIVWDKGRDFATVRKVLSMPQVNIVKKTEVQTGGFSKESILP
KRNSDKLIARKKDWDPKKYGGFDSPTVAYSVLVVAKVEKGKSKKLKSVKELLGITIM
ERSSFEKNPIDFLEAKGYKEVKKDLIIKLPKYSLFELENGRKRMLASAGELQKGNELA
LPSKYVNFLYLASHYEKLKGSPEDNEQKQLFVEQHKHYLDEIIEQISEFSKRVILADAN
LDKVLSAYNKHRDKPIREQAENIIHLFTLTNLGAPAAFKYFDTTIDRKRYTSTKEVLD
ATLIHQSITGLYETRIDLSQLGGDSGGSGGSGGSTNLSDIIEKETGKQLVIQESILMLPEE
VEEVIGNKPESDILVHTAYDESTDENVMLLTSDAPEYKPWALVIQDSNGENKIKMLSG
GSGGSGGSTNLSDIIEKETGKQLVIQESILMLPEEVEEVIGNKPESDILVHTAYDESTDE
NVMLLTSDAPEYKPWALVIQDSNGENKIKMLSGGSPKKKRK

In certain embodiments, the disclosed fusion proteins are made by various modes of manipulation that include, but are not limited to, codon optimization and performance of ancestral reconstruction of components of the fusion proteins (e.g. of a deaminase) to achieve greater expression levels in a cell, and the use of nuclear localization sequences (NLSs), preferably at least two NLSs to increase the localization of the expressed fusion proteins into a cell nucleus. In particular embodiments, the fusion protein contains an ancestrally reconstructed adenosine deaminase (“AncABE”).

TALE Base Editors

Configurations of the TALE base editors utilized in the methods disclosed herein may comprise a TALE domain, a deaminase domain and/or cofactor protein (e.g. FokI endonuclease) domain that comprise fusion proteins having the general structure NH₂-[TALE]-[deaminase domain]-COOH, NH₂-[deaminase domain]-[TALE]-COOH, NH₂-[TALE]-[deaminase domain]-[cofactor protein]-COOH, NH₂-[cofactor protein]-[deaminase domain]-[TALE]-COOH, NH₂-[cofactor protein]-[TALE]-[deaminase]-COOH or NH₂-[deaminase domain]-[TALE]-[cofactor protein]-COOH; wherein each instance of “]-[” comprises an optional linker, e.g. a peptide linker.

The TALE base editors utilized in the disclosed methods may further comprise one, two, or more than two nuclear localization sequences (NLS).

Base Editing Methods—Unique Genomic Loci

Methods are provided for making targeted edits to multiple (e.g. tens to hundreds to thousands) unique loci in the genomic DNA of a cell (e.g. a eukaryotic cell). Such methods involve transducing (e.g. via transfection) cells with a plurality of complexes, each comprising a fusion protein (e.g. a fusion protein comprising a nuclease inactive Cas9 (dCas9) domain and a deaminase domain) and one or more guide RNAs (gRNA).

In multiplexed base editing of unique genomic loci, a plurality of gRNAs having complementarity to different target sequences enables the formation of fusion protein-gRNA complexes at each of several (e.g. 5, 10, 15, 20, 25, or more) target sequences simulataneously, or within a single iteration or cycle.

In some embodiments, the gRNA is associated with the DNA binding domain (e.g. dCas9 domain) of the fusion protein. In some embodiments, each gRNA comprises a guide sequence of at least 10 contiguous nucleotides (e.g. 10, 11, 12, 13, 14, 15, 16, 17, 18, 19, 20, 21, 22, 23, 24, 25, 26, 27, 28, 29, or 30 contiguous nucleotides) that is complementary to a target sequence in the genomic DNA of a eukaryotic cell.

In particular embodiments, the plurality of distinct complexes comprises at least five, at least ten, at least fifteen, at least twenty, at least thirty, or at least fifty such complexes.

In certain embodiments, the plurality of the disclosed fusion protein-gRNA complexes make simultaneous edits (i.e., within a single iteration) at various target loci within a eukaryotic cell, e.g. a mammalian cell.

In certain embodiments of the disclosed methods, the constructs that encode the fusion proteins are transfected into the cell separately from the constructs that encode the gRNAs. In certain embodiments, these components are encoded on a single construct and transfected together. In particular embodiments, these single constructs encoding the fusion proteins and gRNAs may be transfected into the cell iteratively, with each iteration associated with a subset of target sequences. In particular embodiments, these single constructs may be transfected into the cell over a period of days. In other embodiments, they may be transfected into the cell over a period of hours. In other embodiments, they may be transected into the cell over a period of weeks.

In other embodiments, the methods involve the introduction into eukaryotic cells of a plurality of distinct complexes of fusion protein-gRNAs expressed and isolated/prepared outside of the target cells. In particular embodiments, these complexes may be introduced into the cell iteratively, with each iteration associated with a subset of target sequences. In particular embodiments, these complexes may be transfected into the cell over a period of days. In other embodiments, they may be transected into the cell over a period of weeks. In certain embodiments, a single bolus of complexes, or a single bolus of gRNAs is transfected into the cell. In particular, a single bolus of ribonucleoprotein complexes, each containing six or more gRNAs can be co-delivered. Alternatively, a single bolus of thirty-two or more gRNAs may be delivered. Reference is made to M. Serif et al., One-step generation of multiple gene knock-outs in the diatom Phaeodactylum tricornutum by DNA-free genome editing. Nat Commun. 9, 3924 (2018), Y. Li et al., Programmable Single and Multiplex Base-Editing in Bombyx mori Using RNA-Guided Cytidine Deaminases. G3 (Bethesda). 8, 1701-1709 (2018), and Thompson D et al., The future of multiplexed eukaryotic enome engineering, ACS Chem Biol. 2018; 13(2): 313-325, each of which are herein incorporated by reference.

In some embodiments, the disclosed methods involve transducing (e.g. via transfection) cells with a plurality of complexes each comprising a fusion protein comprising a TAL effector domain and a deaminase domain and a cofactor protein, wherein each cofactor protein localizes the fusion protein to a distinct target sequence. Reference is made to Yang L. et al., Engineering and optimizing deaminase fusions for genome editing, Nature Comms., 2016. In particular embodiments, the methods disclosed herein involve TAL effector domains that bind target sites not by Watson-Crick hybridization, but by binding the major groove of the DNA double helix. In certain embodiments, the methods involve the transfection of nucleic acid constructs (e.g. plasmids) that each (or together) encode the components of a plurality of complexes of a TALE base editor comprising a TALE domain and a deaminase domain, and a cofactor protein. In certain embodiments, the disclosed fusion proteins comprise a cofactor protein domain—i.e. the domain is incorporated into the fusion protein construct. In other embodiments, the TALE base editor comprises a TALE domain and a deaminase domain, and the cofactor protein is introduced into the cell separately from the base editor.

In certain embodiments of the disclosed methods, the constructs that encode the TALE base editors are transfected into the cell separately from the constructs that encode the cofactor proteins. In certain embodiments, these components are encoded on a single construct and transfected together. In particular embodiments, these single constructs encoding the TALE base editor and cofactor proteins may be transfected into the cell iteratively, with each iteration associated with a subset of target sequences. In particular embodiments, these single constructs may be transfected into the cell over a period of days. In other embodiments, they may be transfected into the cell over a period of weeks.

The target sequence may be in any suitable nucleic acid molecule. The target sequences in the genomic DNA of the disclosed methods may comprise coding regions. In other embodiments, the target sequences comprise non-coding regions of the genome, or a combination of coding and non-coding sequences. In some embodiments, the target sequences comprise non-coding transposable elements, e.g. LINE-1 or HERV sequences. It should be appreciated that the target sequences of the genomic DNA may comprise any combination of coding regions, non-coding regions, transposable elements, or any other target sequences in the genomic DNA of a cell (e.g. eukaryotic cell).

In certain embodiments, at least 10, 15, 20, 30, 40, 50 or more of the fusion proteins of the plurality are each bound to a unique gRNA comprising a different guide sequence of at least 10 contiguous nucleotides that is complementary to a target sequence. In other embodiments, at least 25, 30, 35, 40, 45, or 50 of the fusion proteins of the plurality are each bound to a unique gRNA that is complementary to a target sequence in the genomic DNA of a eukaryotic cell. Thus, a plurality of at least 10, 15, 20, 25, 30, 35, 40, 45, 50, 100, 200, 300, 500, or 1000 fusion protein-gRNA complexes are provided that may make concurrent edits to target loci within a cell.

In various embodiments, each of the fusion proteins of the plurality of proteins bound to a unique gRNA comprises the amino acid sequence of SEQ ID NO: 3. In various embodiments, each of the fusion proteins of the plurality of proteins bound to a unique gRNA comprises the amino acid sequence of SEQ ID NO: 4. In certain embodiments, each of the fusion proteins of the plurality is the same.

In particular embodiments, the contacting step consists essentially of contacting the cell with a fusion protein comprising (i) a nuclease inactive Cas9 (dCas9) domain and (ii) a deaminase domain, and a guide RNA (gRNA) bound to the dCas9 domain, wherein the guide RNA comprises a sequence of at least 10 contiguous nucleotides that is complementary to a target sequence, and wherein at least 25 copies of the target sequence are present in the genomic DNA of a eukaryotic cell.

### Target Sequences

In certain embodiments, a C to U or a C to T point mutation is effected in the target sequence(s). In other embodiments, an A to G point mutation is effected in the target sequence(s). In various embodiments, the step of contacting results in the replacement of a codon encoded by the target sequence with a different codon. In particular, this step may result in the generation of a plurality of STOP codons, e.g. STOP codons that inactivate a transposable element. In particular, the genome-wide replacement of a plurality of codons may result in the re-writing or recoding of the entire genome of a cell.

In various embodiments, the step of contacting results in less than 20% indel formation upon base editing, and in particular less than 15%, 10%, 5%, 3%, 2% or 1% indel formation. In certain embodiments, the step of contacting results in at least 2:1 intended to unintended product. The step of contacting may result in at least 3:1, 4:1, 5:1, 7:1 or 10:1 intended to unintended product.

In various embodiments, the step of contacting comprises editing more than 50, more than 100, more than 200, more than 500, more than 1,000, more than 2,000, more than 3,000, more than 5,000, more than 10,000, more than 20,000, more than 30,000, more than 50,000, or more than 100,000 target sequences in the genomic DNA of the eukaryotic cell. In particular embodiments, the step of contacting comprises editing more than 11,000, more than 12,000, more than 13,000, more than 14,000, or more than 15,000 target sequences in mammalian cells. In particular embodiments, the step of contacting comprises editing more than 2400, more than 2500, more than 2600, more than 2700, more than 2800, or more than 2900 target sequences in sensitive mammalian cells (where even a single DSB can lead to apoptosis) such as human induced pluripotent stem cells.

In various embodiments, the target sequence of the disclosed methods comprises a transposable element (TE), e.g. an Alu sequence; a Long Interspersed Human Elements-1 (LINE-1) sequence; an SINE-VNTR-Alus (SVA) sequence; a consensus centromere sequence; a chromosome specific centromere sequence; a telomere; a foreign DNA transposon such as PiggyBac, or a Sleeping Beauty transposon; a Human Endogenous Retrovirus-W (HERV-W) sequence; or a Human Endogenous Retrovirus-K (HERV-K) sequence. Reference is made to F. Adikusuma, et al., Targeted Deletion of an Entire Chromosome Using CRISPR/Cas9. Mol. Ther. 25, 1736-1738 (2017), incorporated herein by reference.

Exemplary HERV and HERV-K target sequences are listed in the following table (see Kim T. et al., The Distribution and Expression of HERV Families in the Human Genome, Mol. Cells 2004; 18(1): 87-93, herein incorporated by reference:

| Small HERV family | | Large HERV family | |
|---|---|---|---|
| Superfamily | Family | Superfamily | Family |
| HERV-15 | HERV-15 | HERV-K | HERV-K |
| HERV-16 | HERV-16 | | HERVK3 |
| HERV-17 | HERV-17 | | HERV-KC4 |
| HERV-3 | HERV-3 | | HERV-K9 |
| HERV-30 | HERV-30 | | HERV-K11 |
| HERV-9 | HERV-9 | | HERV-K11D |
| HERV-E | HERV-E | | HERV-K13 |
| HERVS-71 | HERV-S71 | | HERV-K14 |
| HERV-P71A | HERVP-71A | | HERV-14C |
| HERV-R | HERV-R | | HERV-K22 |
| HERV-FH19 | HERV-FH19 | HERV-L | HERV-L |
| | HERV-FH21 | | ERV-L |
| HERV-H | HERV-H | | HERV-L18 |
| | HERV-H48 | | HERV-L32 |
| HERV-I | HERV-I | | HERV-L40 |
| | HERV-IP10F | | HERV-L66 |
| | HERV-IP10FH | | HERV-L74 |

In various embodiments, the step of contacting results in a base editing efficiency of at least about 35%, 40%, 50%, 60%, 70%, 80%, 85%, 90%, 95%, 98%, or 99%. The step of contacting may result in in a base editing efficiency of at least about 51%, 52%, 53%, 54%, 55%, 56% or 57%. In particular, the step of contacting results in base editing efficiencies of greater than 54%. In certain embodiments, base editing efficiencies of 99% may be realized.

In certain embodiments, the step of contacting results in low toxicity when administered to a population of cells. In particular embodiments, less than 30%, less than 20%, less than 15%, less than 10%, less than 5% or less than 1% cell death in the population of cells is observed. In various embodiments, the step of contacting results in a low level of DNA damage when administered to a population of cells, e.g. at least 60%, at least 65%, at least 70%, at least 75%, at least 80%, at least 85%, at least 90%, at least 95%, or at least 99% of the cells are viable 24 hours after the step of contacting. In various embodiments, at least 60% of cells are viable at least 72 hours after contacting.

In various embodiments, the ratio of unique gRNAs to unique target sequences in the disclosed methods is 1:1.

The step of contacting of the disclosed methods may be performed in vitro (e.g. in cell culture), ex vivo, or in vivo (e.g. in an animal).

### Base Editing Methods—Repetitive Elements

Methods are provided for making edits to hundreds to tens of thousands of copies of a single target sequence (e.g. a repetitive element) in the genomic DNA of a eukaryotic cell. In particular embodiments, methods are provided for making edits to at least 25 copies of a target sequence. These methods involve transfecting cells with a plurality of complexes each comprising a fusion protein (each comprising a nuclease inactive Cas9 (dCas9) domain and a deaminase domain) and a guide RNA (gRNA) molecule. The gRNA is bound to the dCas9 domain of the fusion protein. Each gRNA comprises a guide sequence of at least 10 contiguous nucleotides that is complementary to the same target sequence in the genomic DNA of a eukaryotic cell.

In particular embodiments, the contacting step consists essentially of contacting a cell with a fusion protein comprising (i) a nuclease inactive Cas9 (dCas9) domain and (ii) a deaminase domain, and a guide RNA (gRNA) bound to the dCas9 domain, wherein the guide RNA comprises a sequence of at least 10 contiguous nucleotides that is complementary to a target sequence, and wherein at least 25 copies of the target sequence are present in the genomic DNA of a eukaryotic cell.

In certain embodiments, the methods involve the transfection of plasmids that each (or together) express the components of a plurality of complexes of fusion protein-gRNAs, wherein each gRNA has complementarity to the same target sequence. In other embodiments, the methods involve the introduction into eukaryotic cells of a plurality of complexes of fusion protein-gRNAs expressed and prepared/isolated outside of the target cells.

In certain embodiments, the plurality of the disclosed fusion protein-gRNA complexes make concurrent edits to target loci within a eukaryotic cell, e.g. a mammalian cell.

In certain embodiments of the disclosed methods, the fusion proteins are transfected into the cell separately from the gRNAs. In certain embodiments, the fusion protein-gRNA complexes per se are delivered into the cell. In particular embodiments, these complexes may be transfected into the cell iteratively. In particular embodiments, these complexes may be transfected into the cell over a period of days. In other embodiments, they may be transected into the cell over a period of weeks. In other embodiments of the disclosed methods, a single bolus of complexes, or a single bolus of gRNA molecules is transfected into the cell.

Repetitive element gRNA sequences may be designed manually based on the consensus sequence and compared to the non-redundant genome to prevent additional unwanted off-targets. In the foregoing Examples, custom analysis scripts were written to analyze the sequencing results data following transfection of target cells with manually-designed repetitive element gRNAs.

The target sequences in the genomic DNA of the disclosed methods may comprise coding regions. In other embodiments, the target sequences may comprise non-coding regions of the genome. In some embodiments, the target sequences comprise non-coding transposable elements, e.g. LINE-1 or HERV sequences.

In certain embodiments, the target sequence is a repetitive element. In certain embodiments, the gRNA is a single-guide RNA (sgRNA), e.g. a promiscuous gRNA.

Exemplary repetitive elements include Alu, LINE-1, SINE-VNTR-Alus (SVA), consensus centromere, chromosome specific centromere, telomere, foreign DNA transposon such as PiggyBac, or a Sleeping Beauty transposon, HERV-W, and HERV-K sequences. Targeted editing of a sequence with a high copy number is useful in, for instance, the deactivation of harmful TE (e.g. HERV) sequences in a human cell. For instance, targeted editing of repetitive elements is useful in the discriminate introduction of a plurality of codons at harmful TE (e.g. HERV) sequences.

Exemplary repetitive elements are 10, 20, 30, 40, 50, 70, or 100 nucleotides in length. Exemplary repetitive elements may vary in copy numbers from 30 to greater than 160,000 locations across the genome.

### Maximizing Survival of Edited Cells

After the step of transfecting the eukaryotic cells is performed as described above, the disclosed methods provide for the addition of one or more agents that facilitate survival and/or viability of the cells. These additions are made following sufficient exposure of the target sequence to the base editor to allow for base editing. Such agents are described in further detail below. These agents may be added immediately after transfection, 4 hours after transfection, 8 hours after transfection, 12 hours after transfection, 16 hours after transfection, 24 hours after transfection, 30 hours after transfection, 35 hours after transfection, 48 hours after transfections, 3 days after transfection, or 4 days after transfection.

In various embodiments, the base editing methods of the present disclosure further comprise contacting the eukaryotic cell with an anti-apoptotic molecule to promote cell survival. In particular embodiments, the anti-apoptotic molecule is a small molecule p53 inhibitor. In particular embodiments, the anti-apoptotic molecule is pifithrin-α (PFA) or pifithrin-μ (PFμ).

In various embodiments, the methods further comprise contacting the eukaryotic cell with a growth factor, e.g. a basic fibroblast growth factor (bFGF).

In other embodiments, the methods further comprise contacting the eukaryotic cell with an inhibitor of mismatch repair (MMR), e.g. cadmium chloride; or an inhibitor of non-homologous end joining (NHEJ). In certain embodiments, the methods further comprise conditionally knocking out a gene in the cell encoding a protein involved in NHEJ or MMR, e.g. the gene encoding the MutSα complex, or the gene encoding the MutLα complex.

In various embodiments, the disclosed methods further comprise contacting the nucleic acid molecule with an isolated inhibitor of base excision repair (iBER), such as isolated UGI. Optimally, such methods are used with fusion proteins that do not comprise a fused inhibitor of BER, such as a fused UGI. In some embodiments, the isolated UGI inhibits base excision repair of the edited strand or non-edited strand.

The disclosed methods may comprise a combination of all such approaches, including contactin the cell with a growth factor, an anti-aptoptotic molecule, an inhibitor of mismatch repair, an inhibitor of base excision repair and/or an inhibitor of NHEJ. The disclosed methods may comprise a combination of all such approaches and further the step of conditionally knocking out a gene in the cell encoding a protein involved in NHEJ or MMR, e.g. the gene encoding the MutSα complex or the gene encoding the MutLα complex.

Exemplary methods utilize a bolus of gRNAs targeting repetitive elements having a copy number from about 31 to 124,000 per genome. dBEs (e.g. dABEs and dCBEs) enabled survival after large-scale base editing, allowing targeted deamination at up to ˜13,200 and ˜2610 loci, respectively, in HEK 293T and induced pluripotent stem cells. These numbers represent an improvement in scale of editing by three orders of magnitude than previously reported.

### Cas9 Proteins

RNA-guided DNA binding proteins are readily known to those of skill in the art to bind to DNA for various purposes. Such DNA binding proteins may be naturally occurring or engineered. DNA binding proteins having nuclease activity are known to those of skill in the art, and include naturally occurring DNA binding proteins having nuclease activity, such as Cas9 proteins present, for example, in Type II CRISPR systems. Such Cas9 proteins and Type II CRISPR systems are well documented in the art. See Makarova et al., Nature Reviews, Microbiology, Vol. 9, June 2011, pp. 467-477, including all supplementary information, which is herein incorporated by reference in its entirety. Reference is also made to Rees & Liu, Base editing: precision chemistry on the genome and transcriptome of living cells, Nat. Rev. Genet. 2018; 19(12):770-788; as well as. U.S. Patent Publication No. 2018/0073012, published Mar. 15, 2018, which issued as U.S. Pat. No. 10,113,163, on Oct. 30, 2018; U.S. Patent Publication No. 2017/0121693, published May 4, 2017, which issued as U.S. Pat. No. 10,167,457 on Jan. 1, 2019; International Publication No. WO 2017/070633, published Apr. 27, 2017; International Publication No, 2017/197238, published Nov. 16, 2017; U.S. Patent Publication No. 2015/0166980, published Jun. 18, 2015; U.S. Pat. No. 9,840,699, issued Dec. 12, 2017; U.S. Pat. No. 10,077,453, issued Sep. 18, 2018; U.S. Pat. No. 9,587,252, issued Mar. 7, 2017, the contents of each of which are incorporated herein by reference in their entireties.

In general, bacterial and archaeal CRISPR-Cas systems rely on guide RNAs in complex with Cas proteins to direct degradation of complementary sequences present within invading foreign nucleic acid. See Deltcheva, E. et al., CRISPR RNA maturation by transencoded small RNA and host factor RNase III. Nature 471, 602-607 (20); Gasiunas, G., Barrangou, R., Horvath, P. & Siksnys, V. Cas9-crRNA ribonucleoprotein complex mediates specific DNA cleavage for adaptive immunity in bacteria. Proceedings of the National Academy of Sciences of the United States of America 109, E2579-2586 (2012); Jinek, M. et al. A programmable dual-RNA-guided D A endonuclease in adaptive bacterial immunity. Science 337, 816-821 (2012): Sapranauskas, R. et al. The Streptococcus thermophilus CRISPR/Cas system provides immunity in Escherichia coli. Nucleic acids research 39, 9275-9282 (2011); and Bhaya, D., Davison, M. & Barrangou, R. CRISPR-Cas systems in bacteria and archaea: versatile small R As for adaptive defense and regulation.

According to one aspect, the DNA binding proteins of the present disclosure, such as Cas9, unwind the DNA duplex and search for sequences matching the crRNA to cleave. Target recognition occurs upon detection of complementarity between a “protospacer” sequence in the target DNA and the remaining spacer sequence in the crRNA. Importantly, Cas9 modifies the DNA only if a correct protospacer-adjacent motif (PAM) is also present at the 3′ end. According to certain aspects, different protospacer-adjacent motif can be utilized. For example, the S. pyogenes system requires an NGG sequence, where N can be any nucleotide. S. thermophilus Type II systems require an NGGNG sequence (SEQ ID NO: 16) (see P. Horvath, R. Barrangou, CRISPR/Cas, the immune system of bacteria and archaea. Science 327, 167 (Jan. 8, 2010), herein incorporated by reference in its entirety and NNAGAAW (SEQ ID NO: 17) (see H. Deveau et al., Phage response to CRISPR-encoded resistance in Streptococcus thermophilus. Journal of Bacteriology 190, 1390 (February 2008), incorporated herein by reference in its entirety), respectively, while different S. mulans systems tolerate NGG or NAAR (see J. R. van der Ploeg, Analysis of CRISPR in Streptococcus mutatis suggests frequent occurrence of acquired immunity against infection by M102-like bacteriophages. Microbiology 155, 1966 (June 2009), herein incorporated by refernece in its entirety. Bioinformatic analyses have generated extensive databases of CRISPR loci in a variety of bacteria that may serve to identify additional useful PAMs and expand the set of CRISPR-targetable sequences (see M. Rho, Y. W. Wu, H. Tang, T. G. Doak, Y. Ye, Diverse CRISPRs evolving in human microbiomes. PLoS genetics 8, e1002441 (2012) and D. T. Pride et al, Analysis of streptococcal CRISPRs from human saliva reveals substantial sequence diversity within and between subjects over Lime. Genome Research 21, 126 (January 2011), each of which are herein incorporated by reference in their entireties.

Cas9 orthologs have been described in various species, including, but not limited to, S. aureus, S. pyogenes, S. thermophiles, C. ulcerans, S. diphtheria, S. syrphidicola, P. intermedia, S. taiwanense, S. iniae, B. baltica, P. torquis, S. thermophiles, L. innocua, C. jejuni and N. meningitidis. Additional suitable Cas9 nucleases and sequences will be apparent to those of skill in the art based on this disclosure, and such Cas9 nucleases and sequences include Cas9 sequences from the organisms and loci disclosed in Chylinski, Rhun, and Charpentier, “The tracrRNA and Cas9 families of type II CRISPR-Cas immunity systems” (2013) RNA Biology 10:5, 726-737, which is incorporated herein by reference in its entirety. In S. pyogenes, Cas9 generates a blunt-ended double-stranded break 3 bp upstream of the protospacer-adjacent motif (PAM) via a process mediated by two catalytic domains in the protein: an HNH domain that cleaves the complementary strand of the DMA and a RuvC-like domain that cleaves the non-complementary strand. See Jinek et al., Science 337, 816-821 (2012), herein incorporated by reference in its entirety.

An exemplary CRISPR system includes the S. aureus Cas9 nuclease (SaCas9), which recognizes an NNGRRT protospacer adjacent motif (PAM) and can cleave target sequences at high efficiency with a variety of guide RNA (gRNA) spacer lengths (see Friedland A E et al., Characterization of Staphylococcus aureus Cas9: a smaller Cas9 for all-in-one adeno-associated virus delivery and paired nickase applications, Genome Biol. (2015), herein incorporated by reference). As with the S. pyogenes Cas9, the S. aureus Cas9 contains HNH and RuvC1 subdomains: HNH subdomain cleaves the strand complementary to the gRNA (the “targeted strand”), whereas the RuvC1 subdomain cleaves the non-complementary strand containing the PAM sequence (the “non-edited strand”). The RuvC1 mutant D10A generates a nick in the targeted strand, while the HNH mutant N580A generates a nick on the non-edited strand. (See id.; see also Ran F A, et al., In vivo genome editing using Staphylococcus aureus Cas9, Nature (2015), herein incorporated by reference).

Another exemplary CRISPR system includes the S. thermophiles Cas9 nuclease (ST1 Cas9) (see Esvelt K M, et al., Orthogonal Cas9 proteins for RNA-guided gene regulation and editing, Nature Methods, (2013) herein incorporated by reference in its entirety). Another exemplary CRISPR system includes the S. pyogenes Cas9 nuclease (SpCas9), an extremely high-affinity (see Sternberg, S. H., Redding, S., Jinek, M., Greene, E. C. & Doudna, J. A. DNA interrogation by the CRISPR RNA-guided endonuclease Cas9. Nature 507, 62-67 (2014), herein incorporated by reference in its entirety), programmable DNA-binding protein isolated from a type II CRISPR-associated system (see Gameau, J. E. et al. The CRISPR/Cas bacterial immune system cleaves bacteriophage and plasmid DNA. Nature 468, 67-71 (2010) and Jinek, M. et al., A programmable dual-RNA-guided DNA endonuclease in adaptive bacterial immunity. Science 337, 816-821 (2012), each of which are herein incorporated by reference in its entirety).

According to certain aspects, a nuclease null or nuclease inactive Cas9 can be used in the methods described herein. Such nuclease null or nuclease inactive Cas9 proteins are described in Gilbert, L. A. et al., CRISPR-mediated modular RNA-guided regulation of transcription in eukaryotes. Cell 154, 442-451 (2013); Mali, P. et al. CAS9 transcriptional activators for target specificity screening and paired nickases for cooperative genome engineering. Nature Biotechnology 31, 833-838 (2013); Maeder, M. L. et al., CRISPR RNA-guided activation of endogenous human genes. Nature Methods 10, 977-979 (2013); and Perez-Pinera, P. et al., RNA-guided gene activation by CRISPR-Cas9-based transcription factors. Nature Methods 10, 973-976 (2013), each of which are herein incorporated by reference in its entirety.

The DNA locus targeted by Cas9 (and by its nuclease inactive mutant, “dCas9”) precedes a three nucleotide (nt) 5-NGG-3 “PAM” sequence and matches a 15- to 22-nt guide or spacer sequence within a guide RNA.

According to one aspect, the Cas9 protein is an enzymatically active Cas9 protein, a Cas9 protein wild-type protein, a Cas9 protein nickase or a nuclease null or nuclease inactive Cas9 protein. Additional exemplary Cas9 proteins include Cas9 proteins attached to, bound to or fused with functional proteins such as transcriptional regulators, such as transcriptional activators or repressors, a Fok-domain, such as FokI, an aptamer, a binding protein, PP7 MS2 and the like.

According to certain aspects, the Cas9 protein may be delivered directly to a cell by methods known to those of skill in the art, including injection or lipofection, or as translated from its cognate mRNA, or transcribed from its cognate DNA into mRNA (and thereafter translated into protein). Cas9 DNA and mRNA may be themselves introduced into cells through electroporation, transient and stable transfection (e.g. lipofection) and viral transduction or other methods known to those of skill in the art.

In some embodiments, proteins comprising Cas9 or fragments thereof are referred to as “Cas9 variants.” A Cas9 variant shares homology to Cas9, or a fragment thereof. For example a Cas9 variant is at least about 70% identical, at least about 80% identical, at least about 90% identical, at least about 95% identical, at least about 96% identical, at least about 97% identical, at least about 98% identical, at least about 99% identical, at least about 99.5% identical, or at least about 99.9% identical to wild type Cas9. In some embodiments, the Cas9 variant may have 1, 2, 3, 4, 5, 6, 7, 8, 9, 10, 11, 12, 13, 14, 15, 16, 17, 18, 19, 20, 21, 22, 21, 24, 25, 26, 27, 28, 29, 30, 31, 32, 33, 34, 35, 36, 37, 38, 39, 40, 41, 42, 43, 44, 45, 46, 47, 48, 49, 50 or more amino acid changes compared to a wild type Cas9. In some embodiments, the Cas9 variant comprises a fragment of Cas9 (e.g. a gRNA binding domain or a DNA-cleavage domain), such that the fragment is at least about 70% identical, at least about 80% identical, at least about 90% identical, at least about 95% identical, at least about 96% identical, at least about 97% identical, at least about 98% identical, at least about 99% identical, at least about 99.5% identical, or at least about 99.9% identical to the corresponding fragment of wild type Cas9. In some embodiments, the fragment is at least 30%, at least 35%, at least 40%, at least 45%, at least 50%, at least 55%, at least 60%, at least 65%, at least 70%, at least 75%, at least 80%, at least 85%, at least 90%, at least 95% identical, at least 96%, at least 97%, at least 98%, at least 99%, or at least 99.5% of the amino acid length of a corresponding wild type Cas9. In some embodiments, a Cas9 variant has been engineered to be inactive for nucleic acid strand displacement activity during a strand invasion process.

In some embodiments, the Cas9 fragment is at least 100 amino acids in length. In some embodiments, the fragment is at least 100, 150, 200, 250, 300, 350, 400, 450, 500, 550, 600, 650, 700, 750, 800, 850, 900, 950, 1000, 1050, 1100, 1150, 1200, 1250, or at least 1300 amino acids in length. In some embodiments, wild type Cas9 corresponds to Cas9 from Streptococcus pyogenes (NCBI Reference Sequence: NC_017053.1). In other embodiments, wild type Cas9 corresponds to Cas9 from Streptococcus pyogenes (NCBI Reference Sequence: NC_002737.2). In still other embodiments, dCas9 corresponds to, or comprises in part or in whole, a Cas9 amino acid sequence having one or more mutations that inactivate the Cas9 nuclease activity.

In other embodiments, dCas9 variants having mutations other than D10A and H840A are provided, which, e.g. result in nuclease inactivated Cas9 (dCas9). Such mutations, by way of example, include other amino acid substitutions at D10 and H840, or other substitutions within the nuclease domains of Cas9 (e.g. substitutions in the HNH nuclease subdomain and/or the RuvC1 subdomain) with reference to a wild type sequence such as Cas9 from Streptococcus pyogenes (NCBI Reference Sequence: NC_017053.1). In some embodiments, variants or homologues of dCas9 (e.g. variants of Cas9 from Streptococcus pyogenes (NCBI Reference Sequence: NC_017053.1)) are provided which are at least about 70% identical, at least about 80% identical, at least about 90% identical, at least about 95% identical, at least about 98% identical, at least about 99% identical, at least about 99.5% identical, or at least about 99.9% identical to NCBI Reference Sequence: NC_017053.1. In some embodiments, variants of dCas9 (e.g. variants of NCBI Reference Sequence: NC_017053.1) are provided having amino acid sequences which are shorter, or longer than NC_017053.1 by about 5 amino acids, by about 10 amino acids, by about 15 amino acids, by about 20 amino acids, by about 25 amino acids, by about 30 amino acids, by about 40 amino acids, by about 50 amino acids, by about 75 amino acids, by about 100 amino acids or more.

In some embodiments, the fusion proteins as provided herein comprise the full-length amino acid sequence of a Cas9 protein, e.g. one of the Cas9 sequences provided herein. In other embodiments, however, fusion proteins utilized in the methods provided herein do not comprise a full-length Cas9 sequence, but only a fragment thereof. For example, in some embodiments, a Cas9 fusion protein provided herein comprises a Cas9 fragment, wherein the fragment binds crRNA and tracrRNA or sgRNA, but does not comprise a functional nuclease domain, e.g. in that it comprises only a truncated version of a nuclease domain or no nuclease domain at all. Exemplary amino acid sequences of suitable Cas9 domains and Cas9 fragments are provided herein, and additional suitable sequences of Cas9 domains and fragments will be apparent to those of skill in the art.

It should be appreciated that additional Cas9 proteins (e.g. a nuclease dead Cas9 (dCas9), a Cas9 nickase (nCas9), or a nuclease active Cas9), including variants and homologs thereof, are within the scope of this disclosure. Exemplary Cas9 proteins include, without limitation, those provided below. In some embodiments, the Cas9 protein is a nuclease dead Cas9 (dCas9). In some embodiments, the dCas9 comprises the amino acid sequence of SEQ ID NO: 18. In other embodiments, the dCas9 comprises the amino acid sequence of SEQ ID NO: 100. In other aspects, the Cas9 protein is a Cas9 nickase (nCas9), and may comprise the amino acid sequence of any one of SEQ ID NOs: 19 or 101.

In certain embodiments, the fusion proteins of the invention may comprise a catalytically inactive Cas9 (dCas9) derived from S. pyogenes that comprises an amino acid sequence that is at least 80%, 85%, 90%, 95%, 98%, or 99% identical to the amino acid sequence of (D10A and H840A mutations underlined):

(SEQ ID NO: 18)
DKKYSIGLAIGTNSVGWAVITDEYKVPSKKFKVLG
NTDRHSIKKNLIGALLFDSGETAEATRLKRTARRR
YTRRKNRICYLQEIFSNEMAKVDDSFFHRLEESFL
VEEDKKHERHPIFGNIVDEVAYHEKYPTIYHLRKK
LVDSTDKADLRLIYLALAHMIKFRGHFLIEGDLNP
DNSDVDKLFIQLVQTYNQLFEENPINASGVDAKAI
LSARLSKSRRLENLIAQLPGEKKNGLFGNLIALSL
GLTPNFKSNFDLAEDAKLQLSKDTYDDDLDNLLAQ
IGDQYADLFLAAKNLSDAILLSDILRVNTEITKAP
LSASMIKRYDEHHQDLTLLKALVRQQLPEKYKEIF
FDQSKNGYAGYIDGGASQEEFYKFIKPILEKMDGT
EELLVKLNREDLLRKQRTFDNGSIPHQIHLGELHA
ILRRQEDFYPFLKDNREKIEKILTFRIPYYVGPLA
RGNSRFAWMTRKSEETITPWNFEEVVDKGASAQSF
IERMTNFDKNLPNEKVLPKHSLLYEYFTVYNELTK
VKYVTEGMRKPAFLSGEQKKAIVDLLFKTNRKVTV
KQLKEDYFKKIECFDSVETSGVEDRFNASLGTYHD
LLKIIKDKDFLDNEENEDILEDIVLTLTLFEDREM
IEERLKTYAHLFDDKVMKQLKRRRYTGWGRLSRKL
INGIRDKQSGKTILDFLKSDGFANRNFMQLIHDDS
LTFKEDIQKAQVSGQGDSLHEHIANLAGSPAIKKG
ILQTVKVVDELVKVMGRHKPENIVIEMARENQTTQ
KGQKNSRERMKRIEEGIKELGSQILKEHPVENTQL
QNEKLYLYYLQNGRDMYVDQELDINRLSDYDVDAI
VPQSFLKDDSIDNKVLTRSDKNRGKSDNVPSEEVV
KKMKNYWRQLLNAKLITQRKFDNLTKAERGGLSEL
DKAGFIKRQLVETRQITKHVAQILDSRMNTKYDEN
DKLIREVKVITLKSKLVSDFRKDFQFYKVREINNY
HHAHDAYLNAVVGTALIKKYPKLESEFVYGDYKVY
DVRKMIAKSEQEIGKATAKYFFYSNIMNFFKTEIT
LANGEIRKRPLIETNGETGEIVWDKGRDFATVRKV
LSMPQVNIVKKTEVQTGGFSKESILPKRNSDKLIA
RKKDWDPKKYGGFDSPTVAYSVLVVAKVEKGKSKK
LKSVKELLGITIMERSSFEKNPIDFLEAKGYKEVK
KDLIIKLPKYSLFELENGRKRMLASAGELQKGNEL
ALPSKYVNFLYLASHYEKLKGSPEDNEQKQLFVEQ
HKHYLDEIIEQISEFSKRVILADANLDKVLSAYNK
HRDKPIREQAENIIHLFTLTNLGAPAAFKYFDTTI
DRKRYTSTKEVLDATLIHQSITGLYETRIDLSQLG
GD.

In other embodiments, the fusion proteins may comprise a Cas9 nickase (nCas9) derived from S. pyogenes that comprises an amino acid sequence that is at least 80%, 85%, 90%, 95%, 98%, or 99% identical to the amino acid sequence of (D10A mutation underlined):

(SEQ ID NO: 19)
DKKYSIGLAIGTNSVGWAVITDEYKVPSKKFKVLG
NTDRHSIKKNLIGALLFDSGETAEATRLKRTARRR
YTRRKNRICYLQEIFSNEMAKVDDSFFHRLEESFL
VEEDKKHERHPIFGNIVDEVAYHEKYPTIYHLRKK
LVDSTDKADLRLIYLALAHMIKFRGHFLIEGDLNP
DNSDVDKLFIQLVQTYNQLFEENPINASGVDAKAI
LSARLSKSRRLENLIAQLPGEKKNGLFGNLIALSL
GLTPNFKSNFDLAEDAKLQLSKDTYDDDLDNLLAQ
IGDQYADLFLAAKNLSDAILLSDILRVNTEITKAP
LSASMIKRYDEHHQDLTLLKALVRQQLPEKYKEIF
FDQSKNGYAGYIDGGASQEEFYKFIKPILEKMDGT
EELLVKLNREDLLRKQRTFDNGSIPHQIHLGELHA
ILRRQEDFYPFLKDNREKIEKILTFRIPYYVGPLA
RGNSRFAWMTRKSEETITPWNFEEVVDKGASAQSF
IERMTNFDKNLPNEKVLPKHSLLYEYFTVYNELTK
VKYVTEGMRKPAFLSGEQKKAIVDLLFKTNRKVTV
KQLKEDYFKKIECFDSVETSGVEDRFNASLGTYHD
LLKIIKDKDFLDNEENEDILEDIVLTLTLFEDREM
IEERLKTYAHLFDDKVMKQLKRRRYTGWGRLSRKL
INGIRDKQSGKTILDFLKSDGFANRNFMQLIHDDS
LTFKEDIQKAQVSGQGDSLHEHIANLAGSPAIKKG
ILQTVKVVDELVKVMGRHKPENIVIEMARENQTTQ
KGQKNSRERMKRIEEGIKELGSQILKEHPVENTQL
QNEKLYLYYLQNGRDMYVDQELDINRLSDYDVDHI
VPQSFLKDDSIDNKVLTRSDKNRGKSDNVPSEEVV
KKMKNYWRQLLNAKLITQRKFDNLTKAERGGLSEL
DKAGFIKRQLVETRQITKHVAQILDSRMNTKYDEN
DKLIREVKVITLKSKLVSDFRKDFQFYKVREINNY
HHAHDAYLNAVVGTALIKKYPKLESEFVYGDYKVY
DVRKMIAKSEQEIGKATAKYFFYSNIMNFFKTEIT
LANGEIRKRPLIETNGETGEIVWDKGRDFATVRKV
LSMPQVNIVKKTEVQTGGFSKESILPKRNSDKLIA
RKKDWDPKKYGGFDSPTVAYSVLVVAKVEKGKSKK
LKSVKELLGITIMERSSFEKNPIDFLEAKGYKEVK
KDLIIKLPKYSLFELENGRKRMLASAGELQKGNEL
ALPSKYVNFLYLASHYEKLKGSPEDNEQKQLFVEQ
HKHYLDEIIEQISEFSKRVILADANLDKVLSAYNK
HRDKPIREQAENIIHLFTLTNLGAPAAFKYFDTTI
DRKRYTSTKEVLDATLIHQSITGLYETRIDLSQLG
GD.

In still other embodiments, the fusion proteins may comprise a catalytically active Cas9 derived from S. pyogenes that comprises an amino acid sequence that is at least 80%, 85%, 90%, 95%, 98%, or 99% identical to the amino acid sequence of:

(SEQ ID NO: 20)
DKKYSIGLDIGTNSVGWAVITDEYKVPSKKFKVLG
NTDRHSIKKNLIGALLFDSGETAEATRLKRTARRR
YTRRKNRICYLQEIFSNEMAKVDDSFFHRLEESFL
VEEDKKHERHPIFGNIVDEVAYHEKYPTIYHLRKK
LVDSTDKADLRLIYLALAHMIKFRGHFLIEGDLNP
DNSDVDKLFIQLVQTYNQLFEENPINASGVDAKAI
LSARLSKSRRLENLIAQLPGEKKNGLFGNLIALSL
GLTPNFKSNFDLAEDAKLQLSKDTYDDDLDNLLAQ
IGDQYADLFLAAKNLSDAILLSDILRVNTEITKAP
LSASMIKRYDEHHQDLTLLKALVRQQLPEKYKEIF
FDQSKNGYAGYIDGGASQEEFYKFIKPILEKMDGT
EELLVKLNREDLLRKQRTFDNGSIPHQIHLGELHA
ILRRQEDFYPFLKDNREKIEKILTFRIPYYVGPLA
RGNSRFAWMTRKSEETITPWNFEEVVDKGASAQSF
IERMTNFDKNLPNEKVLPKHSLLYEYFTVYNELTK
VKYVTEGMRKPAFLSGEQKKAIVDLLFKTNRKVTV
KQLKEDYFKKIECFDSVETSGVEDRFNASLGTYHD
LLKIIKDKDFLDNEENEDILEDIVLTLTLFEDREM
IEERLKTYAHLFDDKVMKQLKRRRYTGWGRLSRKL
INGIRDKQSGKTILDFLKSDGFANRNFMQLIHDDS
LTFKEDIQKAQVSGQGDSLHEHIANLAGSPAIKKG
ILQTVKVVDELVKVMGRHKPENIVIEMARENQTTQ
KGQKNSRERMKRIEEGIKELGSQILKEHPVENTQL
QNEKLYLYYLQNGRDMYVDQELDINRLSDYDVDHI
VPQSFLKDDSIDNKVLTRSDKNRGKSDNVPSEEVV
KKMKNYWRQLLNAKLITQRKFDNLTKAERGGLSEL
DKAGFIKRQLVETRQITKHVAQILDSRMNTKYDEN
DKLIREVKVITLKSKLVSDFRKDFQFYKVREINNY
HHAHDAYLNAVVGTALIKKYPKLESEFVYGDYKVY
DVRKMIAKSEQEIGKATAKYFFYSNIMNFFKTEIT
LANGEIRKRPLIETNGETGEIVWDKGRDFATVRKV
LSMPQVNIVKKTEVQTGGFSKESILPKRNSDKLIA
RKKDWDPKKYGGFDSPTVAYSVLVVAKVEKGKSKK
LKSVKELLGITIMERSSFEKNPIDFLEAKGYKEVK
KDLIIKLPKYSLFELENGRKRMLASAGELQKGNEL
ALPSKYVNFLYLASHYEKLKGSPEDNEQKQLFVEQ
HKHYLDEIIEQISEFSKRVILADANLDKVLSAYNK
HRDKPIREQAENIIHLFTLTNLGAPAAFKYFDTTI
DRKRYTSTKEVLDATLIHQSITGLYETRIDLSQLG
GD.

In other aspects, the fusion proteins may comprise a catalytically inactive Cas9 (dCas9) derived from S. aureus that comprises an amino acid sequence that is at least 80%, 85%, 90%, 95%, 98%, or 99% identical to the amino acid sequence of (D10A and N580A mutations underlined):

(SEQ ID NO: 100)
KRNYILGLAIGITSVGYGIIDYETRDVIDAGVRLF
KEANVENNEGRRSKRGARRLKRRRRHRIQRVKKLL
FDYNLLTDHSELSGINPYEARVKGLSQKLSEEEFS
AALLHLAKRRGVHNVNEVEEDTGNELSTKEQISRN
SKALEEKYVAELQLERLKKDGEVRGSINRFKTSDY
VKEAKQLLKVQKAYHQLDQSFIDTYIDLLETRRTY
YEGPGEGSPFGWKDIKEWYEMLMGHCTYFPEELRS
VKYAYNADLYNALNDLNNLVITRDENEKLEYYEKF
QIIENVFKQKKKPTLKQIAKEILVNEEDIKGYRVT
STGKPEFTNLKVYHDIKDITARKEIIENAELLDQI
AKILTIYQSSEDIQEELTNLNSELTQEEIEQISNL
KGYTGTHNLSLKAINLILDELWHTNDNQIAIFNRL
KLVPKKVDLSQQKEIPTTLVDDFILSPVVKRSFIQ
SIKVINAIIKKYGLPNDIIIELAREKNSKDAQKMI
NEMQKRNRQTNERIEEIIRTTGKENAKYLIEKIKL
HDMQEGKCLYSLEAIPLEDLLNNPFNYEVDHIIPR
SVSFDNSFNNKVLVKQEEASKKGNRTPFQYLSSSD
SKISYETFKKHILNLAKGKGRISKTKKEYLLEERD
INRFSVQKDFINRNLVDTRYATRGLMNLLRSYFRV
NNLDVKVKSINGGFTSFLRRKWKFKKERNKGYKHH
AEDALIIANADFIFKEWKKLDKAKKVMENQMFEEK
QAESMPEIETEQEYKEIFITPHQIKHIKDFKDYKY
SHRVDKKPNRELINDTLYSTRKDDKGNTLIVNNLN
GLYDKDNDKLKKLINKSPEKLLMYHHDPQTYQKLK
LIMEQYGDEKNPLYKYYEETGNYLTKYSKKDNGPV
IKKIKYYGNKLNAHLDITDDYPNSRNKVVKLSLKP
YRFDVYLDNGVYKFVTVKNLDVIKKENYYEVNSKC
YEEAKKLKKISNQAEFIASFYNNDLIKINGELYRV
IGVNNDLLNRIEVNMIDITYREYLENMNDKRPPRI
IKTIASKTQSIKKYSTDILGNLYEVKSKKHPQIIK
KG

In other aspects, the fusion proteins may comprise a Cas9 nickase (nCas9) derived from S. aureus that comprises an amino acid sequence that is at least 80%, 85%, 90%, 95%, 98%, or 99% identical to the amino acid sequence of (D10A mutation underlined):

(SEQ ID NO: 101)
KRNYILGLAIGITSVGYGIIDYETRDVIDAGVRLF
KEANVENNEGRRSKRGARRLKRRRRHRIQRVKKLL
FDYNLLTDHSELSGINPYEARVKGLSQKLSEEEFS
AALLHLAKRRGVHNVNEVEEDTGNELSTKEQISRN
SKALEEKYVAELQLERLKKDGEVRGSINRFKTSDY
VKEAKQLLKVQKAYHQLDQSFIDTYIDLLETRRTY
YEGPGEGSPFGWKDIKEWYEMLMGHCTYFPEELRS
VKYAYNADLYNALNDLNNLVITRDENEKLEYYEKF
QIIENVFKQKKKPTLKQIAKEILVNEEDIKGYRVT
STGKPEFTNLKVYHDIKDITARKEIIENAELLDQI
AKILTIYQSSEDIQEELTNLNSELTQEEIEQISNL
KGYTGTHNLSLKAINLILDELWHTNDNQIAIFNRL
KLVPKKVDLSQQKEIPTTLVDDFILSPVVKRSFIQ
SIKVINAIIKKYGLPNDIIIELAREKNSKDAQKMI
NEMQKRNRQTNERIEEIIRTTGKENAKYLIEKIKL
HDMQEGKCLYSLEAIPLEDLLNNPFNYEVDHIIPR
SVSFDNSFNNKVLVKQEENSKKGNRTPFQYLSSSD
SKISYETFKKHILNLAKGKGRISKTKKEYLLEERD
INRFSVQKDFINRNLVDTRYATRGLMNLLRSYFRV
NNLDVKVKSINGGFTSFLRRKWKFKKERNKGYKHH
AEDALIIANADFIFKEWKKLDKAKKVMENQMFEEK
QAESMPEIETEQEYKEIFITPHQIKHIKDFKDYKY
SHRVDKKPNRELINDTLYSTRKDDKGNTLIVNNLN
GLYDKDNDKLKKLINKSPEKLLMYHHDPQTYQKLK
LIMEQYGDEKNPLYKYYEETGNYLTKYSKKDNGPV
IKKIKYYGNKLNAHLDITDDYPNSRNKVVKLSLKP
YRFDVYLDNGVYKFVTVKNLDVIKKENYYEVNSKC
YEEAKKLKKISNQAEFIASFYNNDLIKINGELYRV
IGVNNDLLNRIEVNMIDITYREYLENMNDKRPPRI
IKTIASKTQSIKKYSTDILGNLYEVKSKKHPQIIK
KG

In still other aspects, the fusion proteins may comprise a catalytically active Cas9 derived from S. aureus that comprises an amino acid sequence that is at least 80%, 85%, 90%, 95%, 98%, or 99% identical to the amino acid sequence of:

(SEQ ID NO: 102)
KRNYILGLDIGITSVGYGIIDYETRDVIDAGVRLF
KEANVENNEGRRSKRGARRLKRRRRHRIQRVKKLL
FDYNLLTDHSELSGINPYEARVKGLSQKLSEEEFS
AALLHLAKRRGVHNVNEVEEDTGNELSTKEQISRN
SKALEEKYVAELQLERLKKDGEVRGSINRFKTSDY
VKEAKQLLKVQKAYHQLDQSFIDTYIDLLETRRTY
YEGPGEGSPFGWKDIKEWYEMLMGHCTYFPEELRS
VKYAYNADLYNALNDLNNLVITRDENEKLEYYEKF
QIIENVFKQKKKPTLKQIAKEILVNEEDIKGYRVT
STGKPEFTNLKVYHDIKDITARKEIIENAELLDQI
AKILTIYQSSEDIQEELTNLNSELTQEEIEQISNL
KGYTGTHNLSLKAINLILDELWHTNDNQIAIFNRL
KLVPKKVDLSQQKEIPTTLVDDFILSPVVKRSFIQ
SIKVINAIIKKYGLPNDIIIELAREKNSKDAQKMI
NEMQKRNRQTNERIEEIIRTTGKENAKYLIEKIKL
HDMQEGKCLYSLEAIPLEDLLNNPFNYEVDHIIPR
SVSFDNSFNNKVLVKQEENSKKGNRTPFQYLSSSD
SKISYETFKKHILNLAKGKGRISKTKKEYLLEERD
INRFSVQKDFINRNLVDTRYATRGLMNLLRSYFRV
NNLDVKVKSINGGFTSFLRRKWKFKKERNKGYKHH
AEDALIIANADFIFKEWKKLDKAKKVMENQMFEEK
QAESMPEIETEQEYKEIFITPHQIKHIKDFKDYKY
SHRVDKKPNRELINDTLYSTRKDDKGNTLIVNNLN
GLYDKDNDKLKKLINKSPEKLLMYHHDPQTYQKLK
LIMEQYGDEKNPLYKYYEETGNYLTKYSKKDNGPV
IKKIKYYGNKLNAHLDITDDYPNSRNKVVKLSLKP
YRFDVYLDNGVYKFVTVKNLDVIKKENYYEVNSKC
YEEAKKLKKISNQAEFIASFYNNDLIKINGELYRV
IGVNNDLLNRIEVNMIDITYREYLENMNDKRPPRI
IKTIASKTQSIKKYSTDILGNLYEVKSKKHPQIIK
KG

### Guide RNAs

Embodiments of the present disclosure are directed to the use of a guide RNA which may include one or more of a spacer sequence a tracr mate sequence and a tracr sequence. The term spacer sequence is understood by those of skill in the art and may include any polynucleotide having sufficient complementarity with a target nucleic acid sequence to hybridize with the target nucleic acid sequence and direct sequence-specific binding of a CRISPR complex to the target sequence. The guide RNA may be formed from a spacer sequence covalently connected to a tracr mate sequence (which may be referred to as a crRNA) and a separate tracr sequence, wherein the tracr mate sequence is hybridized to a portion of the tracr sequence. According to certain aspects, the tracr mate sequence and the tracr sequence are connected or linked such as by covalent bonds by a linker sequence, which construct may be referred to as a fusion of the tracr mate sequence and the tracr sequence. The linker sequence referred to herein is a sequence of nucleotides, referred to herein as a nucleic acid sequence, which connect the tracr mate sequence and the tracr sequence. Accordingly, a guide RNA may be a two component species (i.e., separate crRNA and tracr RNA which hybridize together) or a unimolecular species (i.e., a crRNA-tracr RNA fusion, often termed an sgRNA). Exemplary gRNAs comprise guide sequences complementary to one or more repetitive elements, or to one or more unique genomic loci, as provided above.

According to certain aspects, the guide RNA is between about 10 to about 500 nucleotides. According to one aspect, the guide RNA is between about 20 to about 100 nucleotides. According to certain aspects, the spacer sequence is between about 10 and about 500 nucleotides in length. According to certain aspects, the tracr mate sequence is between about 10 and about 500 nucleotides in length. According to certain aspects, the tracr sequence is between about 10 and about 100 nucleotides in length. According to certain aspects, the linker nucleic acid sequence is between about 10 and about 100 nucleotides in length.

According to one aspect, embodiments described herein include guide RNA having a length including the sum of the lengths of a spacer sequence, tracr mate sequence, tracr sequence, and linker sequence (if present). Accordingly, such a guide RNA may be described by its total length which is a sum of its spacer sequence, tracr mate sequence, tracr sequence, and linker sequence (if present). According to this aspect, all of the ranges for the spacer sequence, tracr mate sequence, tracr sequence, and linker sequence (if present) are incorporated herein by reference and need not be repeated. A guide RNA as described herein may have a total length based on summing values pro vided by the ranges described herein. Aspects of the present disclosure are directed to methods of making such guide RNAs as described herein by expressing constructs encoding such guide RNA using promoters and terminators and optionally other genetic elements as described herein.

According to certain aspects, the guide RNA may be delivered directly to a cell as a native species by methods known to those of skill in the art, including injection or lipofection, or as transcribed from its cognate DNA, with the cognate DNA introduced into cells through electroporation, transient and stable transfection (including lipofection) and viral transduction.

It will be apparent to those of skill in the art that in order to target any of the fusion proteins comprising a Cas9 domain and a deaminase, as disclosed herein, to a target site, e.g. a site comprising a point mutation to be edited, it is typically necessary to co-express the fusion protein together with a guide RNA, e.g. an sgRNA. As explained in more detail elsewhere herein, a guide RNA typically comprises a tracrRNA framework allowing for Cas9 binding, and a guide sequence, which confers sequence specificity to the Cas9:nucleic acid editing enzyme/domain fusion protein.

In some embodiments, the guide RNA comprises a structure 5′-[guide sequence]-guuuuagagcuagaaauagcaaguuaaaauaaggcuaguccguuaucaacuugaaaaaguggcaccgagucggugcuuu uu-3′ (SEQ ID NO: 21), wherein the guide sequence comprises a sequence that is complementary to the target sequence. See U.S. Publication No. 2015/0166981, published Jun. 18, 2015, the disclosure of which is incorporated by reference herein in its entirety. The guide sequence is typically 20 nucleotides long. The sequences of suitable guide RNAs for targeting Cas9:nucleic acid editing enzyme/domain fusion proteins to specific genomic target sites will be apparent to those of skill in the art based on the instant disclosure. Such suitable guide RNA sequences typically comprise guide sequences that are complementary to a nucleic sequence within 50 nucleotides upstream or downstream of the target nucleotide to be edited. Some exemplary guide RNA sequences suitable for targeting any of the provided fusion proteins to specific target sequences are provided herein. Additional guide sequences are well known in the art and can be used with the fusion proteins described herein. Additional exemplary guide sequences are disclosed in, for example, Jinek M., et al., Science 337:816-821(2012); Mali P, Esvelt K M & Church G M (2013) Cas9 as a versatile tool for engineering biology, Nature Methods, 10, 957-963; Li J F et al., (2013) Multiplex and homologous recombination-mediated genome editing in Arabidopsis and Nicotiana benthamiana using guide RNA and Cas9, Nature Biotechnology, 31, 688-691; Hwang, W. Y. et al., Efficient genome editing in zebrafish using a CRISPR-Cas system, Nature Biotechnology 31, 227-229 (2013); Cong L et al., (2013) Multiplex genome engineering using CRIPSR/Cas systems, Science, 339, 819-823; Cho S W et al., (2013) Targeted genome engineering in human cells with the Cas9 RNA-guided endonuclease, Nature Biotechnology, 31, 230-232; Jinek, M. et al., RNA-programmed genome editing in human cells, eLife 2, e00471 (2013); Dicarlo, J. E. et al., Genome engineering in Saccharomyces cerevisiae using CRISPR-Cas systems. Nucleic Acid Res. (2013); Briner A E et al., (2014) Guide RNA functional modules direct Cas9 activity and orthogonality, Mol Cell, 56, 333-339, the entire contents of each of which are herein incorporated by reference.

Guide RNA sequences may be cloned into a gRNA expression vector, such as pFYF, to encode a gRNA that targets Cas9, or any of the fusion proteins provided herein, to a target site in order to correct a disease-related mutation. gRNAs may be designed based on the disclosure and the knowledge in the art, which would be appreciated by the skilled artisan.

Exemplary gRNA sequences used in the present disclosure are listed in Table 2.

### Cells

Cells according to the present disclosure include any eukaryotic cell into which foreign nucleic acids can be introduced and expressed as described herein it is to be understood that the basic concepts of the present disclosure described herein are not limited by cell type. In some embodiments, the cell is from an embryo. The cell can be a stem cell, zygote, or a germ line cell. In embodiments wherein the cell is a stem cell, the stem cell is an embryonic stem cell or induced pluripotent stem cell. In other embodiments, the cell is a somatic cell. The eukaryotic cell can be an animal cell, such as from a pig, mouse, rat, rabbit, dog, horse, cow, non-human primate, or human. In some embodiments, the animal cell is a human cell. In particular embodiments, the animal cell is an hiPSC or hES cell.

### UGI Domains

The term “uracil glycosylase inhibitor” or “UGI,” as used herein, refers to a protein that is capable of inhibiting a uracil-DNA glycosylase base-excision repair enzyme. In some embodiments, a UGI domain comprises a wild-type UGI or a UGI as set forth in SEQ ID NO: 5. In some embodiments, the UGI proteins provided herein include fragments of UGI and proteins homologous to a UGI or a UGI fragment. For example, in some embodiments, a UGI domain comprises a fragment of the amino acid sequence set forth in SEQ ID NO: 5. In some embodiments, a UGI fragment comprises an amino acid sequence that comprises at least 60%, at least 65%, at least 70%, at least 75%, at least 80%, at least 85%, at least 90%, at least 95%, at least 96%, at least 97%, at least 98%, at least 99%, or at least 99.5% of the amino acid sequence as set forth in SEQ ID NO: 5. In some embodiments, a UGI comprises an amino acid sequence homologous to the amino acid sequence set forth in SEQ ID NO: 5, or an amino acid sequence homologous to a fragment of the amino acid sequence set forth in SEQ ID NO: 5. In some embodiments, proteins comprising UGI or fragments of UGI or homologs of UGI or UGI fragments are referred to as “UGI variants.” A UGI variant shares homology to UGI, or a fragment thereof. For example a UGI variant is at least 70% identical, at least 75% identical, at least 80% identical, at least 85% identical, at least 90% identical, at least 95% identical, at least 96% identical, at least 97% identical, at least 98% identical, at least 99% identical, at least 99.5% identical, or at least 99.9% identical to a wild type UGI or a UGI as set forth in SEQ ID NO: 5. In some embodiments, the UGI variant comprises a fragment of UGI, such that the fragment is at least 70% identical, at least 80% identical, at least 90% identical, at least 95% identical, at least 96% identical, at least 97% identical, at least 98% identical, at least 99% identical, at least 99.5% identical, or at least 99.9% to the corresponding fragment of wild-type UGI or a UGI as set forth in SEQ ID NO: 5. In some embodiments, the UGI comprises the following amino acid sequence:

(SEQ ID NO: 5)
MTNLSDIIEKETGKQLVIQESILMLPEEVEEVIGN
KPESDILVHTAYDESTDENVMLLTSDAPEYKPWAL
VIQDSNGENKIKML.

The fusion proteins described herein may comprise more than one UGI domain, which may be separated by one or more linkers as described herein.

In some embodiments, the methods and compositions disclosed herein comprise an isolated UGI protein added to the eukaryotic cells subsequent to the step of contacting the target sequence(s) with the fusion protein.

### Additional Base Editor Elements

In various embodiments, the fusion proteins disclosed herein further comprise one or more, preferably at least two nuclear localization signals. In a preferred embodiment, the fusion proteins comprise at least two NLSs. In embodiments with at least two NLSs, the NLSs can be the same NLSs or they can be different NLSs. In addition, the NLSs may be expressed as part of a fusion protein with the remaining portions of the fusion proteins. The location of the NLS fusion can be at the N-terminus, the C-terminus, or within a sequence of a fusion protein (e.g. inserted between the encoded Cas9 and a DNA effector moiety (e.g. a deaminase)).

The NLSs may be any known NLS sequence in the art. The NLSs may also be any future-discovered NLSs for nuclear localization. The NLSs also may be any naturally-occurring NLS, or any non-naturally occurring NLS (e.g. an NLS with one or more desired mutations).

A nuclear localization signal or sequence (NLS) is an amino acid sequence that tags, designates, or otherwise marks a protein for import into the cell nucleus by nuclear transport. Typically, this signal consists of one or more short sequences of positively charged lysines or arginines exposed on the protein surface. Different nuclear localized proteins may share the same NLS. An NLS has the opposite function of a nuclear export signal (NES), which targets proteins out of the nucleus. A nuclear localization signal can also target the exterior surface of a cell. Thus, a single nuclear localization signal can direct the entity with which it is associated to the exterior of a cell and to the nucleus of a cell. Such sequences can be of any size and composition, for example more than 25, 25, 15, 12, 10, 8, 7, 6, 5 or 4 amino acids, but will preferably comprise at least a four to eight amino acid sequence known to function as a nuclear localization signal (NLS).

The term “nuclear localization sequence” or “NLS” refers to an amino acid sequence that promotes import of a protein into the cell nucleus, for example, by nuclear transport. Nuclear localization sequences are known in the art and would be apparent to the skilled artisan. For example, NLS sequences are described in Plank et al., international PCT application, PCT/EP2000/011690, filed Nov. 23, 2000, published as WO 2001/038547 on May 31, 2001, the contents of which are incorporated herein by reference for their disclosure of exemplary nuclear localization sequences. In some embodiments, a NLS comprises the amino acid sequence PKKKRKV (SEQ ID NO: 23),

(SEQ ID NO: 24)
MDSLLMNRRKFLYQFKNVRWAKGRRETYLC,
(SEQ ID NO: 25)
KRTADGSEFESPKKKRKV,
or
(SEQ ID NO: 26)
KRTADGSEFEPKKKRKV.

In various aspects of the disclosure, a fusion protein (e.g. CBE1, CBE2, CBE3, or CBE4, or variants thereof) comprises one or more nuclear localization signals (NLS), preferably at least two NLSs. In preferred embodiments, the fusion proteins are modified with two or more NLSs. The invention contemplates the use of any nuclear localization signal known in the art at the time of the invention, or any nuclear localization signal that is identified or otherwise made available in the state of the art after the time of the instant filing. A representative nuclear localization signal is a peptide sequence that directs the protein to the nucleus of the cell in which the sequence is expressed. A nuclear localization signal is predominantly basic, can be positioned almost anywhere in a protein's amino acid sequence, generally comprises a short sequence of four amino acids (Autieri & Agrawal, (1998) J. Biol. Chem. 273: 14731-37, incorporated herein by reference) to eight amino acids, and is typically rich in lysine and arginine residues (Magin et al., (2000) Virology 274: 11-16, incorporated herein by reference). Nuclear localization signals often comprise proline residues. A variety of nuclear localization signals have been identified and have been used to effect transport of biological molecules from the cytoplasm to the nucleus of a cell. See, e.g. Tinland et al., (1992) Proc. Natl. Acad. Sci. U.S.A. 89:7442-46; Moede et al., (1999) FEBS Leff. 461:229-34, which is incorporated by reference. Translocation is currently thought to involve nuclear pore proteins.

In certain embodiments, linkers may be used to link any of the peptides or peptide domains of the disclosure. As defined above, the term “linker,” as used herein, refers to a chemical group or a molecule linking two molecules or moieties, e.g. a binding domain and a cleavage domain of a nuclease. In some embodiments, a linker joins a dCas9 and deaminase domain (e.g. a cytidine or adenosine deaminase). Typically, the linker is positioned between, or flanked by, two groups, molecules, or other moieties and connected to each one via a covalent bond, thus connecting the two. In some embodiments, the linker is an amino acid or a plurality of amino acids (e.g. a peptide or protein). In some embodiments, the linker is an organic molecule, group, polymer, or chemical moiety. In some embodiments, the linker is 5-100 amino acids in length, for example, 5, 6, 7, 8, 9, 10, 11, 12, 13, 14, 15, 16, 17, 18, 19, 20, 21, 22, 23, 24, 25, 26, 27, 28, 29, 30, 30, 31, 32, 33-40, 40-45, 45-50, 50-60, 60-70, 70-80, 80-90, 90-100, 100-150, or 150-200 amino acids in length. Longer or shorter linkers are also contemplated.

In some embodiments, the linker is a peptide linker, such as an XTEN linker, a 16 amino acid linker. In particular embodiments, the linker comprises the amino acid sequence SGSETPGTSESATPES (SEQ ID NO: 27). In other embodiments, the linker is a 32 amino acid (32aa) linker. In particular embodiments, the linker comprises the amino acid sequence of SGGSSGGSSGSETPGTSESATPESSGGSSGGS (SEQ ID NO: 28).

In other embodiments, the linker comprises the amino acid sequence SGGSGGSGGS (SEQ ID NO:29). In other embodiments, the linker comprises the amino acid sequence SGGS (SEQ ID NO: 30).

In some embodiments, the fusion protein described herein may comprise one or more heterologous protein domains, e.g. epitope tags and reporter gene sequences. In some embodiments, the heterologous protein domain comprises a reporter sequence comprising a p2A-GFP insert ((Addgene plasmid #65562; RRID:Addgene_65562), see Li J, et al., Intron targeting-mediated and endogenous gene integrity-maintaining knockin in zebrafish using the CRISPR/Cas9 system. Cell Res. (2015)). Non-limiting examples of epitope tags include histidine (His) tags, V5 tags, FLAG tags, influenza hemagglutinin (HA) tags, Myc tags, VSV-G tags, and thioredoxin (Trx) tags. Examples of reporter genes include, but are not limited to, glutathione-5-transferase (GST), horseradish peroxidase (HRP), chloramphenicol acetyltransferase (CAT), beta-galactosidase, beta-glucuronidase, luciferase, green fluorescent protein (GFP), HcRed, DsRed, cyan fluorescent protein (CFP), yellow fluorescent protein (YFP), and autofluorescent proteins including blue fluorescent protein (BFP). A fusion protein may be fused to a gene sequence encoding a protein or a fragment of a protein that bind DNA molecules or bind other cellular molecules, including, but not limited to, maltose binding protein (MBP), S-tag, Lex A DNA binding domain (DBD) fusions, GAL4 DNA binding domain fusions, and herpes simplex virus (HSV) BP16 protein fusions. Additional domains that may form part of a fusion protein are described in US Patent Publication No. 2011/0059502, published Mar. 10, 2011 and incorporated herein by reference in its entirety.

### Pharmaceutical Compositions

In various other embodiments, the disclosure provides compositions of eukaryotic cells comprising a plurality of the fusion proteins described herein. In particular embodiments, these compositions further comprise an anti-apoptotic molecule and/or a growth factor, and/or an inhibitor of MMR and/or an inhibitor of base excision repair and/or an inhibitor of non-homologous end joining. Compositions may comprise a combination of all such factors and/or inhibitors. Compositions comprising such factors and/or inhibitors may be added to taret cells immediately after transfection, 4 hours after transfection, 8 hours after transfection, 12 hours after transfection, 16 hours after transfection, 24 hours after transfection, 30 hours after transfection, 35 hours after transfection, 48 hours after transfections, 3 days after transfection, or 4 days after transfection.

The present disclosure also provides pharmaceutical compositions comprising any of the fusion proteins described herein and a gRNA, wherein at least five, ten, fifteen, twenty, or more than twenty of the fusion proteins of the plurality are each bound to a unique gRNA, and a pharmaceutically acceptable excipient. In particular, the disclosure provides pharmaceutical compositions comprising a fusion protein comprising a dCas9 domain, nCas9 domain, and a plurality of gRNAs. In other embodiments, the disclosure provides pharmaceutical compositions comprising a plurality of gRNAs (e.g. sgRNAs) that are complementary to a target sequence that has 25, 30, 35, 40, 45, 50, 60, 75, 100, 250, 500, 1,000, 2,000, 3,000 or more than 3,000 copies in the target genome. In particular, these pharmaceutical compositions may comprise promiscuous gRNAs.

In other embodiments, the disclosure provides pharmaceutical compositions comprising a fusion protein comprising a TAL effector domain, and a plurality of cofactor proteins (e.g. FokI endonucleases) to be delivered to target cells separately from the fusion protein.

In particular embodiments, the disclosed pharmaceutical compositions further comprise one or more of an anti-apoptotic molecule, a growth factor, an inhibitor of mismatch repair, inhibitor of base excision repair and an inhibitor of non-homologous end joining. In certain embodiments, administration of the disclosed pharmaceutical compositions results in low toxicity when administered to a population of cells. In particular embodiments, less than 30%, less than 20%, less than 15%, less than 10%, less than 5%, or less than 1% cell death in the population of cells is observed. Other embodiments of the present disclosure relate to pharmaceutical compositions comprising the fusion protein-gRNA complexes described herein. The term “pharmaceutical composition,” as used herein, refers to a composition formulated for pharmaceutical use. In some embodiments, the pharmaceutical composition further comprises a pharmaceutically acceptable carrier. In some embodiments, the pharmaceutical composition comprises additional agents (e.g. for specific delivery, increasing half-life, or other therapeutic compounds).

In some embodiments, any of the fusion proteins, gRNAs, and/or complexes described herein are provided as part of a pharmaceutical composition. In some embodiments, the pharmaceutical composition comprises any of the fusion proteins provided herein. In some embodiments, the pharmaceutical composition comprises any of the complexes provided herein. In some embodiments pharmaceutical composition comprises a gRNA, a dCas9 fusion protein, and a pharmaceutically acceptable excipient. In some embodiments pharmaceutical composition comprises a cofactor protein (e.g. a FokI endonuclease), a TAL effector fusion protein, and a pharmaceutically acceptable excipient. Pharmaceutical compositions may optionally comprise one or more additional therapeutically active substances.

In some embodiments, compositions provided herein are administered to a subject, for example, to a human subject, in order to effect a targeted genomic modification within the subject. In some embodiments, cells are obtained from the subject and contacted with a any of the pharmaceutical compositions provided herein. In some embodiments, cells removed from a subject and contacted ex vivo with a pharmaceutical composition are re-introduced into the subject, optionally after the desired genomic modification has been effected or detected in the cells. Methods of delivering pharmaceutical compositions comprising nucleases are known, and are described, for example, in U.S. Pat. Nos. 6,453,242; 6,503,717; 6,534,261; 6,599,692; 6,607,882; 6,689,558; 6,824,978; 6,933,113; 6,979,539; 7,013,219; and 7,163,824, the disclosures of all of which are incorporated by reference herein in their entireties. Although the descriptions of pharmaceutical compositions provided herein are principally directed to pharmaceutical compositions which are suitable for administration to humans, it will be understood by the skilled artisan that such compositions are generally suitable for administration to animals or organisms of all sorts. Modification of pharmaceutical compositions suitable for administration to humans in order to render the compositions suitable for administration to various animals is well understood, and the ordinarily skilled veterinary pharmacologist can design and/or perform such modification with merely ordinary, if any, experimentation. Subjects to which administration of the pharmaceutical compositions is contemplated include, but are not limited to, humans and/or other primates; mammals, domesticated animals, pets, and commercially relevant mammals such as cattle, pigs, horses, sheep, cats, dogs, mice, and/or rats; and/or birds, including commercially relevant birds such as chickens, ducks, geese, and/or turkeys.

Formulations of the pharmaceutical compositions described herein may be prepared by any method known or hereafter developed in the art of pharmacology. In general, such preparatory methods include the step of bringing the active ingredient(s) into association with an excipient and/or one or more other accessory ingredients, and then, if necessary and/or desirable, shaping and/or packaging the product into a desired single- or multi-dose unit.

Pharmaceutical formulations may additionally comprise a pharmaceutically acceptable excipient, which, as used herein, includes any and all solvents, dispersion media, diluents, or other liquid vehicles, dispersion or suspension aids, surface active agents, isotonic agents, thickening or emulsifying agents, preservatives, solid binders, lubricants and the like, as suited to the particular dosage form desired. Remington's The Science and Practice of Pharmacy, 21^{st} Edition, A. R. Gennaro (Lippincott, Williams & Wilkins, Baltimore, Md., 2006; incorporated in its entirety herein by reference) discloses various excipients used in formulating pharmaceutical compositions and known techniques for the preparation thereof. See also PCT application PCT/US2010/055131 (Publication No. WO/2011053982), filed Nov. 2, 2010, incorporated in its entirety herein by reference, for additional suitable methods, reagents, excipients and solvents for producing pharmaceutical compositions comprising a nuclease. Except insofar as any conventional excipient medium is incompatible with a substance or its derivatives, such as by producing any undesirable biological effect or otherwise interacting in a deleterious manner with any other component(s) of the pharmaceutical composition, its use is contemplated to be within the scope of this disclosure.

As used here, the term “pharmaceutically acceptable carrier” means a pharmaceutically acceptable material, composition or vehicle, such as a liquid or solid filler, diluent, excipient, manufacturing aid (e.g. lubricant, talc magnesium, calcium or zinc stearate, or steric acid), or solvent encapsulating material, involved in carrying or transporting the compound from one site (e.g. the delivery site) of the body, to another site (e.g. organ, tissue or portion of the body). A pharmaceutically acceptable carrier is “acceptable” in the sense of being compatible with the other ingredients of the formulation and not injurious to the tissue of the subject (e.g. physiologically compatible, sterile, physiologic pH, etc.). Some examples of materials which can serve as pharmaceutically acceptable carriers include: (1) sugars, such as lactose, glucose and sucrose; (2) starches, such as corn starch and potato starch; (3) cellulose, and its derivatives, such as sodium carboxymethyl cellulose, methylcellulose, ethyl cellulose, microcrystalline cellulose and cellulose acetate; (4) powdered tragacanth; (5) malt; (6) gelatin; (7) lubricating agents, such as magnesium stearate, sodium lauryl sulfate and talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol (PEG); (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) pH buffered solutions; (21) polyesters, polycarbonates and/or polyanhydrides; (22) bulking agents, such as polypeptides and amino acids (23) serum component, such as serum albumin, HDL and LDL; (22) C2-C12 alcohols, such as ethanol; and (23) other non-toxic compatible substances employed in pharmaceutical formulations. Wetting agents, coloring agents, release agents, coating agents, sweetening agents, flavoring agents, perfuming agents, preservative and antioxidants may also be present in the formulation. The terms such as “excipient”, “carrier”, “pharmaceutically acceptable carrier” or the like are used interchangeably herein.

In some embodiments, the pharmaceutical composition is formulated for delivery to a subject, e.g. for multiplexed gene editing. Suitable routes of administrating the pharmaceutical composition described herein include, without limitation: topical, subcutaneous, transdermal, intradermal, intralesional, intraarticular, intraperitoneal, intravesical, transmucosal, gingival, intradental, intracochlear, transtympanic, intraorgan, epidural, intrathecal, intramuscular, intravenous, intravascular, intraosseus, periocular, intratumoral, intracerebral, and intracerebroventricular administration.

In some embodiments, the pharmaceutical composition described herein is administered locally to a diseased site. In some embodiments, the pharmaceutical composition described herein is administered to a subject by injection, by means of a catheter, by means of a suppository, or by means of an implant, the implant being of a porous, non-porous, or gelatinous material, including a membrane, such as a sialastic membrane, or a fiber.

In some embodiments, the pharmaceutical composition is formulated in accordance with routine procedures as a composition adapted for intravenous or subcutaneous administration to a subject, e.g. a human. In some embodiments, pharmaceutical composition for administration by injection are solutions in sterile isotonic aqueous buffer. Where necessary, the pharmaceutical can also include a solubilizing agent and a local anesthetic such as lignocaine to ease pain at the site of the injection. Generally, the ingredients are supplied either separately or mixed together in unit dosage form, for example, as a dry lyophilized powder or water free concentrate in a hermetically sealed container such as an ampoule or sachette indicating the quantity of active agent. Where the pharmaceutical is to be administered by infusion, it can be dispensed with an infusion bottle containing sterile pharmaceutical grade water or saline. Where the pharmaceutical composition is administered by injection, an ampoule of sterile water for injection or saline can be provided so that the ingredients can be mixed prior to administration.

The pharmaceutical composition can be contained within a lipid particle or vesicle, such as a liposome or microcrystal, which is also suitable for parenteral administration. The particles can be of any suitable structure, such as unilamellar or plurilamellar, so long as compositions are contained therein. Compounds can be entrapped in “stabilized plasmid-lipid particles” (SPLP) containing the fusogenic lipid dioleoylphosphatidylethanolamine (DOPE), low levels (5-10 mol %) of cationic lipid, and stabilized by a polyethyleneglycol (PEG) coating (Zhang Y. P. et al., Gene Ther. 1999, 6:1438-47). Positively charged lipids such as N-[1-(2,3-dioleoyloxi)propyl]-N,N,N-trimethyl-amoniummethylsulfate, or “DOTAP,” are particularly preferred for such particles and vesicles. The preparation of such lipid particles is well known. See, e.g. U.S. Pat. Nos. 4,880,635; 4,906,477; 4,911,928; 4,917,951; 4,920,016; 4,921,757; and 9,737,604, each of which is incorporated herein by reference.

The pharmaceutical composition described herein may be administered or packaged as a unit dose, for example. The term “unit dose” when used in reference to a pharmaceutical composition of the present disclosure refers to physically discrete units suitable as unitary dosage for the subject, each unit containing a predetermined quantity of active material calculated to produce the desired therapeutic effect in association with the required diluent; i.e., carrier, or vehicle.

Further, the pharmaceutical composition may be provided as a pharmaceutical kit comprising (a) a container containing a compound of the invention in lyophilized form and (b) a second container containing a pharmaceutically acceptable diluent (e.g. sterile water) for injection. The pharmaceutically acceptable diluent can be used for reconstitution or dilution of the lyophilized compound of the invention. Optionally associated with such container(s) can be a notice in the form prescribed by a governmental agency regulating the manufacture, use or sale of pharmaceuticals or biological products, which notice reflects approval by the agency of manufacture, use or sale for human administration.

In another aspect, an article of manufacture containing materials useful for the treatment of the diseases described above is included. In some embodiments, the article of manufacture comprises a container and a label. Suitable containers include, for example, bottles, vials, syringes, and test tubes. The containers may be formed from a variety of materials such as glass or plastic. In some embodiments, the container holds a composition that is effective for treating a disease described herein and may have a sterile access port. For example, the container may be an intravenous solution bag or a vial having a stopper pierceable by a hypodermic injection needle. The active agent in the composition is a compound of the invention. In some embodiments, the label on or associated with the container indicates that the composition is used for treating the disease of choice. The article of manufacture may further comprise a second container comprising a pharmaceutically acceptable buffer, such as phosphate-buffered saline, Ringer's solution, or dextrose solution. It may further include other materials desirable from a commercial and user standpoint, including other buffers, diluents, filters, needles, syringes, and package inserts with instructions for use.

### Delivery Methods

In some embodiments, the disclosure provides methods comprising delivering any of the fusion proteins, gRNAs, cofactor proteins, vectors and/or complexes described herein. In other embodiments, the disclosure provides methods comprising delivery of one or more vectors as described herein, one or more transcripts thereof, and/or one or proteins transcribed therefrom, to a host cell (e.g. eukaryotic cell). In some embodiments, the invention further provides cells produced by such methods, and organisms (such as animals, plants, or fungi) comprising or produced from such cells. In some embodiments, a fusion protein as described herein in combination with (and optionally complexed with) a guide sequence is delivered to a cell. Delivery can be to cells (e.g. in vitro or ex vivo administration) or target tissues (e.g. in vivo administration). Delivery may be achieved through the use of RNP complexes.

Conventional viral and non-viral based gene transfer methods may be used to introduce nucleic acids in mammalian cells or target tissues. Such methods may be used to administer nucleic acids encoding components of a fusion protein to cells in culture, or in a host organism. Non-viral vector delivery systems include ribonucleoprotein (RNP) complexes, DNA plasmids, RNA (e.g. a transcript of a vector described herein), naked nucleic acid, and nucleic acid complexed with a delivery vehicle, such as a liposome. Viral vector delivery systems include DNA and RNA viruses, which have either episomal or integrated genomes after delivery to the cell. For a review of gene therapy procedures, see Anderson, Science 256:808-813 (1992); Nabel & Feigner, TIBTECH 11:211-217 (1993); Mitani & Caskey, TIBTECH 11:162-166 (1993); Dillon, TIBTECH 11:167-175 (1993); Miller, Nature 357:455-460 (1992); Van Brunt, Biotechnology 6(10):1149-1154 (1988); Vigne, Restorative Neurology and Neuroscience 8:35-36 (1995); Kremer & Perricaudet, British Medical Bulletin 51(1):31-44 (1995); Haddada et al., in Current Topics in Microbiology and Immunology Doerfler and Bihm (eds) (1995); and Yu et al., Gene Therapy 1:13-26 (1994).

In certain embodiments, the method of delivery provided herein comprises lipofection. Lipofection is described in e.g. U.S. Pat. Nos. 5,049,386, 4,946,787; 4,897,355; and 9,737,604) and lipofection reagents are sold commercially (e.g. Transfectam™ and Lipofectin™). Cationic and neutral lipids that are suitable for efficient receptor-recognition lipofection of polynucleotides include those of Feigner, WO 1991/17424; WO 1991/16024. In certain embodiments, the method of delivery comprises electroporation. In certain embodiments, the method of delivery provided herein comprises stable genome integration (e.g. piggybac).

In other embodiments, the method of delivery and vector provided herein is an RNP complex. RNP delivery of fusion proteins markedly increases the DNA specificity of base editing. RNP delivery of fusion proteins leads to decoupling of on- and off-target editing. RNP delivery ablates off-target editing at non-repetitive sites while maintaining on-target editing comparable to plasmid delivery, and greatly reduces off-target editing even at the highly repetitive VEGFA site 2. See Rees, H. A. et al., Improving the DNA specificity and applicability of base editing through protein engineering and protein delivery, Nat. Commun. 8, 15790 (2017), U.S. Pat. No. 9,526,784, issued Dec. 27, 2016, and U.S. Pat. No. 9,737,604, issued Aug. 22, 2017, each of which is incorporated by reference herein

In other embodiments, the method of delivery provided comprises nucleofection, microinjection, biolistics, virosomes, liposomes, immunoliposomes, polycation or lipid:nucleic acid conjugates, naked DNA, artificial virions, and agent-enhanced uptake of DNA.

The preparation of lipid:nucleic acid complexes, including targeted liposomes such as immunolipid complexes, is well known to one of skill in the art (see, e.g. Crystal, Science 270:404-410 (1995); Blaese et al., Cancer Gene Ther. 2:291-297 (1995); Behr et al., Bioconjugate Chem. 5:382-389 (1994); Remy et al., Bioconjugate Chem. 5:647-654 (1994); Gao et al., Gene Therapy 2:710-722 (1995); Ahmad et al., Cancer Res. 52:4817-4820 (1992); U.S. Pat. Nos. 4,186,183, 4,217,344, 4,235,871, 4,261,975, 4,485,054, 4,501,728, 4,774,085, 4,837,028, and 4,946,787).

The use of RNA or DNA viral based systems for the delivery of nucleic acids take advantage of highly evolved processes for targeting a virus to specific cells in the body and trafficking the viral payload to the nucleus. Viral vectors may be administered directly to patients (in vivo) or they may be used to treat cells in vitro, and the modified cells may optionally be administered to patients (ex vivo). Conventional viral based systems could include retroviral, lentivirus, adenoviral, adeno-associated, and herpes simplex virus vectors for gene transfer. Additionally, high transduction efficiencies have been observed in many different cell types and target tissues.

### Kits, Vectors, Cells

This disclosure provides kits comprising a nucleic acid construct comprising nucleotide sequences encoding the fusion proteins, gRNAs, cofactor proteins, and/or complexes described herein. Some embodiments of this disclosure provide kits comprising a nucleic acid construct comprising a nucleotide sequence encoding a Cas9-deaminase fusion protein capable of deaminating a targeted cytosine in a nucleic acid molecule. In some embodiments, the nucleotide sequence encodes any of the fusion proteins provided herein. In some embodiments, the nucleotide sequence comprises a heterologous promoter that drives expression of the fusion protein.

In addition, the disclosure provides kits comprising a nucleic acid construct that includes (i) a nucleic acid sequence encoding comprising a plurality of fusion proteins described herein, (ii) a heterologous promoter that drives expression of the sequence of (a); (iii) a nucleic acid sequence encoding one or more gRNAs, (iv) a heterologous promoter that drives expression of (b); and (v) an expression construct encoding a plurality of unique guide RNA backbones, wherein the construct comprises a cloning site positioned to allow the cloning of a nucleic acid sequence identical or complementary to a target sequence into each of the guide RNA backbones.

The disclosure further provides kits comprising a plurality of fusion proteins described herein, a plurality of gRNAs with complementarity to the target sequences, and one or more of the following: cofactor proteins, buffers, growth factors, anti-apoptotic factors, inhibitors of base excision repair, inhibitors of MMR, inhibitors of NHEJ, media, and target cells (e.g. human IPSC cells). Kits may comprise combinations of several or all of the aforementioned components.

Some embodiments of this disclosure provide kits comprising a nucleic acid construct, comprising (a) a nucleotide sequence encoding a DNA binding protein (e.g. a Cas9 domain) fused to a deaminase, or a fusion protein comprising a DNA binding protein (e.g. TAL effector domain), a deaminase and a cofactor protein as provided herein; and (b) a heterologous promoter that drives expression of the sequence of (a). In certain embodiments, the kit further comprises an expression construct encoding a guide nucleic acid backbone, e.g. a guide RNA backbone, wherein the construct comprises a cloning site positioned to allow the cloning of a nucleic acid sequence identical or complementary to a target sequence into the guide nucleic acid, e.g. guide RNA backbone.

Some embodiments of this disclosure provide cells comprising any of the fusion proteins or complexes provided herein. In some embodiments, the cells comprise a nucleotide that encodes any of the fusion proteins provided herein. In some embodiments, the cells comprise any of the nucleotides or vectors provided herein.

In some embodiments, a host cell is transiently or non-transiently transfected with one or more vectors described herein. In some embodiments, a cell is transfected as it naturally occurs in a subject. In some embodiments, a cell that is transfected is taken from a subject. In some embodiments, the cell is derived from cells taken from a subject, such as a cell line. A wide variety of cell lines for tissue culture are known in the art. Examples of cell lines include, but are not limited to, C8161, CCRF-CEM, MOLT, mIMCD-3, NHDF, HeLa-S3, Huh1, Huh4, Huh7, HUVEC, HASMC, HEKn, HEKa, MiaPaCell, Panc1, PC-3, TF1, CTLL-2, C1R, Rat6, CV1, RPTE, A10, T24, J82, A375, ARH-77, Calu1, SW480, SW620, SKOV3, SK-UT, CaCo2, P388D1, SEM-K2, WEHI-231, HB56, TIB55, Jurkat, J45.01, LRMB, Bcl-1, BC-3, IC21, DLD2, Raw264.7, NRK, NRK-52E, MRC5, MEF, Hep G2, HeLa B, HeLa T4, COS, COS-1, COS-6, COS-M6A, BS-C-1 monkey kidney epithelial, BALB/3T3 mouse embryo fibroblast, 3T3 Swiss, 3T3-L1, 132-d5 human fetal fibroblasts; 10.1 mouse fibroblasts, 293-T, 3T3, 721, 9L, A2780, A2780ADR, A2780cis, A 172, A20, A253, A431, A-549, ALC, B16, B35, BCP-1 cells, BEAS-2B, bEnd.3, BHK-21, BR 293. BxPC3. C3H-10T1/2, C6/36, Cal-27, CHO, CHO-7, CHO-K1, CHO-K2, CHO-T, CHO Dhfr−/−, COR-L23, COR-L23/CPR, COR-L23/5010, COR-L23/R23, COS-7, COV-434, CMLT1, CMT, CT26, D17, DH82, DU145, DuCaP, EL4, EM2, EM3, EMT6/AR1, EMT6/AR10.0, FM3, H1299, H69, HB54, HB55, HCA2, HEK-293, HeLa, Hepa1c1c7, HL-60, HMEC, HT-29, Jurkat, JY cells, K562 cells, Ku812, KCL22, KG1, KYO1, LNCap, Ma-Mel 1-48, MC-38, MCF-7, MCF-10A, MDA-MB-231, MDA-MB-468, MDA-MB-435, MDCK II, MDCK 11, MOR/0.2R, MONO-MAC 6, MTD-1A, MyEnd, NCI-H69/CPR, NCI-H69/LX10, NCI-H69/LX20, NCI-H69/LX4, NIH-3T3, NALM-1, NW-145, OPCN/OPCT cell lines, Peer, PNT-1A/PNT 2, RenCa, RIN-5F, RMA/RMAS, Saos-2 cells, Sf-9, SkBr3, T2, T-47D, T84, THP1 cell line, U373, U87, U937, VCaP, Vero cells, WM39, WT-49, X63, YAC-1, YAR, and transgenic varieties thereof. Cell lines are available from a variety of sources known to those with skill in the art (see, e.g. the American Type Culture Collection (ATCC) (Manassas, Va.)). In some embodiments, a cell transfected with one or more vectors described herein is used to establish a new cell line comprising one or more vector-derived sequences. In some embodiments, a cell transiently transfected with the components of a CRISPR system as described herein (such as by transient transfection of one or more vectors, or transfection with RNA), and modified through the activity of a CRISPR complex, is used to establish a new cell line comprising cells containing the modification but lacking any other exogenous sequence. In some embodiments, cells transiently or non-transiently transfected with one or more vectors described herein, or cell lines derived from such cells are used in assessing one or more test compounds.

In particular embodiments, the plasmid vectors utilized in the disclosed methods comprise at the 3′ terminus a Bovine Growth Hormone Polyadenylation Signal (bGHpA), which is a specialized termination sequence for protein expression in eukaryotic cells. bGHpA is a polyadenylation signal derived from the gene for bovine growth hormone and is used to obtain optimize expression of the recombinant transgenes operably linked thereto. Reference is made to U.S. Pat. No. 5,122,458, incorporated herein by reference.

The following Examples demonstrate that dCas9 fusion proteins may be used to edit thousands of TE sites concurrently in human cells. Additional modifications, notably the use of bacterial mu-gam, which was originally reported to increase purity, also increased survival of highly edited cells. To demonstrate the safety of the new fusion protein variants disclosed herein, it was shown that high copy TE editing may be conducted in human induced pluripotent stem cells (hiPSCs). Samples were screened for targeted deamination, random indel mutagenesis and their capacity to form stable edited cell lines. A “survival cocktail” of small molecules and growth factors that enhances stable editing was developed for supplementing the target cells immediately to within four days post-transfection. Finally, the best DNA editor and survival conditions were combined to probe the feasibility of large-scale editing in human cells. An estimated 6292 of 26,000 loci, or 24.2% LINE-1 sequences, in hiPSCs were inactivated by the disclosed base editing methods.

Without further elaboration, it is believed that one skilled in the art can, based on the above description, utilize the present invention to its fullest extent. The following specific embodiments are, therefore, to be construed as merely illustrative, and not limitative of the remainder of the disclosure in any way whatsoever. All publications cited herein are incorporated by reference for the purposes or subject matter referenced herein.

These and other aspects of the present invention will be further appreciated upon consideration of the following Examples, which are intended to illustrate certain particular embodiments of the invention but are not intended to limit its scope, as defined by the claims.

### EXAMPLES

In order that the present disclosure may be more fully understood, the following examples are set forth. The synthetic and biological examples described in this application are offered to illustrate the compounds, pharmaceutical compositions, and methods provided herein and are not to be construed in any way as limiting their scope.

### Example 1

### Materials and Methods

Transposable Element gRNA Design

gRNAs targeting Alu were designed by downloading the consensus sequence from repeatmasker (repeatmasker.org/species/hg.html). LINE-1 gRNAs were designed based on the consensus of 146 “Human Full-Length, Intact LINE-1 Elements” available from the L1base 2⁴⁴. HL1gR 1-6 were designed to generate stop codons from C->T deamination mutations. EN, RT and ENRT pairs of gRNAs were designed to create moderate size deletions (200-800 bp) easily distinguishable from their wild type full-length forms by gel visualization. Human Endogenous Retrovirus-W (HERV-W) gRNAs were designed based on the consensus sequence of the 26 sequences identified by Grandi et al.⁴⁵ that can lead to the translation of putative proteins.

qPCR Evaluation of Copy Number Across Repetitive Element Targeting gRNAs

The qPCR reactions were generated using the KAPA SYBR FAST Universal 2X qPCR Master Mix (Catalog #KK4602) according to the manufacturer's instructions. The LightCycler 96 machine from Roche was used to perform the qPCRs and the results were extracted using the LightCycler 96 SW 1.1 software. The following thermocycling conditions were used: “preincubation” stage=95° C. for 180 sec; “2-step cycling” stage: annealing=95° C. for 3 sec and elongation=60° C. for 20 sec; “Melting” stage=keep standard. The following primers were used to perform the qPCRs. Fasta sequences of hg38 reference genome were downloaded from Ensembl (ftp://ftp.ensembl.org/pub/release-95/fasta/homo_sapiens/dna/). Alignment analysis of the gRNA sequences to all chromosomes was performed using the R library Biostrings v2.40.2 and plotted using the R library ggplot2 3.3.0.

TABLE 1
qPCR primers

| Primer name | Sequence | Target |
|---|---|---|
| ZY-JAK2-F | AGCAAGTATG | JAK2 |
| | ATGAGCAAGC | |
| | (SEQ ID | |
| | NO: 31) | |
| SB-JAK2-R | AAAACAGATG | |
| | CTCTGAGAAA | |
| | GGC | |
| | (SEQ ID | |
| | NO: 32) | |
| P1(b)_REBE_F | TAGGAACAGC | LINE-1 |
| | TCCGGTCTAC | promoter |
| | A | |
| | (SEQ ID | |
| | NO: 33) | |
| P1_REBE-ilu_R | AATGCCTCGC | |
| | CCTGCTTCGG | |
| | (SEQ ID | |
| | NO: 34) | |
| P5_REBE-ilu_F | CCAATACAGA | LINE-1 ORF1 |
| | GAAGTGCTTA | |
| | AAGG | |
| | (SEQ ID | |
| | NO: 35) | |
| P5_REBE-ilu_R | CTTGGAGGCT | |
| | TTGCTCATTT | |
| | CT | |
| | (SEQ ID | |
| | NO: 36) | |
| P7_REBE-ilu_F | CCCATCAGTG | LINE-1 ORF2 |
| | TGCTGTATTC | |
| | AGG | |
| | (SEQ ID | |
| | NO: 37) | |
| P7_REBE-ilu_R | GGCCTTCTTT | |
| | GTCTCTTTTG | |
| | (SEQ ID | |
| | NO: 38) | |
| P13_REBE-ilu_F | AACAGGCTCT | LINE-1 ORF2 |
| | GAAATTGTGG | |
| | C | |
| | (SEQ ID | |
| | NO: 39) | |
| P13_REBE-ilu_R | GCTGGCCTCA | |
| | TAAAATGAGT | |
| | TAG | |
| | (SEQ ID | |
| | NO: 40) | |
| P15_REBE-ilu_F | GTTCTGGCCA | LINE-1 ORF2 |
| | GGGCAATCAG | |
| | (SEQ ID | |
| | NO: 41) | |
| P15_REBE-ilu_R | CCTGAGACTT | |
| | TGCTGAAGTT | |
| | GC | |
| | (SEQ ID | |
| | NO: 42) | |
| P3_HERVWenv_F | AATACCACCC | HERV-W cnv |
| | TCACTGGGCT | |
| | (SEQ ID | |
| | NO: 43) | |
| P3_HERVWenv_R | CAGATTGGAA | |
| | ACAAGAGGTC | |
| | C | |
| | (SEQ ID | |
| | NO: 44) | |

SpCas9 and gRNA Plasmids Used for Genome Editing

The following Cas9 plasmids were used: pCas9_GFP (Addgene #44719), hCas9 (Addgene #41815). Base editing plasmids used: pCMV_BE3 (Addgene #73021), pCMV_BE4 (Addgene #100802), pCMV_BE4-gam (Addgene #100806), ABE 7.10 (Addgene #102909). The gRNAs used in the present disclosure were synthesized and cloned as previously described⁴⁶. Briefly, two 24mer oligos with sticky ends compatible for ligation were synthesized from IDT for cloning into the pSB700 plasmid (Addgene Plasmid #64046).

S. aureus Cas9 (SaCas9) and gRNA Plasmids Used for Genome Editing

Cas9 plasmid: pX600-AAV-CMV::NLS-SaCas9-NLS-3×HA-bGHpA (Addgene #61592). Base editing plasmid: SaBE4-gam (Addgene #100809). The gRNAs used in the present disclosure were synthesized and cloned as previously described.⁴⁷ Briefly, two 24mer oligos with sticky ends compatible for ligation were synthesized from IDT for cloning into the BPK2660 plasmid (Addgene Plasmid #70709).

### Maintenance and Transfection of HEK 293T Cells

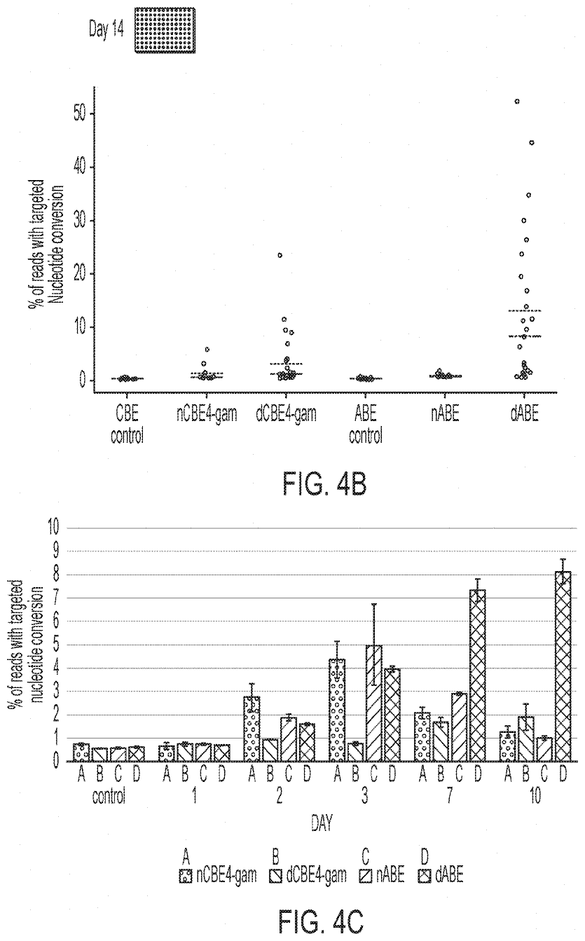

HEK 293T cells were obtained from ATCC with verification of cell line identification and mycoplasma negative results. They were expanded using 10% fetal bovine serum (FBS) in high-glucose DMEM with glutamax passaging at a typical rate of 1:100 and maintained at 37° C. with 5% CO₂. Transfection was conducted using Lipofectamine 2000 (ThermoFisher Catalogue #11668019) using the protocol recommended by the manufacturer with slight modifications outlined below. 24 hours before transfection ˜1.0×10⁵ cells were seeded per well in a 12-well plate along with 1 mL of media. A total of 2 μg of DNA and 2 μL of Lipofectamine 2000 were used per well. For Cas9 plasmids, the DNA content per well was 1 μg of pCas9_GFP mixed with 1 μg of gRNA-expressing plasmid. For BE plasmids, 1.5 μg of BE was mixed with 0.5 μg of gRNA plasmid. In the dBE vs nBE comparison used to generate FIGS. 4A-4C, Pifithrin-α (10 ng/μ1) from Sigma-Aldrich P4359 (source #063M4741V, Batch #0000003019) was added to the media 30 minutes before transfection and maintained in the first day media change.

### FACS Single Cell Direct NGS Preparation

To quantify early genetic editing in cells transfected with Cas9/BE and gRNA expression plasmids, single cells were sorted and prepared as follows. Two days post-transfection, single cells were FACS-sorted into 96-well PCR plates containing 10 μL of QUICKEXTRACT™ DNA Extraction Solution (Epicentre Cat. #QE09050) per well and genomic DNA (gDNA) was extracted using the manufacturer's protocol. Briefly, the sorted plates were sealed, vortexed and heated at 65° C. for 6 minutes then 98° C. for 2 minutes. The NGS library was prepared as described later below.

### Single Cell Clonal Isolation and Sequence Verification

Single cells were FACS-sorted into flat bottom 96-well plates containing 100 μL of DMEM with 10% FBS and 1% Penicillin/Streptomycin per well. Sorted plates were incubated for ˜14 days until well-characterized colonies were visible, with periodic media changes performed as necessary. To extract gDNA, the cells were first detached using 30 μL TRYPLE™ Express (Thermofisher Cat. #12604021) and neutralized with 30 μL growth media. Then, 4 μL of the resulting cell suspension was transferred to 10 μL of QE. Genomic DNA was extracted according to manufacturer's protocol, as described previously.

### Nested PCR Illumina MiSeq Library Preparation and Sequencing

Library preparation was conducted as previously described⁵. Briefly, genomic DNA was amplified using locus-specific primers attached to part of the Illumina adapter sequence. A second round of PCR included the index sequence and the full Illumina adapter. All PCRs were carried out using KAPA HiFi HotStart ReadyMix (KAPA Biosystems KK2602) according to the manufacturer's thermocycler conditions. Libraries were purified using gel extraction (Qiagen Cat. #28706), quantified using Nanodrop and pooled together for deep sequencing on the MiSeq using 150 paired end (PE) reads.

### NGS Indel Analysis

Raw Illumina sequencing data was demultiplexed using bcl2fastq. All paired end reads were aligned to the reference genome using bowtie2⁴⁹ and the resulting alignment files were parsed for their cigar string to determine the position and size of all indels within each read using a custom perl script (https://github.com/CRISPRengineer/mutation_indel). All indels that were sequenced in both the forward and reverse reads were summed across all reads and reported for each sample along with the total number of reads. Indels within a 30 bp window from the 5′ start of the gRNA proceeding through the PAM and extending an additional seven bp's (for a 20 bp gRNA) were counted and summed for each sample.

Dual gRNA Deletion Frequency NGS Analysis

Reads were analyzed for dual gRNA large deletions by detecting sequences in between the gRNAs to indicate the full length unedited (at least not dual gRNA-edited) and sequences beyond the normal wild type amplicon that only appear when the deletion has occurred to identify deletion reads. The custom perl script used for analysis is available at https://github.com/CRISPRengineer/dual gRNA.

### NGS Base Editing Deamination Analysis

All paired end reads were aligned to the reference genome using bowtie2, and the resulting alignment files were converted to bam, sorted, indexed, and variant called using samtools⁷. All SNV data within a 30 bp window from the 5′ start of the gRNA proceeding through the PAM and extending an additional seven bp's (for a 20 bp gRNA) are reported to analyze the editing window and purity of editing. The custom perl script used for analysis is available at https://github.com/CRISPRengineer/deamination_report.

Site Directed Mutagenesis to Remove Remaining Nick from Fusion Proteins

The remaining nuclease domain of Cas9 was deactivated from nCBE4 (Addgene #100802), nCBE4-gam (Addgene #100806), and pCMV-ABE7.10 (Addgene #102919), and SaCas9-BE4-gam (Addgene #100809). Agilent QuikChange XL Site-Directed Mutagenesis Kit (catalogue #200517) was used with the following primer sequences:

SpCas9-fwd-
(SEQ ID NO: 45)
tttatctgattacgacgtcgatgc
cattgtaccccaatcctttttg
SpCas9-rev-
(SEQ ID NO: 46)
caaaaaggattggggtacaatggc
atcgacgtcgtaatcagataaa

### Propidium Iodide and Annexin V Staining and FACS Analysis

Cells were dissociated with TrypLE, diluted in an equal volume of PBS, and then centrifuged at ˜300 g for 5 minutes at room temperature. Samples were resuspended into 500p1 PBS and half of the cells were pelleted for later gDNA analysis. The remainder was centrifuged and resuspended into 100 μl of Annexin V Binding Buffer (ref #V13246) diluted into ultrapure water at a 1:5 ratio. Subsequently, 5p1 of Alexa 647 Annexin V dye (ref #A23204) was added and the samples were incubated in the dark for 15 minutes. Then, 100 μl of Annexin V Binding Buffer was added and 4 μl of Propidium Iodide (ref #P3566) diluted into the Annexin V Binding Buffer was added at a 1:10 ratio. Samples were incubated in the dark for another 15 minutes. Cells were washed with 500p1 of Annexin V Binding Buffer and centrifuged again to be finally resuspended into 400p1 of Annexin V Binding Buffer. All samples were filtered using a cell strainer and were run on the LSR 11 using a 70-μm nozzle. Analysis was conducted using FlowJo software.

Karyotype Analysis of LINE-1 dBE-Edited 293T Single Cell Clones

Stable HEK 293T edited isolated cell lines (BE4-gam, dBE4-gam, ABE and dABE) were expanded and karyotypically compared with the control groups and the wild type HEK 293T. Actively growing cells were passaged 1-2 days prior to sending to BWH CytoGenomics Core Laboratory. The cells were received by the core at 60-80% confluency. Chromosomal count, variances and abnormalities were investigated.

### Whole Genome Sequencing Off-Target Analysis

The top 293T edited clones used for the karyotype analysis were expanded and isolated with the 293T population frozen before initial transfection (pre293T) along with a control 293T population expanded for an equivalent amount of time as the other mutant clones sequenced (post293T). DNA was extracted using the Qiagen DNeasy Blood and Tissue kit (cat-#69506) and were sequenced using Illumina PE 150 to a depth of ˜30×. Alignment and variant calling was provided by the Harvard Chan Bioinformatics Core, Harvard T.H. Chan School of Public Health, Boston, Mass. using an analysis pipeline based on bcbio framework (https://github.com/bcbio/bcbio-nextgen). For WGS data, BWA (v0.7.17) was used to map sequencing reads to the reference human genome (hg38). SNPs and indels using were identified somatic tumor-normal approach (using a control sample as a normal, and edited samples as ‘tumor’), and required 3 variant callers (vardict, v.2019.06.04, mutect2 (from gatk 4.1.2.0), strelka2, v2.9.10) to confirm a variant to be called (a similar approach was taken by Zuo et al⁵⁵. In the case of RNA-seq data, STAR (v.2.6.1d) was used to align reads, and RNA-seq specific gatkbased variant calling pipeline, with parameters and filters recommended by GATK best practices for RNA-seq variant calling (https://software.broadinstitute.org/gatk/documentation/article.php?id=3891), followed by filtering out variants at RNA editing sites according to the RADAR (v.2-20180202) database. GATK 3.8 was used to call variants in RNA-seq data, because validation has shown the superior precision of gatk 3.8 over gatk 4.1.2.0 when using RNA-seq reads. Due to the variability of coverage in RNA-seq data, variants were called in a single batch and only variants called as het, hom, or hom ref in all samples were considered for the downstream analysis. Variants were filtered out at sites matching gRNA using bedtools (2.27.1) and a custom bash script and used Rstudio and ggplot2 for the downstream analysis.

RNA-Seq Analysis after Base Editing

293T cells were transfected with HL1gR4 and either nABE, dABE, nCBE4-gam, or dCBE4-gam and cell pellets were isolated after 48 hours for DNA and RNA extraction. DNA was prepared for targeted amplicon sequencing as previously described. Cells for RNA-seq were lysed with TRIZOL (ThermoFisher 15596026) and total RNA was extracted using Zymo RNA mini prep kit (Zymo R2052). RNA was quantified using Qubit Fluorometer (ThermoFisher Q10211) and RNA integrity was confirmed by presence of two ribosomal bands and absence of degraded smears by gel electrophoresis. mRNA-seq libraries were prepared using KAPA mRNA HyperPrep (KAPA KK8580) using 1 μg total RNA. Libraries were pooled and sequenced on an Illumina MiSeq.

### RNA-Seq Analysis of LINE-1 Edited Living Cell Lines

The RNA of 293T LINE-1 knockout clones (1.37%-3.4%) by nCas9-CBE4-gam RNA of cells were extracted by treatment with TRIzol (ThermoFisher Scientific, cat-#15596018) followed by Direct-zol RNA Kit (Zymo Research, cat #R2072), according to the manufacturer's instructions. All samples were prepared from biological duplicates; the parental culture was divided into two cultures and passaged once before extraction. 500 ng RNA of each of the samples, as quantified by Qubit (Qubit™ RNA HS Assay Kit, ThermoFisher Scientific, cat-#Q32852), were used to prepare the libraries using an NEBNext Directional RNA Library Prep Kit (New England Biolabs, cat-#E7765S), and following the manufacturer's instructions.

Deamination frequency in the RNA of the 293T LINE-1 knockouts (1.37%-3.4%) by nCas9-CBE4-gam was analyzed using the standard deamination analysis pipeline used for genomic DNA. Read counts were generated by mapping reads to a human reference genome (GRCh38.p12, using the PRI version from www.genecodegenes.org) using STAR. Differential gene expression analysis was performed in EdgeR version 3.24.3: Lowly expressed genes with less than 2 counts per million counts in 2 or more samples were filtered out, the libraries were normalized using TMM normalization and differentially expressed genes were identified by using the exact test on the tagwise dispersion to compare the expression of each of the clones to the control sample. The Benjamini-Hochberg method was used to adjust p-values for multiple testing.

Multidimensional Scaling distances were generated by using the plotMDS function of EdgeR on the filtered and normalized libraries and plotted using ggplots.

Maintenance and Expansion of Human iPSCs

Human iPSCs were cultured with mTeSR medium on tissue culture plates coated with Matrigel (BD Biosciences). For routine passaging, iPSCs were digested with TrypLE (Thermofisher #12604013) for 5 minutes and washed with an equal volume PBS by centrifugation at 300 g for 5 minutes. Digested iPSC pellets were physically broken down to form a single cell suspension and then plated onto Matrigel-coated plates at a density of 3×10⁴ per cm² with MTESR™ medium supplemented with 10 μM Y-27632 ROCK inhibitor (Rᵢ) (Millipore, 688001) for the first 24 hours.

Nucleofection in PGP-1 iPSCs

Thirty minutes prior to transfection media was changed to mTeSR supplemented with Pifithrin-α (10 ng/μl) from Sigma-Aldrich P4359 (source #063M4741V, Batch #0000003019); a notable spiky edge colony morphology was observed similar to when Ri is added. Human iPSCs were digested with TrypLE for 5 minutes and the single cells were washed once with PBS. (CS: 4×10⁶, PK: 1×10⁶) iPSCs were then re-suspended in 100 μl of P3 Primary Cell Solution (Lonza) supplemented with (CS: 13.5 PK: 6.75 μg) of dABE plasmid, (CS: 4.5 μg, PK: 2.25 μg) of gRNA plasmid, and (CS: 2 μg, PK: 1 μg) pMax. The combined cells and DNA were then nucleofected in 4D-Nucleofector (Lonza) using the hES H9 program (CB150). The nucleofected iPSCs were then plated onto a single well of a 6-well Matrigel-coated plate in mTeSR medium supplemented with 10 μM Rᵢ and Pifithrin-α (10 ng/μl).

Clonal Isolation of PGP-1 iPSCs

96-well plates were coated with Matrigel (BD Biosciences) at a concentration of 50 μl/well. A cloning medium solution of 10% CLONER™ (StemCell Technologies #05888) and Pifithrin-α (10 ng/μl) in MTESR™ was prepared and added to the coated wells. Cells were digested using TrypLE, which was neutralized by an equal amount of cloning medium. The cell solution was then centrifuged at 300×g for 5 minutes, the supernatant was aspirated, and the cell pellet was resuspended in the cloning medium. The cells were then passed through a 40-μm cell strainer and were FACS-sorted into 1) individual wells containing warm cloning medium at a density of 1 cell/well and 2) 2×96-well PCR plates for direct NGS analysis. To prevent disturbance, there was no media change during the first 48 hours, and the plates were not removed from the incubator during this period. A half-medium change was performed on days 3 and 4 with cloning medium. The growing colonies were monitored and a MTESR™ medium change was done daily for the following days until extracting the DNA using QUICKEXTRACT™ and proceeding with library preparation and sequencing.

### Statistical Analysis

Statistical analysis was conducted using the student's t test using excel. Differences were considered significant if p value was <0.05.*−0.01<p<0.05, **−0.001<p<0.01, ***−p<0.001, ****−p<0.0001.

### Results

gRNA Design and Copy Number Estimation of Transposable Elements

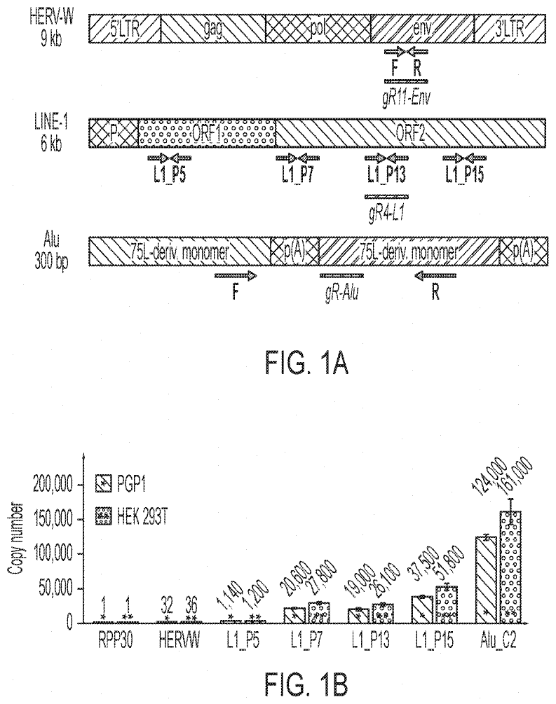

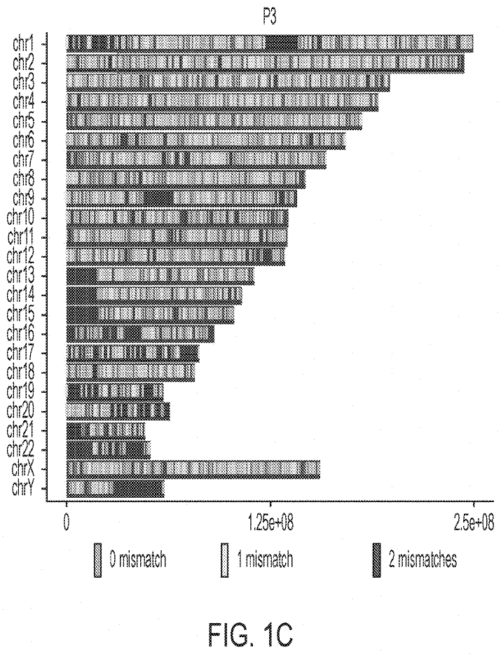

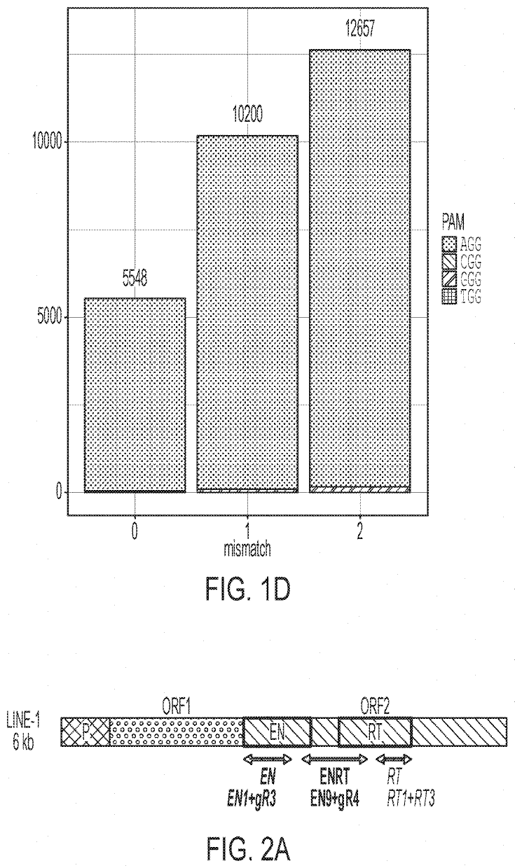

To assess the efficiency and toxicity of current editing technologies as applied to TEs, gRNAs were designed and tested against the TEs Alu, LINE-1, and HERV which vary in copy numbers from 30 to greater than 100,000 across the genome (FIG. 1A). Alu and LINE-1 gRNAs were respectively designed on the consensus sequences obtained from repeatmasker³⁴ (Table 2) and on the consensus of the 146 full-length sequence that encodes both functional ORF1 and ORF2 proteins. Finally, gRNAs against HERV-W were designed on the consensus of putatively active retro-viruses (Table 2).

TABLE 2
gRNAs used in the present disclosure

| | | | Cas9 |
|---|---|---|---|
| Name | gRNA | PAM | species |
| Non human | GAGACGAT | NGG | S. pyogenes |
| | TAATGCGT | | Cas9 |
| | CTCG | | (SpCas9) |
| | (SEQ ID | | |
| | NO: 47) | | |
| JAK2V | AATTATGG | TGG | SpCas9 |
| | AGTATGTG | | |
| | TCTG | | |
| | (SEQ ID | | |
| | NO: 48) | | |
| S1 | GATGACAG | CGG | SpCas9 |
| | GCAGGGGC | | |
| | ACCG | | |
| | (SEQ ID | | |
| | NO: 49) | | |
| ABEgR1 | GAACACAA | GGG | SpCas9 |
| | AGCATAGA | | |
| | CTGC | | |
| | (SEQ ID | | |
| | NO: 50) | | |
| HL1 gR1 | AACGAGAC | AGG | SpCas9 |
| | AGAAAGTC | | |
| | AACA | | |
| | (SEQ ID | | |
| | NO: 51) | | |
| HL1 gR2 | TCAGTTTC | CGG | SpCas9 |
| | CATGTAGT | | |
| | TGAG | | |
| | (SEQ ID | | |
| | NO: 52) | | |
| HL1 gR3 | TATGTACC | AGG | SpCas9 |
| | CAGTAGTC | | |
| | ATTC | | |
| | (SEQ ID | | |
| | NO: 53) | | |
| HL1 gR4 | ATTCTACC | AGG | SpCas9 |
| | AGAGGTAC | | |
| | AAGG | | |
| | (SEQ ID | | |
| | NO: 54) | | |
| HL1 gR5 | TTGAACCA | AGG | SpCas9 |
| | GCCTTGCA | | |
| | TCCC | | |
| | (SEQ ID | | |
| | NO: 55) | | |
| HL1 gR6 | GGGTATTC | AGG | SpCas9 |
| | AATTAGGA | | |
| | AAAG | | |
| | (SEQ ID | | |
| | NO: 56) | | |
| EN gR1 | GACTCCCA | GGG | SpCas9 |
| | CACATTAA | | |
| | TAAT | | |
| | (SEQ ID | | |
| | NO: 57) | | |
| LINE-1 | GCTTAGGT | | SpCas9 |
| gR46 | AAACAAAG | | |
| | CAGC | | |
| | (SEQ ID | | |
| | NO: 58) | | |
| EN gR9 | ATTTTGGA | TGG | SpCas9 |
| | ATAGGTGT | | |
| | GGTG | | |
| | (SEQ ID | | |
| | NO: 59) | | |
| RT gR1 | ATTCAGTA | TGG | SpCas9 |
| | TGATATTG | | |
| | GCTG | | |
| | (SEQ ID | | |
| | NO: 60) | | |
| RT gR3 | CCTAGGAA | GGC. | SpCas9 |
| | TCCAACTT | | |
| | ACAA | | |
| | (SEQ ID | | |
| | NO: 61) | | |
| Z8gR2 | AAAAAGAG | TGG | SpCas9 |
| | TCCAGGAC | | |
| | CAGA | | |
| | (SEQ ID | | |
| | NO: 62) | | |
| Alu | CAGGCGTG | CGG | SpCas9 |
| | AGCCACCG | | |
| | CGCC | | |
| | (SEQ ID | | |
| | NO: 63) | | |
| Sa Non | GAGACGAT | NGG | SaCas9 |
| human | TAATGCGT | | |
| | CTCG | | |
| | (SEQ ID | | |
| | NO: 64) | | |
| HERVenv11 | GAGGCACA | TAGGG | SaCas9 |
| | TCCAACAG | | |
| | TTAG | | |
| | (SEQ ID | | |
| | NO: 65) | | |

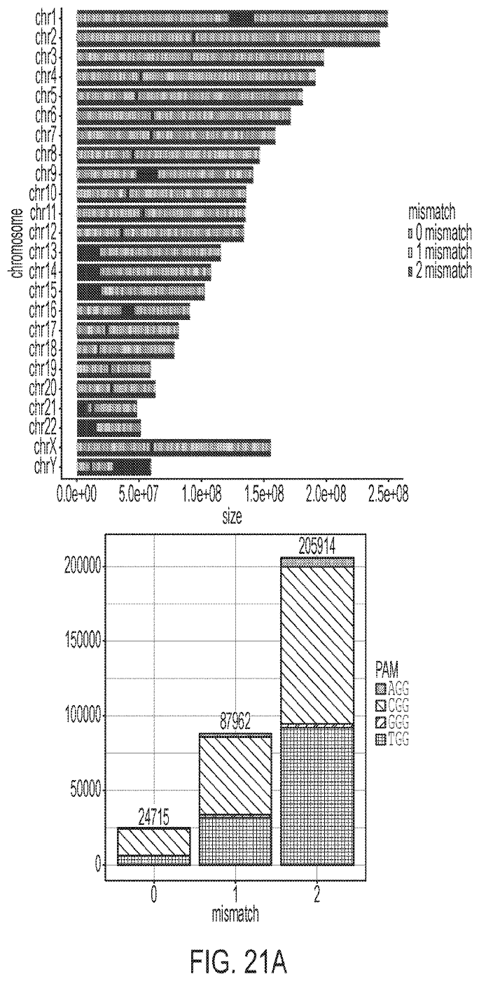

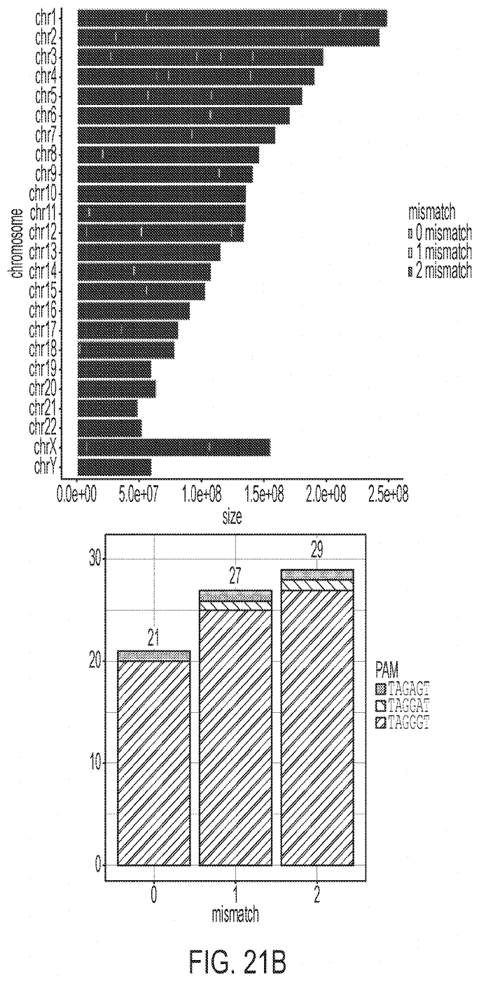

Because high copy TEs are so numerous and similar, they are difficult to definitively identify and count. For this reason, qPCRs of genomic DNA (gDNA) generated using consensus sequence-based primers were performed to estimate the relative abundance in HEK 293T and PGP1 cells (FIG. 1A). The copy number of HERV-W, LINE-1, and Alu elements at the edited sites were respectively estimated at 36, 26100 and 161000 loci in HEK 293T; and 32, 19000 and 124000 loci in PGP1 iPSCs (FIG. 1B). These numbers likely underrepresent HEK 293T TEs compared to PGP1 because the latter cells are largely triploid when PGP1 has a diploid karyotype. A complementary bioinformatic approach as a second estimate of TE abundance was used, in which the designed gRNAs were aligned to the human reference genome (FIGS. 21A-21B). An example of one HL1gR4 gRNA targeting LINE-1 ORF2 is shown in FIG. 1C. The total number of matches for HL1gR4 allowing 2 bp mismatches is 12,657, about half of the qPCR estimate, with the vast majority having an intact PAM (FIG. 1D). The reference sequence likely undercounts TEs because of the well-known problems of assembling, aligning, and mapping these sequences. Going forward, the editing numbers are based on the qPCR copy number estimate.

High Copy-Number CRISPR/Cas9 Editing Induces Cellular Toxicity and Inhibits Survival of edited cells

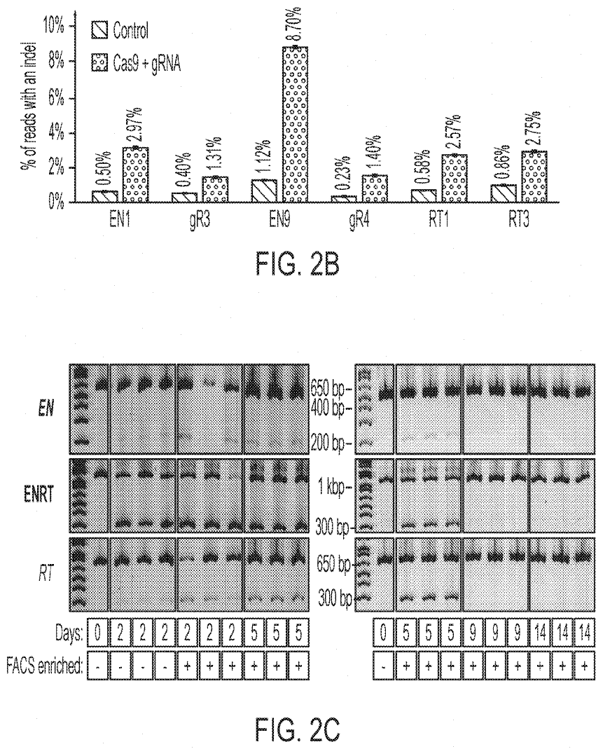

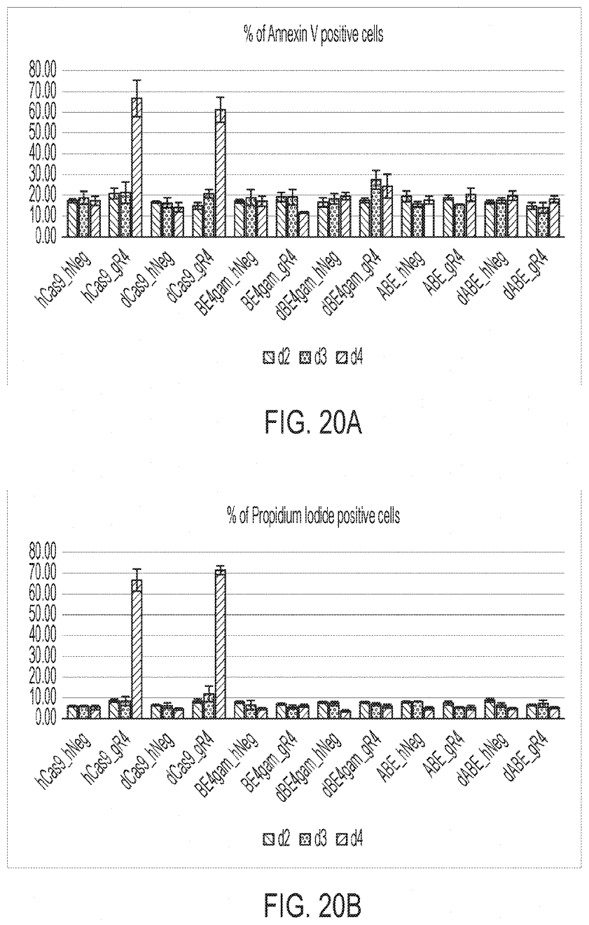

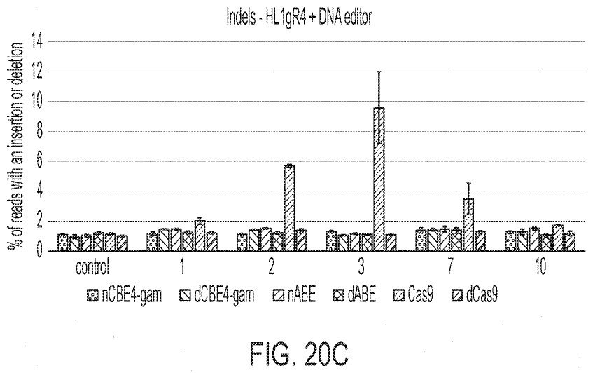

HEK 293T cells were transfected with plasmids expressing pCas9_GFP and LINE-1 targeting gRNAs to disrupt the two key enzymatic domains of ORF-2: endonuclease (EN) and Reverse transcriptase (RT) (FIG. 2A and Table 6). Three days after transfection, indel frequencies were observed at the LINE-1 expected targets ranging from 1.3% to 8.7%, corresponding to an average of respectively 339 and 2271 edits per haploid genome in the population (FIG. 2B). In accord with previous reports that this degree of genetic alteration is toxic, a 7-fold increase in apoptosis through Propidium Iodide and Annexin V staining was confirmed (FIGS. 20A-20C). A follow-up time course experiment demonstrated that cells that undergo editing at hundreds of loci do not survive.

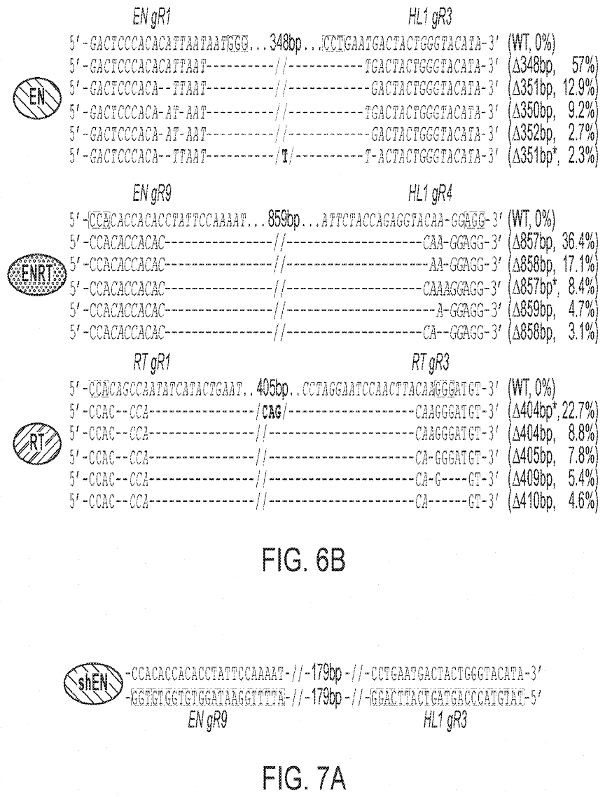

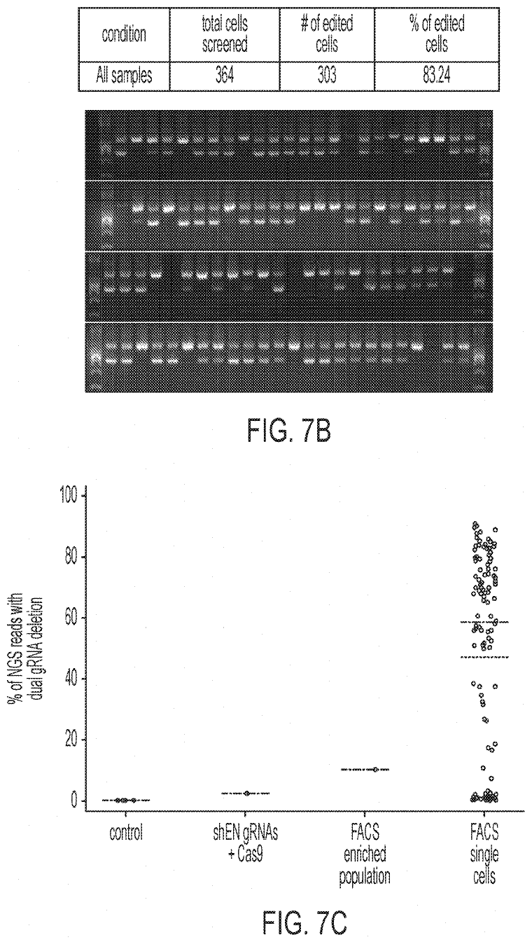

In accord with previous reports that this degree of genetic alteration is toxic, —7-fold increases in cell death and apoptosis were confirmed through Propidium Iodide and Annexin V staining (FIGS. 7A-7C). A follow-up time-course experiment demonstrated that cells that undergo editing at hundreds of loci do not survive. Here pairs of LINE-1 gRNAs targeting the EN, RT or both (ENRT) domains were transfected. Using pairs of gRNAs causes large deletions (˜170-800 bp) that can be detected through gel visualization (FIG. 6A). While samples from day two through five show clear editing with the expected deletion band sizes (FIG. 2C), they were no longer detectable at days 9 and 14 indicating that mutated cells either died out, consistent with a previously performed cell death assay, or were overgrown by wild type cells. Deep sequencing of expected dual gRNA deletion bands confirmed the LINE-1 gRNA breakpoints (FIG. 6B). While there were no visible bands at days 9 and 14, this experiment was repeated in an attempt to isolate clones. Despite early indications of editing, no clones had detectable mutations at day 12 and beyond (data not shown) suggesting that any significant level of indel activity at LINE-1 was toxic or limited growth and clonal isolation. Single cell analysis confirmed the bimodal editing frequency with a mean deletion frequency of 47.1% (FIGS. 7A-7C).

### Example 2

nCBE and nABE Activities Enables Isolation of Stable Cell Lines with Hundreds of Edits

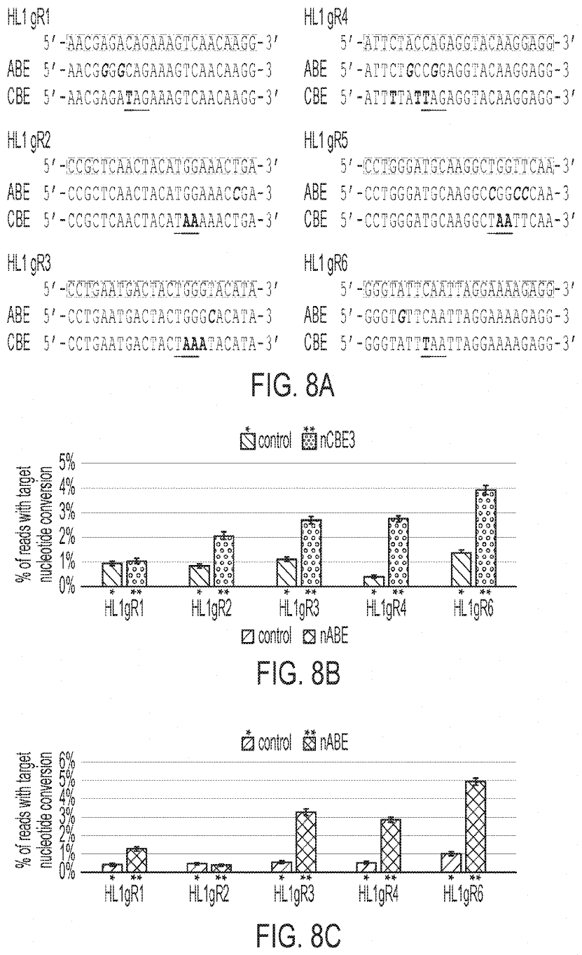

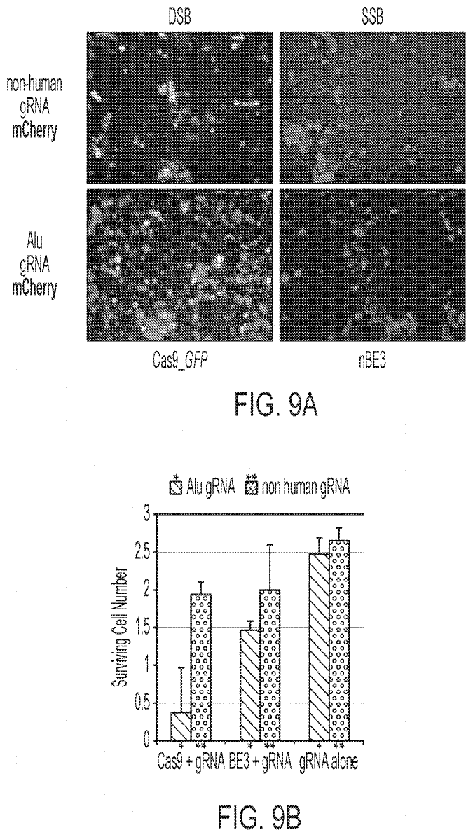

With the thought that use of nicking base editor technologies (nBEs) could help improve the viability of LINE-1 edited cells, LINE-1 targeting gRNAs (HL1gR1-6 [Table 3]) that generate a STOP codon early in ORF-2 using C→T deamination were designed and tested. HEK 293T cells were transfected with nCBE3 and each of these gRNAs. Deamination events were detected at each of the six gRNA target loci that, although small (˜0.05% —0.67%) exceeded levels in mock transfected control cells (FIG. 8A). These same CBE gRNAs could be used with ABEs, as they contain at least one adenine within their deamination window. Above control levels of base editing were detected in genomic DNA in 4 out of 5 gRNAs for both nCBE4-gam (FIG. 8B) and nABE (ABE7.10, Addgene #102919, SEQ ID NO: 15) (FIG. 8C). While nABE with HL1gR6 exhibited the highest editing efficiency (4.94% or ˜1290 loci) 3 days after transfection, HL1gR4 was used going forward because it had the highest signal-to-background error ratio of all the LINE-1 amplicons/gRNAs tested, and the HL1gR4 was among the most efficient. The HL1gR4 target window also contained three efficiently-coedited C's, thus offering a clear signal of directed mutation. signal of directed mutation. An Alu targeting gRNA showed increased cell survival when using nCBE3 compared to Cas9 (FIGS. 9A-9B).

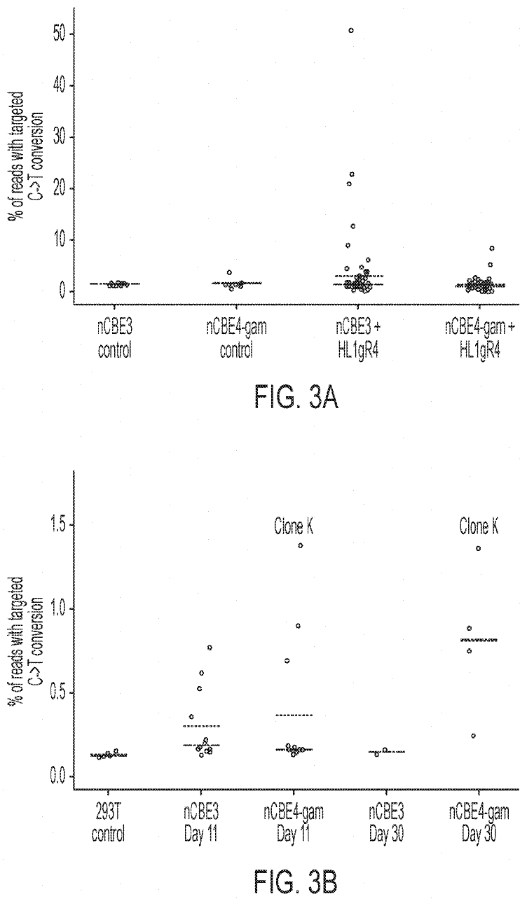

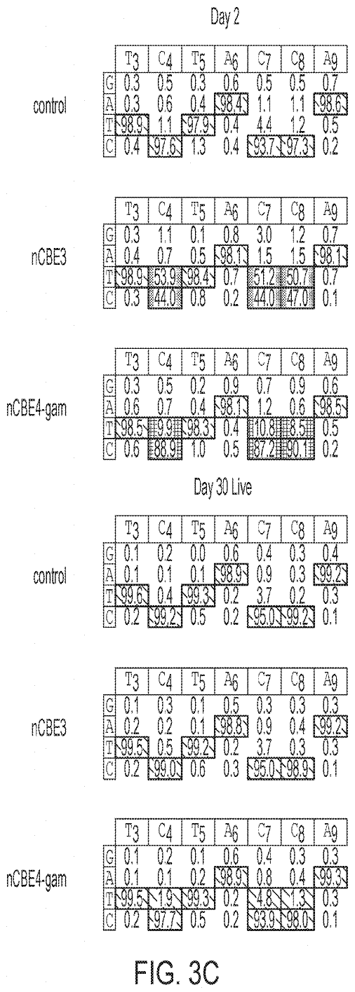

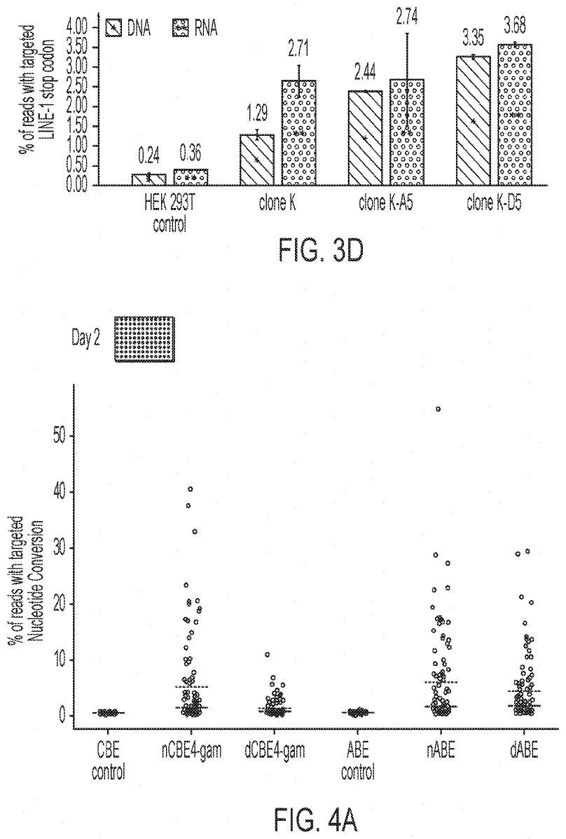

293T cells were transfected with HL1gR4 and either nCBE3 or nCBE4-gam with control samples receiving a non-targeting gRNA. Two days post-transfection, single cells were analyzed, resulting in a high editing efficiency of up to 53.9% C4T deamination, or an estimated 14,000 loci (FIG. 3A), in the most highly edited single cell. nCBE3 had a significantly higher mean deamination frequency than nCBE4-gam at this early timepoint. A parallel plate was sorted to assess viable colony formation and the edited 293T cells' capacity to form stable cell lines. While both nCBE3 and nCBE4-gam had edited cells at day 11, all cell lines with nCBE3 edits died before analysis could be conducted at day 30. Four surviving cell lines were isolated with deamination frequencies up to ˜1.37% of LINE-1 or an estimated ˜356 sites (FIG. 3B). Data presented in FIG. 3C shows both the purity of the desired deamination products and the editing window. CloneK was the most highly edited single cell isolated and was stable in terms of target C→T mutation frequency from day 11 to 30 across multiple independent PCR replicates at each time point.

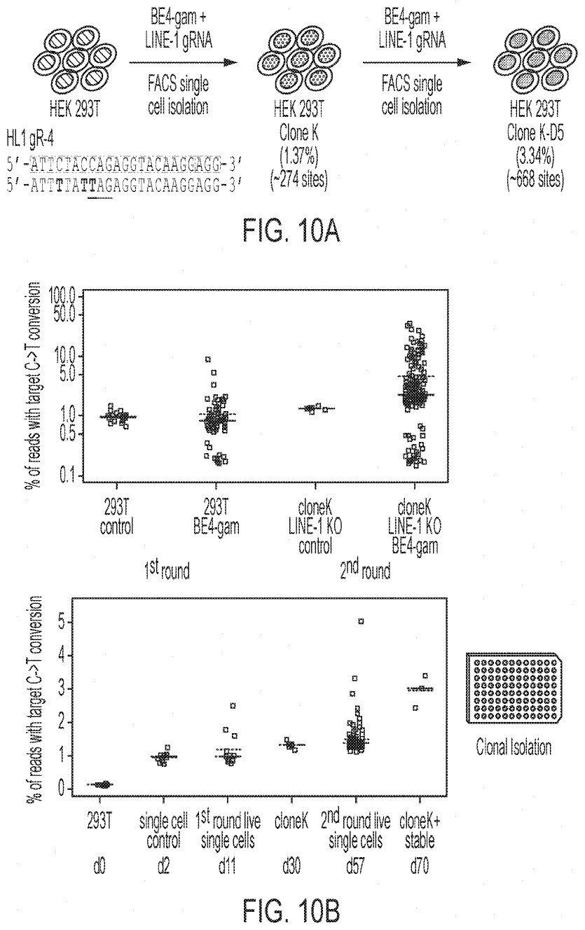

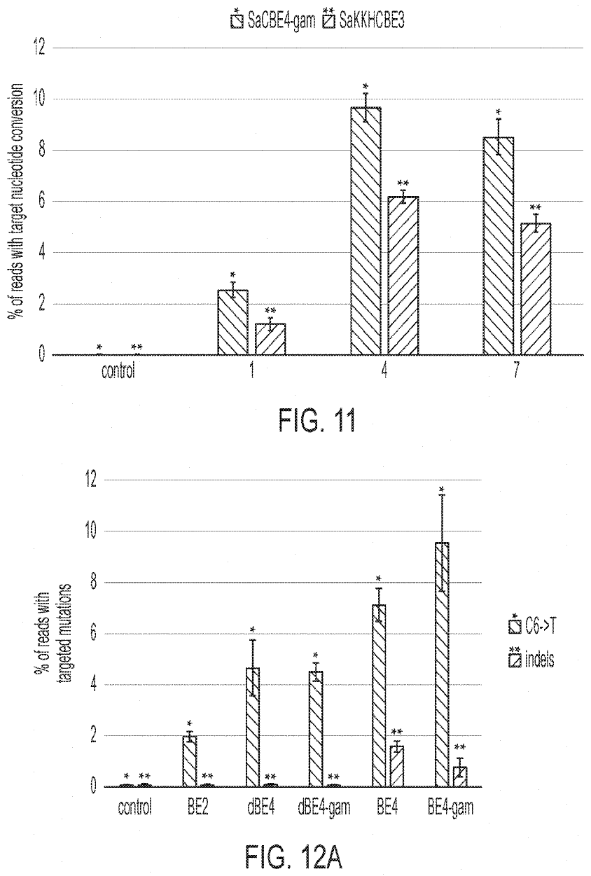

By subjecting the top edited single cell isolate cloneK to another round of nCBE4-gam editing (FIG. 10A) cells were detected with up to 36.26% C→T deamination were detected on day 2, and four living clones with deamination frequencies ranging from 2.43% to 5.04%—corresponding to about 643 to 1315 edits—were isolated (FIG. 10B). While the clone with the highest number of deaminated sites did not grow after a freezing and thawing cycle, the three other cell lines were stable in culture for a period longer than 30 days, and were termed “cloneK-A5”, “cloneK-A2” and “cloneK-D5”, with respectively 643, 749, and 781 edits. This observation of the highest edited clone dying off after initial detection was observed for all types of editors. It was confirmed that nBE activity at the lower copy number target HERV (FIG. 11). Due to the difficulty amplifying and analyzing the Alu target likely because of high subfamily polymorphism and short repeat sequence, (290 bp) experimentation proceeded exclusively with LINE-1 targeting gRNAs for the rest of the study.

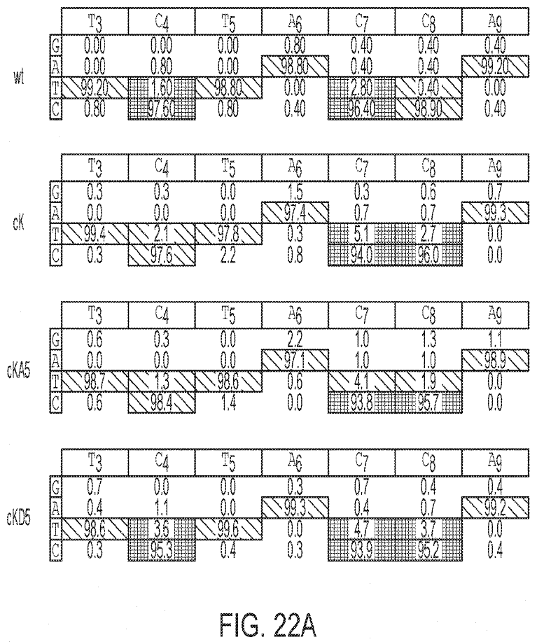

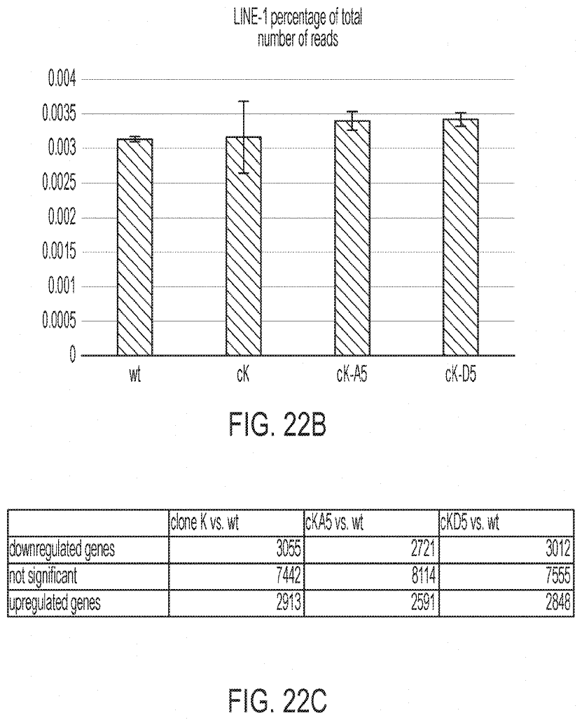

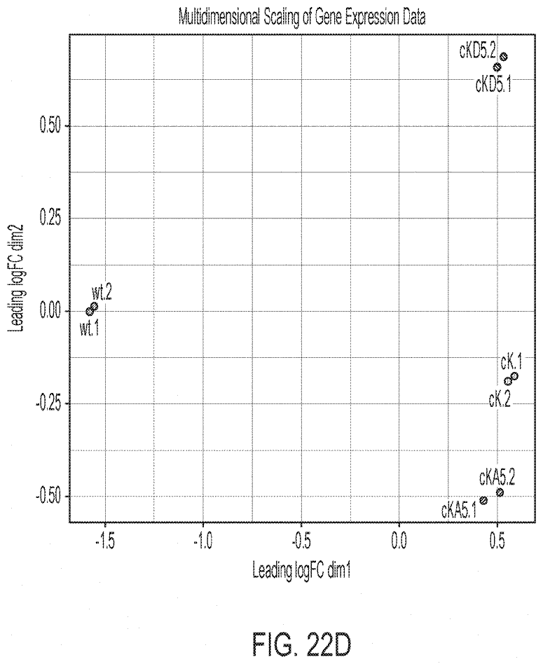

To confirm that LINE-1 editing at the genome level had a repercussion on the corresponding transcripts RNA-seq was performed on cloneK, cloneK-D5, and cloneK-A5 and analyzed the percentage of C→T conversion resulting in a stop codon in ORF2 in the RNA reads (FIGS. 3D, 22A-22D). Theoretically, since most of the active LINE-1 subsets should generate transcripts, the presence of the expected STOP codon at the messenger RNA level may indicate the inactivation of these elements. The results showed that a higher number of edits in the clones was correlated with a higher number of STOP codons at the RNA level, suggesting that transcriptionally active LINE-1 were impacted by the multiplexed editing.

TABLE 3
Evolution of fusion proteins

| | C-deaminase | A-deaminase | UGI | Nick | Mu gam |
|---|---|---|---|---|---|
| BE1¹ | X | | | | |
| BE2¹ | X | | X | | |
| BE3¹ | X | | X | X | |
| BE4² | X | | X2 | X | |
| BE4-gam² | X | | X2 | X | X |
| dBE4* | X | | X2 | | |
| dBE4-gam* | X | | X2 | | X |
| ABE³ | | X | | X | |
| dABE* | | X | | | |

### Nick-Less dBE Targeting of LINE-1 in HEK 293T

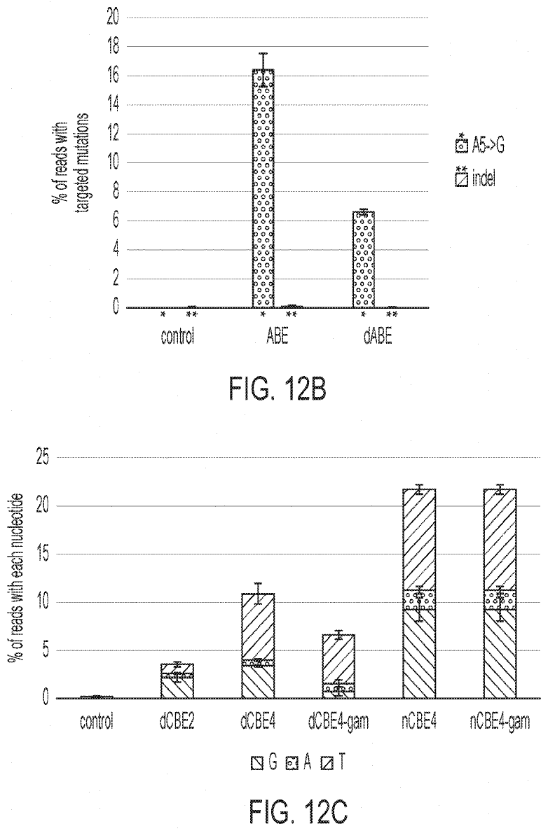

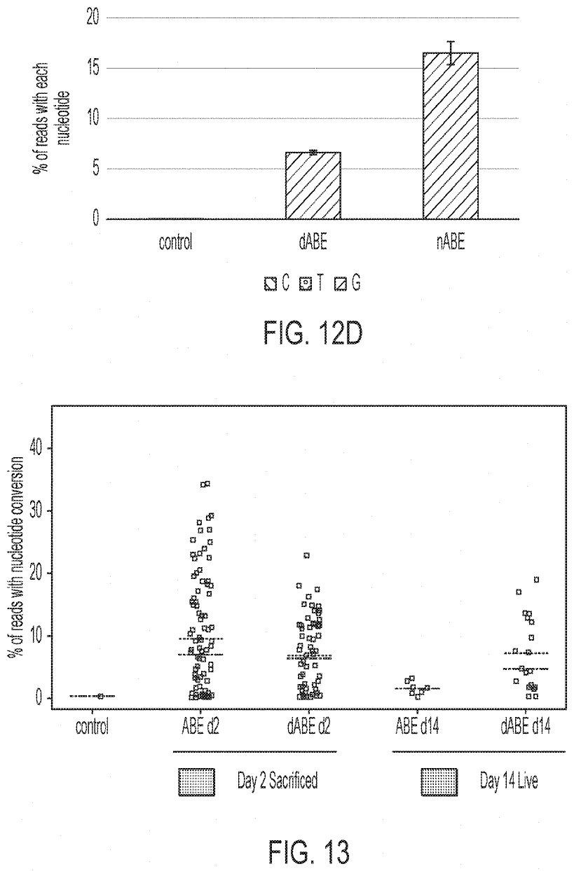

Suspecting that generating single-stranded nicks genome-wide could lead to cytotoxicity, the remaining HNH nuclease domain of Cas9 was inactivated by an H840A mutation in the Cas9 backbone and generate a set of dCas9-BEs including dCas9-CBE4-gam (dCBE4-gam), dCas9-CBE4 (dCBE4), and dCas9-ABE (dABE). Nick-less dCas9-BEs were tested on single-locus targets to confirm their deamination activity and compare them to their nBEs equivalents and the existing dCas9-CBE2 (dCBE2). dCBE4 and dCBE4-gam showed a 2.38- and 2.29-fold improvement in editing efficiency over CBE2 in 293T cells at day five respectively (FIG. 12A). Compared to their nicking counterparts this was a 34.7% or 53.2% reduction in efficiency but indel activity was reduced to background levels. dABE had no previous dead counterparts to compare to but retained 40.2% of nABE's deamination efficiency at a single locus control while reducing indel levels to background (FIG. 12B).

293T cells were then transfected with HL1gR4 and either nCBE4-gam, dCBE4-gam, nABE, or dABE that were individually sorted and analyzed for target nucleotide deamination 2 days after transfection. Single edited cells resulted in high editing efficiency of up to 54.9% with nCBE4-gam, or 14,300 loci, when significant reductions to mean target nucleotide deamination frequency was observed with dCBE and dABE when compared to their nBE equivalents (FIG. 4A). In parallel, single cells were grown to determine whether viable highly edited clones could be isolated. The editing efficiency trend reversed in live cells: dBE showed a significantly increased deamination frequency over nBE (FIG. 4B). dABE produced the mostly highly edited clone with 50.61% targeted nucleotide deamination frequency or an estimated 13,200 loci. Fusion proteins that retain nicking activity only generated a few rare cells with an editing frequency consistent with the prior experiments in FIG. 4B. Results were replicated using another LINE-1 targeting gRNA and similar trends were observed (FIG. 13).

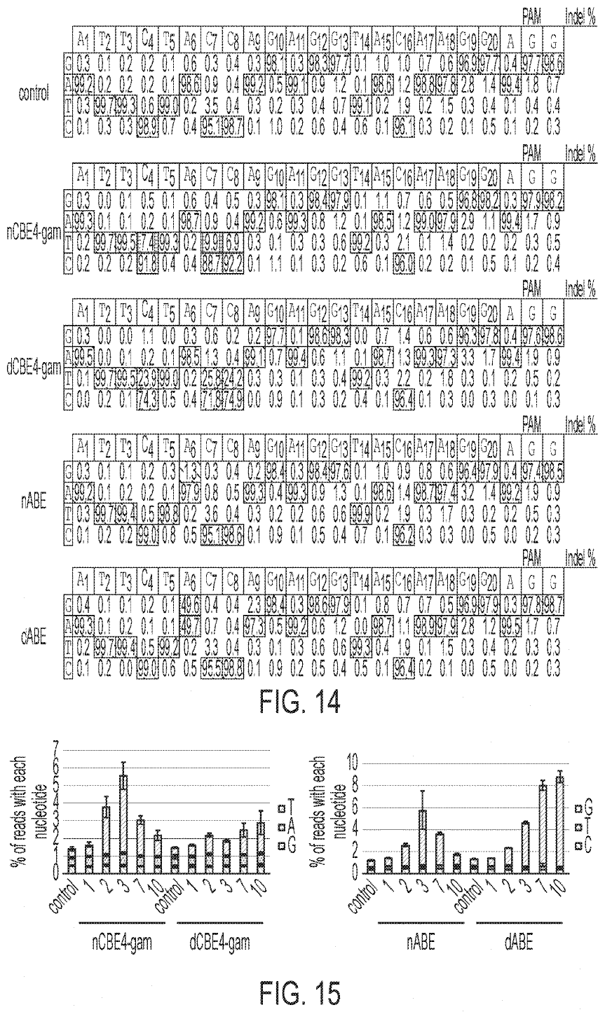

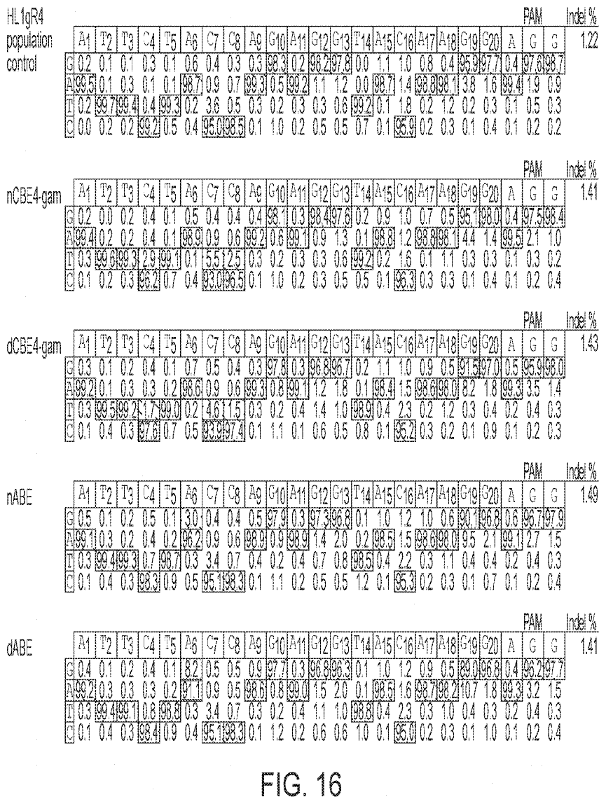

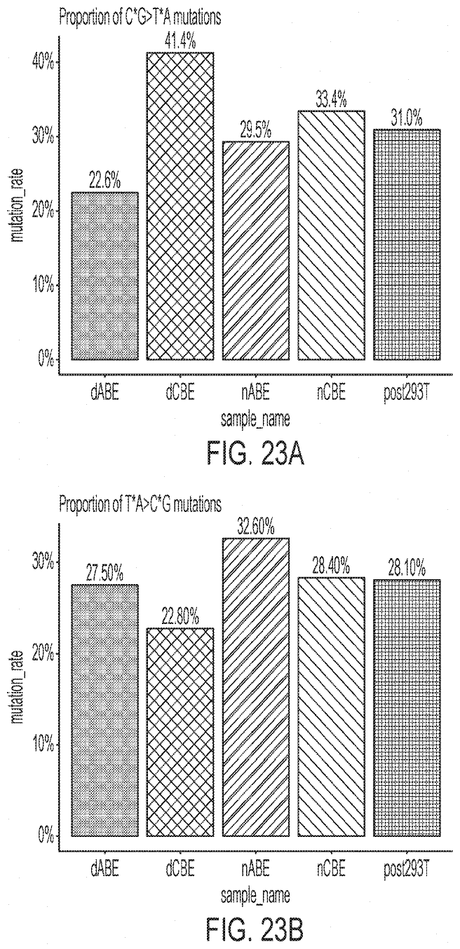

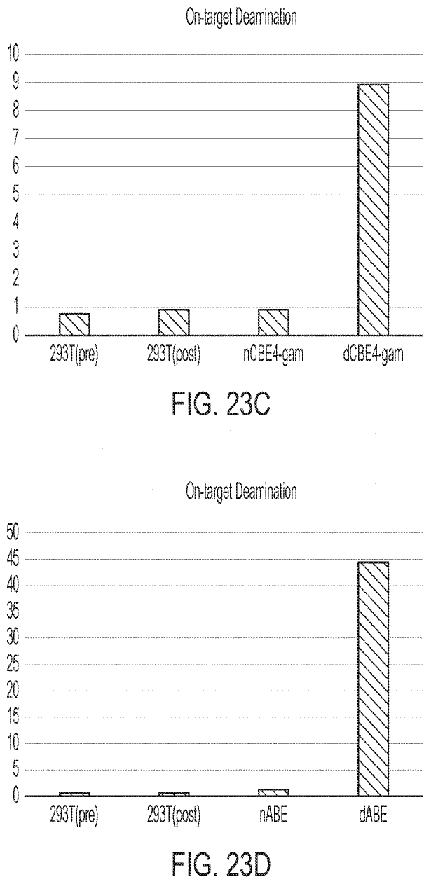

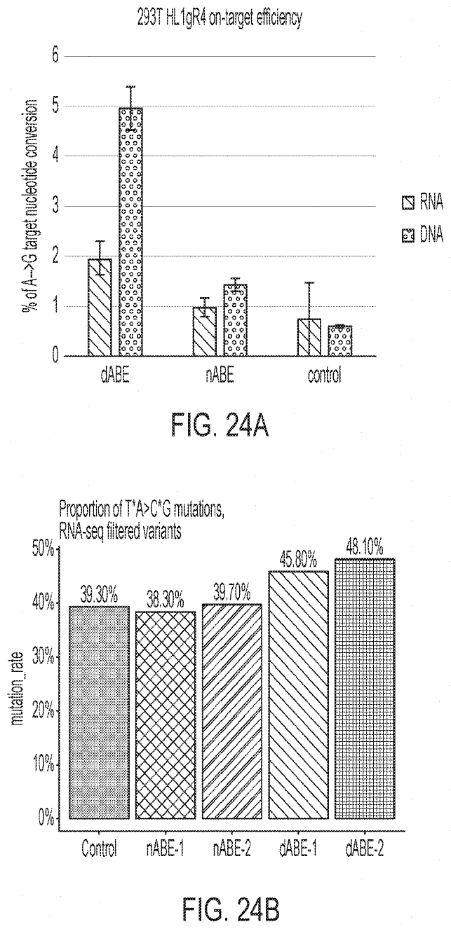

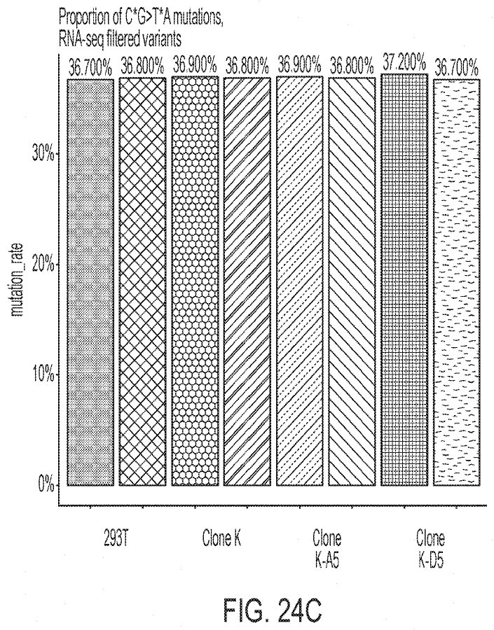

The nucleotide composition of all bases in the gRNA and PAM are displayed for the most highly edited clone and parental 293T control for each BE condition used, indicating some non-specific nucleotide conversions for both nCBE and dCBE but not nABE or dABE (FIG. 14). The mean single cell deamination frequency was reduced from 5.32% using nABE to 1.45% using dABE, indicating that retaining the nick and using nABE resulted in a 3.67-fold decrease in editing efficiency at the early timepoint (FIG. 4B). Cell viability mas measured at day 14, where dBEs gain a marked advantage in the total number of live cells, editing frequency of single cells, and mean target deamination frequency. There was a 14.8-fold increase in mean editing frequency among surviving live clones when using dABE compared to nABE. A 2.38-fold increase was also observed for dCBE4-gam compared to nCBE4-gam. A high base editing purity and no detectable nucleotide conversion beyond the expected range was observed in bulk transfected cells though day ten (FIGS. 15 and 16). During the first three days of editing the dBEs showed a lower editing frequency when compared to nBEs, but after day seven and ten, dABE gained a significant edge over nABE (FIG. 4C). HL1gR4 PCR products were analyzed to determine that only 64.1% of reads had a perfect match for the gRNA, 18.4% had a 1 bp mismatch, 3.2% with 2 mismatches and 13% with >9 mismatches (see Table 8), thus most similar off-targets are actually within the LINE-1 locus. To search for random genome wide deamination off-target analysis was conducted using whole genome sequencing and RNA-seq. As previously reported^{54,55}, identified genome wide off-target variants enriched for C:G→T:A mutations after CBE editing were identified, with dCBE4-gam at 41.4% above ˜30% for the unedited samples (FIGS. 23A-23D). Off-target deamination at the RNA level at day 2 was detected (FIGS. 24A-24B). No long-term effects of RNA mutation spectrum were observed in the stable CBE edited clones after 30 to 70 days (FIG. 24C).

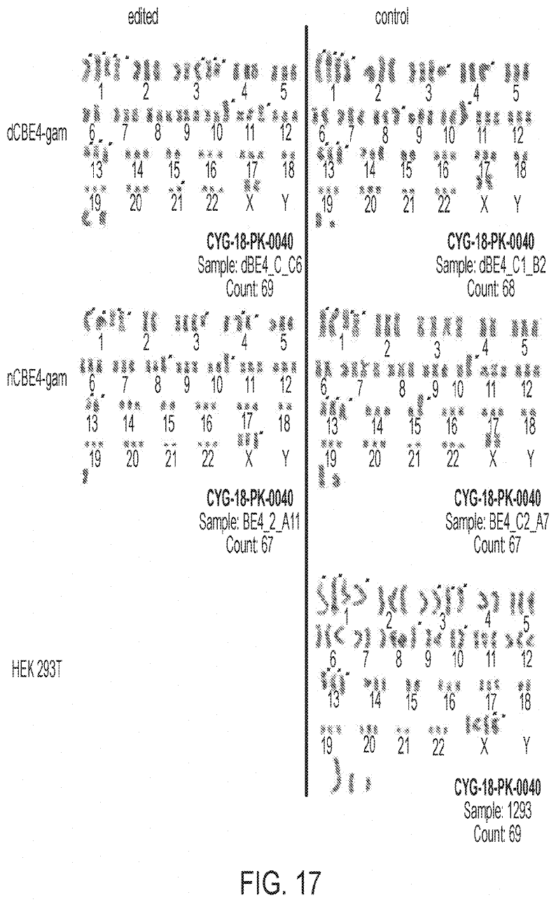

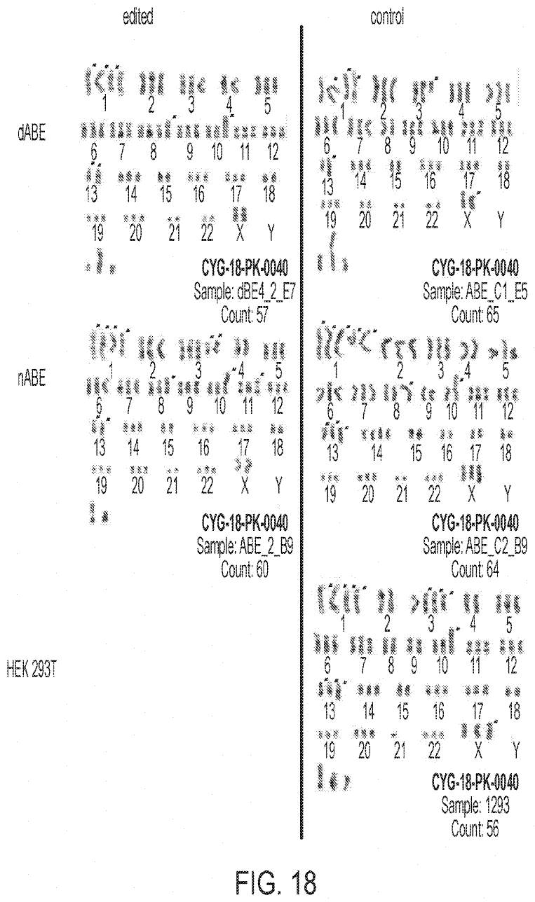

Chromosomal integrity analysis was performed for clones edited at LINE-1 with nABE, dABE, nCBE4-gam, and dCBE4-gam. The karyotype results are shown in Table 5 and show that the top edited clones are not significantly different than control groups in terms of total number of aberrations (FIGS. 17 and 18). Further analysis in a karyotypically normal and stable cell line is required to fully assess chromosomal stability after large-scale genome editing.

TABLE 8
Line-1 subfamily analysis and matches to HL1gR4

| # of Mismatches | Reads | Total Reads | Percentage |
|---|---|---|---|
| 0 | 22780 | 35520 | 64.1% |
| 1 | 6546 | 35520 | 18.4% |
| 2 | 1131 | 35520 | 3.2% |
| 3 | 148 | 35520 | 0.4% |
| 4 | 63 | 35520 | 0.2% |
| 5 | 2 | 35520 | 0.0% |
| 6 | 26 | 35520 | 0.1% |
| 7 | 1 | 35520 | 0.0% |
| 8 | 1 | 35520 | 0.0% |
| 9 | 1 | 35520 | 0.0% |
| >9 | 4600 | 35520 | 13.0% |

### Example 3

Large-Scale Genome Editing with dABE in PGP1 iPSCs

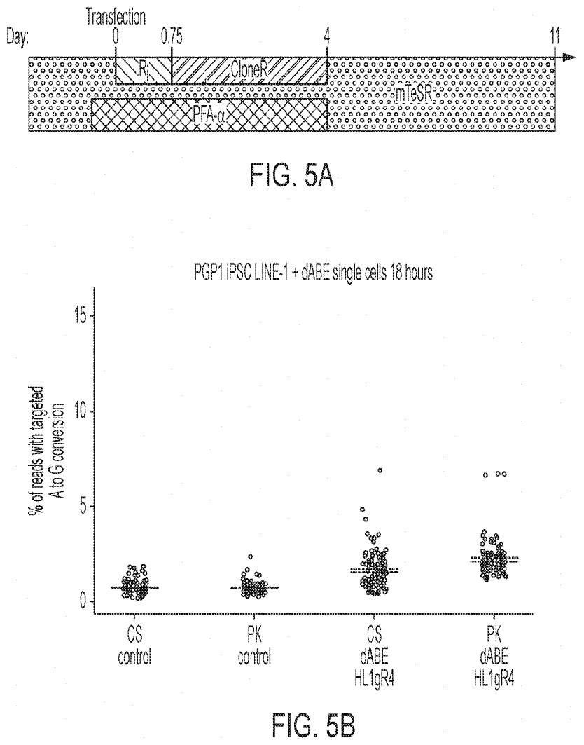

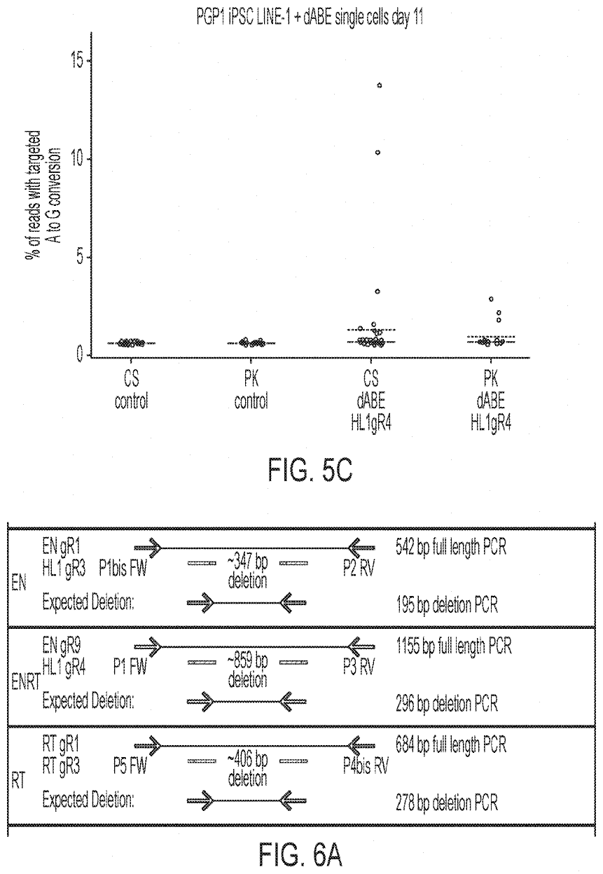

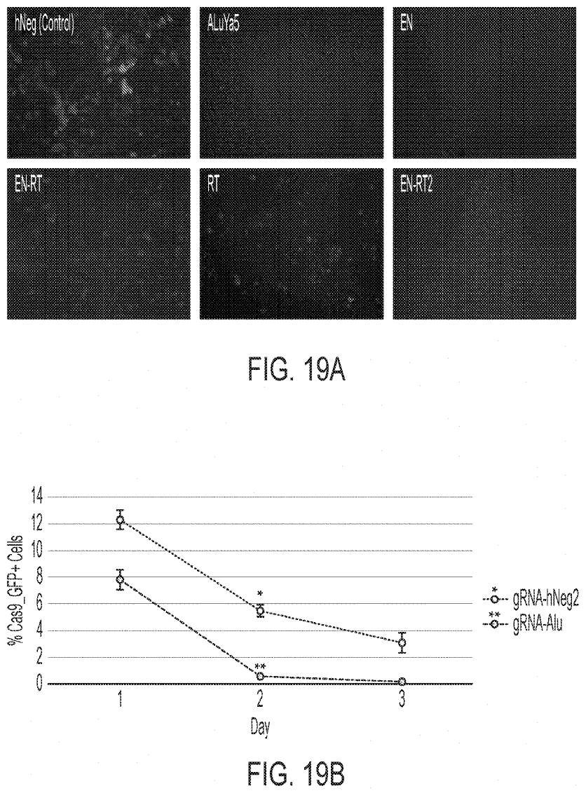

Next, large-scale genome editing of PGP1 induced pluripotent stem cells (iPSCs) was attempted. The survival cocktail and single cell isolation time line is shown in FIG. 5A. The same experiment was conducted with two slight variations of the electroporation protocol differed in terms of total cells transfected and the total amount of DNA used. Single cells were sorted and analyzed for target nucleotide deamination frequency 18 hours post electroporation. The highest edited single cell had ˜6.96% target A→G conversion or ˜1320 sites (FIG. 5B). In parallel live single cells were isolated after stable cell lines formed at 11 days after transfection. Colonies were analyzed for targeted LINE-1 A→G deamination with a 1.30% and 0.96% editing frequency respectively (FIG. 5C). The median editing efficiency of some live clones was higher than others in contrast to the value observed at the earlier time point, suggesting that lower editing efficiency in earlier time points may increase the viability of stably edited cell lines. The most highly edited clone had a deamination frequency of 13.75% which corresponds to 2600 sites genome wide, exceeding by three order of magnitude the number of simultaneous edits previously recorded in iPSCs.³⁵ The increased background that occurs in single cell direct analysis FIG. 5B compared to isolation from an expanded colony FIG. 5C is likely due to the necessary over-amplification required to get enough genomic material from a single cell. Similar observations were made in previous experiments using 293T cells. All other previously tested DNA editors failed to produce any detectable edits at the LINE-1 locus in human iPSCs which are prone to apoptosis after even minor DNA damage and rapidly deplete cells transfected with Cas9 and TE gRNAs (FIGS. 19A-19B).

Single Cell Analysis of LINE-1 Dual gRNA Disrupted Cells

The PCR amplicons of dual gRNA combinations from the previous experiments were too large to include both the mutated and full-length bands together for Illumina NGS. To overcome this, a shorter pair of LINE-1 targeting gRNAs, called short EN (shEN), were used that permits both be sequenced together. 293T cells were transfected with pCas9_GFP and the shEN gRNA pair (ENgR9 and HL1gR3) in the pSB700mCherry gRNA expression vector (FIG. 7A). GFP and mCherry double-positive single cells were FACS-sorted into gDNA extraction solution. 303 individual cells were screened after FACS sorting and LINE-1 NGS analysis. Of those wells with an amplicon, 83.24% had a visually detectable deletion band with a range of intensities from barely observable to stronger than the wild type non-mutated band (FIG. 7B). Bulk-transfected cells had a dual gRNA deletion frequency of 2.7%, the FACS-enriched double-positive cell population was edited at 11.19%, and the mean editing of single-cell-derived amplicons was ˜50.17% (FIG. 7C). The editing frequency appears to be bimodal as previously reported in the set of PERV editing papers, with experiments first in transformed cells⁴⁶—achieving 62 indels—and then later in healthy born piglets¹⁶—with all 25 PERVs knocked-out. At first it seems contradictory that the population bulk gRNA editing is 11.19% and the single cell average is 50.1%, but this assumes that each single cell had a full nuclear genome. The highest edited samples most likely had already degraded their genomes by thousands of concurrent cuts to every chromosome thus each single cell observed was contributing unequally to the bulk population.

### RNA-Seq in LINE-1 Knockout Clones

Downregulation of LINE-1 RNA expression levels in edited clones, wherein the number of RNA reads obtained through the standard deamination analysis pipeline, averaged over the 20 nt protospacer sequence and normalized the read counts by dividing by the size of their respective libraries, are displayed in FIGS. 22A-22B. A list of predicted differentially expressed genes in the edited clones compared to the wild type is found in supplementary data S1, and numbers of up and down regulated genes is found in FIG. 22C. Multidimensional scaling of the gene expression data (FIG. 22D), where the distance between the samples corresponds to leading log fold-changes between the RNA samples, shows a clear separation between the wild type and the three edited samples. Since the wild type control samples, however, did not undergo a comparable procedure of transfection and cell sorting, we cannot conclude that the observed differences in gene expression are due to LINE-1 editing.

TABLE 4
dBE vs BE Survival statistics in HEK 293T
HEK 293T

| | | | |
|---|---|---|---|
| BE4-gam | dBE4-gam | ABE | dABE |

Day 2

| | | | | |
|---|---|---|---|---|
| Number of cells with | 63 | 40 | 58 | 64 |
| targeted deamination | | | | |
| Number of | 96 | 96 | 96 | 96 |
| analyzed cells | | | | |
| Percent of cells with | 65.6 | 41.7 | 60.4 | 66.7 |
| targeted deamination | | | | |
| Mean target | 6.08 | 4.43 | 5.32 | 1.45 |
| deamination % | | | | |

Day 14

| | | | | |
|---|---|---|---|---|
| Number of cells with | 4 | 20 | 4 | 22 |
| targeted deamination | | | | |
| Number of | 12 | 29 | 16 | 25 |
| analyzed cells | | | | |
| Percent of cells with | 33 | 69 | 25 | 88 |
| targeted deamination | | | | |
| Mean target | 1.28 | 3.05 | 0.88 | 13.05 |
| deamination % | | | | |

TABLE 5
Karyotype chromosomal abnormality list

| | BE4_2_ | BE4_C2_ | | | | | | | 293.T9(CYG-18- |
|---|---|---|---|---|---|---|---|---|---|
| | A11 | A7 | dBE4_3_C6 | dBE4_C1_B2 | ABE_2_A4 | ABE_C2_B9 | dABE_2_E7 | dABE_C1_E2 | PK-0040) |
| −X | | | | x | x | | x | x | |
| add(X)(q28) | x | x | x | | x | x | | | x |
| der(X)add(X) | | | | | | | | x | |
| (p11.2)add(X)(q28) | | | | | | | | | |
| add(1)(p36.1) | x | x | x | x | x | x | x | | x |
| add(1)(q42) | xx | xx | xx | xx | xx | xx | xx | | xx |
| del(1)(q31) | x | x | x | x | | x | x | | x |
| i(1)(p10) | | | | | | | | x | |
| add(1)(q21) | | | | | | | | x | |
| −2 | | | | | | | | | |
| add(3)(p13) | | | | | | | | x | |
| add(3)(p24) | | | xx | | | | | | x |
| del(3)(p22) | x | | | | x | | | | |
| add(3)(q12) | | | x | x | | | | | x |
| del(3)(q22) | x | | | | x | x | | x | |
| add(4)(p15) | x | | | | | | | | |
| del(4)(q31) | x | | | | | | | | |
| −4 | | x | | | x | x | x | | x |
| add(8)(p21) | x | x | x | x | x | x | x | | x |
| −9 | | | | | | x | | | |
| add(10)(p11) | | | | | | | | | |
| add(10)(p13) | x | x | x | x | x | x | x | | x |
| add(11)(p15) | | | x | | x | | | | |
| add(13)(p11) | xx | xx | xx | xx | xx | xx | xx | | xx |
| add(13)(q34) | x | x | x | x | x | x | x | | x |
| −13 | | | | | | | | | |
| add(14)(p11.2) | | | x | x | | | | x | |
| −15 | x | x | x | x | x | x | x | | x |
| add(15)(p11.2) | | x | | | | | | | |
| −18 | x | x | x | x | x | x | x | | x |
| −21 | x | x | | x | x | x | x | | x |
| −22 | | | | | | | x | | |
| i(21)(q10) | | | x | | | | | | |
| mar | x-xx | x-xx | x-xxx | x-xx | xx-xxx | x-xx | x-xxx | x-xxx | x-xxxx |

TABLE 6
NGS primers list

ILMN-F: 5′-CTTTCCCTACACGA-1CGCTCTTCCGATCT-3′
(SEQ ID NO: 66)
ILMN-R: 5′-GGAGTTCAGACGTGTGCTCTTCCGATCT-3′
(SEQ ID NO: 67)

| gRNA | | |
|---|---|---|
| Name | Primer F | PrimerR |
| HL1 gR1 | AGACTCCCAC | TGATTTGGGGT |
| | ACATTAATAA | GGAGAGTTCT |
| | TGGG | G (SEQ ID |
| | (SEQ ID | NO: 69) |
| | NO: 68) | |
| HL1 gR2 | AGTGCAATCA | CCCTCTACACA |
| | AACTAGAACT | CTGCTTTGAAT |
| | CAGG | G |
| | (SEQ ID | (SEQ ID |
| | NO: 70) | NO: 71) |
| HL1 gR3 | AGTGCAATCA | CCCTCTACACA |
| | AACTAGAACT | CTGCTTTGAAT |
| | CAGG | G |
| | (SEQ ID | (SEQ ID |
| | NO: 72) | NO: 73) |
| HL1 gR4 | AAGAGTCCAG | CCCGGCTTTGG |
| | GACCAGATGG | TATCAGAATG |
| | AT | (SEQ ID |
| | (SEQ ID | NO: 75) |
| | NO: 74) | |
| HL1 gR5 | CTTATCCACC | CTGCATCTATT |
| | ATGATCAAGT | GAGATAATCAT |
| | GGG | GTGG |
| | (SEQ ID | (SEQ ID |
| | NO: 76) | NO: 77) |
| HL1 gR6 | GTTCTGGCCA | CCTGAGACTTT |
| | GGGCAATCAG | GCTGAAGTTGC |
| | (SEQ ID | (SEQ ID |
| | NO: 78) | NO: 79) |
| HL1 gR46 | AACTGCAAGG | AGAGGTGGAGC |
| | CGGCAACGAG | CTACAGAGG |
| | (SEQ ID | (SEQ ID |
| | NO: 80) | NO: 81) |
| EN gR1 | CCAATACAGG | TGATTTGGGGT |
| | AGCACCCAGA | GGAGAGTTC |
| | TT | (SEQ ID |
| | (SEQ ID | NO: 83) |
| | NO: 82) | |
| EN gR9 | CAGAACTCTC | CCTGAGTTCTA |
| | CACCCCAAAT | GTTTGATTG |
| | (SEQ ID | (SEQ ID |
| | NO: 84) | NO: 85) |
| RT gR1 | CCACATGATT | GAGGGCATCCC |
| | ATCTCAATAG | TGTCTTGTG |
| | (SEQ ID | (SEQ ID |
| | NO: 86) | NO: 87) |
| RT gR3 | GCAACTTCAG | GTAGTTCTCCT |
| | CAAAGTCTCA | TGAAGAGGTCC |
| | (SEQ ID | (SEQ ID |
| | NO: 88) | NO: 89) |
| EN (dual | CCAATACAGG | CCCTCTACACA |
| gRNA) | AGCACCCAGA | CTGCTTTGAAT |
| | TT | G |
| | (SEQ ID | (SEQ ID |
| | NO: 90) | NO: 91) |
| RT (dual | CCACATGATT | GTAGTTCTCCT |
| gRNA) | ATCTCAATAG | TGAAGAGGTCC |
| | (SEQ ID | (SEQ ID |
| | NO: 92) | NO: 93) |
| ENRT | CAGAACTCTC | CCCGGCTTTGG |
| (dual | CACCCCAAAT | TATCAGAATG |
| gRNA) | C | (SEQ ID |
| | (SEQ ID | NO: 95) |
| | NO: 94) | |
| shEN | CAGAACTCTC | CCCTCTACACA |
| (dual | CACCCCAAAT | CTGCTTTGAAT |
| gRNA) | C | G |
| | (SEQ ID | (SEQ ID |
| | NO: 96) | NO: 97) |
| HERV | AATACCACCC | CAGATTGGAAA |
| env11 | TCACTGGGCT | CAAGAGGTCC |
| | (SEQ ID | (SEQ ID |
| | NO: 98) | NO: 99) |

TABLE 7
List of DNA editors

| Plasmid name (as | | | |
|---|---|---|---|
| described in this | | | |
| application) | Addgene name | Addgene # | citation |
| pSB700 | pSB700 | 64046 | |
| pSB700_mCherry | — | — | |
| pSB700_Puro | — | — | |
| SaCas9_gRNA | BPK2660 | 70709 | |
| pCas9_GFP | pCas9_GFP | 44719 | |
| hCas9 | hCas9 | 41815 | |
| nCBE2 | pCMV_BE2 | 73020 | |
| nCBE3 | pCMV_BE3 | 73021 | |
| nCBE4 | BE4 | 100802 | |
| nCBE4-gam | BE4-gam | 100806 | |
| nABE | pCMV_ABE7.10 | 102919 | |
| SaCas9 | pX600-AAV- | 61592 | |
| | CMV::NLS-SaCas9- | | |
| | NLS-3xHA-bGHpA | | |
| Sa-nCBE4-gam | SaBE4-gam | 100809 | |
| SaKKH-nCBE4 | pJL-SaKKH-BE3 | 85170 | |
| dCBE4 | dCBE4 | | |
| dCBE4-gam | dCBE4-gam | | |
| dABE | dABE | | |

### DISCUSSION

CRISPR has recently brought a radical transformation in the basic and applied biological sciences, leading to commercial applications a multitude of clinical trials³⁶, and even the controversial tests of human germline modification³⁷⁻⁴¹. While the use of CRISPR and its myriad derivatives has greatly reduced the activation energy and technical skill required to perform genome editing several needs in the art must to be addressed before its full potential can be properly realized: 1) the need for custom RNA, and perhaps DNA for each target, 2) difficult delivery, 3) inefficiencies once delivered, 4) off-target errors, 5) on-target errors, 6) the toxicity of DNA damage, 7) the challenge of multiplexing beyond 62 loci³, 8) the limitation of insertion sizes below 7.4 kb⁴², 9) immune reactions to Cas, gRNA and vector. The present disclosure aims to develop tools that satisfy needs relating to on-target errors, toxicity of DNA damage, and multiplexing beyond 62 loci.

Improving multiplexed eukaryotic genome editing capabilities by several orders of magnitude holds the potential of revolutionizing human health. Combinatorial functional genomic assays would enable the study of complex genetic traits with applications in evolutionary biology, population genetics, and human disease pathology. In addition, analyzing the functional significance of any generated set of mutations through editing would empower the field of cancer biology. Multiplex editing has also permitted the development of successful engineered cell treatments such as the chimeric antigen receptor (CAR) therapies, which require the simultaneous editing of three target genes. Future treatments may require many more modifications to augment cancer immunotherapies, slow down oncogenic growth, and reduce adverse effects such as graft versus host disease. Furthermore, customizing host-versus-graft antigens in human- or nonhuman-donor tissues may require more modifications than have been done so far, for which the development of genome-wide editing technologies is needed. Special attention will be required to the safety of the editing and its impact on the functional activity of the transplants, since donor tissues may persist in the patient for decades.

To complete genome-wide recoding and enable projects such as GP-write ultra-safe cells¹, the de-extinction efforts to regain the lost biodiversity, or the codon reduction to confer pan-virus resistance, safe DNA editors must be developed to increase the number of genetic modifications to several orders of magnitude without triggering overwhelming DNA damage, as well as overcoming the delivery of multiple distinct gRNAs per cell, the latter of which are not addressed herein. E. coli MG1655 has all instances of the Amber stop codon replaced and has shown to be resistant to a range of viruses⁶. To attempt such a feat on the human genome, 4438 Amber codons⁸ will need to be modified. It has been shown that gene editors that do not cause double- or single-stranded DNA breaks can generate a number of edits sufficient to theoretically achieve this genome recoding and pave the way towards making pan-virus resistant human cells. This could have commercial application towards cell-based production of monoclonal antibodies, recombinant protein therapeutics, and synthetic meat production.

As the study demonstrates, genome wide disruption of high copy number repetitive elements is now possible and opens new opportunities to study the “dark matter” of the genome. CBEs that allow the generation of STOP codons within an open reading frame will be a great tool to probe at the functions of transposable elements, potentially turning observed associations with physio-pathological phenotypes into causations. For instance, large-scale inactivation of HERV-W and LINE-1 elements could help investigate their respective role in multiple sclerosis and neurological processes.

In the study, it was observed that dABE increases the viability of highly edited clones as compared to dCBE. This difference may be explained by two factors: First, when using HL1gR4, CBE has three target nucleotides within its deamination window as compared to one for ABE, and as a consequence, CBE converts three times more nucleotide than ABE, potentially causing additional cytotoxicity. Second, when using CBE, the uracil N-glycosylase (UNG) actively catalyzes the removal of the deaminated cytosine, generating several nicks genome-wide that promote DNA damage and potential cell death. The conversion of adenosine into inosine using ABE may not be detected as efficiently by the DNA repair machinery therefore increasing the viability of large-scale editing. For this reason, the conditional modulation of DNA repair processes such as mismatch repair (MMR) or base excision repair (BER)—that trigger downstream single- and double-stranded breaks in the genome—further improves the extent of dBEs' performance.

Finally, since dBEs do not generate direct breaks into the genome, they decrease indel frequency to background and may not trigger DNA sensors such as p53, while retaining about 34% to 53% deamination frequencies as compared to their nBE counterparts. As a consequence, successful genetic modifications with dBEs may not enrich for pro-oncogenic cells that have disrupted DNA-damage guardians as it has been reported for Cas9.⁴³ Even at low level of multiplexing, this feature may promote dBEs as an essential tool for therapeutic applications such as gene therapies.

In summary, this work optimized large-scale genome editing to enable cell viability after the simultaneous editing of thousands of loci per single cell. The ability to safely edit many loci may facilitate the true potential of personalized medicine as understanding of gene interactions and epistasis is further developed. These new safe DNA editors may be combined with further improvements in multiplex delivery of gRNAs to usher in a new phase of synthetic biology where it is possible to imagine recoding whole mammalian genomes. When combined with further modulation of DNA repair and pro-survival factors there may be no practical limit to the number of bases that can be altered in a single round of editing, opening up new possibilities that were previously not thought possible. The toxicity limitation that prevented large-scale genome editing in human iPSCs has been overcome by expanding its boundary by three orders of magnitude. The continued development of multiplex delivery along with non-toxic, high-efficiency DNA editors without DSBs or SSBs is paramount to the success of genome-wide recoding efforts to probe the inner workings of life itself, ultimately leading to the radical redesign of nature and ourselves.

### REFERENCES

1. Boeke, J. D. et al. The Genome Project-Write. Science 353, 126-127 (2016).
2. Ruella, M. & Kenderian, S. S. Next Generation Chimeric Antigen Receptor T Cell Therapy: Going off the Shelf. BioDrugs 31, 473-481 (2017).
3. Yang, L. et al. Genome-wide inactivation of porcine endogenous retroviruses (PERVs). Science 350, 1101-1104 (2015).
4. Kazazian, H. H. & Moran, J. V. Mobile DNA in Health and Disease. N. Engl. J. Med. 377, 361-370 (2017).
5. Chenais, B. Transposable Elements in Cancer and Other Human Diseases. (2015). Available at:
   https://www.ingentaconnect.com/content/ben/ccdt/2015/00000015/00000003/art00010. (Accessed: 14 Jan. 2019)
6. Lajoie, M. J. et al. Genomically recoded organisms expand biological functions. Science 342, 357-360 (2013).
7. Ostrov, N. et al. Design, synthesis, and testing toward a 57-codon genome. Science 353, 819-822 (2016).
8. Sun, J., Chen, M., Xu, J. & Luo, J. Relationships among stop codon usage bias, its context, isochores, and gene expression level in various eukaryotes. J. Mol. Evol. 61, 437-444 (2005).
9. Jinek, M. et al. A programmable dual-RNA-guided DNA endonuclease in adaptive bacterial immunity. Science 337, 816-821 (2012).
10. Mali, P. et al. RNA-guided human genome engineering via Cas9. Science 339, 823-826 (2013).
11. Cong, L. et al. Multiplex genome engineering using CRISPR/Cas systems. Science 339, 819-823 (2013).
12. Shipman, S. L., Nivala, J., Macklis, J. D. & Church, G. M. Molecular recordings by directed CRISPR spacer acquisition. Science 353, aaf1175 (2016).
13. Waltz, E. Gene-edited CRISPR mushroom escapes US regulation. Nature News 532, 293 (2016).
14. Boyiadzis, M. M. et al. Chimeric antigen receptor (CAR) T therapies for the treatment of hematologic malignancies: clinical perspective and significance. J Immunother Cancer 6, (2018).
15. Wang, H. et al. One-step generation of mice carrying mutations in multiple genes by CRISPR/Cas-mediated genome engineering. Cell 153, 910-918 (2013).
16. Niu, D. et al. Inactivation of porcine endogenous retrovirus in pigs using CRISPR-Cas9. Science (2017). doi:10.1126/science.aan4187
17. Wang, J. et al. Inhibition of activated pericentromeric SINE/Alu repeat transcription in senescent human adult stem cells reinstates self-renewal. Cell Cycle 10, 3016-3030 (2011).
18. Coufal, N. G. et al. L1 retrotransposition in human neural progenitor cells. Nature 460, 1127-1131 (2009).
19. Coufal, N. G. et al. Ataxia telangiectasia mutated (ATM) modulates long interspersed element-1 (L1) retrotransposition in human neural stem cells. Proc. Natl. Acad. Sci. U.S.A. 108, 20382-20387 (2011).
20. Kazazian, H. H. et al. Haemophilia A resulting from de novo insertion of L1 sequences represents a novel mechanism for mutation in man. Nature 332, 164-166 (1988).
21. Male, P. et al. Rescuing the negative impact of human endogenous retrovirus envelope protein on oligodendroglial differentiation and myelination. Glia (2018). doi:10.1002/glia.23535
22. Burns, K. H. & Boeke, J. D. Human transposon tectonics. Cell 149, 740-752 (2012).
23. Ostertag, E. M. et al. A mouse model of human L1 retrotransposition. Nat. Genet. 32, 655-660 (2002).
24. Bodea, G. O., McKelvey, E. G. Z. & Faulkner, G. J. Retrotransposon-induced mosaicism in the neural genome. Open Biol 8, (2018).
25. Muotri, A. R. et al. Somatic mosaicism in neuronal precursor cells mediated by L1 retrotransposition. Nature 435, 903-910 (2005).
26. Muotri, A. R. et al. L1 retrotransposition in neurons is modulated by MeCP2. Nature 468, 443-446 (2010).
27. Kuscu, C. et al. CRISPR-STOP: gene silencing through base-editing-induced nonsense mutations. Nature Methods (2017). doi:10.1038/nmeth.4327
28. Thompson, D. B. et al. The Future of Multiplexed Eukaryotic Genome Engineering. ACS Chem. Biol. 13, 313-325 (2018).
29. Aguirre, A. J. et al. Genomic Copy Number Dictates a Gene-Independent Cell Response to CRISPR/Cas9 Targeting. Cancer Discov 6, 914-929 (2016).
30. Komor, A. C., Kim, Y. B., Packer, M. S., Zuris, J. A. & Liu, D. R. Programmable editing of a target base in genomic DNA without double-stranded DNA cleavage. Nature 533, 420-424 (2016).
31. Gaudelli, N. M. et al. Programmable base editing of A•T to G•C in genomic DNA without DNA cleavage. Nature 551, 464-471 (2017).
32. Zhou, C. et al. Highly efficient base editing in human tripronuclear zygotes. Protein Cell 8, 772-775 (2017).
33. Komor, A. C. et al. Improved base excision repair inhibition and bacteriophage Mu Gam protein yields C:G-to-T:A fusion proteins with higher efficiency and product purity. Sci Adv 3, (2017).
34. Tarailo-Graovac, M. & Chen, N. Using RepeatMasker to identify repetitive elements in genomic sequences. Curr Protoc Bioinformatics Chapter 4, Unit 4.10 (2009).
35. Riesenberg, S., Maricic, T. & Pääbo, S. ‘Ancestralization’ of human pluripotent stem cells by multiplexed precise genome editing. bioRxiv 342311 (2018). doi:10.1101/342311
36. Baylis, F. & McLeod, M. First-in-human Phase 1 CRISPR Gene Editing Cancer Trials: Are We Ready? Curr Gene Ther 17, 309-319 (2017).
37. Liang, P. et al. CRISPR/Cas9-mediated gene editing in human tripronuclear zygotes. Protein Cell 6, 363-372 (2015).
38. Kang, X. et al. Introducing precise genetic modifications into human 3PN embryos by CRISPR/Cas-mediated genome editing. J Assist Reprod Genet 33, 581-588 (2016).
39. Tang, L. et al. CRISPR/Cas9-mediated gene editing in human zygotes using Cas9 protein. Mol. Genet. Genomics 292, 525-533 (2017).
40. Ma, H. et al. Correction of a pathogenic gene mutation in human embryos. Nature advance online publication, (2017).
41. Zeng, Y. et al. Correction of the Marfan Syndrome Pathogenic FBN1 Mutation by Base Editing in Human Cells and Heterozygous Embryos. Molecular Therapy 26, 2631-2637 (2018).
42. Wang, B. et al. Highly efficient CRISPR/HDR-mediated knock-in for mouse embryonic stem cells and zygotes. BioTechniques 59, 201-202,204, 206-208 (2015).
43. Ihry, R. J. et al. p53 inhibits CRISPR-Cas9 engineering in human pluripotent stem cells. Nature Medicine 24, 939 (2018).
44. Penzkofer, T. et al. L1Base 2: more retrotransposition-active LINE-1s, more mammalian genomes. Nucleic Acids Res 45, D68-D73 (2017).
45. Grandi, N., Cadeddu, M., Blomberg, J. & Tramontano, E. Contribution of type W human endogenous retroviruses to the human genome: characterization of HERV-W proviral insertions and processed pseudogenes. Retrovirology 13,67 (2016).
46. Chavez, A. et al. Highly efficient Cas9-mediated transcriptional programming. Nat Meth 12, 326-328 (2015).
47. Kleinstiver, B. P. et al. Broadening the targeting range of Staphylococcus aureus CRISPR-Cas9 by modifying PAM recognition. Nat. Biotechnol. 33, 1293-1298 (2015).
48. Byrne, S. M. & Church, G. M. Crispr-mediated Gene Targeting of Human Induced Pluripotent Stem Cells. Curr Protoc Stem Cell Biol 35, 5A.8.1-22 (2015).
49. Langmead, B. & Salzberg, S. L. Fast gapped-read alignment with Bowtie 2. Nature Methods 9, 357-359 (2012).
50. Li, H. et al. The Sequence Alignment/Map format and SAMtools. Bioinformatics 25, 2078-2079 (2009).
51. Chadwick, A. C., Wang, X. & Musunuru, K. In Vivo Base Editing of PCSK9 (Proprotein Convertase Subtilisin/Kexin Type 9) as a Therapeutic Alternative to Genome Editing. Arterioscler. Thromb. Vasc. Biol. 37, 1741-1747 (2017).
52. Wang G, Zhao N, Berkhout B, Das A T. CRISPR-Cas9 Can Inhibit HIV-1 Replication but NHEJ Repair Facilitates Virus Escape. Mol. Ther. 2016; 24:522-526.
53. Sakuma T, Masaki K, Abe-Chayama H, Mochida K, Yamamoto T, Chayama K.

Highly multiplexed CRISPR-Cas9-nuclease and Cas9-nickase vectors for inactivation of hepatitis B virus. Genes Cells. 2016; 21:1253-1262.
54. Zuo, E., Sun, Y., Wei, W., Yuan, T., Ying, W., Sun, H., Yuan, L., Steinmetz, L. M., Li, Y. and Yang, H. (2019) Cytosine base editor generates substantial off-target single-nucleotide variants in mouse embryos. Science, 10.1126/science.aav9973.
55. Jin, S., Zong, Y., Gao, Q., Zhu, Z., Wang, Y., Qin, P., Liang, C., Wang, D., Qiu, J.-L., Zhang, F., et al. (2019) Cytosine, but not adenine, base editors induce genome-wide off-target mutations in rice. Science, 364, 292-295.
56. Zhou, C., Sun, Y., Yan, R., Liu, Y., Zuo, E., Gu, C., Han, L., Wei, Y., Hu, X., Zeng, R., et al. (2019) Off-target RNA mutation induced by DNA base editing and its elimination by mutagenesis. Nature, 571, 275-278.
57. Grunewald, J., Zhou, R., Garcia, S. P., Iyer, S., Lareau, C. A., Aryee, M. J. and Joung, J. K. (2019) Transcriptome-wide off-target RNA editing induced by CRISPR-guided DNA base editors. Nature, 569, 433.

### EQUIVALENTS AND SCOPE

In the claims articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The disclosure includes embodiments in which exactly one member of the group is present in, employed in, or otherwise relevant to a given product or process. The disclosure includes embodiments in which more than one, or all of the group members are present in, employed in, or otherwise relevant to a given product or process.

Furthermore, the disclosure encompasses all variations, combinations, and permutations in which one or more limitations, elements, clauses, and descriptive terms from one or more of the listed claims is introduced into another claim. For example, any claim that is dependent on another claim can be modified to include one or more limitations found in any other claim that is dependent on the same base claim. Where elements are presented as lists, e.g. in Markush group format, each subgroup of the elements is also disclosed, and any element(s) can be removed from the group. It should it be understood that, in general, where the disclosure, or aspects described herein, is/are referred to as comprising particular elements and/or features, certain embodiments described herein or aspects described herein consist, or consist essentially of, such elements and/or features. For purposes of simplicity, those embodiments have not been specifically set forth in haec verba herein. It is also noted that the terms “comprising” and “containing” are intended to be open and permits the inclusion of additional elements or steps. Where ranges are given, endpoints are included. Furthermore, unless otherwise indicated or otherwise evident from the context and understanding of one of ordinary skill in the art, values that are expressed as ranges can assume any specific value or sub-range within the stated ranges in different embodiments described herein, to the tenth of the unit of the lower limit of the range, unless the context clearly dictates otherwise.

This application refers to various issued patents, published patent applications, journal articles, and other publications, all of which are incorporated herein by reference. If there is a conflict between any of the incorporated references and the instant specification, the specification shall control. In addition, any particular embodiment of the present disclosure that falls within the prior art may be explicitly excluded from any one or more of the claims. Because such embodiments are deemed to be known to one of ordinary skill in the art, they may be excluded even if the exclusion is not set forth explicitly herein. Any particular embodiment described herein can be excluded from any claim, for any reason, whether or not related to the existence of prior art.

Those skilled in the art will recognize or be able to ascertain using no more than routine experimentation many equivalents to the specific embodiments described herein. The scope of the present embodiments described herein is not intended to be limited to the above Description, but rather is as set forth in the appended claims. Those of ordinary skill in the art will appreciate that various changes and modifications to this description may be made without departing from the spirit or scope of the present disclosure, as defined in the following claims.

What is claimed is:

1. A method of base editing comprising:
contacting a nucleic acid molecule with a plurality of fusion proteins, wherein each of the fusion proteins of the plurality comprises (i) a nuclease inactive Cas9 (dCas9) domain and (ii) a deaminase domain, and a guide RNA (gRNA) bound to the dCas9 domain,
wherein at least five of the fusion proteins of the plurality are each bound to a unique gRNA comprising a different guide sequence of at least 10 contiguous nucleotides that is complementary to a target sequence in the genomic DNA of a eukaryotic cell.

2. The method of claim 1, wherein at least 10, 15, 20, 25, 30, 35, 40, 45, or 50 of the fusion proteins of the plurality are each bound to a unique gRNA comprising a different guide sequence of at least 10 contiguous nucleotides that is complementary to a target sequence.

3. The method of claim 1 or 2, wherein each of the fusion proteins of the plurality comprises an amino acid sequence of SEQ ID NO: 3 or SEQ ID NO: 4.

4. The method of any one of claims 1-3, wherein each of the fusion proteins of the plurality is the same.

5. The method of any one of claims 1-4, wherein the nuclease inactive Cas9 (dCas9) domain comprises a D10A and an H840A mutation in the amino acid sequence provided in SEQ ID NO: 20, or corresponding mutations in the amino acid sequence provided in SEQ ID NO: 102.

6. The method of any one of claims 1-5, wherein the nuclease inactive Cas9 (dCas9) domain comprises an amino acid sequence that is at least 80%, 85%, 90%, 95%, 98%, or 99% identical to any one of SEQ ID NOs: 18 or 100.

7. The method of any one of claims 1-6, wherein the nuclease inactive Cas9 (dCas9) comprises the amino acid sequence of any one of SEQ ID NOs: 18 or 100.

8. The method of claim any one of claims 1-7, wherein each of the fusion proteins of the plurality comprises an amino acid sequence selected from SEQ ID NOs: 6, 7, 8, 9, 10, 11, 12, 13, 14, or 15.

9. The method of claim 1, wherein the deaminase domain is a cytidine deaminase.

10. The method of claim 9, wherein the deaminase domain is an apolipoprotein B mRNA-editing complex 1 (APOBEC1) deaminase domain.

11. The method of claim 1, wherein the deaminase domain is an adenosine deaminase.

12. The method of any one of claims 1-11, wherein the fusion protein comprises the structure NH₂-[dCas9]-[deaminase domain]-COOH, NH₂-[deaminase domain]-[dCas9]-COOH, NH₂-[dCas9]-[deaminase domain]-[uracil glycosylase inhibitor]-COOH, or NH₂-[deaminase domain]-[dCas9]-[uracil glycosylase inhibitor]-COOH; wherein each instance of “]-[” comprises an optional linker.

13. The method of any one of claims 1-12, wherein the deaminase domain of (ii) and the dCas9 domain of (i) are linked via a peptide linker comprising the amino acid sequence of any one of SGSETPGTSESATPES (SEQ ID NO: 27) or SGGSSGGSSGSETPGTSESATPESSGGSSGGS (SEQ ID NO: 28).

14. The method of any one of claims 1-13, wherein the fusion protein further comprises one or more nuclear localization sequences (NLS).

15. A method of base editing comprising:
contacting a nucleic acid molecule with a plurality of fusion proteins, wherein each of the fusion proteins of the plurality comprises (i) a transcription activator-like (TAL) effector domain, (ii) a deaminase domain, and (iii) a cofactor protein associated with the TAL effector domain,
wherein at least five of the fusion proteins of the plurality are each bound to a unique cofactor protein that binds to a target sequence in the genomic DNA of a eukaryotic cell.

16. A method of base editing comprising:
contacting a nucleic acid molecule with a fusion protein comprising (i) a nuclease inactive Cas9 (dCas9) domain and (ii) a deaminase domain, and a guide RNA (gRNA) bound to the dCas9 domain,
wherein the guide RNA comprises a sequence of at least 10 contiguous nucleotides that is complementary to a target sequence, and wherein at least 25 copies of the target sequence are present in the genomic DNA of a eukaryotic cell.

17. The method of claim 16, wherein the target sequence is a repetitive element.

18. The method of claim 16 or claim 17, wherein the gRNA is a single-guide RNA (sgRNA).

19. The method of claim 18, wherein the sgRNA is a promiscuous gRNA.

20. The method of any one of claims 1-15, wherein at least ten of the fusion proteins of the plurality are each bound to a unique gRNA comprising a different guide sequence of at least 10 contiguous nucleotides that is complementary to a target sequence in the genomic DNA of a eukaryotic cell.

21. The method of any one of claims 1-15, wherein at least twenty of the fusion proteins of the plurality are each bound to a unique gRNA comprising a different guide sequence of at least 10 contiguous nucleotides that is complementary to a target sequence in the genomic DNA of a eukaryotic cell.

22. The method of any one of claims 1-21, wherein the step of contacting comprises editing more than 50, more than 100, more than 200, more than 500, more than 1,000, more than 2,000, more than 3,000, more than 5,000, more than 10,000, or more than 20,000 target sequences in the genomic DNA of the eukaryotic cell.

23. The method of any one of claims 1-22, wherein the target sequence comprises a transposable element.

24. The method of claim 23, wherein the target sequence comprises an Alu sequence.

25. The method of claim 23, wherein the target sequence comprises a Long Interspersed Human Elements-1 (LINE-1) sequence.

26. The method of claim 23, wherein the target sequence comprises a Human Endogenous Retrovirus-W (HERV-W) sequence or a Human Endogenous Retrovirus-K (HERV-K) sequence.

27. The method of any one of claims 1-26, wherein the eukaryotic cell is a vertebrate cell.

28. The method of claim 27, wherein the vertebrate cell is a mammalian cell.

29. The method of claim 28, wherein the mammalian cell is a human cell.

30. The method of claim 29, wherein the human cell is a human iPS or ES cell.

31. The method of any one of claims 27-30, wherein the cell is mismatch repair-deficient.

32. The method of any one of claims 1-31, wherein the step of contacting comprises effecting a C to U or a C to T point mutation.

33. The method of any one of claims 1-32, wherein the step of contacting comprises effecting an A to G point mutation.

34. The method of any one of claims 1-33, wherein the step of contacting results in the replacement of a codon encoded by the target sequence with a different codon.

35. The method of claim 34, wherein the step of contacting results in the generation of a plurality of STOP codons.

36. The method of any one of claims 1-35, wherein the step of contacting results in less than 20% indel formation upon base editing.

37. The method of any one of claims 1-36, wherein the step of contacting results in less than 15%, 10%, or 5% indel formation.

38. The method of any one of claims 1-37, wherein the step of contacting results in at least 2:1 intended to unintended product.

39. The method of any one of claims 1-38, wherein the step of contacting results in a base editing efficiency of at least 35%, 40%, 50%, 60%, 70%, 80%, 85%, 90%, 95%, 98%, or 99%.

40. The method of any one of claims 1-39, wherein the step of contacting results in low toxicity when administered to a population of cells.

41. The method of claim 40, wherein the step of contacting results in less than 30%, less than 20%, less than 15%, less than 10%, less than 5% or less than 1% cell death in the population of cells.

42. The method of any one of claims 1-41, wherein the step of contacting results in a low level of DNA damage when administered to a population of cells.

43. The method of claims 40-42, wherein at least 60%, at least 65%, at least 70%, at least 75%, at least 80%, at least 85%, at least 90%, at least 95%, or at least 99% of the cells are viable 24 hours after the step of contacting.

44. The method of any one of claims 1-43, wherein the step of contacting is performed in vitro.

45. The method of any one of claims 1-43, wherein the step of contacting is performed in vivo.

46. The method of any one of claims 1-45, wherein the step of contacting is performed in the absence of cleavage or nicking of the nucleic acid molecule.

47. The method of claim 1-46, wherein the ratio of unique gRNAs to unique target sequences is 1:1.

48. The method of any one of claims 1-47, wherein the gRNA is administered to the cell in a single batch.

49. The method of any one of claims 1-47, wherein the gRNA is administered to the cell in multiple iterations.

50. The method of any of claims 1-49, further comprising contacting the nucleic acid molecule with an isolated inhibitor of base excision repair (BER).

51. The method of claim 50, wherein the inhibitor of BER is a UGI.

52. The method of any of claims 1-51 further comprising contacting the eukaryotic cell with an anti-apoptotic molecule.

53. The method of claim 52, wherein the anti-apoptotic molecule is a pifithrin-α (PFA) or a pifithrin-μ (PFμ).

54. The method of any of claims 1-53 further comprising contacting the eukaryotic cell with a growth factor.

55. The method of claim 54, wherein the growth factor is basic fibroblast growth factor (bFGF).

56. The method of any of claims 1-55 further comprising contacting the eukaryotic cell with an inhibitor of mismatch repair (MMR).

57. The method of claim 56, wherein the inhibitor of MMR is cadmium chloride.

58. The method of any of claims 1-57 further comprising contacting the eukaryotic cell with an inhibitor of non-homologous end joining (NHEJ).

59. The method of any of claims 1-58 further comprising conditionally knocking out a gene in the cell encoding a protein involved in NHEJ or MMR.

60. The method of claim 59, wherein the gene encodes the MutSα complex.

61. The method of claim 59, wherein the gene encodes the MutLα complex.

62. A method of base editing comprising:
contacting a nucleic acid molecule with a plurality of fusion proteins, wherein each of the fusion proteins of the plurality consists essentially of (i) a nuclease inactive Cas9 (dCas9) domain and (ii) a deaminase domain, and a guide RNA (gRNA) bound to the dCas9 domain,
wherein at least five of the fusion proteins of the plurality are each bound to a unique gRNA comprising a different guide sequence of at least 10 contiguous nucleotides that is complementary to a target sequence in the genomic DNA of a eukaryotic cell.

63. A method of base editing comprising:
contacting a nucleic acid molecule with a fusion protein consisting essentially of (i) a nuclease inactive Cas9 (dCas9) domain and (ii) a deaminase domain, and a guide RNA (gRNA) bound to the dCas9 domain,
wherein the guide RNA comprises a sequence of at least 10 contiguous nucleotides that is complementary to a target sequence, and wherein at least 25 copies of the target sequence are present in the genomic DNA of a eukaryotic cell.

64. A method of base editing comprising:
contacting a nucleic acid molecule with a plurality of fusion proteins, wherein each of the fusion proteins of the plurality consists essentially of (i) a transcription activator-like (TAL) effector domain, (ii) a deaminase domain, and (iii) a cofactor protein associated with the TAL effector domain,
wherein at least five of the fusion proteins of the plurality are each bound to a unique cofactor protein that binds to a target sequence in the genomic DNA of a eukaryotic cell.

65. A composition of eukaryotic cells comprising a plurality of fusion proteins, wherein each of the fusion proteins of the plurality comprises (i) a nuclease inactive Cas9 (dCas9) domain and (ii) a deaminase domain, and a guide RNA (gRNA) bound to the dCas9 domain, wherein at least five of the fusion proteins of the plurality are each bound to a unique gRNA comprising a different guide sequence of at least 10 contiguous nucleotides that is complementary to a target sequence in the genomic DNA of the cells.

66. The composition of claim 65 further comprising an anti-apoptotic molecule and a growth factor.

67. The composition of claim 66, wherein the anti-apoptotic molecule is PFA, and the growth factor is bFGF.

68. The composition of any one of claims 65-67 further comprising an inhibitor of MMR.

69. A pharmaceutical composition comprising a plurality of fusion proteins, wherein each of the fusion proteins of the plurality comprises (i) a nuclease inactive Cas9 (dCas9) domain and (ii) a deaminase domain, and a guide RNA (gRNA) bound to the dCas9 domain, wherein at least five of the fusion proteins of the plurality are each bound to a unique gRNA comprising a different guide sequence of at least 10 contiguous nucleotides that is complementary to a target sequence in the genomic DNA of a eukaryotic cell, and a pharmaceutically acceptable excipient.

70. A pharmaceutical composition comprising a plurality of fusion proteins, wherein each of the fusion proteins of the plurality comprises (i) a TAL effector domain, (ii) a deaminase domain, and (iii) a cofactor protein associated with the TAL effector domain, wherein at least five of the fusion proteins of the plurality are each bound to a unique cofactor protein that binds to a target sequence in the genomic DNA of a eukaryotic cell, and a pharmaceutically acceptable excipient.

71. The pharmaceutical composition of claim 69 or claim 70 further comprising an anti-apoptotic molecule and a growth factor.

72. The pharmaceutical composition of claim 71, wherein the anti-apoptotic molecule is PFA and the growth factor is bFGF.

73. The pharmaceutical composition of any one of claims 69-72 further comprising an isolated inhibitor of base excision repair (BER).

74. The pharmaceutical composition of any one of claims 69-73 further comprising an inhibitor of MMR.

75. The pharmaceutical composition of claim 74, wherein the inhibitor of MMR is cadmium chloride.

76. The pharmaceutical composition of any one of claims 69-75 further comprising an inhibitor of non-homologous end joining (NHEJ).

77. The pharmaceutical composition of any one of claims 69-76, wherein administration of the composition to a population of cells results in low toxicity.

78. The pharmaceutical composition of claim 77, wherein at least 60%, at least 65%, at least 70%, at least 75%, at least 80%, at least 85%, at least 90%, at least 95%, or at least 99% of the cells are viable 24 hours after administration.

79. The pharmaceutical composition of claim 77 or claim 78, wherein at least 60%, at least 65%, at least 70%, at least 75%, at least 80%, at least 85%, at least 90%, at least 95%, or at least 99% of the cells are viable 72 hours after administration.

80. The pharmaceutical composition of any one of claims 69-79, wherein administration of the composition to a population of cells results in less than 30%, less than 20%, less than 15%, less than 10%, less than 5% or less than 1% cell death in the population of cells.

81. The pharmaceutical composition of any one of claims 69-80, wherein administration of the composition to a population of cells results in a low level of DNA damage.

82. A kit comprising a nucleic acid construct, comprising
(a) a nucleic acid sequence encoding a plurality of fusion proteins, wherein each of the fusion proteins of the plurality comprises (i) a nuclease inactive Cas9 (dCas9) domain and (ii) a deaminase domain, and nucleic acid sequence encoding a guide RNA (gRNA);
(b) a heterologous promoter that drives expression of the sequence of (a); and
(c) an expression construct encoding a plurality of unique guide RNA backbones, wherein the construct comprises a cloning site positioned to allow the cloning of a nucleic acid sequence identical or complementary to a target sequence into each of the guide RNA backbones.

83. A kit comprising a nucleic acid construct, comprising
(a) a nucleic acid sequence encoding a plurality of fusion proteins, wherein each of the fusion proteins of the plurality comprises (i) a nuclease inactive Cas9 (dCas9) domain and (ii) a deaminase domain,
(b) a nucleic acid sequence encoding a guide RNA (gRNA);
(c) a heterologous promoter that drives expression of the sequence of (a);
(d) a heterologous promoter that drives expression of the sequence of (b); and
(e) an expression construct encoding a plurality of unique guide RNA backbones, wherein the construct comprises a cloning site positioned to allow the cloning of a nucleic acid sequence identical or complementary to a target sequence into each of the guide RNA backbones.

84. A kit comprising a nucleic acid construct, comprising
(a) a nucleic acid sequence encoding a plurality of fusion proteins, wherein each of the fusion proteins of the plurality comprises (i) a TAL effector domain, (ii) a deaminase domain, and (iii) a cofactor protein associated with the TAL effector domain,
(b) a heterologous promoter that drives expression of the sequence of (a); and
(c) an expression construct encoding a plurality of unique cofactor proteins.
